SEQUENCE LISTING (236 sequences)

SEQ ID NO: 1 - protein, 213 aa, Artificial Sequence
  Clone #11074 Full
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - DNA, 639 bp, Artificial Sequence
  Clone #11074 Full
gatattcagatgacccagtctcccagcacactgtccgcctctgtgggcgaccgggtgaccatcacatgcaagtgtcagctgagcgtgggctacatgcactggtatcagcagaagcccggcaaggcccctaagctgctgatctacgataccagcaagctggcctccggcgtgccatctagattcagcggctccggctctggcaccgagtttaccctgacaatcagctccctgcagcccgacgatttcgccacatactattgctttcaggggagcggctacccattcacattcggagggggaactaaactggaaatcaagaggaccgtcgcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 3 - protein, 106 aa, Artificial Sequence
  Clone #11074 VL
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIK

SEQ ID NO: 4 - protein, 449 aa, Artificial Sequence
  Clone #11011 Full
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 5 - DNA, 1347 bp, Artificial Sequence
  Clone # 11011 Full
caggtgacactgagggagagcggaccagccctggtgaagccaacccagacactgaccctgacatgcaccttctccggctttagcctgtccacatctggcatgtctgtgggctggatcagacagccacctggcaaggccctggagtggctggccgacatctggtgggacgataagaaggattacaaccctagcctgaagtccagactgacaatctctaaggacaccagcaagaaccaggtggtgctgaaggtgaccaatatggaccccgccgatacagccacctactattgtgcccggtccatgattactaactggtattttgatgtctggggggcaggaacaaccgtgaccgtctcttctgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 6 - protein, 120 aa, Artificial Sequence
  Clone #11011 VH
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 7 - protein, 450 aa, Artificial Sequence
  Clone #12644 Full
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 8 - DNA, 1350 bp, Artificial Sequence
  Clone #12644 Full
caggtgcagctgcagcagagcggagccgagctggccaggccaggggccagcgtgaagatgagctgcaaggcctccggctacaccttcaccacatatacaatgcactgggtgaagcagcggcccggacagggcctggagtggatcggctacatcaaccctagctccggctacaccaactataatcagaagtttaaggacaaggccaccctgacagccgataagtctagctccaccgcctctatgcagctgtctagcctgacaagcgaggactccgccgtgtactattgtgcccgggagagagccgtgctggtgccatacgccatggattattggggccagggcacctccgtgacagtgtcctctgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 9 - protein, 121 aa, Artificial Sequence
  Clone #12644 VH
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSS

SEQ ID NO: 10 - protein, 213 aa, Artificial Sequence
  Clone #12645 Full
QIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 639 bp, Artificial Sequence
  Clone #12645 Full
cagatcgtgctgacccagtccccagccgtgatgagcgcctccccaggagagaaggtgaccatcacatgcaccgccagctcctctctgagctacatgcactggttccagcagaagcccggcacatcccctaagctgtggctgtattctaccagcatcctggcctctggcgtgcctacaaggttttccggctctggcagcggcacatcctactctctgaccatcagccggatggaggcagaggacgcagcaacctactattgtcagcagagaagctcctctcccttcacatttggcagcggcaccaagctggagatcaagcggacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 12 - protein, 106 aa, Artificial Sequence
  Clone #12645 VL
QIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIK

SEQ ID NO: 13 - protein, 448 aa, Artificial Sequence
  Clone #12646 Full
EVQLQQSGPELEKPGASVKISCKASGYSFTGYNMNWVKQSNGKSLEWIGNIDPYYGDTNYNQKFKGKATLTVDKSSSTAYMHLKSLTSEDSAVYYCARPYGSEAYFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 14 - DNA, 1344 bp, Artificial Sequence
  CLone #12646 Full
gaggtgcagctgcagcagtctggaccagagctggagaagcctggggccagcgtgaagatcagctgcaaggccagcggctactccttcaccggctataacatgaattgggtgaagcagtccaacggcaagtctctggagtggatcggcaatatcgacccatactatggcgatacaaactacaatcagaagtttaagggcaaggccaccctgacagtggacaagagctcctctaccgcctatatgcacctgaagtctctgacaagcgaggattccgccgtgtactattgtgccagaccctacggcagcgaggcctacttcgcctattggggccagggcaccctggtgacagtgtccgccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 15 - protein, 119 aa, Artificial Sequence
  Clone #12646 VH
EVQLQQSGPELEKPGASVKISCKASGYSFTGYNMNWVKQSNGKSLEWIGNIDPYYGDTNYNQKFKGKATLTVDKSSSTAYMHLKSLTSEDSAVYYCARPYGSEAYFAYWGQGTLVTVSA

SEQ ID NO: 16 - protein, 214 aa, Artificial Sequence
  Clone #12647 Full
DIVMTQSPATLSVTPGDRVSLSCRASQSISDYLHWYQQKSHESPRLLIKYAAQSISGIPSRFSGSGSGSDFTLSINGVEPEDVGVYYCQNGHSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 642 bp, Artificial Sequence
  Clone #12647 Full
gacatcgtgatgacccagtcccccgccaccctgtctgtgacacctggcgaccgggtgagcctgtcctgcagagcctctcagagcatctccgattacctgcactggtatcagcagaagtctcacgagagcccaaggctgctgatcaagtacgccgcccagtctatcagcggcatccccagccgcttctccggctctggcagcggctccgactttaccctgtccatcaacggcgtggagcctgaggatgtgggcgtgtactattgtcagaatggccactctttcccctatacctttggcggcggcacaaagctggagatcaagcggacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 18 - protein, 107 aa, Artificial Sequence
  Clone #12647 VL
DIVMTQSPATLSVTPGDRVSLSCRASQSISDYLHWYQQKSHESPRLLIKYAAQSISGIPSRFSGSGSGSDFTLSINGVEPEDVGVYYCQNGHSFPYTFGGGTKLEIK

SEQ ID NO: 19 - protein, 447 aa, Artificial Sequence
  Clone #12648 Full
QVQLKESGPGLVAPSQSLSITCSVSGFSLSNYDISWIRQPPGKGLEWLGVMWTGGGANYNSAFMSRLSINKDNSKSQVFLKMNNLQTDDTAIYYCVRDAVRYWNFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 20 - DNA, 1341 bp, Artificial Sequence
  Clone #12648 Full
caggtgcagctgaaggagtccggaccaggcctggtggccccctctcagagcctgtccatcacctgctctgtgagcggcttctccctgtctaactacgacatctcctggatcaggcagccacctggcaagggcctggagtggctgggcgtgatgtggacaggaggaggagccaactataattctgccttcatgtctcggctgagcatcaacaaggataatagcaagtcccaggtgtttctgaagatgaacaatctgcagaccgacgatacagccatctactattgcgtgcgggacgccgtgagatactggaattttgacgtgtggggggcagggaccacagtgaccgtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 21 - protein, 118 aa, Artificial Sequence
  Clone #12648 VH
QVQLKESGPGLVAPSQSLSITCSVSGFSLSNYDISWIRQPPGKGLEWLGVMWTGGGANYNSAFMSRLSINKDNSKSQVFLKMNNLQTDDTAIYYCVRDAVRYWNFDVWGAGTTVTVSS

SEQ ID NO: 22 - protein, 213 aa, Artificial Sequence
  Clone #12649 Full
QIVLSQSPAILSASPGEKVTMTCRASSSVSYIHWYQQKPGSSPKPWIYATSHLASGVPARFSGSGSGTSYSLTISRVEAEDTATYYCQQWSSNPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 639 bp, Artificial Sequence
  Clone #12649 Full
cagatcgtgctgtcccagtctccagccatcctgagcgcctccccaggagagaaggtgaccatgacatgcagggccagctcctctgtgagctacatccactggtatcagcagaagcctggcagctcccccaagccttggatctacgccacctcccacctggcctctggagtgccagcccggttctctggcagcggctccggcacctcttatagcctgacaatcagcagagtggaggccgaggacaccgccacatactattgtcagcagtggtctagcaaccccttcacctttggctccggcacaaagctggagatcaagcggacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 24 - protein, 106 aa, Artificial Sequence
  Clone #12649 VL
QIVLSQSPAILSASPGEKVTMTCRASSSVSYIHWYQQKPGSSPKPWIYATSHLASGVPARFSGSGSGTSYSLTISRVEAEDTATYYCQQWSSNPFTFGSGTKLEIK

SEQ ID NO: 25 - protein, 477 aa, Artificial Sequence
  Clone #11082 Full
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSVEGGSGGSGGSGGSGGVDDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 26 - DNA, 1431 bp, Artificial Sequence
  Clone #11082 Full
caggtgaccctgagagagagcggacccgccctggtgaagcctacccagacactgaccctgacatgcaccttcagcggctttagcctgtccacctctggcatgtccgtgggatggatcaggcagccacctggcaaggccctggagtggctggccgacatctggtgggacgataagaaggattacaacccttccctgaagtctcgcctgacaatctccaaggacacctctaagaaccaggtggtgctgaaggtgaccaatatggacccagccgatacagccacctactattgtgcccggtccatgatcacaaattggtatttcgacgtgtggggagccggaaccacagtgaccgtgagctccgtggagggaggcagcggaggctccggaggctctggaggcagcggaggagtggacgatatccagatgacacagagcccctccaccctgtctgccagcgtgggcgaccgggtgacaatcacctgcaagtgtcagctgtccgtgggctacatgcactggtatcagcagaagcctggcaaggccccaaagctgctgatctacgataccagcaagctggcctccggcgtgccttctaggttctccggctctggcagcggcacagagtttacactgaccatctctagcctgcagccagacgatttcgccacctactattgctttcagggcagcggctatcccttcacatttggcggcggcaccaagctggagatcaaggccgccgagcctaagtcctctgacaagacacacacctgcccaccctgtccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgaaccacaggtctacgtgctgcctccatcccgggacgagctgacaaagaaccaggtctctctgctgtgcctggtgaaaggcttctatccatcagatattgctgtggagtgggaaagcaatgggcagcccgagaacaattacctgacttggccccctgtgctggactctgatgggagtttctttctgtattctaagctgaccgtggataaaagtaggtggcagcagggaaatgtctttagttgttcagtgatgcatgaagccctgcataaccactacacccagaaaagcctgtccctgtcccccgga

SEQ ID NO: 27 - protein, 120 aa, Artificial Sequence; identical to SEQ ID NO: 6
  Clone #11082 VH

SEQ ID NO: 28 - protein, 214 aa, Artificial Sequence
  Clone #12651 Full
EIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 642 bp, Artificial Sequence
  Clone #12651 Full
gagatcgtgctgacccagtctccagccacactgtccctgtctccaggagagagggccaccctgagctgcagggccagccagtccgtgagctcctacctggcctggtatcagcagaagccaggacaggccccccggctgctgatctacgacgcctccaacagggcaaccggcatccccgcaagattctctggcagcggctccggcacagactttaccctgacaatctctagcctggagcctgaggatttcgccgtgtactattgtcagcagcggagaaattggccactgacctttggcggcggcacaaaggtggagatcaagagaacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 30 - protein, 107 aa, Artificial Sequence
  Clone #12651 VL
EIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIK

SEQ ID NO: 31 - protein, 448 aa, Artificial Sequence
  Clone #12652 Full
EVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 32 - DNA, 1344 bp, Artificial Sequence
  Clone #12652 Full
gaggtgaagctggtggagagcggaggaggcctggtgcagccaggaggctctctgaagctgagctgcgccacctccggcttcacattttccgactactatatgtactgggtgcggcagaccccagagaagaggctggagtgggtggcctatatcaactctggcggcggcagcacctactatcctgacacagtgaagggcaggttcaccatcagccgggacaacgccaagaatacactgtacctgcagatgtcccggctgaagtctgaggacacagccatgtactattgtgcccggagaggcctgccctttcacgccatggattattggggccagggcaccagcgtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 33 - protein, 119 aa, Artificial Sequence
  Clone #12652 VH
EVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSS

SEQ ID NO: 34 - protein, 214 aa, Artificial Sequence
  Clone #12653 Full
DIQMTQTTSSLSASLGDRVTISCSASQGISNYLNWYQQKPDGTVKLLIYYTSILHSGVPSRFSGSGSGTDYSLTIGNLEPEDIATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 642 bp, Artificial Sequence
  Clone #12653 Full
gacatccagatgacccagaccacaagctccctgtctgccagcctgggcgatcgggtgacaatctcctgctctgccagccagggcatctccaactacctgaattggtatcagcagaagccagacggcaccgtgaagctgctgatctactatacatccatcctgcactctggcgtgcccagcagattctccggctctggcagcggcaccgactactctctgacaatcggcaacctggagcccgaggatatcgccacctactattgtcagcagttcaataagctgccccctacctttggcggcggcacaaagctggagatcaagcggacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 36 - protein, 107 aa, Artificial Sequence
  Clone #12653 VL
DIQMTQTTSSLSASLGDRVTISCSASQGISNYLNWYQQKPDGTVKLLIYYTSILHSGVPSRFSGSGSGTDYSLTIGNLEPEDIATYYCQQFNKLPPTFGGGTKLEIK

SEQ ID NO: 37 - protein, 477 aa, Artificial Sequence
  Clone #12654 Full
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPATFGQGTKVEIKVEGGSGGSGGSGGSGGVDEVQLVESGGGLVQPGGSLRLSCAASGFTFADYTMDWVRQAPGKGLEWVGDVNPNSGGSIYNQRFKGRFTFSVDRSKNTLYLQMNSLRAEDTAVYYCARNLGPSFYFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 38 - DNA, 642 bp, Artificial Sequence; identical to SEQ ID NO: 35
  Clone #12654 Full

SEQ ID NO: 39 - protein, 107 aa, Artificial Sequence
  Clone #12654 VL
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPATFGQGTKVEIK

SEQ ID NO: 40 - protein, 483 aa, Artificial Sequence
  Clone #12655 Full
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTVEGGSGGSGGSGGSGGVDQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 41 - DNA, 1449 bp, Artificial Sequence
  Clone #12655 Full
gagctggtgctgacacagtccccttctgtgagcgccgccctgggctccccagccaagatcacctgcacactgagctccgcccacaagaccgacacaatcgattggtaccagcagctgcagggagaggcacccagatatctgatgcaggtgcagtctgacggcagctacaccaagcggcccggagtgcctgacagattctccggctctagctccggagccgatcgctatctgatcatcccatctgtgcaggccgacgatgaggccgactactattgcggagccgattacatcggaggatacgtgttcggaggaggaacccagctgaccgtgacagtggagggaggctccggaggctctggaggcagcggcggctccggcggcgtggaccaggagcagctggtggagagcggcggcagactggtgaccccaggaggctccctgacactgtcttgtaaggccagcggcttcgatttttccgcctactatatgtcttgggtgagacaggcaccaggcaagggcctggagtggatcgccaccatctacccctctagcggcaagacctactatgccacatgggtgaacggcagattcaccatctcctctgacaacgcccagaatacagtggatctgcagatgaatagcctgaccgccgccgacagggccacatacttctgcgcccgcgattcctatgccgacgatggggccctgttcaacatctggggccctggcaccctggtgacaatcagctccgccgccgagccaaagtctagcgacaagacccacacatgcccaccttgtccggcgccagaggccgccggaggaccaagcgtgttcctgtttccacccaagcctaaggataccctgatgatctccagaaccccagaggtgacatgcgtggtggtgtccgtgtctcacgaggaccccgaggtgaagtttaactggtatgtggatggcgtggaggtgcacaatgccaagacaaagcccagagaggagcagtacaatagcacctatagagtggtgtccgtgctgacagtgctgcaccaggactggctgaacggcaaggagtacaagtgcaaggtgtctaataaggccctgcctgccccaatcgagaagaccatcagcaaggcaaagggacagcctcgcgaaccacaggtgtatgtgctgcctccaagccgcgacgagctgacaaagaaccaggtgtccctgctgtgcctggtgaagggcttctacccctccgatatcgccgtggagtgggagtctaatggccagcctgagaacaattatctgacctggccccctgtgctggactctgatggcagcttctttctgtactctaagctgacagtggataagagccggtggcagcagggcaacgtgtttagctgttccgtgatgcacgaggccctgcacaatcactacacccagaagtctctgagcttaagccctggc

SEQ ID NO: 42 - protein, 111 aa, Artificial Sequence
  Clone #12655 VL
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVT

SEQ ID NO: 43 - protein, 121 aa, Artificial Sequence
  Clone #12655 VH
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISS

SEQ ID NO: 44 - protein, 448 aa, Artificial Sequence
  Clone #12657 Full
EVQLVESGGGLVQPGGSLRLSCAASGFTFADYTMDWVRQAPGKGLEWVGDVNPNSGGSIYNQRFKGRFTFSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 45 - DNA, 1344 bp, Artificial Sequence
  Clone #12657 Full
gaggtgcagctggtggaatcaggagggggcctggtgcagcccggagggtctctgcgactgtcatgtgccgcttctgggttcactttcgcagactacacaatggattgggtgcgacaggcccccggaaagggactggagtgggtgggcgatgtcaaccctaattctggcgggagtatctacaaccagcggttcaaggggagattcactttttcagtggacagaagcaaaaacaccctgtatctgcagatgaacagcctgagggccgaagataccgctgtctactattgcgctcgcaatctgggccccagtttctactttgactattgggggcagggaaccctggtgacagtcagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 46 - protein, 119 aa, Artificial Sequence
  Clone #12657 VH
EVQLVESGGGLVQPGGSLRLSCAASGFTFADYTMDWVRQAPGKGLEWVGDVNPNSGGSIYNQRFKGRFTFSVDRSKNTLYLQMNSLRAEDTAVYYCARNLGPSFYFDYWGQGTLVTVSS

SEQ ID NO: 47 - protein, 214 aa, Artificial Sequence
  Clone #12658 Full
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 642 bp, Artificial Sequence
  Clone #12658 Full
gacatccagatgacccagtcccctagctccctgtccgcctctgtgggcgacagggtgaccatcacatgcaaggcctctcaggatgtgagcatcggagtggcatggtaccagcagaagccaggcaaggcccctaagctgctgatctatagcgcctcctaccggtataccggcgtgccctctagattctctggcagcggctccggcacagactttaccctgacaatctctagcctgcagccagaggatttcgccacctactattgtcagcagtactatatctaccccgccacctttggccagggcacaaaggtggagatcaagcggacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 49 - protein, 107 aa, Artificial Sequence; identical to SEQ ID NO: 39
  Clone #12658 VL

SEQ ID NO: 50 - protein, 450 aa, Artificial Sequence
  Clone #12659 Full
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 51 - DNA, 1350 bp, Artificial Sequence
  Clone #12659 Full
caggagcagctggtggagtccggcggcaggctggtgaccccaggaggcagcctgacactgtcctgcaaggcctctggcttcgactttagcgcctactatatgtcctgggtgcgccaggcccccggcaagggcctggagtggatcgccaccatctaccctagctccggcaagacctactatgccacatgggtgaacggcagattcaccatctctagcgacaacgcccagaatacagtggatctgcagatgaacagcctgaccgccgccgacagggcaacatacttctgtgccagagatagctatgccgacgatggggccctgttcaacatctggggaccaggcaccctggtgacaatctcctctgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 52 - protein, 121 aa, Artificial Sequence; identical to SEQ ID NO: 43
  Clone #12659 VH

SEQ ID NO: 53 - protein, 218 aa, Artificial Sequence
  Clone #12660 Full
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - DNA, 654 bp, Artificial Sequence
  Clone #12660 Full
gagctggtgctgacacagtctccaagcgtgtccgccgccctgggcagccccgccaagatcacctgcacactgagctccgcccacaagaccgacacaatcgattggtaccagcagctgcagggagaggccccccggtatctgatgcaggtgcagtctgacggcagctacacaaagcggcccggagtgcctgacagattctccggctctagctccggagccgatcgctatctgatcatcccctctgtgcaggccgacgatgaggccgactactattgtggagccgattacatcggaggatacgtgttcggaggaggaacccagctgaccgtgacacggaccgtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 55 - protein, 111 aa, Artificial Sequence; identical to SEQ ID NO: 42
  Clone #12660 VL

SEQ ID NO: 56 - protein, 629 aa, Artificial Sequence
  Clone #12667 Full
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 57 - DNA, 1887 bp, Artificial Sequence
  Clone #12667 Full
gagcctgccgtgtatttcaaggagcagtttctggacggcgatggctggacaagcagatggatcgagtctaagcacaagagcgacttcggcaagtttgtgctgagctccggcaagttctatggcgatgaggagaaggacaagggcctgcagacctctcaggatgccaggttttacgccctgtccgcctctttcgagcccttcagcaacaagggccagaccctggtggtgcagttcacagtgaagcacgagcagaacatcgactgcggcggcggctatgtgaagctgtttcccaatagcctggatcagaccgacatgcacggcgactccgagtacaacatcatgttcggccctgatatctgcggcccaggcacaaagaaggtgcacgtgatctttaattacaagggcaagaacgtgctgatcaataaggacatcaggtgtaaggacgatgagttcacccacctgtacacactgatcgtgcgccctgacaacacatatgaggtgaagatcgataattcccaggtggagagcggctccctggaggacgattgggattttctgccccctaagaagatcaaggaccccgatgcctccaagcctgaggactgggatgagcgcgccaagatcgacgatccaaccgactctaagcccgaggactgggataagcccgagcacatccccgaccctgatgccaagaagccagaagactgggatgaggagatggatggcgagtgggagccacccgtgatccagaacccagagtacaagggcgagtggaagcccagacagatcgataatcctgactataagggcacctggattcaccctgagatcgataacccagagtactccccagacccctctatctacgcctatgataatttcggcgtgctgggcctggacctgtggcaggtgaagagcggcaccatcttcgacaactttctgatcacaaatgatgaggcctacgccgaggagtttggcaacgagacatggggcgtgacaaaggccgccgagaagcagatgaaggataagcaggacgaggagcagaggctgaaggaagaggaggaggacaagaagcgcaaggaggaggaggaggccgaggataaggaggacgatgaggacaaggatgaggacgaggaggatgaggaggacaaggaggaggatgaggaggaggacgtgccaggacaggccgccgccgagcccaagtctagcgacaagacccacacatgccctccatgtccggcgccggaggccgccggaggacctagcgtgttcctgtttccccctaagccaaaggatacactgatgatctccagaacccctgaggtgacatgcgtggtggtgtctgtgagccacgaggacccagaggtgaagttcaactggtatgtggatggcgtggaggtgcacaatgccaagaccaagccccgggaggagcagtacaatagcacctatagagtggtgtccgtgctgacagtgctgcaccaggactggctgaacggcaaggagtacaagtgcaaggtgtccaataaggccctgccggcacctatcgagaagaccatctctaaggcaaagggacagccacgggagccacaggtgtatgtgctgccaccctctagagacgagctgacaaagaaccaggtgagcctgctgtgcctggtgaagggcttctacccatccgatatcgccgtggagtgggagtctaatggccagcccgagaacaattatctgacctggcctccagtgctggatagcgacggctccttctttctgtactctaagctgacagtggacaagagccggtggcagcagggcaacgtgttttcctgttctgtgatgcacgaggccctgcacaatcactacacccagaagagcctgtccctgtctcctggc

SEQ ID NO: 58 - protein, 396 aa, Artificial Sequence
  Clone #12667 Calreticulin
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQA

SEQ ID NO: 59 - DNA, 1191 bp, Artificial Sequence
  Clone #12667 Calreticulin
ggcgagcctgccgtgtatttcaaggagcagtttctggacggcgatggctggacaagcagatggatcgagtctaagcacaagagcgacttcggcaagtttgtgctgagctccggcaagttctatggcgatgaggagaaggacaagggcctgcagacctctcaggatgccaggttttacgccctgtccgcctctttcgagcccttcagcaacaagggccagaccctggtggtgcagttcacagtgaagcacgagcagaacatcgactgcggcggcggctatgtgaagctgtttcccaatagcctggatcagaccgacatgcacggcgactccgagtacaacatcatgttcggccctgatatctgcggcccaggcacaaagaaggtgcacgtgatctttaattacaagggcaagaacgtgctgatcaataaggacatcaggtgtaaggacgatgagttcacccacctgtacacactgatcgtgcgccctgacaacacatatgaggtgaagatcgataattcccaggtggagagcggctccctggaggacgattgggattttctgccccctaagaagatcaaggaccccgatgcctccaagcctgaggactgggatgagcgcgccaagatcgacgatccaaccgactctaagcccgaggactgggataagcccgagcacatccccgaccctgatgccaagaagccagaagactgggatgaggagatggatggcgagtgggagccacccgtgatccagaacccagagtacaagggcgagtggaagcccagacagatcgataatcctgactataagggcacctggattcaccctgagatcgataacccagagtactccccagacccctctatctacgcctatgataatttcggcgtgctgggcctggacctgtggcaggtgaagagcggcaccatcttcgacaactttctgatcacaaatgatgaggcctacgccgaggagtttggcaacgagacatggggcgtgacaaaggccgccgagaagcagatgaaggataagcaggacgaggagcagaggctgaaggaagaggaggaggacaagaagcgcaaggaggaggaggaggccgaggataaggaggacgatgaggacaaggatgaggacgaggaggatgaggaggacaaggaggaggatgaggaggaggacgtgccaggacaggcc

SEQ ID NO: 60 - protein, 447 aa, Artificial Sequence
  Clone #12650 Full
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 61 - DNA, 1341 bp, Artificial Sequence
  Clone #12650 Full
caggtgcagctggtggagagcggaggaggagtggtgcagcccggcagaagcctgcggctgagctgcgcagcctccggcttcaccttttccaactacggcatgtattgggtgcggcaggcccctggcaagggcctggagtgggtggccgtgatctggtacgacggctccaataagtactatgccgattctgtgaagggcaggttcaccatcagccgggacaacagcaagaatacactgtatctgcagatgaactctctgcgggccgaggatacagccgtgtactattgtgccagggacctgtggggctggtactttgattattggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 62 - protein, 118 aa, Artificial Sequence
  Clone #12650 VH
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSS

SEQ ID NO: 63 - protein, 444 aa, Artificial Sequence
  Clone #12661 Full
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 64 - DNA, 1332 bp, Artificial Sequence
  Clone #12661 Full
gaggtccagctggtccagagcggccccgaggtgaagaagcctggcgctactgtgaagatctcatgcaaaacatccggctacactttcaccgagtacacaatccactgggtgaagcaggcacccggaaaaggcctggaatggatcgggaacattaatcctaacaatggcgggaccacatacaaccagaagttcgaggacaaagccactctgaccgtggacaagtctacagatactgcttatatggagctgagctccctgcggagcgaagataccgccgtctactattgcgccgctggatggaatttcgattattggggacagggcaccctgctgacagtctcaagcgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 65 - protein, 115 aa, Artificial Sequence
  Clone #12661 VH
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 66 - protein, 214 aa, Artificial Sequence
  Clone #12662 Full
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 717 bp, Artificial Sequence
  Clone #12662 Full
atggccgtgatggcaccccggaccctggtgctgctgctgagcggggccctggccctgacccagacatgggccggcgacatccagatgacccagtcccctagctccctgtctacaagcgtgggcgatagggtgaccctgacatgcaaggcctcccaggacgtgggaaccgccgtggattggtaccagcagaagccaggcccctctcctaagctgctgatctattgggcctctacccggcacacaggcatccctagcagattctccggctctggcagcggcacagactttaccctgacaatctctagcctgcagccagaggacttcgccgattactattgccagcagtacaactcctatccactgacctttggccccggcacaaaggtggacatcaagaggaccgtggcggcgcccagcgtgttcatctttcccccttccgatgagcagctgaagtccggcacagcctctgtggtgtgcctgctgaacaatttctacccccgcgaggccaaggtgcagtggaaggtggacaacgccctgcagtccggcaattctcaggagagcgtgaccgagcaggactccaaggattctacatatagcctgtcctctaccctgacactgtctaaggccgattacgagaagcacaaggtgtatgcatgcgaggtgacccaccagggcctgagctcccctgtgacaaagagctttaatcggggcgagtgt

SEQ ID NO: 68 - protein, 107 aa, Artificial Sequence
  Clone #12662 VL
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIK

SEQ ID NO: 69 - protein, 217 aa, Artificial Sequence
  Human IgG1 Fc sequence 231-447 (EU numbering)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 213 aa, Artificial Sequence
  Clone #10565 Full
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 639 bp, Artificial Sequence
  Clone #10565 Full
gacatccagatgacacagagcccaagctccctgtccgcctctgtgggcgatagagtgaccatcacatgcagcgcctctagctccgtgtcctacatgcactggtatcagcagaagtccggcaaggcccccaagctgctgatctacgacaccagcaagctggcctccggagtgccttctaggttcagcggctccggctctggcaccgactttaccctgacaatctctagcctgcagccagaggatttcgccacatactattgtcagcagtggagcaagcaccccctgacctttggccagggcacaaagctggagatcaagcggacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 72 - protein, 214 aa, Artificial Sequence
  Clone #11150 Full
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 642 bp, Artificial Sequence
  Clone #11150 Full
gacatccagatgacacagtccccaagctccctgtccgcctctgtgggcgacagggtgaccatcacatgccgcgcctctcaggatgtgaacaccgccgtggcctggtaccagcagaagccaggcaaggcccccaagctgctgatctacagcgcctccttcctgtattctggcgtgcccagccggttttctggcagcagatccggcaccgacttcaccctgacaatctctagcctgcagcctgaggattttgccacatactattgtcagcagcactataccacaccccctaccttcggccagggcacaaaggtggagatcaagcggacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 74 - protein, 231 aa, Artificial Sequence
  Clone #12153 Full
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 75 - DNA, 693 bp, Artificial Sequence
  Clone #12153 Full
gagccaaagagctccgacaagacccacacatgccccccttgtccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgaaccacaggtctacgtgctgcctccatcccgggacgagctgacaaagaaccaggtctctctgctgtgcctggtgaaaggcttctatccatcagatattgctgtggagtgggaaagcaatgggcagcccgagaacaattacctgacttggccccctgtgctggactctgatgggagtttctttctgtattctaagctgaccgtggataaaagtaggtggcagcagggaaatgtctttagttgttcagtgatgcatgaagccctgcataaccactacacccagaaaagcctgtccctgtcccccgga

SEQ ID NO: 76 - protein, 231 aa, Artificial Sequence
  Clone #12155 Full
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 77 - DNA, 693 bp, Artificial Sequence
  Clone #12155 Full
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 78 - protein, 213 aa, Artificial Sequence; identical to SEQ ID NO: 10
  Clone #12645 Full

SEQ ID NO: 79 - DNA, 639 bp, Artificial Sequence; identical to SEQ ID NO: 11
  Clone #12645 Full

SEQ ID NO: 80 - protein, 214 aa, Artificial Sequence; identical to SEQ ID NO: 28
  Clone #12651 Full

SEQ ID NO: 81 - DNA, 642 bp, Artificial Sequence; identical to SEQ ID NO: 29
  Clone #12651 Full

SEQ ID NO: 82 - protein, 214 aa, Artificial Sequence; identical to SEQ ID NO: 34
  Clone #12653 Full

SEQ ID NO: 83 - DNA, 642 bp, Artificial Sequence; identical to SEQ ID NO: 35
  Clone #12653 Full

SEQ ID NO: 84 - protein, 450 aa, Artificial Sequence; identical to SEQ ID NO: 50
  Clone #12659 Full

SEQ ID NO: 85 - DNA, 1350 bp, Artificial Sequence; identical to SEQ ID NO: 51
  Clone #12659 Full

SEQ ID NO: 86 - protein, 218 aa, Artificial Sequence; identical to SEQ ID NO: 53
  Clone 12660 Full

SEQ ID NO: 87 - DNA, 654 bp, Artificial Sequence; identical to SEQ ID NO: 54
  Clone #12660 Full

SEQ ID NO: 88 - protein, 629 aa, Artificial Sequence; identical to SEQ ID NO: 56
  Clone #12667 Full

SEQ ID NO: 89 - DNA, 1887 bp, Artificial Sequence; identical to SEQ ID NO: 57
  Clone #12667 Full

SEQ ID NO: 90 - protein, 448 aa, Artificial Sequence
  Clone #12966 Full
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 91 - DNA, 1344 bp, Artificial Sequence
  Clone #12966 Full
caggtgcagctggtgcagagcggagccgaggtgaagaagccaggggccagcgtgaaggtgtcttgcaaggcctctggctacagcttcacaggctataccatgaactgggtgcggcaggcccccggacagggcctggagtggatgggcctgatcacaccttacaacggggccagctcctataatcagaagtttcggggcaaggccaccatgacagtggacaccagcacatccaccgtgtacatggagctgtctagcctgaggtccgaggataccgccgtgtactattgtgccagaggcggctacgacggcagaggctttgattattggggccagggcacactggtgaccgtgtcctctgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 92 - protein, 483 aa, Artificial Sequence
  Clone #16711 Full
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTVEGGSGGSGGSGGSGGVDQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 93 - DNA, 1449 bp, Artificial Sequence
  Clone #16711 Full
gagctggtgctgacacagtccccttctgtgagcgccgccctgggctccccagccaagatcacctgcacactgagctccgcccacaagaccgacacaatcgattggtaccagcagctgcagggagaggcacccagatatctgatgcaggtgcagtctgacggcagctacaccaagcggcccggagtgcctgacagattctccggctctagctccggagccgatcgctatctgatcatcccatctgtgcaggccgacgatgaggccgactactattgcggagccgattacatcggaggatacgtgttcggaggaggaacccagctgaccgtgacagtggagggaggctccggaggctctggaggcagcggcggctccggcggcgtggaccaggagcagctggtggagagcggcggcagactggtgaccccaggaggctccctgacactgtcttgtaaggccagcggcttcgatttttccgcctactatatgtcttgggtgagacaggcaccaggcaagggcctggagtggatcgccaccatctacccctctagcggcaagacctactatgccacatgggtgaacggcagattcaccatctcctctgacaacgcccagaatacagtggatctgcagatgaatagcctgaccgccgccgacagggccacatacttctgcgcccgcgattcctatgccgacgatggggccctgttcaacatctggggccctggcaccctggtgacaatcagctccgccgccgagccaaagtctagcgacaagacccacacatgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 94 - protein, 473 aa, Artificial Sequence
  Clone #16712 Full
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSGGGGSGGGGSGGGGSDIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 95 - DNA, 1419 bp, Artificial Sequence
  Clone #16712 Full
caggtgcagctggtgcagagcggagccgaggtgaagaagcctggggccagcgtgaaggtgtcctgcaaggcctccggctactctttcacaggctataccatgaactgggtgcggcaggccccaggacagggcctggagtggatgggcctgatcacaccctacaacggggccagctcctataatcagaagtttcggggcaaggccaccatgacagtggacaccagcacatccaccgtgtacatggagctgtctagcctgagatccgaggataccgccgtgtactattgcgccagaggcggatacgacggcagaggctttgattattggggccagggcacactggtgaccgtgtcctctggcggcggcggctctggaggaggaggcagcggcggaggaggctccgacatccagatgacacagtccccaagctccctgtctgccagcgtgggcgatagggtgacaatcacctgttctgcctctagctccgtgagctacatgcactggtatcagcagaagtctggcaaggcccctaagctgctgatctatgacacctctaagctggccagcggagtgccatcccgcttctccggctctggcagcggaacagactttacactgaccatctctagcctgcagcccgaggatttcgccacctactattgtcagcagtggagcaagcaccctctgacatttggccagggcaccaagctggagatcaaggccgccgagcccaagtcctctgataagacacacacctgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 96 - protein, 449 aa, Artificial Sequence
  Clone #16713 Full
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 97 - DNA, 1347 bp, Artificial Sequence
  Clone #16713 Full
gaggtgcagctggtggagagcggcggcggcctggtgcagcccggcggctctctgcggctgagctgcgccgcctccggctttaacatcaaggacacatacatccactgggtgcggcaggcccccggcaagggcctggagtgggtggccagaatctatcctaccaatggctacacacggtatgccgactccgtgaagggcagattcaccatctctgccgataccagcaagaacacagcctacctgcagatgaacagcctgcgggccgaggatacagccgtgtactattgttctcgctggggcggcgacggcttttacgccatggattattggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 98 - protein, 701 aa, Artificial Sequence
  Clone #16714 Full
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSGGGGSGGGGSGGGGSGGGGSQIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 99 - DNA, 2103 bp, Artificial Sequence
  Clone #16714 Full
caggtgcagctgcagcagagcggagccgagctggccagacctggggccagcgtgaagatgtcttgcaaggccagcggctacacattcaccacatataccatgcactgggtgaagcagagacctggccagggcctggagtggatcggctacatcaacccaagctccggctacaccaactataatcagaagtttaaggacaaggccaccctgacagccgataagtctagctccacagcctccatgcagctgtctagcctgacctctgaggacagcgccgtgtactattgcgcccgggagagagccgtgctggtgccttacgccatggattattggggccagggcacaagcgtgaccgtgtcctctggaggaggaggcagcggcggaggaggctccggaggcggcggctctggcggcggcggcagccagatcgtgctgacccagtccccagccgtgatgtctgccagcccaggagagaaggtgaccatcacatgtaccgccagctcctctctgagctacatgcactggttccagcagaagcccggcacatcccctaagctgtggctgtattccacctctatcctggcctccggcgtgcccacaaggtttagcggctccggctctggcacaagctactccctgaccatctctaggatggaggccgaggacgccgccacctactattgccagcagcgcagctcctctccattcacatttggcagcggcaccaagctggagatcaagggaggaggaggctccgaggtgcagctggtggagtctggaggaggactggtgcagccaggaggctccctgcggctgtcttgtgccgccagcggctttaacatcaaggacacatacatccactgggtgaggcaggcccccggcaagggactggagtgggtggcccgcatctatcctacaaatggctacaccagatatgccgactccgtgaagggccgcttcaccatctccgccgatacatctaagaacaccgcctacctgcagatgaacagcctgcgggccgaggatacagccgtgtactattgtagcagatggggcggcgacggcttttacgctatggactactggggacagggcacactggtgaccgtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 100 - protein, 700 aa, Artificial Sequence
  Clone #16716 Full
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSGGGGSGGGGSGGGGSGGGGSQIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 101 - DNA, 2100 bp, Artificial Sequence
  Clone #16716
caggtgcagctgcagcagtccggagccgagctggccagacctggggccagcgtgaagatgtcctgcaaggcctctggctacaccttcaccacatatacaatgcactgggtgaagcagcgccctggacagggactggagtggatcggctacatcaacccaagctccggctacaccaactataatcagaagtttaaggacaaggccaccctgacagccgataagtctagctccaccgccagcatgcagctgtctagcctgacatctgaggacagcgccgtgtactattgcgcccgggagagagccgtgctggtgccttacgccatggattattggggccagggcacctccgtgacagtgtcctctggaggaggaggctctggaggaggaggcagcggcggaggaggctccggcggcggcggctctcagatcgtgctgacccagagcccagccgtgatgagcgcctccccaggagagaaggtgaccatcacatgtaccgccagctcctctctgtcttacatgcactggttccagcagaagcccggcaccagccctaagctgtggctgtattctacaagcatcctggcctccggagtgccaacccggttttccggctctggcagcggcacctcctactctctgacaatctctaggatggaggccgaggacgccgccacctactattgccagcagcgcagctcctctccattcacctttggctccggcacaaagctggagatcaagggaggaggaggcagccaggtgcagctggtgcagtccggagccgaggtgaagaagccaggggccagcgtgaaggtgtcctgtaaggcctccggctactctttcaccggctatacaatgaattgggtgagacaggcccccggccagggcctggagtggatgggcctgatcacaccttacaacggggccagctcctataatcagaagtttcggggcaaggccacaatgaccgtggacacaagcacctccacagtgtacatggagctgtctagcctgagaagcgaggataccgccgtgtactattgtgccaggggcggatacgacggcagaggctttgactactggggccagggcaccctggtgacagtgtcctctgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 102 - protein, 699 aa, Artificial Sequence
  Clone #16717 Full
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 103 - DNA, 2097 bp, Artificial Sequence
  Clone #16717 Full
caggtgcagctggtggagtccggcggcggcgtggtgcagcctggcaggagcctgcgcctgtcctgcgcagcctctggcttcaccttcagcaactacggcatgtattgggtgagacaggcccctggcaagggactggagtgggtggccgtgatctggtacgacggctctaataagtactatgccgatagcgtgaagggccggttcaccatcagcagagacaactccaagaatacactgtatctgcagatgaactccctgcgggccgaggataccgccgtgtactattgcgccagagacctgtggggctggtactttgattattggggccagggcaccctggtgacagtgagcagcggaggaggaggctccggcggcggaggctctggcggcggcggcagcggaggcggcggctccgagatcgtgctgacccagtctccagccacactgtctctgagcccaggagagagggccaccctgagctgtcgcgcctcccagagcgtgagcagctacctggcctggtatcagcagaagccaggacaggcccctcggctgctgatctacgacgccagcaacagggcaaccggcatcccagccagattcagcggctccggctctggcacagactttaccctgacaatctcctctctggagcccgaggatttcgccgtgtactattgccagcagcggagaaattggcctctgacctttggcggcggcacaaaggtggagatcaagggaggaggaggctccgaagtccagctggtggagtctggaggaggactggtgcagccaggaggctctctgcggctgagctgtgccgcctccggctttaacatcaaggacacctacatccactgggtgcggcaggcccctggcaagggcctggagtgggtggccagaatctatccaaccaatggctacacaagatatgccgactccgtgaagggccgcttcaccatctctgccgataccagcaagaacacagcctacctgcagatgaatagcctgagggccgaggatacagccgtgtactattgttcccgctggggaggcgacggcttttacgcaatggactactggggacagggcaccctggtcacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 104 - protein, 698 aa, Artificial Sequence
  Clone #16719 Full
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIKGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 105 - DNA, 2097 bp, Artificial Sequence; identical to SEQ ID NO: 103
  Clone #16719 Full

SEQ ID NO: 106 - protein, 700 aa, Artificial Sequence
  Clone #16720 Full
EVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSSGGGGSGGGGSGGGGSGGGGSDIQMTQTTSSLSASLGDRVTISCSASQGISNYLNWYQQKPDGTVKLLIYYTSILHSGVPSRFSGSGSGTDYSLTIGNLEPEDIATYYCQQFNKLPPTFGGGTKLEIKGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 107 - DNA, 2100 bp, Artificial Sequence
  Clone #16720 Full
gaggtgaagctggtggagtccggaggaggactggtgcagccaggaggctctctgaagctgagctgcgccacctccggcttcacattttctgactactatatgtactgggtgcggcagacccccgagaagagactggagtgggtggcctatatcaactctggcggcggcagcacctactatcctgacacagtgaagggcaggttcaccatctcccgcgataacgccaagaatacactgtacctgcagatgtcccggctgaagtctgaggacacagccatgtactattgcgcccggagaggcctgccttttcacgccatggattattggggccagggcaccagcgtgacagtgagcagcggcggcggcggctctggaggaggaggcagcggcggaggaggctccggaggaggcggctctgacatccagatgacccagaccacatctagcctgagcgcctccctgggcgatagggtgacaatctcttgtagcgcctcccagggcatctccaactacctgaattggtatcagcagaagcctgatggcaccgtgaagctgctgatctactatacaagcatcctgcactccggcgtgccatctcgcttctctggcagcggctccggaaccgactacagcctgacaatcggcaacctggagccagaggatatcgccacctactattgccagcagttcaataagctgccccctacctttggcggcggcacaaagctggagatcaagggcggcggcggcagcgaggtgcagctggtcgaaagcggcggcggcctggtccagcctggaggcagcctgaggctgtcctgtgccgcctctggctttaacatcaaggacacctacatccactgggtgaggcaggccccaggcaagggactggagtgggtggcccgcatctatcccaccaatggctacacaagatatgccgacagcgtgaagggccgcttcaccatcagcgccgatacctccaagaacacagcctacctgcagatgaacagcctgcgggccgaggatacagccgtgtactattgtagcagatggggcggcgacggcttttacgctatggactactggggacagggcaccctggtgacagtgtcctctgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 108 - protein, 699 aa, Artificial Sequence
  Clone #16722 Full
EVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSSGGGGSGGGGSGGGGSGGGGSDIQMTQTTSSLSASLGDRVTISCSASQGISNYLNWYQQKPDGTVKLLIYYTSILHSGVPSRFSGSGSGTDYSLTIGNLEPEDIATYYCQQFNKLPPTFGGGTKLEIKGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 109 - DNA, 2100 bp, Artificial Sequence; identical to SEQ ID NO: 107
  Clone #16722 Full

SEQ ID NO: 110 - protein, 862 aa, Artificial Sequence
  Clone #16733 Full
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 111 - DNA, 2586 bp, Artificial Sequence
  Clone #16733 Full
gaggtgcagctggtggagagcggcggcggcctggtgcagcccggcggctctctgcggctgagctgcgccgcctccggctttaacatcaaggacacatacatccactgggtgcggcaggcccccggcaagggcctggagtgggtggccagaatctatcctaccaatggctacacacggtatgccgactccgtgaagggcagattcaccatctctgccgataccagcaagaacacagcctacctgcagatgaacagcctgcgggccgaggatacagccgtgtactattgttctcgctggggcggcgacggcttttacgccatggattattggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaggtggagcctaagagctgcgacaagacccacaccggaggaggaggctccgagccagccgtgtatttcaaggagcagtttctggacggcgatggctggaccagcaggtggatcgagtccaagcacaagtctgacttcggcaagtttgtgctgagctccggcaagttctatggcgatgaggagaaggacaagggcctgcagacaagccaggatgcccgcttttacgccctgtccgcctctttcgagcccttttccaacaagggccagaccctggtggtgcagttcacagtgaagcacgagcagaacatcgactgtggcggcggctatgtgaagctgtttcctaattccctggatcagaccgacatgcacggcgactctgagtacaacatcatgttcggccctgatatctgcggcccaggcacaaagaaggtgcacgtgatctttaattacaagggcaagaacgtgctgatcaataaggacatccggtgtaaggacgatgagttcacccacctgtacacactgatcgtgagaccagacaacacctatgaggtgaagatcgataatagccaggtggagagcggctccctggaggacgattgggattttctgccccctaagaagatcaaggaccccgatgcctctaagcctgaggactgggatgagcgggccaagatcgacgatccaacagactccaagcccgaggactgggataagcccgagcacatcccagaccccgatgccaagaagccagaagactgggatgaggagatggatggcgagtgggagccacccgtgatccagaaccctgagtacaagggcgagtggaagcccagacagatcgataatcctgactataagggcacctggattcaccctgagatcgataacccagagtacagccctgacccatccatctacgcctatgataatttcggcgtgctgggactggacctgtggcaggtgaagtccggcaccatcttcgacaactttctgatcacaaatgatgaggcctacgccgaggagtttggcaacgagacctggggcgtgacaaaggccgccgagaagcagatgaaggataagcaggacgaggagcagaggctgaaggaagaagaggaggacaagaagcgcaaggaggaggaggaggccgaggataaggaggacgatgaggacaaggatgaggacgaggaggatgaggaggacaaggaggaggatgaggaggaggacgtgccaggacaggccgccgccgagcccaagtctagcgacaagacccacacatgccctccatgtccggcgccagaggccgccggaggaccttccgtgttcctgtttccccctaagccaaaggataccctgatgatctctagaaccccagaggtgacatgcgtggtggtgtctgtgagccacgaggaccccgaggtgaagttcaactggtatgtggatggcgtggaggtgcacaatgccaagacaaagcctagggaggagcagtacaattctacctatagagtggtgagcgtgctgacagtgctgcaccaggactggctgaacggcaaggagtacaagtgtaaggtgtctaataaggccctgccagcccccatcgagaagaccatcagcaaggccaagggccagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 112 - protein, 861 aa, Artificial Sequence
  Clone #16735 Full
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 113 - DNA, 2583 bp, Artificial Sequence
  Clone #16735 Full
caggtgcagctggtgcagagcggagccgaggtgaagaagccaggggccagcgtgaaggtgtcttgcaaggcctctggctacagcttcacaggctataccatgaactgggtgcggcaggcccccggacagggcctggagtggatgggcctgatcacaccttacaacggggccagctcctataatcagaagtttcggggcaaggccaccatgacagtggacaccagcacatccaccgtgtacatggagctgtctagcctgaggtccgaggataccgccgtgtactattgtgccagaggcggctacgacggcagaggctttgattattggggccagggcacactggtgaccgtgtcctctgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaggtggagcccaagtcttgcgacaagacccacaccggaggaggaggcagcgagcctgccgtgtatttcaaggagcagtttctggacggcgatggatggaccagccggtggatcgagtctaagcacaagagcgacttcggcaagtttgtgctgagctccggcaagttctatggcgatgaggagaaggacaagggcctgcagacatcccaggatgcccggttctacgccctgtccgcctctttcgagccattttctaacaagggccagaccctggtggtgcagttcacagtgaagcacgagcagaacatcgactgtggcggcggctatgtgaagctgtttcccaatagcctggatcagaccgacatgcacggcgactccgagtacaacatcatgttcggccctgatatctgcggcccaggcacaaagaaggtgcacgtgatctttaattacaagggcaagaacgtgctgatcaataaggacatcaggtgtaaggacgatgagttcacccacctgtacacactgatcgtgcgccctgacaacacctatgaggtgaagatcgataattctcaggtggagagcggctccctggaggacgattgggattttctgccccctaagaagatcaaggaccccgatgccagcaagcctgaggactgggatgagagggccaagatcgacgatccaacagactccaagcccgaggactgggataagcctgagcacatccccgaccctgatgccaagaagccagaggactgggatgaggagatggatggcgagtgggagccacccgtgatccagaaccccgagtacaagggcgagtggaagcccagacagatcgataatcctgactataagggcacctggattcaccctgagatcgataacccagagtactccccagacccctctatctacgcctatgataatttcggcgtgctgggcctggacctgtggcaggtgaagtccggcaccatcttcgacaactttctgatcacaaatgatgaggcctatgccgaggagtttggcaatgagacctggggcgtgacaaaggccgccgagaagcagatgaaggataagcaggacgaggagcagcggctgaaggaagaagaggaggacaagaagagaaaggaggaggaggaggccgaggataaggaggacgatgaggacaaggatgaggacgaggaggatgaggaggacaaggaggaggatgaggaggaggacgtgccaggacaggccgccgccgagcccaagtctagcgacaagacccacacatgccctccatgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 114 - protein, 732 aa, Artificial Sequence
  Clone #16743 Full
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSGGGGSGGGGSGGGGSGGGGSQIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSQVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSGGGGSGGGGSGGGGSGGGGSQIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIK

SEQ ID NO: 115 - DNA, 2196 bp, Artificial Sequence
  Clone #16743 Full
caggtgcagctgcagcagtccggagccgagctggccagacccggagccagcgtgaagatgtcctgcaaggcctctggctacaccttcaccacatatacaatgcactgggtgaagcagagacccggacagggactggagtggatcggatacatcaaccctagctccggctacaccaactataatcagaagtttaaggacaaggccaccctgacagccgataagtctagctccaccgccagcatgcagctgtctagcctgacaagcgaggactccgccgtgtactattgtgcccgggagagagccgtgctggtgccatacgccatggattattggggccagggcacctccgtgacagtgtcctctggaggaggaggcagcgggggaggaggctccggaggcggcggctctggcggcggcggcagccagatcgtgctgacccagagccccgccgtgatgtctgccagccctggagagaaggtgaccatcacatgcaccgccagctcctctctgagctacatgcactggttccagcagaagccaggcacctcccccaagctgtggctgtattccacatctatcctggcctccggagtgccaaccaggtttagcggctccggctctggcaccagctactccctgacaatcagcaggatggaggcagaggacgcagcaacctactattgtcagcagcgcagctcctctccattcacctttggcagcggcacaaagctggagatcaaggccgccgagcccaagagctccgacaagacacacacctgcccaccttgtccggcgccagaggccgccggaggaccttccgtgttcctgtttccacccaagccaaaggataccctgatgatcagcaggaccccagaggtgacatgcgtggtggtgtctgtgagccacgaggaccctgaggtgaagtttaactggtacgtggatggcgtggaggtgcacaatgccaagacaaagcctcgggaggagcagtacaactctacctatagagtggtgagcgtgctgacagtgctgcaccaggactggctgaacggcaaggagtataagtgcaaggtgtccaataaggccctgcctgccccaatcgagaagaccatctctaaggccaagggccagcctcgcgaacctcaggtgtacgtgctgcctccatcccgcgacgagctgacaaagaaccaggtgtctctgctgtgcctggtgaagggcttctatccttctgatatcgccgtggagtgggagagcaatggccagccagagaacaattacctgacctggccccctgtgctggactctgatggcagcttctttctgtattccaagctgacagtggataagtctcggtggcagcagggcaacgtgttttcctgctctgtgatgcacgaggccctgcacaatcactacacccagaagagcctgagcttaagccctggaggaggaggaggcagccaggtccagctgcagcagagcggagccgagctggccaggccaggagccagcgtcaagatgtcctgtaaagcctctggatataccttcaccacctacaccatgcattgggtcaagcagcgcccaggccagggcctggagtggatcggctatatcaatccctctagcggctacacaaattacaaccagaagtttaaggataaggccacactgaccgccgataagtcctctagcacagccagcatgcagctgtcctctctgacctccgaggactctgccgtgtactattgtgcaagggagagggccgtgctggtcccttatgctatggactactggggacagggcacctccgtcacagtgagctctggcggaggaggctccggaggaggaggctctggaggaggcggcagcggcggcggcggctcccagatcgtgctgactcagagcccagccgtgatgagcgcctccccaggagagaaggtgacaatcacctgcacagcctctagctccctgtcttatatgcattggttccagcagaagcctggcacaagcccaaagctgtggctgtattctaccagcatcctggcctccggcgtcccaacacggttttccggctctggcagcggcacctcctactctctgaccatttccagaatggaggcagaggatgccgccacttattattgtcagcagagatctagctcccctttcacctttggcagcggaaccaaactggagatcaag

SEQ ID NO: 116 - protein, 726 aa, Artificial Sequence
  Clone #16744 Full
QIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKGGGGSGGGGSGGGGSGGGGSQVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSQIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKGGGGSGGGGSGGGGSGGGGSQVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSS

SEQ ID NO: 117 - DNA, 2178 bp, Artificial Sequence
  Clone #16744 Full
cagatcgtgctgacacagtcccccgccgtgatgagcgcctcccctggagagaaggtgaccatcacatgcaccgccagctcctctctgtcttacatgcactggttccagcagaagccaggcaccagccccaagctgtggctgtattctacaagcatcctggcctccggagtgcctacccggttttccggctctggcagcggcacctcctactctctgacaatcagcaggatggaggcagaggacgcagcaacctactattgccagcagagaagctcctctccattcacctttggcagcggcacaaagctggagatcaagggaggaggaggctccgggggaggaggctctggcggcggcggcagcggaggcggcggctcccaggtgcagctggtggagtccggcggcggcgtggtgcagcccggcagaagcctgagactgtcctgtgccgcctctggcttcacctttagcaactacggcatgtattgggtgagacaggcacctggcaagggactggagtgggtggccgtgatctggtacgacggctctaataagtactatgccgatagcgtgaagggccggttcacaatcagcagagacaactccaagaataccctgtatctgcagatgaacagcctgagggccgaggataccgccgtgtactattgcgcccgcgacctgtggggctggtactttgattattggggccagggcaccctggtgacagtgagctccgccgccgagccaaagtctagcgacaagacacacacctgcccaccttgtccggcgccagaggccgccggaggacctagcgtgttcctgtttccacccaagccaaaggataccctgatgatcagcaggaccccagaggtgacatgcgtggtggtgagcgtgtcccacgaggaccccgaggtgaagttcaactggtacgtggatggcgtggaggtgcacaatgccaagacaaagcctcgggaggagcagtacaatagcacctatagagtggtgtccgtgctgacagtgctgcaccaggactggctgaacggcaaggagtacaagtgcaaggtgagcaataaggccctgcctgccccaatcgagaagaccatctccaaggccaagggccagcctcgcgaacctcaggtgtacgtgctgcctccaagcagagacgagctgacaaagaaccaggtgtccctgctgtgcctggtgaagggcttctatccctccgatatcgccgtggagtgggagtctaatggccagcctgagaacaattacctgacctggccccctgtgctggactccgatggctctttctttctgtattccaagctgacagtggataagtctaggtggcagcagggcaacgtgttttcttgcagcgtgatgcacgaggccctgcacaatcactacacccagaagtccctgagcttaagcccaggaggaggaggaggcagccagatcgtgctgacccagtccccagccgtgatgtccgcctctccaggagagaaggtgacaatcacctgtacagcctcctctagcctgtcctatatgcattggttccagcagaagcctggcacatctccaaagctgtggctgtatagcacctccatcctggcctccggcgtcccaacacgcttttctggcagcggctccggcacctcttacagcctgaccattagcaggatggaggccgaggatgccgccacttattattgccagcagcggagctctagccctttcacctttggctccggaaccaagctggagatcaagggcggcggcggctctggaggaggaggcagcggaggaggaggctccggcggcggcggctctcaggtccagctggtcgagtccggaggaggagtggtgcagccaggcaggtctctgaggctgagctgtgcagcctccggcttcacctttagcaattacggaatgtattgggtgcggcaggcaccaggcaagggcctggaatgggtcgccgtgatctggtatgatggctctaataagtattacgctgacagcgtgaagggcaggttcaccatctcccgcgacaacagcaagaatacattatatctgcaaatgaacagcctgagagctgaagacaccgccgtgtactattgtgctagagacctgtggggatggtatttcgactactggggacagggcaccctggtcacagtgtcctct

SEQ ID NO: 118 - protein, 728 aa, Artificial Sequence
  Clone #16745 Full
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSQVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIK

SEQ ID NO: 119 - DNA, 2184 bp, Artificial Sequence
  Clone #16745 Full
caggtgcagctggtggagtccggaggaggagtggtgcagcctggccggtccctgagactgtcttgcgcagccagcggcttcaccttcagcaactacggcatgtattgggtgaggcaggcaccaggcaagggactggagtgggtggccgtgatctggtacgacggcagcaataagtactatgccgattccgtgaagggccggttcaccatctccagagacaactctaagaatacactgtatctgcagatgaactccctgagggccgaggataccgccgtgtactattgcgcccgcgacctgtggggctggtactttgattattggggccagggcaccctggtgacagtgagcagcggcggcggcggctctggaggaggaggcagcgggggaggaggctccggaggaggcggctctgagatcgtgctgacccagtctcccgccacactgtctctgagccctggagagagggccaccctgagctgtagagcctcccagagcgtgagcagctacctggcctggtatcagcagaagccaggccaggcccccagactgctgatctacgacgccagcaacagggcaaccggcatccctgccagattcagcggctccggctctggcacagactttaccctgacaatctcctctctggagcctgaggatttcgccgtgtactattgccagcagcggagaaattggccactgacctttggcggcggcacaaaggtggagatcaaggccgccgagccaaagagctccgacaagacccacacatgcccaccttgtccggcgccagaggccgccggaggaccttccgtgttcctgtttccacccaagccaaaggataccctgatgatcagcagaaccccagaggtgacatgcgtggtggtgagcgtgtcccacgaggaccccgaggtgaagttcaactggtacgtggatggcgtggaggtgcacaatgccaagacaaagcccagagaggagcagtacaactccacctatagagtggtgtctgtgctgacagtgctgcaccaggactggctgaacggcaaggagtacaagtgcaaggtgagcaataaggccctgcctgccccaatcgagaagaccatctccaaggccaagggccagcctcgcgaacctcaggtgtacgtgctgcctccatccagagacgagctgacaaagaaccaggtgtctctgctgtgcctggtgaagggcttctatccctctgatatcgccgtggagtgggagagcaatggccagcctgagaacaattacctgacctggccccctgtgctggactctgatggcagcttctttctgtattctaagctgacagtggataagagcaggtggcagcagggcaacgtgttttcttgcagcgtgatgcacgaggccctgcacaatcactacacccagaagtccctgagcttaagcccaggaggaggaggaggctcccaggtccagctggtcgagtctggcggcggagtggtgcagcccggcaggagcctgaggctgtcctgtgcagcctctggcttcacattttccaactacggaatgtattgggtgcgccaggcccctggcaagggcctggaatgggtcgccgtgatctggtatgatggcagcaataagtattacgctgactccgtgaagggcaggttcaccatcagccgcgacaactccaaaaacaccctgtatctgcagatgaatagcctgagagctgaagacaccgccgtgtactattgtgctagagacctgtggggatggtatttcgactactggggacagggcaccctggtcacagtgtctagcggcggcggcggcagcggcggcggaggctccggagggggcggctctggcggcggcggcagcgaaatcgtgctgactcagtccccagccacactgtccctgtctccaggcgaaagggccaccctgagctgcagggccagccagtccgtgtcctcttacctggcttggtaccagcagaagcctggacaggcaccacggctgctgatctacgatgccagcaatagagcaaccggcatccctgcacgcttctctggcagcggctccggaaccgactttaccctgaccattagctccctggagcccgaagacttcgccgtgtactattgtcagcagaggcgcaattggcctctgacctttggcggaggaaccaaagtggagatcaag

SEQ ID NO: 120 - protein, 702 aa, Artificial Sequence
  Clone #16772 Full
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSGGGGSGGGGSGGGGSGGGGSQIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKGGGGSQVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 121 - DNA, 2106 bp, Artificial Sequence
  Clone #16722 Full
caggtgcagctgcagcagtccggagccgagctggccagacctggggccagcgtgaagatgtcttgcaaggccagcggctacacattcaccacatataccatgcactgggtgaagcagcgccctggacagggactggagtggatcggctacatcaacccaagctccggctacacaaactataatcagaagtttaaggacaaggccaccctgacagccgataagtctagctccacagccagcatgcagctgtctagcctgaccagcgaggactccgccgtgtactattgcgcccgggagagagccgtgctggtgccttacgccatggattattggggccagggcacatctgtgaccgtgtcctctggcggcggcggctccggaggcggcggctctggaggaggaggcagcggcggaggaggctcccagatcgtgctgacccagagcccagccgtgatgagcgcctccccaggagagaaggtgaccatcacatgtaccgccagctcctctctgtcctacatgcactggttccagcagaagcccggcacatctcctaagctgtggctgtattctaccagcatcctggccagcggcgtgccaacacggttttccggctctggcagcggcacatcctactctctgaccatctccaggatggaggcagaggacgcagcaacctactattgccagcagcgcagctcctctccattcacatttggctccggcaccaagctggagatcaagggaggaggaggctctcaggtccagctgcagcagagcggagccgagctggcccggcccggggccagcgtcaaaatgtcttgtaaagccagcggatatacattcaccacctacactatgcattgggtcaagcagagacccggccagggcctggagtggatcggatacatcaatcctagctccggctacaccaattacaaccagaagtttaaggataaggccacactgaccgccgataaatccagctccaccgcctccatgcagctgtcctccctgacatctgaggacagcgccgtgtactattgtgccagggagagggccgtgctggtcccatatgctatggactactggggccagggcacaagcgtgaccgtgtcctctgctagcaccaagggaccatccgtgttcccactggcaccaagctccaagtctacaagcggaggaaccgccgccctgggctgtctggtgaaggattacttcccagagcccgtgaccgtgtcttggaacagcggggccctgaccagcggagtgcacacctttcctgccgtgctgcagtctagcggcctgtatagcctgtcctctgtggtcacagtgccaagctcctctctgggcacacagacctacatctgcaacgtgaatcacaagccatccaataccaaggtcgacaagaaggtggagcccaagtcttgtgataagacacacacctgcccaccttgtccggcgccagaggccgccggaggaccaagcgtgttcctgtttccacccaagcctaaggacacactgatgatcagcaggacaccagaggtgacctgcgtggtggtgtccgtgtctcacgaggaccccgaggtgaagtttaactggtacgtggatggcgtggaggtgcacaatgccaagaccaagccaagggaggagcagtataactctacataccgcgtggtgagcgtgctgaccgtgctgcaccaggattggctgaacggcaaggagtacaagtgcaaggtgagcaataaggccctgcccgcccctatcgagaagacaatctccaaggccaagggccagcctcgcgaaccacaggtgtatgtgctgcctccatctagagacgagctgaccaagaaccaggtgagcctgctgtgcctggtgaagggcttctaccccagcgatatcgccgtggagtgggagtccaatggccagcctgagaacaattatctgacatggccccctgtgctggactccgatggctctttctttctgtactccaagctgaccgtggacaagtctcgctggcagcagggcaacgtgtttagctgttccgtgatgcacgaggccctgcacaatcactacacccagaagtctctgagcttaagccctggc

SEQ ID NO: 122 - protein, 697 aa, Artificial Sequence
  Clone #16773 Full
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIKGGGGSQVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 123 - DNA, 2091 bp, Artificial Sequence
  Clone #16773 Full
caggtgcagctggtggagtccggcggcggcgtggtgcagccaggcaggagcctgcgcctgtcctgcgcagcctctggcttcacattttctaactacggcatgtattgggtgagacaggccccaggcaagggactggagtgggtggccgtgatctggtacgacggctctaataagtactatgccgatagcgtgaagggcaggttcaccatcagccgcgacaactccaagaatacactgtatctgcagatgaactccctgagggccgaggataccgccgtgtactattgcgcccgcgacctgtggggctggtactttgattattggggccagggcaccctggtgacagtgagcagcggaggaggaggctccggcggcggaggctctggcggcggcggcagcggaggcggcggctccgagatcgtgctgacccagtctccagccacactgtctctgagcccaggagagagggccaccctgagctgtcgcgcctcccagagcgtgagcagctacctggcctggtatcagcagaagccaggacaggcccctcggctgctgatctacgacgccagcaacagggcaaccggcatccccgcaagattcagcggctccggctctggcacagactttaccctgacaatctcctctctggagcctgaggatttcgccgtgtactattgccagcagcggagaaattggccactgacctttggcggcggcacaaaggtggagatcaagggaggaggaggctcccaggtccagctggtcgagtctggaggaggagtggtgcagcccggcagaagcctgcggctgagctgtgcagcctccggcttcaccttttccaattatggcatgtattgggtgcggcaggcccctggcaagggcctggaatgggtcgccgtgatctggtatgatggcagcaataagtattacgccgattccgtgaagggccggttcaccatctctagagacaacagcaagaatacactgtacctgcagatgaatagcctgcgggccgaggatacagccgtgtactattgtgccagagacctgtggggatggtatttcgactactggggacagggcaccctggtcacagtgagctccgctagcaccaagggaccatccgtgttcccactggcaccaagctccaagtctacaagcggaggaaccgccgccctgggctgtctggtgaaggattacttcccagagcccgtgaccgtgtcttggaacagcggggccctgaccagcggagtgcacacctttcctgccgtgctgcagtctagcggcctgtatagcctgtcctctgtggtcacagtgccaagctcctctctgggcacacagacctacatctgcaacgtgaatcacaagccatccaataccaaggtcgacaagaaggtggagcccaagtcttgtgataagacacacacctgcccaccttgtccggcgccagaggccgccggaggaccaagcgtgttcctgtttccacccaagcctaaggacacactgatgatcagcaggacaccagaggtgacctgcgtggtggtgtccgtgtctcacgaggaccccgaggtgaagtttaactggtacgtggatggcgtggaggtgcacaatgccaagaccaagccaagggaggagcagtataactctacataccgcgtggtgagcgtgctgaccgtgctgcaccaggattggctgaacggcaaggagtacaagtgcaaggtgagcaataaggccctgcccgcccctatcgagaagacaatctccaaggccaagggccagcctcgcgaaccacaggtgtatgtgctgcctccatctagagacgagctgaccaagaaccaggtgagcctgctgtgcctggtgaagggcttctaccccagcgatatcgccgtggagtgggagtccaatggccagcctgagaacaattatctgacatggccccctgtgctggactccgatggctctttctttctgtactccaagctgaccgtggacaagtctcgctggcagcagggcaacgtgtttagctgttccgtgatgcacgaggccctgcacaatcactacacccagaagtctctgagcttaagccctggc

SEQ ID NO: 124 - protein, 699 aa, Artificial Sequence
  Clone #16774 Full
EVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSSGGGGSGGGGSGGGGSGGGGSDIQMTQTTSSLSASLGDRVTISCSASQGISNYLNWYQQKPDGTVKLLIYYTSILHSGVPSRFSGSGSGTDYSLTIGNLEPEDIATYYCQQFNKLPPTFGGGTKLEIKGGGGSEVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 125 - DNA, 2097 bp, Artificial Sequence
  Clone #16774 Full
gaggtgaagctggtggagtccggaggaggactggtgcagcctggaggctctctgaagctgagctgcgccacctccggcttcacattttctgactactatatgtactgggtgcggcagacccctgagaagagactggagtgggtggcctatatcaactctggcggcggcagcacctactatccagacacagtgaagggccggttcaccatctccagagataacgccaagaatacactgtacctgcagatgtcccggctgaagtctgaggacacagccatgtactattgcgcccggagaggcctgccttttcacgccatggattattggggccagggcaccagcgtgacagtgagcagcggaggaggaggctccggcggcggaggctctggcggcggcggcagcggaggcggcggctccgacatccagatgacccagaccacatctagcctgagcgcctccctgggcgatagggtgacaatctcttgtagcgcctcccagggcatctctaactacctgaattggtatcagcagaagccagacggcaccgtgaagctgctgatctactatacaagcatcctgcactccggcgtgccctctcgcttttctggcagcggctccggaaccgactacagcctgacaatcggcaacctggagccagaggatatcgccacctactattgccagcagttcaataagctgccccctacctttggcggcggcacaaagctggagatcaagggaggaggaggctctgaagtcaagctggtggagagtggcggaggactggtgcagccaggaggcagcctgaagctgtcctgtgccacctctggcttcaccttcagcgattattacatgtactgggtgaggcagaccccagagaagcgcctggaatgggtcgcctatatcaatagcggcggcggctccacctactatcctgacacagtgaagggcaggttcaccatctcccgcgataatgctaaaaacaccctgtacctgcagatgtctaggctgaagagcgaggacaccgccatgtactattgtgcaaggcgcggcctgccatttcacgcaatggattactggggccagggcacctccgtgacagtgtcctctgctagcaccaagggaccatccgtgttcccactggcaccaagctccaagtctacaagcggaggaaccgccgccctgggctgtctggtgaaggattacttcccagagcccgtgaccgtgtcttggaacagcggggccctgaccagcggagtgcacacctttcctgccgtgctgcagtctagcggcctgtatagcctgtcctctgtggtcacagtgccaagctcctctctgggcacacagacctacatctgcaacgtgaatcacaagccatccaataccaaggtcgacaagaaggtggagcccaagtcttgtgataagacacacacctgcccaccttgtccggcgccagaggccgccggaggaccaagcgtgttcctgtttccacccaagcctaaggacacactgatgatcagcaggacaccagaggtgacctgcgtggtggtgtccgtgtctcacgaggaccccgaggtgaagtttaactggtacgtggatggcgtggaggtgcacaatgccaagaccaagccaagggaggagcagtataactctacataccgcgtggtgagcgtgctgaccgtgctgcaccaggattggctgaacggcaaggagtacaagtgcaaggtgagcaataaggccctgcccgcccctatcgagaagacaatctccaaggccaagggccagcctcgcgaaccacaggtgtatgtgctgcctccatctagagacgagctgaccaagaaccaggtgagcctgctgtgcctggtgaagggcttctaccccagcgatatcgccgtggagtgggagtccaatggccagcctgagaacaattatctgacatggccccctgtgctggactccgatggctctttctttctgtactccaagctgaccgtggacaagtctcgctggcagcagggcaacgtgtttagctgttccgtgatgcacgaggccctgcacaatcactacacccagaagtctctgagcttaagccctggc

SEQ ID NO: 126 - protein, 480 aa, Artificial Sequence
  Clone #16778 Full
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSGGGGSGGGGSGGGGSGGGGSQIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 127 - DNA, 1440 bp, Artificial Sequence
  Clone #16778 Full
caggtgcagctgcagcagtccggagccgagctggcccgccccggggccagcgtgaagatgtcttgcaaggccagcggctacacattcaccacatataccatgcactgggtgaagcagagacccggacagggactggagtggatcggatacatcaaccctagctccggctacacaaactataatcagaagtttaaggacaaggccaccctgacagccgataagtctagctccacagccagcatgcagctgtctagcctgacctctgaggacagcgccgtgtactattgtgcccgggagagagccgtgctggtgccttacgccatggattattggggccagggcacatccgtgaccgtgtcctctggcggcggcggctccggaggcggcggctctggaggaggaggcagcggcggaggaggctcccagatcgtgctgacccagagccctgccgtgatgtctgccagcccaggagagaaggtgaccatcacatgcaccgccagctcctctctgtcttacatgcactggttccagcagaagccaggcacaagccccaagctgtggctgtattccacctctatcctggcctccggagtgccaacacggtttagcggctccggctctggcacaagctattccctgaccatctctcggatggaggcagaggacgcagcaacctactattgtcagcagagaagctcctctccattcacatttggcagcggcaccaagctggagatcaaggccgccgagcccaagagctccgataagacacacacctgccccccttgtccggcgccagaggccgccggaggaccaagcgtgttcctgtttccacccaagcctaaggacacactgatgatcagcaggacaccagaggtgacctgcgtggtggtgtccgtgtctcacgaggaccccgaggtgaagtttaactggtacgtggatggcgtggaggtgcacaatgccaagaccaagccaagggaggagcagtataactctacataccgcgtggtgagcgtgctgaccgtgctgcaccaggattggctgaacggcaaggagtacaagtgcaaggtgagcaataaggccctgcccgcccctatcgagaagacaatctccaaggccaagggccagcctcgcgaaccacaggtgtatgtgctgcctccatctagagacgagctgaccaagaaccaggtgagcctgctgtgcctggtgaagggcttctaccccagcgatatcgccgtggagtgggagtccaatggccagcctgagaacaattatctgacatggccccctgtgctggactccgatggctctttctttctgtactccaagctgaccgtggacaagtctcgctggcagcagggcaacgtgtttagctgttccgtgatgcacgaggccctgcacaatcactacacccagaagtctctgagcttaagccctggc

SEQ ID NO: 128 - protein, 478 aa, Artificial Sequence
  Clone #16779 Full
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 129 - DNA, 1434 bp, Artificial Sequence
  Clone #16779 Full
caggtgcagctggtggagtccggaggaggagtggtgcagcctggcaggagcctgcgcctgtcctgtgcagcctctggcttcacattttctaactacggcatgtattgggtgaggcaggcccctggcaagggactggagtgggtggccgtgatctggtacgacggcagcaataagtactatgccgattccgtgaagggccggttcaccatcagcagagacaactccaagaatacactgtatctgcagatgaacagcctgagggccgaggataccgccgtgtactattgcgcccgcgacctgtggggctggtactttgattattggggccagggcaccctggtgacagtgagctccggcggcggcggctctggaggaggaggcagcggcggaggaggctccggaggaggcggctctgagatcgtgctgacccagtctcctgccacactgtctctgagcccaggagagagggccaccctgagctgtagggcctcccagagcgtgagcagctacctggcctggtatcagcagaagccaggacaggccccccggctgctgatctacgacgcctccaacagggcaaccggcatcccagccagattcagcggctccggctctggcacagactttaccctgacaatctcctctctggagcccgaggatttcgccgtgtactattgccagcagcggagaaattggcctctgacctttggcggcggcacaaaggtggagatcaaggccgccgagcccaagagctccgataagacccacacatgccccccttgtccggcgccagaggccgccggaggaccaagcgtgttcctgtttccacccaagcctaaggacacactgatgatcagcaggacaccagaggtgacctgcgtggtggtgtccgtgtctcacgaggaccccgaggtgaagtttaactggtacgtggatggcgtggaggtgcacaatgccaagaccaagccaagggaggagcagtataactctacataccgcgtggtgagcgtgctgaccgtgctgcaccaggattggctgaacggcaaggagtacaagtgcaaggtgagcaataaggccctgcccgcccctatcgagaagacaatctccaaggccaagggccagcctcgcgaaccacaggtgtatgtgctgcctccatctagagacgagctgaccaagaaccaggtgagcctgctgtgcctggtgaagggcttctaccccagcgatatcgccgtggagtgggagtccaatggccagcctgagaacaattatctgacatggccccctgtgctggactccgatggctctttctttctgtactccaagctgaccgtggacaagtctcgctggcagcagggcaacgtgtttagctgttccgtgatgcacgaggccctgcacaatcactacacccagaagtctctgagcttaagccctggc

SEQ ID NO: 130 - protein, 479 aa, Artificial Sequence
  Clone #16780 Full
EVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSSGGGGSGGGGSGGGGSGGGGSDIQMTQTTSSLSASLGDRVTISCSASQGISNYLNWYQQKPDGTVKLLIYYTSILHSGVPSRFSGSGSGTDYSLTIGNLEPEDIATYYCQQFNKLPPTFGGGTKLEIKAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 131 - DNA, 1434 bp, Artificial Sequence; identical to SEQ ID NO: 129
  Clone #16780 Full

SEQ ID NO: 132 - protein, 629 aa, Artificial Sequence
  Clone #16781 Full
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRSKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 133 - DNA, 1887 bp, Artificial Sequence
  Clone #16781 Full
gagccagccgtgtatttcaaggagcagtttctggacggcgatggctggacctctaggtggatcgagtctaagcacaagagcgacttcggcaagtttgtgctgagctccggcaagttctatggcgatgaggagaaggacaagggcctgcagacatctcaggatgcccggttttacgccctgtccgcctctttcgagcccttcagcaacaagggccagaccctggtggtgcagttcacagtgaagcacgagcagaacatcgactgcggcggcggctatgtgaagctgtttcccaatagcctggatcagaccgacatgcacggcgactccgagtacaacatcatgttcggccccgatatctgtggccctggcacaaagaaggtgcacgtgatctttaattacaagggcaagaacgtgctgatcaataaggacatcaggagcaaggacgatgagttcacccacctgtacacactgatcgtgcgccctgacaacacctatgaggtgaagatcgataattcccaggtggagagcggctccctggaggacgattgggattttctgccccctaagaagatcaaggacccagatgcctccaagcccgaggactgggatgagcgcgccaagatcgacgatcctacagactctaagccagaggactgggataagcccgagcacatccccgaccctgatgccaagaagcctgaggactgggatgaggagatggatggcgagtgggagccacccgtgatccagaaccccgagtacaagggcgagtggaagccacggcagatcgataatcccgactataagggcacctggattcaccccgagatcgataaccctgagtactccccagacccctctatctacgcctatgataatttcggcgtgctgggcctggacctgtggcaggtgaagtccggcaccatcttcgacaactttctgatcacaaatgatgaggcctatgccgaggagtttggcaatgagacctggggcgtgacaaaggccgccgagaagcagatgaaggataagcaggacgaggagcagcggctgaaggaagaggaggaggacaagaagagaaaggaggaggaggaggccgaggataaggaggacgatgaggacaaggatgaggacgaggaggatgaggaggacaaggaggaggatgaggaggaggacgtgcctggacaggccgccgccgagccaaagtctagcgacaagacccacacatgccctccatgtccggcgccagaggccgccggaggaccaagcgtgttcctgtttccacccaagcctaaggacacactgatgatcagcaggacaccagaggtgacctgcgtggtggtgtccgtgtctcacgaggaccccgaggtgaagtttaactggtacgtggatggcgtggaggtgcacaatgccaagaccaagccaagggaggagcagtataactctacataccgcgtggtgagcgtgctgaccgtgctgcaccaggattggctgaacggcaaggagtacaagtgcaaggtgagcaataaggccctgcccgcccctatcgagaagacaatctccaaggccaagggccagcctcgcgaaccacaggtgtatgtgctgcctccatctagagacgagctgaccaagaaccaggtgagcctgctgtgcctggtgaagggcttctaccccagcgatatcgccgtggagtgggagtccaatggccagcctgagaacaattatctgacatggccccctgtgctggactccgatggctctttctttctgtactccaagctgaccgtggacaagtctcgctggcagcagggcaacgtgtttagctgttccgtgatgcacgaggccctgcacaatcactacacccagaagtctctgagcttaagccctggc

SEQ ID NO: 134 - protein, 493 aa, Artificial Sequence
  Clone #16782 Full
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPGSGDPSIYAYDNFGVLGLDLWQVKSGTIFDNFLITNDEAYAEEFGNETWGVTKAAEKQMKDKQDEEQRLKGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 135 - DNA, 1479 bp, Artificial Sequence
  Clone #16782 Full
gagcccgccgtgtacttcaaggagcagtttctggacggcgatggatggaccagccggtggatcgagtctaagcacaagagcgatttcggcaagtttgtgctgagctccggcaagttctacggcgacgaagagaaggataagggcctgcagacatctcaggacgccaggttttatgccctgtccgcctctttcgagcccttcagcaacaagggccagaccctggtggtgcagttcacagtgaagcacgagcagaacatcgattgcggcggcggctacgtgaagctgtttcccaatagcctggaccagaccgatatgcacggcgattccgagtataacatcatgttcggccctgacatctgcggcccaggcacaaagaaggtgcacgtgatctttaattacaagggcaagaacgtgctgatcaataaggacatccggtgtaaggacgatgagttcacccacctgtacacactgatcgtgagacctgataacacctatgaggtgaagatcgacaattcccaggtggagagcggctccctggaggacgattgggacttcctgcccggctccggcgatccttctatctacgcctatgacaactttggcgtgctgggcctggatctgtggcaggtgaagtctggcaccatcttcgataactttctgatcacaaatgacgaggcctatgccgaggagtttggcaatgagacctggggcgtgacaaaggccgccgagaagcagatgaaggacaagcaggatgaggagcagcggctgaagggaggaggaggctccgagccaaagtctagcgacaagacccacacatgccccccttgtccggcgccagaggccgccggaggaccaagcgtgttcctgtttccacccaagcctaaggacacactgatgatcagcaggacaccagaggtgacctgcgtggtggtgtccgtgtctcacgaggaccccgaggtgaagtttaactggtacgtggatggcgtggaggtgcacaatgccaagaccaagccaagggaggagcagtataactctacataccgcgtggtgagcgtgctgaccgtgctgcaccaggattggctgaacggcaaggagtacaagtgcaaggtgagcaataaggccctgcccgcccctatcgagaagacaatctccaaggccaagggccagcctcgcgaaccacaggtgtatgtgctgcctccatctagagacgagctgaccaagaaccaggtgagcctgctgtgcctggtgaagggcttctaccccagcgatatcgccgtggagtgggagtccaatggccagcctgagaacaattatctgacatggccccctgtgctggactccgatggctctttctttctgtactccaagctgaccgtggacaagtctcgctggcagcagggcaacgtgtttagctgttccgtgatgcacgaggccctgcacaatcactacacccagaagtctctgagcttaagccctggc

SEQ ID NO: 136 - protein, 587 aa, Artificial Sequence
  Clone #16783 Full
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 137 - DNA, 1761 bp, Artificial Sequence
  Clone #16783 Full
gagccagccgtgtatttcaaggagcagtttctggacggcgatggctggacctctcggtggatcgagtctaagcacaagagcgatttcggcaagtttgtgctgagctccggcaagttctatggcgacgaggagaaggataagggcctgcagacatctcaggacgcccgcttttacgccctgtccgcctctttcgagccctttagcaacaagggccagaccctggtggtgcagttcacagtgaagcacgagcagaacatcgactgcggcggcggctatgtgaagctgtttcctaatagcctggaccagaccgatatgcacggcgattccgagtacaacatcatgttcggaccagacatctgcggacctggaacaaagaaggtgcacgtgatctttaattacaagggcaagaacgtgctgatcaataaggatatccggtgtaaggacgatgagttcacccacctgtacacactgatcgtgagaccagataacacctatgaggtgaagatcgacaattcccaggtggagagcggctccctggaggacgattgggactttctgccccctaagaagatcaaggacccagatgcctccaagcccgaggactgggatgagagagccaagatcgacgatcctacagattctaagccagaggactgggataagcctgagcacatccccgaccctgatgccaagaagcctgaagactgggatgaggagatggacggcgagtgggagccacccgtgatccagaaccccgagtacaagggcgagtggaagccaaggcagatcgacaatcccgattataagggcacctggattcaccccgagatcgacaaccctgagtactccccagatccctctatctacgcctatgacaatttcggcgtgctgggcctggatctgtggcaggtgaagagcggcaccatcttcgataactttctgatcacaaatgacgaggcctatgccgaggagtttggcaatgagacctggggcgtgacaaaggccgccgagaagcagatgaaggacaagcaggatgaagagcagcggctgaagggaggaggaggctccgagcccaagtctagcgacaagacccacacatgccctccatgtccggcgccagaggccgccggaggaccaagcgtgttcctgtttccacccaagcctaaggacacactgatgatcagcaggacaccagaggtgacctgcgtggtggtgtccgtgtctcacgaggaccccgaggtgaagtttaactggtacgtggatggcgtggaggtgcacaatgccaagaccaagccaagggaggagcagtataactctacataccgcgtggtgagcgtgctgaccgtgctgcaccaggattggctgaacggcaaggagtacaagtgcaaggtgagcaataaggccctgcccgcccctatcgagaagacaatctccaaggccaagggccagcctcgcgaaccacaggtgtatgtgctgcctccatctagagacgagctgaccaagaaccaggtgagcctgctgtgcctggtgaagggcttctaccccagcgatatcgccgtggagtgggagtccaatggccagcctgagaacaattatctgacatggccccctgtgctggactccgatggctctttctttctgtactccaagctgaccgtggacaagtctcgctggcagcagggcaacgtgtttagctgttccgtgatgcacgaggccctgcacaatcactacacccagaagtctctgagcttaagccctggc

SEQ ID NO: 138 - protein, 1030 aa, Artificial Sequence
  Clone #16784 Full
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAGGGGSEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 139 - DNA, 3090 bp, Artificial Sequence
  Clone #16784 Full
gagcctgccgtgtacttcaaggagcagtttctggacggcgatggctggaccagcaggtggatcgagtctaagcacaagagcgacttcggcaagtttgtgctgagctccggcaagttctacggcgacgaggagaaggataagggcctgcagacatctcaggatgccaggttttatgccctgagcgcctccttcgagccctttagcaacaagggccagaccctggtggtgcagttcacagtgaagcacgagcagaacatcgactgcggcggcggctacgtgaagctgtttcctaattccctggaccagaccgatatgcacggcgactctgagtataacatcatgttcggcccagatatctgcggccccggcacaaagaaggtgcacgtgatctttaattataagggcaagaacgtgctgatcaataaggacatccggtgtaaggacgatgagttcacccacctgtacacactgatcgtgagacctgacaacacctatgaggtgaagatcgataatagccaggtggagtctggcagcctggaggacgattgggattttctgccccctaagaagatcaaggaccctgatgccagcaagccagaggactgggatgagagagccaagatcgacgatcccacagactccaagcctgaggactgggataagccagagcacatccctgacccagatgccaagaagcccgaggactgggatgaggagatggatggcgagtgggagccacccgtgatccagaacccagagtacaagggcgagtggaagcccaggcagatcgacaatcctgattataagggcacctggattcacccagagatcgacaaccccgagtactcccccgatccttctatctacgcctatgacaatttcggcgtgctgggcctggacctgtggcaggtgaagtccggcaccatcttcgataactttctgatcacaaatgacgaggcctacgccgaggagtttggcaacgagacctggggcgtgacaaaggccgccgagaagcagatgaaggacaagcaggatgaagagcagcggctgaaggaagaggaggaggacaagaagagaaaggaggaggaggaggccgaggataaggaggacgatgaggacaaggatgaggacgaggaggacgaggaggataaggaggaggacgaggaggaggatgtgccaggacaggccggaggcggaggctccgagcctgccgtgtatttcaaggaacagtttctggatggcgacggctggacctctcgctggatcgagagcaagcacaagtctgattttggcaagtttgtgctgtctagtggcaagttctacggcgacgaagaaaaagacaaaggcctgcagacatcccaggatgcccggttttatgccctgtccgcctctttcgagccattttctaataagggacagaccctggtcgtccagttcacagtcaaacatgagcagaacatcgactgtggaggaggatatgtgaagctgtttcccaatagcctggatcagactgatatgcacggcgactccgaatacaacatcatgttcggccctgatatctgcggcccaggaacaaagaaggtccacgtgatctttaattacaaaggcaagaacgtgctgatcaataaggatatcagatgcaaagatgacgagttcacccacctgtatacactgatcgtgcgccccgataatacttacgaagtcaaaattgacaacagccaggtggagagcggctccctggaagatgattgggacttcctgcctcccaagaagatcaaggaccccgacgcctctaagcctgaggattgggacgagcgcgccaagatcgacgatccaacagacagcaagcccgaggattgggacaagcctgagcacatcccagatcccgacgccaagaagccagaggattgggacgaagaaatggacggagagtgggagccccctgtgatccagaaccctgagtataagggcgagtggaagccacggcagatcgacaatcccgattacaaaggaacctggattcaccctgagatcgataacccagagtattctcctgacccaagcatctacgcctatgataactttggcgtgctgggcttagacctgtggcaggtcaaatccggcaccatcttcgacaactttctgattaccaatgatgaagcttatgctgaagagtttggaaatgaaacttggggagtcaccaaagccgccgagaaacagatgaaagataaacaggacgaggagcagaggctgaaggaagaagaggaggacaagaagcgcaaagaagaagaagaagctgaagacaaggaggacgatgaggataaggacgaggatgaagaagatgaagaagacaaagaagaagatgaggaggaggatgtgcctggacaggccgccgccgagccaaagtcctctgacaagacccacacatgcccaccctgtccggcgccagaggccgccggaggaccaagcgtgttcctgtttccacccaagcctaaggacacactgatgatcagcaggacaccagaggtgacctgcgtggtggtgtccgtgtctcacgaggaccccgaggtgaagtttaactggtacgtggatggcgtggaggtgcacaatgccaagaccaagccaagggaggagcagtataactctacataccgcgtggtgagcgtgctgaccgtgctgcaccaggattggctgaacggcaaggagtacaagtgcaaggtgagcaataaggccctgcccgcccctatcgagaagacaatctccaaggccaagggccagcctcgcgaaccacaggtgtatgtgctgcctccatctagagacgagctgaccaagaaccaggtgagcctgctgtgcctggtgaagggcttctaccccagcgatatcgccgtggagtgggagtccaatggccagcctgagaacaattatctgacatggccccctgtgctggactccgatggctctttctttctgtactccaagctgaccgtggacaagtctcgctggcagcagggcaacgtgtttagctgttccgtgatgcacgaggccctgcacaatcactacacccagaagtctctgagcttaagccctggc

SEQ ID NO: 140 - protein, 480 aa, Artificial Sequence
  Clone #16795 Full
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 141 - DNA, 1440 bp, Artificial Sequence
  Clone #16795 Full
gacatccagatgacacagagcccaagctccctgtctgccagcgtgggcgacagggtgaccatcacatgcagggcctcccaggatgtgaacaccgccgtggcctggtaccagcagaagcctggcaaggccccaaagctgctgatctactccgcctctttcctgtattccggcgtgccttctcggtttagcggctccagatctggcaccgacttcaccctgacaatctctagcctgcagccagaggattttgccacatactattgccagcagcactataccacaccccctaccttcggccagggcacaaaggtggagatcaagggaggcagcggaggaggctccggaggaggctctggcggaggcagcggcggcggctccggcgaggtgcagctggtggagagcggcggcggcctggtgcagcctggaggctctctgaggctgagctgtgcagcctccggctttaacatcaaggacacctacatccactgggtgcggcaggcacctggcaagggactggagtgggtggccagaatctatccaaccaatggctacacacggtatgccgactccgtgaagggccggttcaccatctctgccgataccagcaagaacacagcctacctgcagatgaatagcctgcgggccgaggatacagccgtgtactattgctccagatggggcggcgacggcttctacgccatggattattggggccagggcaccctggtgacagtgtcctctgccgccgagcccaagagctccgacaagacccacacatgcccaccatgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 142 - protein, 680 aa, Artificial Sequence
  Clone #16801 Full
EVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSEVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 143 - DNA, 2040 bp, Artificial Sequence
  Clone #16801 Full
gaggtgaagctggtggagagcggaggaggactggtgcagccaggaggctctctgaagctgagctgcgccacctccggcttcacattttccgactactatatgtactgggtgcggcagaccccagagaagagactggagtgggtggcctatatcaactctggcggcggcagcacctactatcccgacacagtgaagggccggtttaccatctccagagataacgccaagaatacactgtacctgcagatgtccaggctgaagtctgaggacaccgccatgtactattgcgcacggagaggcctgccattccacgcaatggattattggggccagggcaccagcgtgacagtgagctccgcctccacaaagggacctagcgtgttcccactggccccctctagcaagtccacctctggaggaacagccgccctgggctgtctggtgaaggactacttccccgagcctgtgaccgtgagctggaactccggggccctgaccagcggagtgcacacatttcccgccgtgctgcagtcctctggcctgtactctctgagctccgtggtgaccgtgccttctagctccctgggcacccagacatatatctgcaacgtgaatcacaagccttctaatacaaaggtggacaagaaggtggagccaaagagctgtgataagacccacacaggaggaggaggcagcgaagtcaagctggtggagtctggcggcggcctggtccagcctggaggcagcctgaagctgtcctgcgccacctctggcttcacattttctgattattacatgtactgggtgaggcagacccctgagaagcgcctggaatgggtcgcctatatcaatagcggcggcggctccacctactatccagacacagtgaagggcaggttcaccatcagccgcgataatgctaaaaacaccctgtacctgcagatgtctcggctgaagagcgaggacacagccatgtactattgtgcaaggcgcggcctgccatttcacgcaatggattactggggccagggcacctccgtgacagtgtctagcgctagcaccaagggaccatccgtgttcccactggcaccaagctccaagtctacaagcggaggaaccgccgccctgggctgtctggtgaaggattacttcccagagcccgtgaccgtgtcttggaacagcggggccctgaccagcggagtgcacacctttcctgccgtgctgcagtctagcggcctgtatagcctgtcctctgtggtcacagtgccaagctcctctctgggcacacagacctacatctgcaacgtgaatcacaagccatccaataccaaggtcgacaagaaggtggagcccaagtcttgtgataagacacacacctgcccaccttgtccggcgccagaggccgccggaggaccaagcgtgttcctgtttccacccaagcctaaggacacactgatgatcagcaggacaccagaggtgacctgcgtggtggtgtccgtgtctcacgaggaccccgaggtgaagtttaactggtacgtggatggcgtggaggtgcacaatgccaagaccaagccaagggaggagcagtataactctacataccgcgtggtgagcgtgctgaccgtgctgcaccaggattggctgaacggcaaggagtacaagtgcaaggtgagcaataaggccctgcccgcccctatcgagaagacaatctccaaggccaagggccagcctcgcgaaccacaggtgtatgtgctgcctccatctagagacgagctgaccaagaaccaggtgagcctgctgtgcctggtgaagggcttctaccccagcgatatcgccgtggagtgggagtccaatggccagcctgagaacaattatctgacatggccccctgtgctggactccgatggctctttctttctgtactccaagctgaccgtggacaagtctcgctggcagcagggcaacgtgtttagctgttccgtgatgcacgaggccctgcacaatcactacacccagaagtctctgagcttaagccctggc

SEQ ID NO: 144 - protein, 678 aa, Artificial Sequence
  Clone #16802 Full
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSQVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 145 - DNA, 2034 bp, Artificial Sequence
  Clone 16802 Full
caggtgcagctggtggagtccggaggaggagtggtgcagccaggccggtctctgagactgagctgcgcagcctccggcttcaccttcagcaactacggcatgtattgggtgaggcaggcccctggcaagggactggagtgggtggccgtgatctggtacgacggctctaataagtactatgccgatagcgtgaagggccggtttaccatctctagagacaacagcaagaatacactgtatctgcagatgaacagcctgcgggccgaggataccgccgtgtactattgcgccagagacctgtggggctggtacttcgattattggggccagggcaccctggtgacagtgagctccgccagcacaaagggaccatccgtgtttccactggccccctctagcaagtccacctctggaggaacagccgccctgggctgtctggtgaaggactacttccccgagcctgtgaccgtgagctggaactccggggccctgaccagcggagtgcacacatttcccgccgtgctgcagtcctctggcctgtactctctgagctccgtggtgaccgtgccttctagctccctgggcacccagacatatatctgcaacgtgaatcacaagccttctaatacaaaggtggacaagaaggtggagccaaagagctgtgataagacccacacaggaggaggaggctcccaggtccagctggtcgagtctggcggcggcgtcgtgcagccaggcaggtccctgcgcctgtcttgcgcagccagcggcttcaccttttccaactacggaatgtattgggtgcggcaggcccccggcaagggcctggaatgggtcgccgtgatctggtatgatggcagcaataagtattacgccgattccgtgaagggcaggttcaccatctcccgcgacaactctaagaatacactgtacctgcagatgaatagcctgagggctgaagacaccgccgtgtactactgtgcccgcgacctgtggggatggtattttgactactggggacagggcaccctggtcacagtgtctagcgctagcaccaagggaccatccgtgttcccactggcaccaagctccaagtctacaagcggaggaaccgccgccctgggctgtctggtgaaggattacttcccagagcccgtgaccgtgtcttggaacagcggggccctgaccagcggagtgcacacctttcctgccgtgctgcagtctagcggcctgtatagcctgtcctctgtggtcacagtgccaagctcctctctgggcacacagacctacatctgcaacgtgaatcacaagccatccaataccaaggtcgacaagaaggtggagcccaagtcttgtgataagacacacacctgcccaccttgtccggcgccagaggccgccggaggaccaagcgtgttcctgtttccacccaagcctaaggacacactgatgatcagcaggacaccagaggtgacctgcgtggtggtgtccgtgtctcacgaggaccccgaggtgaagtttaactggtacgtggatggcgtggaggtgcacaatgccaagaccaagccaagggaggagcagtataactctacataccgcgtggtgagcgtgctgaccgtgctgcaccaggattggctgaacggcaaggagtacaagtgcaaggtgagcaataaggccctgcccgcccctatcgagaagacaatctccaaggccaagggccagcctcgcgaaccacaggtgtatgtgctgcctccatctagagacgagctgaccaagaaccaggtgagcctgctgtgcctggtgaagggcttctaccccagcgatatcgccgtggagtgggagtccaatggccagcctgagaacaattatctgacatggccccctgtgctggactccgatggctctttctttctgtactccaagctgaccgtggacaagtctcgctggcagcagggcaacgtgtttagctgttccgtgatgcacgaggccctgcacaatcactacacccagaagtctctgagcttaagccctggc

SEQ ID NO: 146 - protein, 684 aa, Artificial Sequence
  Clone #16803 Full
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSQVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 147 - DNA, 2052 bp, Artificial Sequence
  Clone #16803 Full
caggtgcagctgcagcagtccggagccgagctggccagacccggggccagcgtgaagatgagctgcaaggcctccggctacaccttcaccacatatacaatgcactgggtgaagcagagacccggacagggactggagtggatcggatacatcaaccctagctccggctacaccaactataatcagaagtttaaggacaaggccaccctgacagccgataagtctagctccaccgcctccatgcagctgtctagcctgacatctgaggacagcgccgtgtactattgcgcccgggagagagccgtgctggtgccatacgccatggattattggggccagggcaccagcgtgacagtgtcctctgcctctaccaagggccctagcgtgtttccactggcccccagctccaagagcacctccggaggaacagccgccctgggctgtctggtgaaggactatttccccgagccagtgacagtgtcctggaactctggggccctgaccagcggagtgcacacatttcctgccgtgctgcagtctagcggcctgtacagcctgtcctctgtggtgaccgtgccaagctcctctctgggcacccagacatatatctgcaacgtgaatcacaagcctagcaatacaaaggtggacaagaaggtggagccaaagtcctgtgataagacccacacaggaggaggaggctcccaggtccagctgcagcagtctggagccgagctggccaggccaggggccagcgtcaaaatgtcctgtaaagcctccggatataccttcaccacctacaccatgcattgggtcaagcagcgcccaggccagggcctggagtggatcggctacatcaatccctccagcggatatactaattacaaccagaagtttaaggataaagccaccctgacagccgataaatccagctccaccgcctccatgcaactgtctagcctgacaagcgaggactccgccgtgtactattgtgccagggagagggccgtgctggtcccttatgctatggactactggggacagggcaccagcgtcacagtgtcctctgctagcaccaagggaccatccgtgttcccactggcaccaagctccaagtctacaagcggaggaaccgccgccctgggctgtctggtgaaggattacttcccagagcccgtgaccgtgtcttggaacagcggggccctgaccagcggagtgcacacctttcctgccgtgctgcagtctagcggcctgtatagcctgtcctctgtggtcacagtgccaagctcctctctgggcacacagacctacatctgcaacgtgaatcacaagccatccaataccaaggtcgacaagaaggtggagcccaagtcttgtgataagacacacacctgcccaccttgtccggcgccagaggccgccggaggaccaagcgtgttcctgtttccacccaagcctaaggacacactgatgatcagcaggacaccagaggtgacctgcgtggtggtgtccgtgtctcacgaggaccccgaggtgaagtttaactggtacgtggatggcgtggaggtgcacaatgccaagaccaagccaagggaggagcagtataactctacataccgcgtggtgagcgtgctgaccgtgctgcaccaggattggctgaacggcaaggagtacaagtgcaaggtgagcaataaggccctgcccgcccctatcgagaagacaatctccaaggccaagggccagcctcgcgaaccacaggtgtatgtgctgcctccatctagagacgagctgaccaagaaccaggtgagcctgctgtgcctggtgaagggcttctaccccagcgatatcgccgtggagtgggagtccaatggccagcctgagaacaattatctgacatggccccctgtgctggactccgatggctctttctttctgtactccaagctgaccgtggacaagtctcgctggcagcagggcaacgtgtttagctgttccgtgatgcacgaggccctgcacaatcactacacccagaagtctctgagcttaagccctggc

SEQ ID NO: 148 - protein, 702 aa, Artificial Sequence
  Clone #16811 Full
QVQLQQSGAELARPGASVKMSCKASGYTFTTYTMHWVKQRPGQGLEWIGYINPSSGYTNYNQKFKDKATLTADKSSSTASMQLSSLTSEDSAVYYCARERAVLVPYAMDYWGQGTSVTVSSGGGGSGGGGSGGGGSGGGGSQIVLTQSPAVMSASPGEKVTITCTASSSLSYMHWFQQKPGTSPKLWLYSTSILASGVPTRFSGSGSGTSYSLTISRMEAEDAATYYCQQRSSSPFTFGSGTKLEIKGGGGSQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 149 - DNA, 2106 bp, Artificial Sequence
  Clone #16811 Full
caggtgcagctgcagcagagcggagccgagctggccagacctggggccagcgtgaagatgagctgcaaggcctccggctacacattcaccacatataccatgcactgggtgaagcagcgccctggacagggactggagtggatcggctacatcaacccaagctccggctacacaaactataatcagaagtttaaggacaaggccaccctgacagccgataagtctagctccacagcctccatgcagctgtctagcctgaccagcgaggactccgccgtgtactattgcgcccgggagagagccgtgctggtgccttacgccatggattattggggccagggcacaagcgtgaccgtgtcctctggcggcggcggctctggaggaggaggcagcggcggaggaggctccggaggcggcggctctcagatcgtgctgacccagtccccagccgtgatgagcgcctccccaggagagaaggtgaccatcacatgtaccgccagctcctctctgtcctacatgcactggttccagcagaagcccggcacatctcctaagctgtggctgtattctaccagcatcctggcctctggcgtgccaacacggttttccggctctggcagcggcacatcctactctctgaccatctccaggatggaggcagaggacgcagcaacctactattgccagcagcgcagctcctctccattcacatttggcagcggcaccaagctggagatcaagggaggaggaggctctcaggagcagctggtggagagcggcggcagactggtgacaccaggaggctctctgaccctgagctgtaaggcctccggcttcgacttcagcgcctactatatgtcctgggtgagacaggcccccggcaagggcctggaatggatcgccaccatctatcctagctccggcaagacatactatgccacctgggtgaacggcagattcaccatctctagcgacaacgcccagaatacagtggatctgcagatgaatagcctgacagccgccgacagggccacctacttctgtgcccgcgattcctatgccgacgatggggccctgttcaacatctggggccctggcacactggtgaccatctcctctgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 150 - protein, 700 aa, Artificial Sequence
  Clone #16812 Full
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIKGGGGSQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 151 - DNA, 2100 bp, Artificial Sequence
  Clone #16812 Full
caggtgcagctggtggagtccggcggcggcgtggtgcagcctggcaggtccctgcgcctgtcttgcgcagccagcggcttcaccttcagcaactacggcatgtattgggtgcggcaggcccctggcaagggactggagtgggtggccgtgatctggtacgacggcagcaataagtactatgccgattccgtgaagggccggttcaccatctccagagacaactctaagaatacactgtatctgcagatgaactccctgcgggccgaggataccgccgtgtactattgcgccagagacctgtggggctggtactttgattattggggccagggcaccctggtgacagtgagcagcggaggaggaggcagcggaggaggaggctccggaggcggcggctctggcggcggcggcagcgagatcgtgctgacccagtccccagccacactgagcctgtccccaggagagagggccaccctgtcttgtcgcgcctctcagagcgtgtctagctacctggcctggtatcagcagaagccaggacaggccccccggctgctgatctacgacgccagcaacagggcaaccggcatcccagccagattctccggctctggcagcggcacagactttaccctgacaatctcctctctggagcccgaggatttcgccgtgtactattgccagcagcggagaaattggcctctgacctttggcggcggcacaaaggtggagatcaagggaggaggaggctctcaggagcagctggtggagagcggcggcagactggtgaccccaggaggcagcctgacactgtcctgtaaggcctctggcttcgatttttccgcctactatatgtcttgggtgagacaggcccctggcaagggcctggagtggatcgccaccatctacccaagctccggcaagacctactatgccacatgggtgaacggcagattcaccatctctagcgacaacgcccagaatacagtggatctgcagatgaacagcctgaccgccgccgacagggcaacatacttctgtgcccgcgatagctatgccgacgatggggccctgttcaacatctggggaccaggcaccctggtgacaatctcctctgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 152 - protein, 701 aa, Artificial Sequence
  Clone #16813 Full
EVKLVESGGGLVQPGGSLKLSCATSGFTFSDYYMYWVRQTPEKRLEWVAYINSGGGSTYYPDTVKGRFTISRDNAKNTLYLQMSRLKSEDTAMYYCARRGLPFHAMDYWGQGTSVTVSSGGGGSGGGGSGGGGSGGGGSDIQMTQTTSSLSASLGDRVTISCSASQGISNYLNWYQQKPDGTVKLLIYYTSILHSGVPSRFSGSGSGTDYSLTIGNLEPEDIATYYCQQFNKLPPTFGGGTKLEIKGGGGSQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 153 - DNA, 2103 bp, Artificial Sequence
  Clone #16813 Full
gaggtgaagctggtggagtctggaggaggactggtgcagccaggaggcagcctgaagctgtcctgcgccacctctggcttcaccttcagcgactactatatgtactgggtgcggcagacccccgagaagagactggagtgggtggcctatatcaacagcggcggcggctccacctactatcctgacacagtgaagggcaggttcaccatctcccgcgataacgccaagaatacactgtacctgcagatgtctaggctgaagagcgaggacacagccatgtactattgcgcccggagaggcctgccttttcacgccatggattattggggccagggcaccagcgtgacagtgagcagcggaggaggaggctccggcggcggaggctctggcggcggcggcagcggaggcggcggctccgacatccagatgacccagaccacatctagcctgtccgcctctctgggcgatcgggtgacaatcagctgttccgcctctcagggcatctccaactacctgaattggtatcagcagaagcctgacggcaccgtgaagctgctgatctactatacatccatcctgcactctggcgtgccaagcagattcagcggctccggctctggaaccgactacagcctgacaatcggcaacctggagccagaggatatcgccacctactattgccagcagttcaataagctgccccctacctttggcggcggcacaaagctggagatcaagggaggaggaggctcccaggagcagctggtggagtctggcggcaggctggtgaccccaggaggctccctgacactgtcttgtaaggccagcggcttcgatttttctgcctactatatgagctgggtgcgccaggccccaggcaagggactggagtggatcgccaccatctacccctcctctggcaagacctactatgccacatgggtgaacggcagattcaccatcagctccgacaacgcccagaatacagtggatctgcagatgaatagcctgaccgccgccgacagggccacatacttctgtgcccgcgattcctatgccgacgatggggccctgttcaacatctggggaccaggcaccctggtgacaatctctagcgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 154 - protein, 863 aa, Artificial Sequence
  Clone #16814 Full
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 155 - DNA, 2589 bp, Artificial Sequence
  Clone #16814 Full
caggagcagctggtggagagcggcggcagactggtgaccccaggaggcagcctgacactgtcctgcaaggcctctggcttcgacttttccgcctactatatgtcttgggtgcggcaggcccccggcaagggactggagtggatcgccaccatctaccctagctccggcaagacctactatgccacatgggtgaacggcagattcaccatctctagcgataacgcccagaatacagtggacctgcagatgaatagcctgaccgccgccgacagggcaacatacttctgcgccagagattcctatgccgacgatggggccctgttcaacatctggggcccaggcaccctggtgacaatctcctctgctagcaccaagggaccatccgtgtttccactggcccctagctccaagtccacctctggaggaacagccgccctgggctgtctggtgaaggactatttccccgagcctgtgacagtgtcctggaactctggggccctgaccagcggagtgcacacatttcctgccgtgctgcagtctagcggcctgtatagcctgtcctctgtggtgaccgtgccaagctcctctctgggcacccagacatacatctgcaacgtgaatcacaagccaagcaatacaaaggtcgacaagaaggtggagcccaagtcctgtgataagacccacaccggcggaggaggctctgagcctgccgtgtacttcaaggagcagtttctggacggcgatggctggacctccaggtggatcgagagcaagcacaagtccgacttcggcaagtttgtgctgagctccggcaagttctatggcgatgaggagaaggacaagggcctgcagacatcccaggatgcccgcttttacgccctgagcgcctccttcgagcccttttctaataagggccagaccctggtggtgcagttcacagtgaagcacgagcagaacatcgactgtggcggcggctatgtgaagctgtttcctaattctctggatcagaccgacatgcacggcgacagcgagtacaacatcatgttcggcccagatatctgcggccccggcacaaagaaggtgcacgtgatctttaattataagggcaagaacgtgctgatcaataaggacatcaggtgtaaggacgatgagttcacccacctgtacacactgatcgtgcgcccagacaacacctatgaggtgaagatcgataatagccaggtggagtctggcagcctggaggacgattgggattttctgccccctaagaagatcaaggaccctgatgccagcaagccagaggactgggatgagcgggccaagatcgacgatcccaccgactccaagcctgaggactgggataagcctgagcacatcccagaccccgatgccaagaagcccgaagactgggatgaggagatggatggcgagtgggagccacccgtgatccagaaccccgagtacaagggcgagtggaagcctagacagatcgataatccagactataagggcacctggattcacccagagatcgataaccccgagtactctcctgacccaagcatctacgcctatgataatttcggcgtgctgggcctggacctgtggcaggtgaagtccggcaccatcttcgacaactttctgatcacaaatgatgaggcctacgccgaggagtttggcaacgagacctggggcgtgacaaaggccgccgagaagcagatgaaggataagcaggacgaggagcagaggctgaaggaagaggaggaggacaagaagcgcaaggaggaggaggaggccgaggataaggaggacgatgaggacaaggatgaggacgaggaggatgaggaggacaaggaggaggatgaggaggaggacgtgccaggacaggccgccgccgagcctaagtctagcgataagacccacacatgccctccatgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 156 - protein, 4 aa, Artificial Sequence
  linker
AAGG

SEQ ID NO: 157 - protein, 4 aa, Artificial Sequence
  linker
GGGS

SEQ ID NO: 158 - protein, 4 aa, Artificial Sequence
  Linker
GGGG

SEQ ID NO: 159 - protein, 9 aa, Artificial Sequence
  MelanA peptide
ELGIGILTV

SEQ ID NO: 160 - protein, 10 aa, Artificial Sequence
  K-ras peptide
KLVVVGAGGV

SEQ ID NO: 161 - protein, 1280 aa, Artificial Sequence
  Clone #17904 Full
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAGGGGSEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGSGGGSGGGSGGGSGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVS

SEQ ID NO: 162 - protein, 764 aa, Artificial Sequence
  Clone #17858 Full
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAAAGGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAA

SEQ ID NO: 163 - protein, 1165 aa, Artificial Sequence
  Clone #17859 Full
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAAAGGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAAGGGGSEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQA

SEQ ID NO: 164 - protein, 867 aa, Artificial Sequence
  Clone #17860 Full
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGSGGGSGGGSGGGSGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQA

SEQ ID NO: 165 - protein, 364 aa, Artificial Sequence
  Clone #9157 Full
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAA

SEQ ID NO: 166 - protein, 765 aa, Artificial Sequence
  Clone #17862 Full
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAAGGGGSEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQA

SEQ ID NO: 167 - protein, 231 aa, Artificial Sequence; identical to SEQ ID NO: 76
  Clone #12155 Full

SEQ ID NO: 168 - protein, 482 aa, Artificial Sequence
  Clone #17901 Full
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGSGGGSGGGSGGGSGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 169 - protein, 880 aa, Artificial Sequence
  Clone #17902 Full
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGSGGGSGGGSGGGSGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 170 - protein, 880 aa, Artificial Sequence
  Clone #17903 Full
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGSGGGSGGGSGGGSGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 171 - protein, 1030 aa, Artificial Sequence; identical to SEQ ID NO: 138
  Clone #16784 Full

SEQ ID NO: 172 - protein, 482 aa, Artificial Sequence
  Clone #17905 Full
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGSGGGSGGGSGGGSGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 173 - protein, 629 aa, Artificial Sequence
  Clone #17941 Full
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAAAEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 174 - protein, 224 aa, Artificial Sequence
  Clone #9158 Full
AAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 175 - protein, 231 aa, Artificial Sequence; identical to SEQ ID NO: 74
  Clone #12153 Full

SEQ ID NO: 176 - protein, 629 aa, Artificial Sequence; identical to SEQ ID NO: 56
  Clone #12667 Full

SEQ ID NO: 177 - protein, 471 aa, Artificial Sequence
  Clone #9182 Full
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGSGGGSGGGSGGGSGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 178 - protein, 364 aa, Artificial Sequence; identical to SEQ ID NO: 165
  Clone #9157 Albucore3A

SEQ ID NO: 179 - DNA, 1092 bp, Artificial Sequence
  Clone #9157 Albucore3A
gatgctcataagagcgaggtggcccacaggttcaaggacctaggcgaggagaactttaaggccctggtgctgatcgccttcgcccagtacctgcagcagtccccctttgaggaccacgtgaagctggtgaacgaggtgaccgagttcgccaagacatgcgtggccgacgagtccgccgagaattgtgataagtctctgcacaccctgtttggcgataagctgtgcaccgtggccacactgagggagacatatggcgagatggccgactgctgtgccaagcaggagcccgagcgcaacgagtgcttcctgcagcacaaggacgataaccccaatctgcctcggctggtgagacctgaggtggacgtgatgtgcaccgccttccacgataatgaggagacatttctgaagaagtacctgtatgagatcgcccggagacacccttacttttatgccccagagctgctgttctttgccaagcggtacaaggccgccttcaccgagtgctgtcaggcagcagataaggcagcatgcctgctgccaaagctggacgagctgcgggatgagggcaaggccagctccgccaagcagagactgaagtgtgcctctctgcagaagttcggagagcgggcctttaaggcatgggcagtggccaggctgtctcagcggttccccaaggccgagtttgccgaggtgagcaagctggtgaccgacctgacaaaggtgcacacagagtgctgtcacggcgacctgctggagtgcgccgacgatagagccgatctggccaagtatatctgtgagaatcaggactccatctctagcaagctgaaggagtgctgtgagaagcctctgctggagaagtctcactgcatcgccgaggtggagaacgacgagatgccagccgatctgccaagcctggccgcagactttgtggagtccaaggacgtgtgcaagaattacgccgaggccaaggacgtgttcctgggcatgtttctgtacgagtatgcccggcggcacccagactattccgtggtgctgctgctgagactggctaaaacctacgaaactactctggaaaaatgttgtgccgcggcc

SEQ ID NO: 180 - protein, 221 aa, Artificial Sequence
  Clone 9158 Albucore3B
DPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 181 - DNA, 663 bp, Artificial Sequence
  Clone 9158 Albucore3B
gacccccacgaatgctatgccaaggtgttcgatgagtttaagcctctggtggaggagccacagaacctgatcaagcagaattgtgagctgttcgagcagctgggcgagtacaagtttcagaacgccctgctggtgaggtataccaagaaggtgccccaggtgtccacccctacactggtggaggtgtctcggaatctgggcaaggtcggcagcaagtgctgtaagcacccagaggccaagaggatgccctgcgccgaggactacctgtctgtggtgctgaatcagctgtgcgtgctgcacgagaagacccccgtgagcgatagggtgaccaagtgctgtacagagtccctggtcaaccggagaccctgcttttctgccctggaggtggacgagacatatgtgcctaaggagttcaatgccgagaccttcacatttcacgccgatatctgtaccctgagcgagaaggagcgccagatcaagaagcagacagccctggtggagctggtgaagcacaagcctaaggccaccaaggagcagctgaaggccgtgatggacgatttcgccgcctttgtggagaagtgctgtaaggccgacgataaggagacatgcttcgcagaggagggcaagaagctggtggcagcctcccaggccgccctaggcctg

SEQ ID NO: 182 - protein, 247 aa, Artificial Sequence
  Clone #17901 Trast scFv
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGSGGGSGGGSGGGSGGGSGEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 183 - protein, 8 aa, Artificial Sequence
  Clone 12E12 CDRH1
GFTFSDYY

SEQ ID NO: 184 - protein, 8 aa, Artificial Sequence
  Clone 12E12 CDR H2
INSGGGST

SEQ ID NO: 185 - protein, 12 aa, Artificial Sequence
  Clone 12E12 CDR H3
ARRGLPFHAMDY

SEQ ID NO: 186 - protein, 6 aa, Artificial Sequence
  Clone 12E12 CDR L1
QGISNY

SEQ ID NO: 187 - protein, 3 aa, Artificial Sequence
  Clone 12E12 CDR L2
YTS

SEQ ID NO: 188 - protein, 9 aa, Artificial Sequence
  Clone 12E12 CDR L3
QQFNKLPPT

SEQ ID NO: 189 - protein, 8 aa, Artificial Sequence
  Clone 3G9 CDR H1
GFTFSNYG

SEQ ID NO: 190 - protein, 8 aa, Artificial Sequence
  Clone 3G9 CDR H2
IWYDGSNK

SEQ ID NO: 191 - protein, 11 aa, Artificial Sequence
  Clone 3G9 CDR H3
ARDLWGWYFDY

SEQ ID NO: 192 - protein, 6 aa, Artificial Sequence
  Clone 3G9 CDR L1
QSVSSY

SEQ ID NO: 193 - protein, 3 aa, Artificial Sequence
  Clone 3G9 CDR L2
DAS

SEQ ID NO: 194 - protein, 9 aa, Artificial Sequence
  Clone 3G9 CDR L3
QQRRNWPLT

SEQ ID NO: 195 - protein, 8 aa, Artificial Sequence
  Clone 15E2.5 CDR H1
GYTFTTYT

SEQ ID NO: 196 - protein, 8 aa, Artificial Sequence
  Clone 15E2.5 CDR H2
INPSSGYT

SEQ ID NO: 197 - protein, 14 aa, Artificial Sequence
  Clone 15E2.5 CDR H3
ARERAVLVPYAMDY

SEQ ID NO: 198 - protein, 5 aa, Artificial Sequence
  Clone 15E2.5 CDR L1
SSLSY

SEQ ID NO: 199 - protein, 3 aa, Artificial Sequence
  Clone 15E2.5 CDR L2
STS

SEQ ID NO: 200 - protein, 9 aa, Artificial Sequence
  Clone 15E2.5 CDR L3
QQRSSSPFT

SEQ ID NO: 201 - protein, 8 aa, Artificial Sequence
  Clone 2D8.2D4 CDR H1
GYSFTGYN

SEQ ID NO: 202 - protein, 8 aa, Artificial Sequence
  Clone 2D8.2D4 CDR H2
IDPYYGDT

SEQ ID NO: 203 - protein, 12 aa, Artificial Sequence
  Clone 2D8.2D4 CDR H3
ARPYGSEAYFAY

SEQ ID NO: 204 - protein, 6 aa, Artificial Sequence
  Clone 2D8.2D4 CDR L1
QSISDY

SEQ ID NO: 205 - protein, 3 aa, Artificial Sequence
  Clone 2D8.2D4 CDR L2
YAA

SEQ ID NO: 206 - protein, 9 aa, Artificial Sequence
  Clone 2D8.2D4 CDR L3
QNGHSFPYT

SEQ ID NO: 207 - protein, 8 aa, Artificial Sequence
  Clone 11B6.4 CDR H1
GFSLSNYD

SEQ ID NO: 208 - protein, 7 aa, Artificial Sequence
  Clone 11B6.4 CDR H2
MWTGGGA

SEQ ID NO: 209 - protein, 12 aa, Artificial Sequence
  Clone 11B6.4 CDR H3
VRDAVRYWNFDV

SEQ ID NO: 210 - protein, 5 aa, Artificial Sequence
  Clone 11B6.4 CDR L1
SSVSY

SEQ ID NO: 211 - protein, 3 aa, Artificial Sequence
  CLone 11B6.4 CDR L2
ATS

SEQ ID NO: 212 - protein, 9 aa, Artificial Sequence
  Clone 11B6.4 CDR L3
QQWSSNPFT

SEQ ID NO: 213 - protein, 8 aa, Artificial Sequence
  Pertuzumab CDR H1
GFTFTDYT

SEQ ID NO: 214 - protein, 8 aa, Artificial Sequence
  Pertuzumab CDR H2
VNPNSGGS

SEQ ID NO: 215 - protein, 12 aa, Artificial Sequence
  Pertuzumab CDR H3
ARNLGPSFYFDY

SEQ ID NO: 216 - protein, 6 aa, Artificial Sequence
  Pertuzumab CDR L1
QDVSIG

SEQ ID NO: 217 - protein, 3 aa, Artificial Sequence
  Pertuzumab CDR L2
SAS

SEQ ID NO: 218 - protein, 9 aa, Artificial Sequence
  Pertuzumab CDR L3
QQYYIYPYT

SEQ ID NO: 219 - protein, 8 aa, Artificial Sequence
  Clone RG7787 CDR H1
GYSFTGYT

SEQ ID NO: 220 - protein, 8 aa, Artificial Sequence
  Clone RG7787 CDR H2
ITPYNGAS

SEQ ID NO: 221 - protein, 12 aa, Artificial Sequence
  Clone RG7787 CDR H3
ARGGYDGRGFDY

SEQ ID NO: 222 - protein, 5 aa, Artificial Sequence
  Clone RG7787 CDR L1
SSVSY

SEQ ID NO: 223 - protein, 3 aa, Artificial Sequence
  Clone RG7787 CDR L2
DTS

SEQ ID NO: 224 - protein, 9 aa, Artificial Sequence
  Clone RG7787 CDR L3
QQWSKHPLT

SEQ ID NO: 225 - protein, 8 aa, Artificial Sequence
  Clone MLN2704 CDR H1
GYTFTEYT

SEQ ID NO: 226 - protein, 8 aa, Artificial Sequence
  Clone MLN2704 CDR H2
INPNNGGT

SEQ ID NO: 227 - protein, 8 aa, Artificial Sequence
  Clone MLN2704 CDR H3
AAGWNFDY

SEQ ID NO: 228 - protein, 6 aa, Artificial Sequence
  Clone MLN2704 CDR L1
QDVGTA

SEQ ID NO: 229 - protein, 3 aa, Artificial Sequence
  Clone MLN2704 CDR L2
WAS

SEQ ID NO: 230 - protein, 9 aa, Artificial Sequence
  Clone MLN2704 CDR L3
QQYNSYPLT

SEQ ID NO: 231 - protein, 8 aa, Artificial Sequence
  Clone R12 CDR H1
GFDFSAYY

SEQ ID NO: 232 - protein, 8 aa, Artificial Sequence
  Clone R12 CDR H2
IYPSSGKT

SEQ ID NO: 233 - protein, 14 aa, Artificial Sequence
  Clone R12 CDR H3
ARDSYADDGALFNI

SEQ ID NO: 234 - protein, 7 aa, Artificial Sequence
  Clone R12 CDR L1
SAHKTDT

SEQ ID NO: 235 - protein, 7 aa, Artificial Sequence
  Clone R12 CDR L2
VQSDGSY

SEQ ID NO: 236 - protein, 9 aa, Artificial Sequence
  Clone R12 CDR L3
GADYIGGYV

(19) United States
(12) Patent Application Publication
(10) Pub. No.: US 2020/0048371 A1
(43) Pub. Date: Feb. 13, 2020

(54) TUMOR ANTIGEN PRESENTATION INDUCER CONSTRUCTS AND USES THEREOF

(71) Applicant: Zymeworks Inc., Vancouver (CA)

(72) Inventors: David M. Mills, Seattle, WA (US); Eric Escobar-Cabrera, Burnaby (CA)

(21) Appl. No.: 16/499,808

(22) PCT Filed: Mar. 29, 2018
(86) PCT No.: PCT/CA2018/050401

§ 371 Date: Sep. 30, 2019

(51) Int. Cl. C07K 16/46; C07K 16/28; C07K 16/30; C07K 16/32; C12N 5/0783; C12N 15/10; G01N 33/50; A61P 35/00; A61K 35/17

(52) U.S. Cl. CPC C07K 16/468; A61K 2039/505; C07K 16/2878; C07K 16/28; C07K 16/30; C07K 16/32; C07K 16/2803; C07K 16/3069; C12N 5/0636; C12N 15/1093; G01N 33/505; A61P 35/00; A61K 35/17; C07K 2317/31; C07K 2317/52; C07K 2317/522; C07K 2317/55; C07K 2317/622; C12N 2502/30; C07K 16/2851

(57) ABSTRACT

Provided herein are tumor-associated antigen (TAA) presentation inducer constructs comprising at least one innate stimulatory receptor (ISR)-binding construct that binds to an ISR expressed on an antigen-presenting cell (APC), and at least one TAA-binding construct that binds directly to a first TAA that is physically associated with tumor cell-derived material (TCDM) comprising one or more other TAAs. The ISR-binding construct and TAA-binding construct are linked to each other, and the TAA presentation inducer construct induces a polyclonal T cell response to the first TAA and to the one or more other TAAs. Also provided are methods of using the TAA presentation inducer constructs, for example, in the treatment of cancer.

### BACKGROUND

Although neoplastic transformation invariably involves tumor-associated antigen (TAA) emergence, self-tolerance mechanisms often limit TAA-specific T lymphocyte activation. Accordingly, though immune checkpoint blockade (e.g. anti-CTLA-4 and anti-PD-1/PD-L1) has revolutionized cancer immunotherapy, a large patient percentage remains non-responsive due to lack of pre-existing TAA-specific T cells (Yuan et al., 2011 PNAS 108:16723-16728). Treatments that increase endogenous TAA-directed T cell responses may be required for long-lasting, broad-acting anti-tumor immunity.

Numerous tumor vaccine approaches have attempted to overcome TAA tolerance, but have exhibited limited efficacy due to heterogeneity in expression of TAAs. For example, transformed cells that lack or downregulate TAA expression can persist post-vaccination and promote relapse. Because neoplastic cell TAA landscapes are heterogeneous and dynamic, vaccine approaches that rely on pre-defined TAA mixtures have been minimally efficacious, and therapies that overcome immunologic tolerance to multiple, diverse TAAs, and adapt with evolving TAA expression patterns are needed.

### SUMMARY

Described herein are tumor-associated antigen (TAA) presentation inducer constructs and uses thereof. One aspect of the present disclosure relates to tumor-associated antigen (TAA) presentation inducer constructs comprising: a) at least one innate stimulatory receptor (ISR)-binding construct that binds to an ISR expressed on an antigen-presenting cell (APC), and b) at least one TAA-binding construct that binds directly to a first TAA that is physically associated with tumor cell-derived material (TCDM) comprising one or more other TAAs, wherein said ISR-binding construct and said TAA-binding construct are linked to each other, and wherein the TAA presentation inducer construct induces a polyclonal T cell response to the one or more other TAAs.

Another aspect of the present disclosure relates to a pharmaceutical composition comprising the TAA presentation inducer construct described herein.

Another aspect of the present disclosure relates to one or more nucleic acids encoding the TAA presentation inducer construct described herein.

Another aspect of the present disclosure relates to one or more vectors comprising one or more nucleic acids encoding the TAA presentation inducer construct described herein.

Another aspect of the present disclosure relates to a host cell comprising one or more nucleic acids encoding the TAA presentation inducer construct described herein, or comprising one or more vectors comprising one or more nucleic acids encoding the TAA presentation inducer construct described herein.

Another aspect of the present disclosure relates to a method of making the tumor-associated antigen (TAA) presentation inducer construct described herein comprising: expressing one or more nucleic acids encoding the TAA presentation inducer construct described herein, or one or more vectors comprising one or more nucleic acids encoding the TAA presentation inducer construct described herein, in a cell.

Another aspect of the present disclosure relates to a method of treating cancer comprising administering the tumor-associated antigen (TAA) presentation inducer construct described herein to a subject in need thereof.

Another aspect of the present disclosure relates to a method of inducing major histocompatibility complex (MHC) presentation of peptides from two or more tumor-associated antigens (TAAs) by a single innate stimulatory receptor-expressing cell simultaneously in a subject, comprising administering to the subject the TAA presentation inducer construct described herein.

Another aspect of the present disclosure relates to a method of inducing innate stimulatory receptor-expressing cell activation in a subject, comprising administering to the subject, the tumor-associated antigen (TAA) presentation inducer construct described herein.

Another aspect of the present disclosure relates to a method of inducing a polyclonal T cell response in a subject, comprising administering to the subject the tumor-associated antigen (TAA) presentation inducer construct described herein.

Another aspect of the present disclosure relates to a method of expanding, activating, or differentiating T cells specific for two or more tumor-associated antigens (TAAs) simultaneously, comprising: obtaining T cells and innate stimulatory receptor (ISR)-expressing cells from a subject; and culturing the T cells and the ISR-expressing cells with the TAA presentation inducer construct described herein in the presence of tumor cell-derived material (TCDM), to produce expanded, activated or differentiated T cells.

Another aspect of the present disclosure relates to a method of treating cancer in a subject, comprising administering to the subject the expanded, activated or differentiated T cells prepared according to the method described herein.

Another aspect of the present disclosure relates to a method of identifying tumor-associated antigens in tumor cell-derived material (TCDM) comprising: isolating T cells and enriched innate stimulatory receptor (ISR)-expressing cells from a subject; culturing the ISR-expressing cells and the T cells with the TAA presentation inducer construct described herein in the presence of tumor cell-derived material (TCDM), to produce TAA presentation inducer construct-activated ISR-expressing cells, and determining the sequence of TAA peptides eluted from MHC complexes of the TAA presentation inducer construct-activated ISR-expressing cells; and identifying the TAAs corresponding to the TAA peptides.

Another aspect of the present disclosure relates to a method of identifying T cell receptor (TCR) target polypeptides, comprising: isolating T cells and enriched innate stimulatory receptor (ISR)-expressing cells from a subject; culturing the ISR-expressing cells and the T cells with the TAA presentation inducer construct described herein in the presence of tumor cell-derived material (TCDM), to produce TAA presentation inducer construct-activated ISR-expressing cells and activated T cells, and screening the activated T cells against a library of candidate TAAs to identify the TCR target polypeptides.

### BRIEF DESCRIPTION OF THE FIGURES

FIG. 1 illustrates how an exemplary TAA presentation inducer construct may target an APC to TCDM or vice-versa. In this figure, the TAA presentation inducer construct is a bispecific antibody that binds to an ISR expressed on an APC, and to TAA1. Neoplastic cells give rise to exosomes and apoptotic/necrotic debris, also called tumor cell-derived material (TCDM) when they die. TCDM contains multiple TAAs, for example, TAA1-6, and neoTAA1-2. Binding of the TAA presentation inducer construct to TAA1 and the ISR targets an innate immune cell such as an APC to the TCDM (or vice-versa). The APC may then internalize the TCDM to promote a polyclonal T cell response to one or more of TAA2-6 and neoTAA1-2. In some embodiments, the APC may also promote a polyclonal T cell response to TAA1 in addition to one or more of TAA2-6 and neoTAA1-2. The preceding description is for illustrative purposes and is not meant to be limited in any way to the type of TAA presentation inducer construct or type of number of TAAs, or other aspect of this Figure.

FIG. 2 illustrates exemplary general formats for TAA presentation inducer constructs in a bispecific antibody format. The constructs in FIGS. 2A, 2B, and 2D comprise an Fc, while the construct in FIG. 2C does not. FIG. 2A depicts a Fab-scFv format in which one antigen-binding domain is a Fab and the other is an scFv. FIG. 2B depicts a Fab-Fab format in which both antigen-binding domains are Fabs. This format is also referred to as full-size format (FSA). FIGS. 2C and 2D depict dual scFv formats in which two scFvs are either linked to each other (FIG. 2C) or linked to an Fc (FIG. 2D).

FIG. 3 illustrates additional exemplary formats for TAA presentation inducer constructs in a bispecific antibody format. The legend identifies different segments of the constructs and different fills (black versus grey) are used to represent segments that bind to distinct targets, or to represent a heterodimeric Fc. In some cases, these formats exhibit more than one valency for a target TAA or ISR. FIG. 3A depicts Format A: A_scFv_B_scFv_Fab, where Heavy Chain A includes an scFv and Heavy Chain B includes an scFv and a Fab. FIG. 3B depicts Format B: A_scFv_Fab_B_scFv, where Heavy Chain A includes an scFv and a Fab and Heavy Chain B includes an scFv. FIG. 3C depicts Format C: A_Fab_B_scFv_scFv, where Heavy Chain A includes a Fab and Heavy Chain B includes two scFvs. FIG. 3D depicts Format D: A_scFv_B_Fab_Fab, where Heavy Chain A includes an scFv and Heavy Chain B includes two Fabs. FIG. 3E depicts Format E: Hybrid, where Heavy Chain A includes a Fab and Heavy Chain B includes an scFv. FIG. 3F depicts Format F: A_Fab_CRT_B_CRT, where Heavy Chain A includes a Fab and calreticulin and Heavy Chain B includes calreticulin (CRT). FIG. 3G depicts Format G: A_Fab_CRT_B_CRT_CRT, where Heavy Chain A includes a Fab and calreticulin and Heavy Chain B includes two calreticulin polypeptides.

FIG. 4 illustrates exemplary formats for TAA presentation inducer constructs designed using split-albumin scaffolds, where “T” represents a trastuzumab scFv and “CRT” represents residues 18-417 of calreticulin. The formats of variants 15019, 15025, and 22923-22927 are illustrated.

FIG. 5 illustrates exemplary formats for TAA presentation inducer constructs designed using a heterodimeric Fc as a scaffold, where “T” represents a trastuzumab scFv and “CRT” represents residues 18-417 of calreticulin. The formats of variants 22976-22982, 21479, 23044, 22275, and 23085 are illustrated. Black versus grey fill is used to distinguish individual Fc polypeptides of the heterodimeric Fc.

FIG. 6 depicts native target binding of constructs targeting HER2, ROR1, DECTIN1, CD40, or DEC205 transiently expressed in HEK293 cells. FIG. 6A depicts HER2 binding, FIG. 6B depicts ROR1 binding, FIG. 6C depicts dectin-1 binding, FIG. 6D depicts CD40 binding, and FIG. 6E and FIG. 6F both depict DEC205 binding.

FIG. 7 depicts native binding of constructs targeting mesothelin (MSLN) endogeneously expressed in H226 cells.

FIG. 8 depicts soluble binding of mouse anti-calreticulin (CRT) MAB3898 antibody from R&D Systems to TAA presentation inducer constructs containing a CRT-arm.

FIG. 9 illustrates TAA presentation inducer construct potentiation of tumor cell material phagocytosis.

FIG. 10 depicts the ability of TAA presentation inducer constructs to potentiate monocyte cytokine production in tumor cell co-cultures. FIG. 10A depicts the ability of construct Her2×CD40 (v18532) to potentiate cytokine production and FIG. 10B depicts the ability of construct Her2×CRT (v18535) to potentiate cytokine production.

FIG. 11 depicts the effect of TAA presentation inducer constructs on IFNγ production of MelanA-enriched CD8⁺ T cells. FIG. 11A depicts the effect in APCs incubated with OVCAR3 cells containing the MelanA peptide while FIG. 11B depicts the effect in APCs incubated with OVCAR3 cells containing a plasmid encoding a MelanA-GFP fusion protein.

### DETAILED DESCRIPTION

Described herein is a multispecific tumor-associated antigen (TAA) presentation inducer construct that binds to at least one innate stimulatory receptor (ISR) expressed on an antigen-presenting cell (APC), and also directly binds to at least one first TAA. In some embodiments, the ISR may be a C-type lectin receptor, a tumor necrosis factor family receptor, or a lipoprotein receptor. The at least one first TAA may be an antigen that is physically associated with tumor cell-derived material (TCDM) comprising, or physically associated, with one or more other TAAs distinct from the first TAA. The TAA presentation inducer constructs can bind to the at least one ISR on the APC and to the at least one first TAA to induce a polyclonal T cell response to at least the one or more other TAAs physically associated with the TCDM. In one embodiment, the TAA presentation inducer construct can induce a polyclonal T cell response to the at least one first TAA as well as to the one or more other TAAs physically associated with the TCDM. The TAA presentation inducer construct may also promote TAA cross presentation in the APC. The at least one first TAA can act as a “handle” to facilitate polyclonal immunity to diverse TAAs in the presence of a TAA presentation inducer construct. In one embodiment, the TAA presentation inducer construct may be able to maintain the ability to induce a polyclonal T cell response to multiple TAAs as the TAA composition of the TCDM changes.

The TAA presentation inducer constructs may be used to treat cancer in a subject. The TAA presentation inducer described here may also be used to expand, activate, or differentiate T-cells specific for two or more TAAs simultaneously, identify TAAs in TCDM, and identify T-cell receptor target polypeptides.

### Definitions

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means±1%, 2%, 3%, 4%, 5%, 6%, 7%, 8%, 9% or 10% of the indicated range, value, sequence, or structure, unless otherwise indicated. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives. As used herein, the terms “include” and “comprise” are used synonymously.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

In the present application, amino acid names and atom names (e.g. N, O, C, etc.) are used as defined by the Protein DataBank (PDB) (www.pdb.org), which is based on the IUPAC nomenclature (IUPAC Nomenclature and Symbolism for Amino Acids and Peptides (residue names, atom names etc.), Eur. J. Biochem., 138, 9-37 (1984) together with their corrections in Eur. J. Biochem., 152, 1 (1985). The term “amino acid residue” is primarily intended to indicate an amino acid residue contained in the group consisting of the 20 naturally occurring amino acids, i.e. alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues.

Terms understood by those in the art of antibody technology are each given the meaning acquired in the art, unless expressly defined differently herein. Antibodies are known to have variable regions, a hinge region, and constant domains. Immunoglobulin structure and function are reviewed, for example, in Harlow et al, Eds., Antibodies: A Laboratory Manual, Chapter 14 (Cold Spring Harbor Laboratory, Cold Spring Harbor, 1988).

The terms “variant” and “construct” are used interchangeably herein. For example, variant 22211, construct 22211, and v22211 refer to the same TAA presentation inducer construct.

As used herein, the terms “antibody” and “immunoglobulin” or “antigen-binding construct” are used interchangeably. An “antigen-binding construct” refers to a polypeptide substantially encoded by an immunoglobulin gene or immunoglobulin genes, or one or more fragments thereof, which specifically bind an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin isotypes, IgG, IgM, IgA, IgD, and IgE, respectively. Further, the antibody can belong to one of a number of subtypes, for instance, the IgG can belong to the IgG1, IgG2, IgG3, or IgG4 subtypes.

An exemplary immunoglobulin (antibody) structural unit is composed of two pairs of polypeptide chains, each pair having one immunoglobulin “light” (about 25 kD) and one immunoglobulin “heavy” chain (about 50-70 kD). This type of immunoglobulin or antibody structural unit is considered to be “naturally occurring.” The term “light chain” includes a full-length light chain and fragments thereof having sufficient variable domain sequence to confer binding specificity. A full-length light chain includes a variable domain, VL, and a constant domain, CL. The variable domain of the light chain is at the amino-terminus of the polypeptide. Light chains include kappa chains and lambda chains. The term “heavy chain” includes a full-length heavy chain and fragments thereof having sufficient variable region sequence to confer binding specificity. A full-length heavy chain includes a variable domain, VH, and three constant domains, CH1, CH2, and CH3. The VH domain is at the amino-terminus of the polypeptide, and the CH domains are at the carboxyl-terminus, with the CH3 being closest to the carboxy-terminus of the polypeptide. Heavy chains can be of any isotype, including IgG (including IgG1, IgG2, IgG3 and IgG4 subclasses), IgA (including IgA1 and IgA2 subclasses), IgM, IgD and IgE. The term “variable region” or “variable domain” refers to a portion of the light and/or heavy chains of an antibody generally responsible for antigen recognition, typically including approximately the amino-terminal 120 to 130 amino acids in the heavy chain (VH) and about 100 to 110 amino terminal amino acids in the light chain (VL).

A “complementarity determining region” or “CDR” is an amino acid sequence that contributes to antigen-binding specificity and affinity. “Framework” regions (FR) can aid in maintaining the proper conformation of the CDRs to promote binding between the antigen-binding region and an antigen. Structurally, framework regions can be located in antibodies between CDRs. The variable regions typically exhibit the same general structure of relatively conserved framework regions (FR) joined by three hyper variable regions, CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3, and FR4. The assignment of amino acids to each domain is typically in accordance with the definitions of Kabat Sequences of Proteins of Immunological Interest (National Institutes of Health, Bethesda, Md. (1987 and 1991)), unless stated otherwise.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

An “antigen-binding construct” or “antibody” is one that targets or binds to at least one distinct antigen or epitope. A “bispecific,” “dual-specific” or “bifunctional” antigen-binding construct or antibody is a species of antigen-binding construct that targets or binds to two different antigens or epitopes. In general, a bispecific antigen-binding construct can have two different antigen-binding domains. The two antigen-binding domains of a bispecific antigen-binding construct or antibody will bind to two different epitopes, which can reside on the same or different molecular targets. In one embodiment, the bispecific antigen-binding construct is in a naturally occurring format, also referred to herein as a full-sized (FSA) format. In other words, the bispecific antigen-binding construct has the same format as a naturally occurring IgG, IgA, IgM, IgD, or IgE antibody.

As is known in the art, antigen-binding domains can be of different formats, and some non-limiting examples include Fab fragment, scFv, VHH, or sdAb, described below. Furthermore, methods of converting between types of antigen-binding domains are known in the art (see, for example, methods for converting an scFv to a Fab format described in Zhou et al (2012) Mol Cancer Ther 11:1167-1476). Thus, if an antibody is available in a format that includes an antigen-binding domain that is an scFv, but the TAA presentation inducer construct requires that the antigen-binding domain be Fab, one of skill in the art would be able to make such conversion, and vice-versa.

A “Fab fragment” (also referred to as fragment antigen-binding) contains the constant domain (CL) of the light chain and the constant domain 1 (CH1) of the heavy chain along with the variable domains VL and VH on the light and heavy chains, respectively. The variable domains comprise the CDRs, which are involved in antigen-binding. Fab′ fragments differ from Fab fragments by the addition of a few amino acid residues at the C-terminus of the heavy chain CH1 domain, including one or more cysteines from the antibody hinge region.

A “single-chain Fv” or “scFv” includes the VH and VL domains of an antibody in a single polypeptide chain. The scFv polypeptide may optionally further comprise a polypeptide linker between the VH and VL domains which enables the scFv to form a desired structure for antigen binding. For a review of scFv's see Pluckthun in The Pharmacology of Monoclonal Antibodies, vol. 113, Rosenburg and Moore eds., Springer-Verlag, New York, pp. 269-315 (1994).

A “single domain antibody” or “sdAb” format refers to a single immunoglobulin domain. The sdAb may be, for example, of camelid origin. Camelid antibodies lack light chains and their antigen-binding sites consist of a single domain, termed a “VHH.” An sdAb comprises three CDR/hypervariable loops that form the antigen-binding site: CDR1, CDR2 and CDR3. SdAbs are fairly stable and easy to express as in fusion with the Fc chain of an antibody (see, for example, Harmsen M M, De Haard H J (2007) “Properties, production, and applications of camelid single-domain antibody fragments,” Appl. Microbiol Biotechnol. 77(1): 13-22).

Antibody heavy chains pair with antibody light chains and meet or contact one another at one or more “interfaces.” An “interface” includes one or more “contact” amino acid residues in a first polypeptide that interact with one or more “contact” amino acid residues of a second polypeptide. For example, an interface exists between the two CH3 domains of a dimerized Fc region, between the CH1 domain of the heavy chain and CL domain of the light chain, and between the VH domain of the heavy chain and the VL domain of the light chain. The “interface” can be derived from an IgG antibody and for example, from a human IgG1 antibody.

The term “amino acid modifications” as used herein includes, but is not limited to, amino acid insertions, deletions, substitutions, chemical modifications, physical modifications, and rearrangements.

The amino acid residues for the immunoglobulin heavy and light chains may be numbered according to several conventions including Kabat (as described in Kabat and Wu, 1991; Kabat et al, Sequences of proteins of immunological interest. 5th Edition—US Department of Health and Human Services, NIH publication no. 91-3242, p 647 (1991)), IMGT (as set forth in Lefranc, M.-P., et al. IMGT®, the international ImMunoGeneTics information System® Nucl. Acids Res, 37, D1006-D1012 (2009), and Lefranc, M.-P., IMGT, the International ImMunoGeneTics Information System, Cold Spring Harb Protoc. 2011 Jun. 1; 2011(6)), 1JPT (as described in Katja Faelber, Daniel Kirchhofer, Leonard Presta, Robert F Kelley, Yves A Muller, The 1.85 Å resolution crystal structures of tissue factor in complex with humanized fab d3h44 and of free humanized fab d3h44: revisiting the solvation of antigen combining sites1, Journal of Molecular Biology, Volume 313, Issue 1, Pages 83-97) and EU (according to the EU index as in Kabat referring to the numbering of the EU antibody (Edelman et al., 1969, Proc Natl Acad Sci USA 63:78-85)). Kabat numbering is used herein for the VH, CHL CL, and VL domains unless otherwise indicated. EU numbering is used herein for the CH3 and CH2 domains, and the hinge region unless otherwise indicated.

### TAA Presentation Inducer Constructs

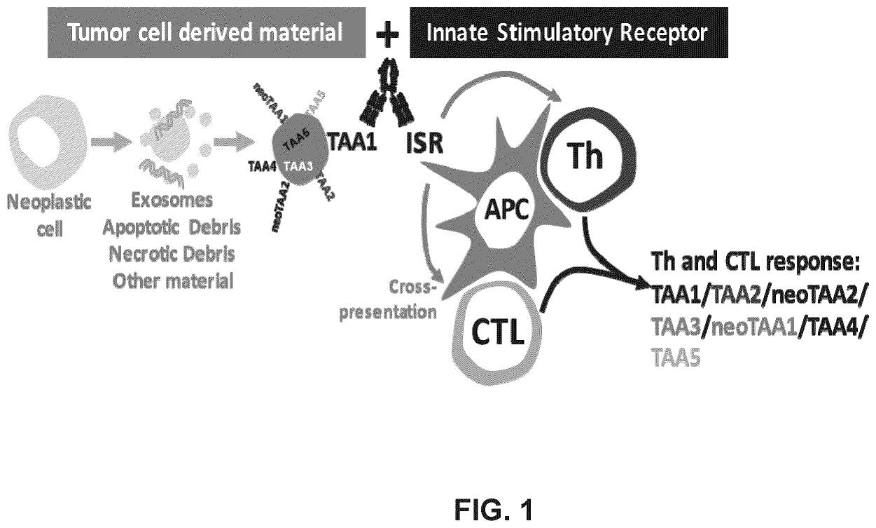

Described herein is a tumor-associated antigen (TAA) presentation inducer construct that comprises at least one innate stimulatory receptor (ISR)-binding construct and least one TAA-binding construct, linked to each other. The ISR-binding construct binds to an ISR expressed on an APC, and the TAA-binding construct binds to at least one first TAA, or “handle TAA” that is physically associated with tumor cell-derived material (TCDM) comprising, or physically associated with, one or more other TAAs, also referred to herein as “one or more secondary TAAs.” Without being limited to theory or mechanism, the TAA presentation inducer construct may act to target the APC to the TCDM, or vice-versa, to induce a polyclonal T cell response to one or more of the secondary TAAs. In some embodiments, the TAA presentation inducer construct may act to target the APC to the TCDM, or vice-versa, to induce a polyclonal T cells response to the first TAA in addition to one or more of the secondary TAAs. FIG. 1 provides a diagram illustrating how a TAA presentation inducer construct may target an APC to TCDM or vice-versa. In some embodiments, the TAA presentation inducer construct may also direct acquisition of the TCDM by the APC, i.e. promote physical attachment of TCDM to the surface of the APC. In one embodiment, the TAA presentation inducer construct may direct acquisition and internalization of the TCDM by the APC.

In one embodiment, the TAA presentation inducer construct may be capable of inducing a polyclonal T cell response that is capable of adapting to the heterogeneity and dynamic nature of neoplastic cells.

In some embodiments, the TAA presentation inducer construct can promote MHC cross-presentation of one or more TCDM-derived peptides from multiple different TAAs. In one embodiment, the TAA presentation inducer construct can induce APC activation and/or maturation of APCs presenting the one or more TCDM-derived peptides.

In one embodiment, the TAA presentation inducer construct may induce a polyclonal T cell response to both the first TAA or handle TAA and to the one or more secondary TAAs. The term “polyclonal T cell response” refers to the activation of multiple T cell clones recognizing a specific antigen. In one embodiment, the polyclonal T cell response may be MHC class I-, II-, or non-classical MHC restricted. In various embodiments, the TAA presentation inducer construct may induce a polyclonal T cell response wherein the T cells are selected from CD8+ alpha-beta T cells, CD4+ alpha-beta T cells, gamma-delta T cells, or NKT (natural killer T) cells. In some embodiments, the TAA presentation inducer construct may induce a polyclonal T cell response that involves clonal expansion and proliferation and may involve acquisition of cytotoxic and/or “helper” functions. Helper functions may involve cytokine, chemokine, growth factor, and/or costimulatory cell surface receptor expression.

The term “tumor cell-derived material” or “TCDM” refers to sub-cellular material, such as proteins, lipids, carbohydrates, nucleic acids, glycans, or combinations thereof, that originates from neoplastic or transformed cells. TCDM may also include damage-associated molecular patterns (DAMPs). Exosomes, apoptotic debris, and necrotic debris are non-limiting examples of TCDM. Thus, TCDM comprises numerous TAAs, including the handle TAAs and secondary TAAs described herein.

### Innate Stimulatory Receptor (ISR)-Binding Construct

The at least one ISR-binding construct of the TAA presentation inducer constructs described herein binds to an ISR that is expressed on the surface of an innate immune cell, or other cell expressing MI-1C class I and/or MI-1C class II, and capable of mediating T-lymphocyte activation. The ISR may be a cell surface receptor capable of inducing an activating signal in innate immune cells. Activating signals may include those that increase survival, proliferation, maturation, cytokine secretion, phagocytosis, pinocytosis, receptor internalization, ligand processing for antigen presentation, adhesion, extravasation, and/or trafficking to lymphatic or blood circulation. ISRs may be expressed by innate immune cells and other cell types, including mast cells, phagocytic cells, basophils, eosinophils, natural killer cells, and γδ T cells. In one embodiment, the TAA presentation inducer construct comprises at least one ISR-binding construct that binds to an ISR expressed on the surface of an innate immune cell. In one embodiment, the TAA presentation inducer construct comprises at least one ISR-binding construct that binds to an ISR expressed on the surface of a human innate immune cell, cynomolgous monkey innate immune cell, rhesus monkey innate immune cell, or mouse innate immune cell.

In one embodiment, the TAA presentation inducer construct comprises at least one ISR-binding construct that binds to an ISR expressed on the surface of a phagocytic innate immune cell, or other cell type expressing MI-1C class I and/or MI-1C class II. In one embodiment, the innate immune cell is an antigen-presenting cell (APC). In one embodiment, the TAA presentation inducer construct comprises at least one ISR-binding construct that binds to an ISR expressed on the surface of a hematopoietic APC. Examples of hematopoietic APCs include dendritic cells, macrophages, or monocytes. In one embodiment, the TAA presentation inducer construct comprises at least one ISR-binding construct that binds to an ISR expressed on the surface of an APC of lymphoid origin. B cells are one example of an APC of lymphoid origin. In some inflammatory contexts, non-immune cells, such as epithelial or endothelial cells, may acquire APC capacity. Thus, in some embodiments, the at least one ISR-binding construct binds to a receptor expressed on the surface of epithelial or endothelial cells that acts as APCs.

In one embodiment the APC may be an APC that is capable of cross-presenting cell-associated TAAs.

ISRs are expressed on the surface of APCs and play a role in the innate immune response, often in the response to pathogens. Upon natural or artificial ligand binding, ISRs can promote numerous cellular responses, including, but not limited to: APC activation, cytokine production, chemokine production, adhesion, phagocytosis, pinocytosis, antigen presentation, and/or costimulatory cell-surface receptor upregulation. As is known in the art, there are different types of ISRs. In one embodiment, the TAA presentation inducer construct comprises at least one ISR-binding construct that binds to a C-type lectin receptor, a member of the tumor necrosis factor (TNF) receptor superfamily, or a member of the toll-like receptor (TLR) family, expressed on the surface of the APC. Suitable C-type lectin receptors include, but are not limited to, Dectin-1, Dectin-2, DEC205, Mincle, and DC-SIGN. Suitable members of the TNF receptor (TNFR) superfamily include, but are not limited to, TNFRI, TNFRII, 4-1BB, DR3, CD40, OX40, CD27, HVEM, and RANK. Suitable members of the TLR family include TLR1, TLR2, TLR3, TLR4, TLR5, TLR6, TLR8, and TLR11. In another embodiment, the TAA presentation inducer comprises at least one ISR-binding construct that binds to a lipoprotein receptor such as, for example, LRP-1 (LDL receptor-related protein-1), CD36, LOX-1, or SR-B1.

In one embodiment, the TAA presentation inducer construct comprises at least one ISR-binding construct that binds to a C-type lectin receptor that is expressed on a dendritic cell. In one embodiment the TAA presentation inducer construct comprises at least one ISR-binding construct that binds to Dectin-1. In one embodiment the TAA presentation inducer construct comprises at least one ISR-binding construct that binds to DEC205.

In one embodiment, the TAA presentation inducer construct comprises at least one ISR-binding construct that binds to an ISR other than CLEC9A (also known as DNGR1, or CD370). In one embodiment, the TAA presentation inducer comprises at least one ISR-binding construct that binds to a C-type lectin receptor other than CLEC9A. In one embodiment, the TAA presentation inducer construct comprises at least one ISR-binding construct that binds to a member of the TNFR superfamily other than CD40. In one embodiment, the TAA presentation inducer construct comprises at least one ISR-binding construct that binds to an ISR from a family other than the Toll-like Receptor family.

In one embodiment, the TAA presentation inducer construct comprises at least one ISR-binding construct that bind to LRP-1.

In one embodiment, the TAA presentation inducer construct comprises at least one ISR-binding construct that can promote activation of the ISR that it binds to. “Activation of the ISR” refers to the initiation of intracellular signaling within the APC expressing the ISR, which may result in antigen uptake, processing, and presentation.

The at least one ISR-binding construct may be a ligand for the ISR, or other moiety that can bind to the ISR. Thus, in one embodiment, the at least one ISR-binding construct is an endogenous, pathogenic, or synthetic ligand for the ISR. Such ligands are known in the art and described, for example, in Apostolopoulos et al. in Journal of Drug Delivery, Volume 2013, Article ID 869718, or Deisseroth et al. in Cancer Gene Therapy 2013 February; 20(2):65-9, Article ID 23238593. For example, if the ISR is Dectin-1, the at least one ISR-binding construct may be a β-glucan or vimentin. As another example, if the ISR is DC-SIGN, the at least one ISR-binding construct may be a mannan, ICAM, or CEACAM. Finally, if the ISR is LRP-1, the at least one ISR-binding construct may be calreticulin.

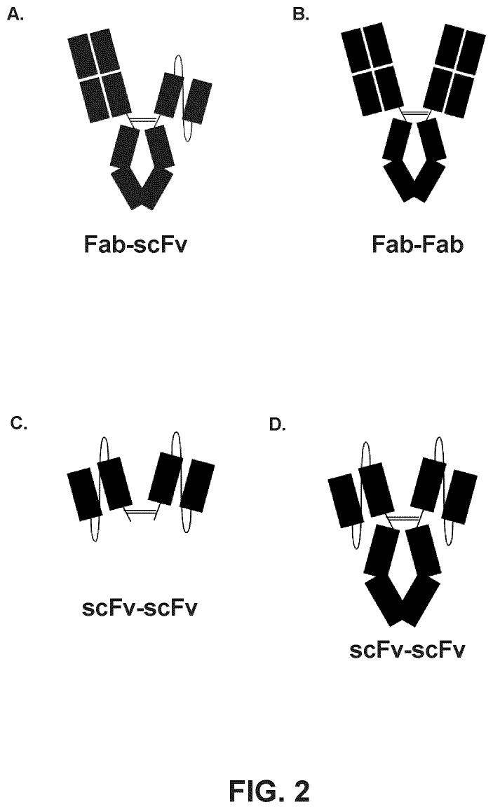

Alternatively, the at least one ISR-binding construct may be a moiety that is capable of targeting the ISR, and may be an antibody or a non-antibody form. In one embodiment, the at least one ISR-binding construct is an antibody. In another embodiment, the at least one ISR-binding construct is an antigen-binding domain. The term “antigen-binding domain” includes an antibody fragment, a Fab, an scFv, an sdAb, a VHH, and the like. In some embodiments, the at least one ISR-binding construct can include one or more antigen-binding domains (e.g., Fabs, VHHs or scFvs) linked to one or more Fc. The term “antibody” is described in more detail elsewhere herein, and exemplary antibody formats for the at least one ISR-binding constructs are described in the Examples and depicted in FIG. 2.

Antibodies that can bind to ISRs are known in the art. For example, monoclonal antibodies to the C-type lectin receptor dectin-1 are described in International Patent Publication No. WO2008/118587; antibodies to DEC205 are described in International Patent Publication No. WO2009/061996; and antibodies to CD40 are described in U.S. Patent Publication No. 2010/0239575. Other such antibodies are commercially available from companies such as Invivogen and Sigma-Aldrich, for example. If human antibodies are desired, and mouse antibodies are available, the mouse antibodies can be “humanized” by methods known in the art, and as described elsewhere herein.

Alternatively, antibodies to a specific ISR of interest may be generated by standard techniques and used as a basis for the preparation of the at least one ISR-binding construct of the TAA presentation inducer construct. Briefly, an antibody to a known ISR can be prepared by immunizing the purified ISR protein into rabbits, preparing serum from blood of the rabbits and absorbing the sera to a normal plasma fraction to produce an antibody specific to the ISR protein. Monoclonal antibody preparations to the ISR protein may be prepared by injecting the purified protein into mice, harvesting the spleen and lymph node cells, fusing these cells with mouse myeloma cells and using the resultant hybridoma cells to produce the monoclonal antibody. Both of these methods are well-known in the art. In some embodiments, antibodies resulting from these methods may be humanized as described elsewhere herein.

As an alternative to humanization, human antibodies can be generated. For example, transgenic animals (e.g., mice) can be used that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. Transfer of the human germ-line immunoglobulin gene array in such germ-line mutant mice will result in the production of human antibodies upon antigen challenge. See, e.g., Jakobovits et al., 1993, Proc. Natl. Acad. Sci. USA 90:2551; Jakobovits et al., 1993, Nature 362:255-258; Bruggermann et al., 1993, Year in Immuno. 7:33; and U.S. Pat. Nos. 5,591,669; 5,589,369; 5,545,807; 6,075,181; 6,150,584; 6,657,103; and 6,713,610.

Alternatively, phage display technology (see, e.g., McCafferty et al., 1990, Nature 348:552-553) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson and Chiswell, 1993, Current Opinion in Structural Biology 3:564-571. Several sources of V-gene segments can be used for phage display. Clackson et al., 1991, Nature 352:624-628 isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., 1991, J. Mol. Biol. 222:581-597, or Griffith et al., 1993, EMBO J. 12:725-734. See also U.S. Pat. Nos. 5,565,332 and 5,573,905. Human antibodies may also be generated by in vitro activated B cells (see U.S. Pat. Nos. 5,567,610 and 5,229,275).

Thus, in one embodiment the TAA presentation inducer construct comprises at least one ISR-binding construct that is derived from an anti-Dectin-1 antibody. In one embodiment, the TAA presentation inducer construct comprises at least one ISR-binding construct that is derived from an anti-DEC205 antibody. In one embodiment, the TAA presentation inducer construct comprises at least one ISR-binding construct that is derived from an anti-CD40 antibody. In one embodiment, the TAA presentation inducer construct comprises at least one ISR-binding construct that is derived from an anti-LRP-1 antibody.

In other embodiments, the at least one ISR-binding construct may be in a non-antibody form. Several non-antibody forms are known in the art, such as affibodies, affilins, anticalins, atrimers, DARPins, FN3 scaffolds (for example, adnectins and centyrins), fynomers, Kunitz domains, pronectins and OBodies. These and other non-antibody forms can be engineered to provide molecules that have target-binding affinities and specificities that are similar to those of antibodies (Vazquez-Lombardi et al. (2015) Drug Discovery Today 20: 1271-1283, and Fiedler et al. (2014) pp. 435-474, in Handbook of Therapeutic Antibodies, 2^{nd} ed., edited by Stefan Dubel and Janice M. Reichert, Wiley-VCH Verlag GmbH&Co. KGaA).

### Tumor-Associated Antigen (TAA)-Binding Constructs

The at least one TAA-binding construct of the TAA presentation inducer construct described herein binds directly to a first TAA that is physically associated with tumor cell-derived material (TCDM) comprising one or more other TAAs. The “other TAAs” may also be referred to herein as “secondary TAAs.” Secondary TAAs may also be physically associated with TCDM. The term “physically associated with TCDM” is intended to include covalent and/or non-covalent interactions between the first TAA and the TCDM or between the secondary TAAs and the TCDM. Non-covalent interactions may include electrostatic or van der Waals interactions, for example. The term “binds directly” is intended to describe a direct interaction between the first TAA and the TAA-binding construct of the TAA presentation inducer construct, in the absence of bridging components between the first TAA and the TAA-binding construct. In contrast, in some embodiments, the at least one TAA-binding construct may bind one or more secondary TAAs “indirectly” via the first TAA, where the first TAA may act as a bridging component.

As used herein “tumor-associated antigen” or “TAA” refers to an antigen that is expressed by cancer cells. A tumor-associated antigen may or may not be expressed by normal cells. When a TAA is not expressed by normal cells (i.e. when it is unique to tumor cells) it may also be referred to as a “tumor-specific antigen.” When a TAA is not unique to a tumor cell, it is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development (also called oncofetal antigens) when the immune system is immature and unable to respond, or they may be antigens that are normally present at low levels on normal cells but which are expressed at much higher levels on tumor cells. Those TAAs of greatest clinical interest are differentially expressed compared to the corresponding normal tissue and allow for a preferential recognition of tumor cells by specific T-cells or immunoglobulins. TAAs can include membrane-bound antigens, or antigens that are localized within a tumor cell.

In one embodiment, the TAA presentation inducer construct comprises at least one TAA-binding construct that binds to a first TAA that is expressed at high levels in tumor cells. For example, the tumor cells may express the first TAA at greater than about 1 million copies per cell. In another embodiment, the TAA presentation inducer construct comprises at least one TAA-binding construct that binds to a first TAA that is expressed at medium levels in tumor cells. For example, the tumor cells may express the first TAA at greater than about 100,000 to about 1 million copies per cell. In one embodiment, the first TAA presentation inducer construct comprises at least one TAA-binding construct that binds to a first TAA that is expressed at low levels in tumor cells. For example, the tumor cells may express the first TAA at less than about 100,000 copies per cell. In one embodiment, the TAA presentation inducer construct comprises at least one TAA-binding construct that binds to a first TAA that is present in tumors with relatively few infiltrating immune cells (low immunoscore TAA). In one embodiment, the TAA presentation inducer construct comprises at least one TAA-binding construct that binds to a first TAA that is an oncofetal antigen.

As indicated above, the at least one TAA-binding construct of the TAA presentation inducer construct described herein binds directly to a first TAA that is physically associated with tumor cell-derived material (TCDM) comprising one or more secondary TAAs. The secondary TAAs may be complexed in the TCDM.

In one embodiment, the TAA presentation inducer comprises at least one TAA-binding construct that binds to a first TAA selected from, but not limited to, carbonic anhydrase IX, alpha-fetoprotein (AFP), alpha-actinin-4, A3, antigen specific for A33 antibody, ART-4, B7, Ba 733, BAGE, BCMA, BrE3-antigen, CA125, CAMEL, CAP-1, CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CTLA-4, CXCR4, CXCR7, CXCL12, HIF-1a, colon-specific antigen-p (CSAp), CEA, CEACAM5, CEACAM6, c-Met, DAM, DL3, EGFR, EGFRvIII, EGP-1 (TROP-2), EGP-2, ELF2-M, Ep-CAM, EphA2, fibroblast growth factor (FGF), Flt-1, Flt-3, folate receptor, G250 antigen, GAGE, GD2, gp100, GPC3, GRO-13, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, mCRP, MCP-1, melanoma glycoprotein, mesothelin, MIP-1A, MIP-1B, MIF, MUC1, MUC2, MUC3, MUC4, MUC5ac, MUC13, MUC16, MUM-1/2, MUM-3, NaPi2B, NCA66, NCA95, NCA90, NY-ESO-1, PAM4 antigen, pancreatic cancer mucin, PD-1, PD-L1, PD-1 receptor, placental growth factor, p53, PLAGL2, prostatic acid phosphatase, PSA, PRAME, PSMA, P1GF, ILGF, ILGF-1R, IL-6, IL-25, RS5, RANTES, ROR1, T101, SAGE, 5100, survivin, survivin-2B, TAC, TAG-72, tenascin, TRAG-3, TRAIL receptors, TNF-alpha, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, an oncogene marker and an oncogene product (see, e.g., Sensi et al., Clin Cancer Res 2006, 12:5023-32; Parmiani et al., J Immunol 2007, 178:1975-79; Novellino et al. Cancer Immunol Immunother 2005, 54:187-207).

The at least one TAA-binding construct may be a ligand that binds to the first TAA, or some other moiety that can bind to the first TAA. Thus, in one embodiment, the at least one TAA-binding construct may an endogenous or synthetic ligand for the TAA. For example, heregulin and NRG-2 are ligands for HER3, WNT5A is a ligand for ROR1, and folate is a ligand for folate receptor.

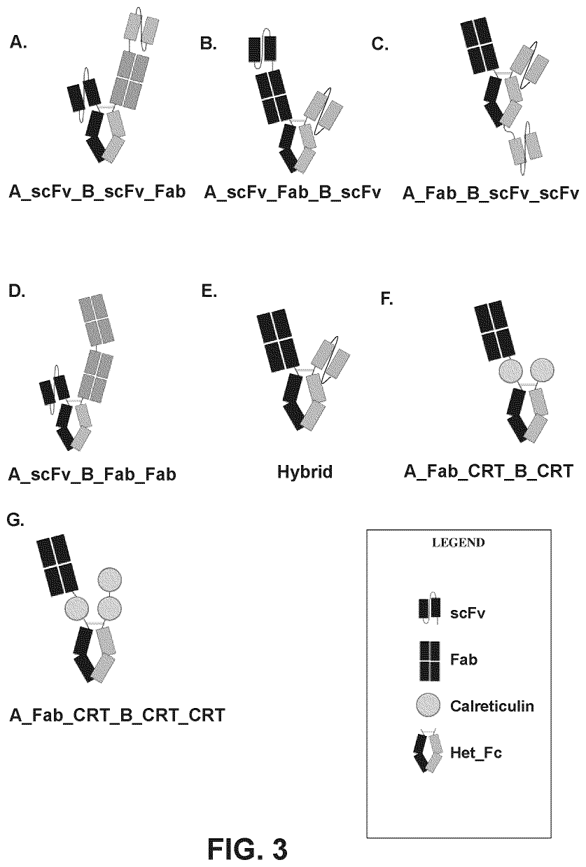

Alternatively, the at least one TAA-binding construct may be a moiety that is capable of targeting the first TAA, and may be an antibody or a non-antibody form. In one embodiment, the at least one TAA-binding construct is an antibody or antigen-binding domain. The term “antigen-binding domain” includes an antibody fragment, a Fab, an scFv, an sdAb, a VHH, and the like. In some embodiments, the at least one TAA-binding construct can include one or more antigen-binding domains (e.g., Fabs, VHHs or scFvs) linked to one or more Fc. The term “antibody” is described in more detail elsewhere and exemplary formats for the at least one TAA-binding constructs are provided in the Examples and depicted in FIG. 2 and FIG. 3.

Antibodies directed against tumor-associated antigens are known in the art and may be commercially obtained from a number of sources. For example, a variety of antibody secreting hybridoma lines are available from the American Type Culture Collection (ATCC, Manassas, Va.). A number of antibodies against various tumor-associated antigens have been deposited at the ATCC and/or have published variable region sequences and may be used to prepare the TAA presentation inducer constructs in certain embodiments. The skilled artisan will appreciate that antibody sequences or antibody-secreting hybridomas against various tumor-associated antigens may be obtained by a simple search of the ATCC, NCBI and/or USPTO databases.

Particular tumor-associated antigen targeted antibodies that may be of use in preparing the TAA presentation inducer constructs described herein include, but are not limited to, LL1 (anti-CD74), LL2 or RFB4 (anti-CD22), veltuzumab (hA20, anti-CD20), rituxumab (anti-CD20), obinutuzumab (GA101, anti-CD20), lambrolizumab (anti-PD-1 receptor), nivolumab (anti-PD-1 receptor), ipilimumab (anti-CTLA-4), RS7 (anti-TROP-2), PAM4 or KC4 (both anti-mucin), MN-14 (anti-CEA), MN-15 or MN-3 (anti-CEACAM6), Mu-9 (anti-colon-specific antigen-p), Immu 31 (an anti-alpha-fetoprotein), R1 (anti-IGF-1R), A19 (anti-CD19), TAG-72 (e.g., CC49), Tn, J591, MLN2704 or HuJ591 (anti-PSMA), AB-PG1-XG1-026 (anti-PSMA dimer), D2/B (anti-PSMA), G250 (anti-carbonic anhydrase IX), L243 (anti-HLA-DR) alemtuzumab (anti-CD52), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (aka clivatuzumab, anti-mucin), trastuzumab (anti-HER2), pertuzumab (anti-HER2), polatuzumab (anti-CD79b), R2 (anti-ROR1), 2A2 (anti-ROR1), and anetumab (anti-mesothelin).

In certain embodiments, the at least one TAA-binding construct is derived from a humanized, or chimeric version of a known antibody. In one embodiment, the at least one TAA-binding construct is derived from an antibody that binds to a human, cynomolgous monkey, rhesus monkey, or mouse TAA.

Alternatively, antibodies to a specific TAA of interest may be generated by standard techniques in a similar manner as described for preparing antibodies to ISRs, but using purified TAA proteins, and used as a basis for the preparation of the at least one TAA-binding construct of the TAA presentation inducer construct.

Thus, in one embodiment the TAA presentation inducer comprises at least one TAA-binding construct derived from an anti-HER2 antibody. In one embodiment, the TAA presentation inducer comprises at least one TAA-binding construct derived from trastuzumab or pertuzumab. In another embodiment, the TAA presentation inducer comprises at least one TAA-binding construct that is derived from an anti-ROR1 antibody. In one embodiment, the TAA presentation inducer construct comprises at least one TAA-binding construct that is derived from an anti-PSMA antibody. In one embodiment, the TAA presentation inducer construct comprises at least one TAA-binding construct that is derived from an anti-mesothelin antibody.

In other embodiments, the at least one TAA-binding construct may be in a non-antibody form, as described elsewhere herein with respect to the ISR-binding construct.

### Format of TAA Presentation Inducer Constructs

In one embodiment, the TAA presentation inducer construct comprises one ISR-binding construct and at least one TAA-binding construct. In various embodiments, the TAA presentation inducer construct comprises two, three, or more ISR-binding constructs and at least one TAA-binding construct. In some embodiments, the two, three, or more ISR-binding constructs may be identical to each other. In some embodiments, the two, three, or more ISR-binding constructs may bind to the same ISR, but the constructs may comprise ISR-binding constructs with different formats of antigen-binding domains, i.e. scFvs, Fabs, or may include one or more ligand that binds to the ISR. In other embodiments, the two, three, or more ISR-binding constructs may bind to at least two different ISRs. In such embodiments, the ISR-binding constructs may be antigen-binding domains, or may be ligands that recognize the target ISR, or may be combinations of same.

In one embodiment, the TAA presentation inducer construct comprises at least one ISR-binding construct and one TAA-binding construct. In various embodiments, the TAA presentation inducer construct comprises at least one ISR-binding construct and two or more TAA-binding constructs. In these embodiments, the TAA-binding constructs may be identical to each other, or they may be different from each other. In embodiments where the TAA-binding constructs are different from each other, the TAA-binding constructs may bind to different TAAs, or to different regions of the same TAA, or may include antigen-binding domains or ligands binding to the TAA that are different from each other, or may include antigen-binding domains that are combinations of formats such as scFvs and Fabs.

In certain embodiments, the TAA presentation inducer construct is a multispecific antibody, wherein the multispecific antibody can bind to at least one ISR expressed on an APC and to at least one first TAA that is physically associated with TCDM. In this embodiment, the TAA presentation inducer construct comprises at least one ISR-binding construct and at least one TAA-binding construct linked to each other with an Fc scaffold. In other embodiments, the TAA presentation inducer construct is a bispecific antibody comprising an ISR binding construct that is expressed on an APC and at least one TAA-binding construct that binds directly to a first TAA that is physically associated with TCDM comprising one or more other TAAs. The bispecific antibody may comprise an Fc or a heterodimeric Fc as described elsewhere herein.

As indicated elsewhere herein, the at least one ISR-binding constructs and at least one TAA-binding constructs of the TAA presentation inducer constructs may be ligands, antibodies, antigen-binding domains, or non-antibody forms. The TAA presentation inducer constructs may comprise ISR-binding constructs and TAA-binding constructs that are combinations of these forms. In various embodiments, the TAA presentation inducer construct comprises at least one ISR-binding construct that is a ligand for the ISR, and at least one TAA-binding construct that is a ligand for the TAA. In a related embodiment, the TAA presentation inducer construct comprises at least one ISR-binding construct that is a ligand for the ISR, and at least one TAA-binding construct that is an antigen-binding domain. In a related embodiment, the TAA presentation inducer construct comprises at least one ISR-binding construct that is a ligand for the ISR, and at least one TAA-binding construct that is a non-antibody form. In one embodiment, the TAA presentation inducer construct comprises at least one ISR-binding construct that is an antigen-binding domain, and at least one TAA-binding construct that is an antigen-binding domain. In another embodiment, the TAA presentation inducer construct comprises at least one ISR-binding construct that is a non-antibody form, and at least one TAA-binding construct that is an antigen-binding domain. In a one embodiment, the TAA presentation inducer construct comprises at least one ISR-binding construct that is an antigen-binding domain, and at least one TAA-binding construct that is a ligand for the TAA. In a one embodiment, the TAA presentation inducer construct comprises at least one ISR-binding construct that is non-antibody form, and at least one TAA-binding construct that is a ligand. In a one embodiment, the TAA presentation inducer construct comprises at least one ISR-binding construct that is non-antibody form, and at least one TAA-binding construct that is a non-antibody form. In a one embodiment, the TAA presentation inducer construct comprises at least one ISR-binding construct that is an antigen-binding domain, and at least one TAA-binding construct that is a non-antibody form.

In embodiments where the TAA presentation inducer construct is a bispecific antibody, the ISR-binding construct may be a Fab and the TAA-binding construct may be a Fab. Alternatively, in embodiments where the TAA presentation inducer construct is a bispecific antibody, the ISR-binding construct may be a Fab and the TAA-binding construct may be a scFv. In other embodiments where the TAA presentation inducer construct is a bispecific antibody, the ISR-binding construct may be an scFv and the TAA-binding construct may be an scFv. In other embodiments where the TAA presentation inducer construct is a bispecific antibody, the ISR-binding construct may be an scFv and the TAA-binding construct may be a Fab. Examples of bispecific antibody formats are shown in FIG. 2 and FIG. 3. In some embodiments, the TAA presentation inducer is a bispecific antibody in full-size antibody format (FSA).

In some embodiments, the TAA presentation inducer construct comprises an ISR that is a ligand for an LDL receptor, and at least one TAA-binding construct, linked to each other. In some embodiments, the TAA presentation inducer construct comprises an ISR that is a ligand for LRP-1, and at least one TAA-binding construct, linked to each other. In some embodiments, the TAA presentation inducer construct comprises an ISR that is calreticulin, and at least one TAA-binding construct, linked to each other.

In various embodiments, the TAA presentation inducer construct comprises at least one ISR-binding construct that binds to a C-type lectin receptor and at least one TAA-binding construct that binds to a first TAA that is expressed at high levels in tumor cells, at low levels in tumor cells, at medium levels in tumor cells, is an oncofetal antigen, or is a low immunoscore TAA. In other embodiments, the TAA presentation inducer construct comprises at least one ISR-binding construct that binds to a TNF family receptor and at least one TAA-binding construct that binds to a first TAA that is expressed at high levels in tumor cells, at low levels in tumor cells, at medium levels in tumor cells, is an oncofetal antigen, or is a low immunoscore TAA. In some embodiments, the TAA presentation inducer construct comprises at least one ISR-binding construct that binds to an LDL receptor and at least one TAA-binding construct that binds to a first TAA that is expressed at high levels in tumor cells, at low levels in tumor cells, at medium levels in tumor cells, is an oncofetal antigen, or is a low immunoscore TAA. In some embodiments, the first TAA is HER2, ROR1, or PSMA.

In additional embodiments, the TAA presentation inducer construct comprises an ISR-binding construct that binds to dectin-1 and a TAA-binding construct that binds to one of HER2, ROR1, or PSMA. In other embodiments, the TAA presentation inducer construct comprises an ISR-binding construct that binds to DEC205 and a TAA-binding construct that binds to one of HER2, ROR1, or PSMA. In further embodiments, the TAA presentation inducer construct comprises an ISR-binding construct that binds to LRP-1 and a TAA-binding construct that binds to one of HER2, ROR1, or PSMA. In still further embodiments, the TAA presentation inducer construct comprises an ISR-binding construct that binds to CD40 and a TAA-binding construct that binds to one of HER2, ROR1, or PSMA.

In some embodiments, the TAA presentation inducer construct comprises an ISR-binding construct that binds to dectin-1 and a TAA-binding construct that binds to mesothelin. In some embodiments, the TAA presentation inducer construct comprises an ISR-binding construct that binds to dectin-1 and a TAA-binding construct that binds to HER2. In other embodiments, the TAA presentation inducer construct comprises an ISR-binding construct that binds to DEC205 and a TAA-binding construct that binds to mesothelin. In further embodiments, the TAA presentation inducer construct comprises an ISR-binding construct that binds to LRP-1 and a TAA-binding construct that binds to mesothelin. In one of these embodiments, the TAA presentation inducer construct comprises an ISR-binding construct that is a recombinant form of calreticulin and a TAA binding construct that binds to mesothelin. In still further embodiments, the TAA presentation inducer construct comprises an ISR-binding construct that binds to CD40 and a TAA-binding construct that binds to mesothelin.

### Linkage Between the ISR-Binding Construct and the TAA-Binding Construct

The at least one ISR-binding construct and the at least one TAA-binding construct of the TAA presentation inducer construct may be linked to each other directly or indirectly. Direct linkage between the at least one ISR-binding construct and the at least one TAA-binding construct results when the two constructs are directly connected to each other without a linker or scaffold. Indirect linkage between the at least one ISR-binding construct and the at least one TAA-binding construct is achieved through use of linkers or scaffolds.

In some embodiments, the TAA presentation inducer constructs described herein comprise a scaffold. A scaffold may be a peptide, polypeptide, polymer, nanoparticle or other chemical entity. In one embodiment, the TAA presentation inducer comprises at least one ISR-binding construct that binds to an ISR expressed on an APC, and at least one TAA-binding construct, wherein the at least one ISR-binding construct and the at least one TAA-binding construct are linked to each other through a scaffold that is other than a cohesin-dockerin scaffold. Cohesin-dockerin scaffolds are described, for example in International Patent Publication No. WO2008/097817. The ISR- or TAA-binding constructs of the TAA presentation inducer construct may be linked to either the N- or C-terminus of the scaffold, where the scaffold is a polypeptide, such as an Fc, e.g., a dimeric Fc. A dimeric Fc can be homodimeric or heterodimeric. In one embodiment, the scaffold is a heterodimeric Fc. In other embodiments, the scaffold is a split albumin polypeptide pair described in WO 2012/116453 and WO 2014/012082.

In embodiments where the scaffold is a peptide or polypeptide, the ISR- or TAA-binding constructs of the TAA presentation inducer construct may be linked to the scaffold by genetic fusion. In other embodiments, where the scaffold is a polymer or nanoparticle, the ISR- or TAA-binding constructs of the TAA presentation inducer construct may be linked to the scaffold by chemical conjugation. In other embodiments, the ISR-binding construct and the TAA-binding construct are linked by a scaffold other than styrene-, propylene-, silica-, metal-, or carbon-based nanoparticles.

The term “Fc” as used herein refers to a C-terminal region of an immunoglobulin heavy chain that contains at least a portion of the constant region (also referred to as an “Fc domain” or “Fc region”). The term includes native sequence Fc regions and variant Fc regions. Unless otherwise specified herein, numbering of amino acid residues in the Fc region or constant region is according to the EU numbering system, also called the EU index, as described in Edelman, G. M. et al., Proc. Natl. Acad. USA, 63, 78-85 (1969). An “Fc polypeptide” of a dimeric Fc refers to one of the two polypeptides forming the dimeric Fc domain, i.e. a polypeptide comprising C-terminal constant regions of an immunoglobulin heavy chain that is capable of stable self-association. For example, an Fc polypeptide of a dimeric IgG Fc comprises an IgG CH2 and an IgG CH3 constant domain sequence.

An Fc domain comprises either a CH3 domain or a CH3 and a CH2 domain. The CH3 domain comprises two CH3 sequences, one from each of the two Fc polypeptides of the dimeric Fc. The CH2 domain comprises two CH2 sequences, one from each of the two Fc polypeptides of the dimeric Fc.

In some embodiments, the TAA presentation inducer construct comprises an Fc comprising one or two CH3 sequences. In some embodiments, the Fc is coupled, with or without one or more linkers, to the at least one ISR-binding construct and the at least one TAA-binding construct. In some embodiments, the Fc is a human Fc. In some embodiments, the Fc is a human IgG or IgG1 Fc. In some embodiments, the Fc is a heterodimeric Fc. In some embodiments, the Fc comprises one or two CH2 sequences.

In some embodiments, the Fc comprises one or two CH3 sequences at least one of which comprises one or more modifications. In some embodiments, the Fc comprises one or two CH2 sequences, at least one of which comprises one or more modifications. In some embodiments, an Fc is composed of a single polypeptide. In some aspects, an Fc is composed of multiple peptides, e.g., two polypeptides.

In some embodiments, the TAA presentation inducer construct comprises an Fc as described in International Patent Application No. PCT/CA2011/001238 or International Patent Application No. PCT/CA2012/050780, the entire disclosure of each of which is hereby incorporated by reference in its entirety for all purposes.

### Modified CH3 Domains

In some embodiments, the TAA presentation inducer construct described herein comprises a heterodimeric Fc comprising a modified CH3 domain, wherein the modified CH3 domain is an asymmetrically modified CH3 domain. The heterodimeric Fc may comprise two heavy chain constant domain polypeptides: a first Fc polypeptide and a second Fc polypeptide, which can be used interchangeably provided that the Fc comprises one first Fc polypeptide and one second Fc polypeptide. Generally, the first Fc polypeptide comprises a first CH3 sequence and the second Fc polypeptide comprises a second CH3 sequence.

Two CH3 sequences that comprise one or more amino acid modifications introduced in an asymmetric fashion generally results in a heterodimeric Fc, rather than a homodimer, when the two CH3 sequences dimerize. As used herein, “asymmetric amino acid modifications” refers to any modification where an amino acid at a specific position on a first CH3 sequence is different from the amino acid on a second CH3 sequence at the same position, and the first and second CH3 sequence preferentially pair to form a heterodimer, rather than a homodimer. This heterodimerization can be a result of modification of only one of the two amino acids at the same respective amino acid position on each sequence, or modification of both amino acids on each sequence at the same respective position on each of the first and second CH3 sequences. The first and second CH3 sequence of a heterodimeric Fc can comprise one or more than one asymmetric amino acid modification.

Table A provides the amino acid sequence of the human IgG1 Fc sequence, corresponding to amino acids 231 to 447 of the full-length human IgG1 heavy chain. The CH3 sequence comprises amino acid 341-447 of the full-length human IgG1 heavy chain.

Typically, an Fc includes two contiguous heavy chain sequences (A and B) that are capable of dimerizing. In some embodiments, one or both sequences of an Fc may include one or more mutations or modifications at the following locations: L351, F405, Y407, T366, K392, T394, T350, S400, and/or N390, using EU numbering. In some embodiments, an Fc may include a mutant sequence as shown in Table B. In some embodiments, an Fc may include the mutations of Variant 1 A-B. In some embodiments, an Fc may include the mutations of Variant 2 A-B. In some embodiments, an Fc may include the mutations of Variant 3 A-B. In some embodiments, an Fc may include the mutations of Variant 4 A-B. In some embodiments, an Fc may include the mutations of Variant 5 A-B.

TABLE A
IgG1 Fc sequences

| Human IgG1 Fc sequence | APELLGGPSVFLFPPKPKDTLMISRTPEVTCVVVDVSH | |
|---|---|---|
| 231-447 (EU-numbering) | EDPEVKFNWYVDGVEVHNAKTKPREEQYNSTYRVVS | |
| | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| | (SEQ ID NO: 69) | |
| Variant IgG1 Fc | | |
| sequence (231-447) | Chain | Mutations |
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

In certain embodiments, the first and second CH3 sequences comprised by the heterodimeric Fc may comprise amino acid mutations as described herein, with reference to amino acids 231 to 447 of the full-length human IgG1 heavy chain. In some embodiments, the heterodimeric Fc comprises a modified CH3 domain with a first CH3 sequence having amino acid modifications at positions F405 and Y407, and a second CH3 sequence having amino acid modifications at position T394. In some embodiments, the heterodimeric Fc comprises a modified CH3 domain with a first CH3 sequence having one or more amino acid modifications selected from L351Y, F405A, and Y407V, and the second CH3 sequence having one or more amino acid modifications selected from T366L, T366I, K392L, K392M, and T394W.

In some embodiments, a heterodimeric Fc comprises a modified CH3 domain with a first CH3 sequence having amino acid modifications at positions L351, F405 and Y407, and a second CH3 sequence having amino acid modifications at positions T366, K392, and T394, and one of the first or second CH3 sequences further comprising amino acid modifications at position Q347, and the other CH3 sequence further comprising amino acid modification at position K360. In some embodiments, a heterodimeric Fc comprises a modified CH3 domain with a first CH3 sequence having amino acid modifications at positions L351, F405 and Y407, and a second CH3 sequence having amino acid modifications at position T366, K392, and T394, one of the first or second CH3 sequences further comprising amino acid modifications at position Q347, and the other CH3 sequence further comprising amino acid modification at position K360, and one or both of said CH3 sequences further comprise the amino acid modification T350V.

In some embodiments, a heterodimeric Fc comprises a modified CH3 domain with a first CH3 sequence having amino acid modifications at positions L351, F405 and Y407, and a second CH3 sequence having amino acid modifications at positions T366, K392, and T394 and one of said first and second CH3 sequences further comprising amino acid modification of D399R or D399K and the other CH3 sequence comprising one or more of T411E, T411D, K409E, K409D, K392E and K392D. In some embodiments, a heterodimeric Fc comprises a modified CH3 domain with a first CH3 sequence having amino acid modifications at positions L351, F405 and Y407, and a second CH3 sequence having amino acid modifications at positions T366, K392, and T394, one of said first and second CH3 sequences further comprises amino acid modification of D399R or D399K and the other CH3 sequence comprising one or more of T411E, T411D, K409E, K409D, K392E and K392D, and one or both of said CH3 sequences further comprise the amino acid modification T350V.

In some embodiments, a heterodimeric Fc comprises a modified CH3 domain with a first CH3 sequence having amino acid modifications at positions L351, F405 and Y407, and a second CH3 sequence having amino acid modifications at positions T366, K392, and T394, wherein one or both of said CH3 sequences further comprise the amino acid modification of T350V.

In some embodiments, a heterodimeric Fc comprises a modified CH3 domain comprising the following amino acid modifications, where “A” represents the amino acid modifications to a first CH3 sequence, and “B” represents the amino acid modifications to a second CH3 sequence:

| | |
|---|---|
| A: L351Y_F405A_Y407V | B: T366L_K392M_T394W |
| A: L351Y_F405A_Y407V | B: T366L_K392L_T394W |
| A: T350V_L351Y_F405A_Y407V | B: T350V_T366L_K392L_T394W |
| A: T350V_L351Y_F405A_Y407V | B: T350V_T366L_K392M_T394W |
| A: T350V_L351Y_S400E_F405A_Y407V | B: T350V_T366L_N390R_K392M_T394W. |

The one or more asymmetric amino acid modifications can promote the formation of a heterodimeric Fc in which the heterodimeric CH3 domain has a stability that is comparable to a wild-type homodimeric CH3 domain. In some embodiments, the one or more asymmetric amino acid modifications promote the formation of a heterodimeric Fc domain in which the heterodimeric Fc domain has a stability that is comparable to a wild-type homodimeric Fc domain. In some embodiments, the one or more asymmetric amino acid modifications promote the formation of a heterodimeric Fc domain in which the heterodimeric Fc domain has a stability observed via the melting temperature (Tm) in a differential scanning calorimetry study, and where the melting temperature is within 4° C. of that observed for the corresponding symmetric wild-type homodimeric Fc domain. In some embodiments, the Fc comprises one or more modifications in at least one of the CH3 sequences that promote the formation of a heterodimeric Fc with stability comparable to a wild-type homodimeric Fc.

In some embodiments, the stability of the CH3 domain can be assessed by measuring the melting temperature of the CH3 domain, for example by differential scanning calorimetry (DSC). Thus, in various embodiments, the CH3 domain may have a melting temperature of about 68° C. or higher, about 70° C. or higher, about 72° C. or higher, 73° C. or higher, about 75° C. or higher, or about 78° C. or higher. In some embodiments, the dimerized CH3 sequences have a melting temperature (Tm) of about 68, 69, 70, 71, 72, 73, 74, 75, 76, 77, 77.5, 78, 79, 80, 81, 82, 83, 84, or 85° C. or higher.

In some embodiments, a heterodimeric Fc comprising modified CH3 sequences can be formed with a purity of at least about 75% as compared to homodimeric Fc in the expressed product. In some embodiments, the heterodimeric Fc is formed with a purity greater than about 80%, greater than about 85%, greater than about 90%, greater than about 95% or greater than about 97%. In some embodiments, the Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98, or 99% when expressed. In some embodiments, the Fc is a heterodimer formed with a purity greater than about 75, 76, 77, 78, 79, 80, 81, 82, 83, 84, 85, 86, 87, 88, 89, 90, 91, 92, 93, 94, 95, 96, 97, 98, or 99% when expressed via a single cell.

Additional methods for modifying monomeric Fc polypeptides to promote heterodimeric Fc formation are known in the art and include, for example, those described in International Patent Publication No. WO 96/027011 (knobs into holes), in Gunasekaran et al. (Gunasekaran K. et al. (2010) J Biol Chem. 285, 19637-46, electrostatic design to achieve selective heterodimerization), in Davis et al. (Davis, J H. et al. (2010) Prot Eng Des Sel; 23(4): 195-202, strand exchange engineered domain (SEED) technology), and in Labrijn et al [Efficient generation of stable bispecific IgG1 by controlled Fab-arm exchange. Labrijn A F, Meesters J I, de Goeij B E, van den Bremer E T, Neijssen J, van Kampen M D, Strumane K, Verploegen S, Kundu A, Gramer M J, van Berkel P H, van de Winkel J G, Schuurman J, Parren P W. Proc Natl Acad Sci USA. 2013 Mar. 26; 110(13):5145-50.

### CH2 Domains

In some embodiments, the TAA presentation inducer construct comprises an Fc comprising a CH2 domain. One example of a CH2 domain of an Fc is amino acids 231-340 of the sequence shown in Table A. Several effector functions are mediated by Fc receptors (FcRs), which bind to the Fc of an antibody.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIM contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

Modifications in the CH2 domain can affect the binding of FcRs to the Fc. A number of amino acid modifications in the Fc region are known in the art for selectively altering the affinity of the Fc for different Fcgamma receptors. In some aspects, the Fc comprises one or more modifications to promote selective binding of Fc-gamma receptors.

Exemplary mutations that alter the binding of FcRs to the Fc are listed below:
S298A/E333A/K334A, S298A/E333A/K334A/K326A (Lu Y, Vernes J M, Chiang N, et al. J Immunol Methods. 2011 Feb. 28; 365(1-2):132-41);
F243L/R292P/Y300L/V305I/P396L, F243L/R292P/Y300L/L235V/P396L (Stavenhagen J B, Gorlatov S, Tuaillon N, et al. Cancer Res. 2007 Sep. 15; 67(18):8882-90; Nordstrom J L, Gorlatov S, Zhang W, et al. Breast Cancer Res. 2011 Nov. 30; 13(6):R123);
F243L (Stewart R, Thom G, Levens M, et al. Protein Eng Des Sel. 2011 September; 24(9):671-8.)
S298A/E333A/K334A (Shields R L, Namenuk A K, Hong K, et al. J Biol Chem. 2001 Mar. 2; 276(9):6591-604);
S239D/I332E/A330L, S239D/I332E (Lazar G A, Dang W, Karki S, et al. Proc Natl Acad Sci USA. 2006 Mar. 14; 103(11):4005-10);
S239D/S267E, S267E/L328F (Chu S Y, Vostiar I, Karki S, et al. Mol Immunol. 2008 September; 45(15):3926-33);
S239D/D265S/S298A/I332E, S239E/S298A/K326A/A327H, G237F/S298A/A330L/I 332, S239D/I332E/S298A, S239D/K326E/A330L/I332E/S298A, G236A/S239D/D270L/I332E, S239E/S267E/H268D, L234F/S267E/N325L, G237F/V266L/S267D and other mutations listed in WO2011/120134 and WO2011/120135, herein incorporated by reference.
   Therapeutic Antibody Engineering (by William R. Strohl and Lila M. Strohl, Woodhead Publishing series in Biomedicine No 11, ISBN 1 907568 37 9, October 2012) lists mutations on page 283.

In some embodiments, a TAA presentation inducer construct described herein comprises a dimeric Fc that has superior biophysical properties, for example stability and/or ease of manufacture, relative to an TAA presentation inducer construct which does not include the same dimeric Fc. In some embodiments, the dimeric Fc comprises a CH2 domain comprising one or more asymmetric amino acid modifications. Exemplary asymmetric mutations are described in International Patent Application No. PCT/CA2014/050507.

### Additional Modifications to Improve Effector Function

In some embodiments, a TAA presentation inducer construct including an Fc described herein includes modifications to the Fc to improve its ability to mediate effector function. Such modifications are known in the art and include afucosylation, or engineering of the affinity of the Fc towards an activating receptor, mainly FCgRIIIa for ADCC, and towards C1q for CDC. The following Table B summarizes various designs reported in the literature for effector function engineering.

Methods of producing antibody Fc regions with little or no fucose on the Fc glycosylation site (Asn 297 EU numbering) without altering the amino acid sequence are well known in the art. The GlymaX® technology (ProBioGen AG) is based on the introduction of a gene for an enzyme which deflects the cellular pathway of fucose biosynthesis into cells used for antibody Fc region production. This prevents the addition of the sugar “fucose” to the N-linked antibody carbohydrate part by cells. (von Horsten et al. (2010) Glycobiology. 20 (12):1607-18). Another approach to obtaining TAA presentation inducer constructs with Fc regions, with lowered levels of fucosylation can be found in U.S. Pat. No. 8,409,572, which teaches selecting cell lines for antibody production based on their ability to yield lower levels of fucosylation on antibodies. The Fc of TAA presentation inducers can be fully afucosylated (meaning they contain no detectable fucose) or they can be partially afucosylated, meaning that the TAA presentation inducer in bispecific antibody format contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

Thus, in some embodiments, a TAA presentation inducer construct described herein can include a dimeric Fc that comprises one or more amino acid modifications as noted in Table B that confer improved effector function. In some embodiments, the construct can be afucosylated to improve effector function.

TABLE B
CH2 domains and effector function engineering

| Reference | Mutations | Effect |
|---|---|---|
| Lu, 2011, | Afucosylated | Increased ADCC |
| Ferrara 2011, | | |
| Mizushima 2011 | | |
| Lu, 2011 | S298A/E333A/K334A | Increased ADCC |
| Lu, 2011 | S298A/E333A/K334A/K326A | Increased ADCC |
| Stavenhagen, 2007 | F243L/R292P/Y300L/V305I/ | Increased ADCC |
| | P396L | |
| Nordstrom, 2011 | F243L/R292P/Y300L/L235V/ | Increased ADCC |
| | P396L | |
| Stewart, 2011 | F243L | Increased ADCC |
| Shields, 2001 | S298A/E333A/K334A | Increased ADCC |
| Lazar, 2006 | S239D/I332E/A330L | Increased ADCC |
| Lazar, 2006 | S239D/I332E | Increased ADCC |
| Bowles, 2006 | AME-D, not specified mutations | Increased ADCC |
| Heider, 2011 | 37.1, mutations not disclosed | Increased ADCC |
| Moore, 2010 | S267E/H268F/S324T | Increased CDC |

Fc modifications reducing FcγR and/or complement binding and/or effector function are known in the art. Various publications describe strategies that have been used to engineer antibodies with reduced or silenced effector activity (see Strohl, W R (2009), Curr Opin Biotech 20:685-691, and Strohl, W R and Strohl L M, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, U.S. Patent Publication No. 2011/0212087 (Strohl), International Patent Publication No. WO 2006/105338 (Xencor), U.S. Patent Publication No. 2012/0225058 (Xencor), U.S. Patent Publication No. 2012/0251531 (Genentech), and Strop et al ((2012) J. Mol. Biol. 420: 204-219) describe specific modifications to reduce FcγR or complement binding to the Fc.

Specific, non-limiting examples of known amino acid modifications to reduce FcγR or complement binding to the Fc include those identified in Table C.

TABLE C
Modifications to reduce FcγR or complement binding to the Fc

| Company | Mutations |
|---|---|
| GSK | N297A |
| Ortho Biotech | L234A/L235A |
| Protein Design labs | IGG2 V234A/G237A |
| Wellcome Labs | IGG4 L235A/G237A/E318A |
| GSK | IGG4 S228P/L236E |
| Alexion | IGG2/IGG4combo |
| Merck | IGG2 H268Q/V309L/A330S/A331S |
| Bristol-Myers | C220S/C226S/C229S/P238S |
| Seattle Genetics | C226S/C229S/E3233P/L235V/L235A |
| Amgen | E. coli production, non glyco |
| Medimune | L234F/L235E/P331S |
| Trubion | Hinge mutant, possibly C226S/P230S |

In some embodiments, the Fc comprises at least one amino acid modification identified in Table C. In some embodiments, the Fc comprises amino acid modification of at least one of L234, L235, or D265. In some embodiments, the Fc comprises amino acid modification at L234, L235 and D265. In some embodiments, the Fc comprises the amino acid modification L234A, L235A and D265S.

### Linkers and Linker Polypeptides

In some embodiments, the TAA presentation inducer construct comprises at least one ISR-binding construct and at least one TAA-binding construct that are linked to each other with a linker. The linker may be a linker peptide, a linker polypeptide, or a non-polypeptide linker. In some embodiments, the TAA presentation inducer constructs described herein include at least one ISR-binding construct and at least one TAA-binding construct that are each operatively linked to a linker polypeptide wherein the linker polypeptides are capable of forming a complex or interface with each other. In some embodiments, the linker polypeptides are capable of forming a covalent linkage with each other. The spatial conformation of the constructs with the linker polypeptides is similar to the relative spatial conformation of the paratopes of a F(ab′)2 fragment generated by papain digestion, albeit in the context of an TAA presentation inducer construct with 2 antigen-binding polypeptide constructs.

In one embodiment, the linker polypeptides are selected from IgG1, IgG2, IgG3, or IgG4 hinge regions.

In some embodiments, the linker polypeptides are selected such that they maintain the relative spatial conformation of the paratopes of a F(ab′) fragment, and are capable of forming a covalent bond equivalent to the disulphide bond in the core hinge of IgG. Suitable linker polypeptides include IgG hinge regions such as, for example those from IgG1, IgG2, or IgG4. Modified versions of these exemplary linkers can also be used. For example, modifications to improve the stability of the IgG4 hinge are known in the art (see for example, Labrijn et al. (2009) Nature Biotechnology 27, 767-771).

In one embodiment, the linker polypeptides are operatively linked to a scaffold as described here, for example an Fc. In some aspects, an Fc is coupled to the one or more antigen-binding polypeptide constructs with one or more linkers. In some aspects, Fc is coupled to the heavy chain of each antigen-binding polypeptide by a linker.

In other embodiments, the linker polypeptides are operatively linked to scaffolds other than an Fc. A number of scaffolds based on alternate protein or molecular domains are known in the art and can be used to form selective pairs of two different target-binding polypeptides. Examples of such alternate domains are the split albumin scaffolds described in WO 2012/116453 and WO 2014/012082. A further example is the leucine zipper domains such as Fos and Jun that selectively pair together [S A Kostelny, M S Cole, and J Y Tso. Formation of a bispecific antibody by the use of leucine zippers. J Immunol 1992 148:1547-53; Bernd J. Wranik, Erin L. Christensen, Gabriele Schaefer, Janet K. Jackman, Andrew C. Vendel, and Dan Eaton. LUZ-Y, a Novel Platform for the Mammalian Cell Production of Full-length IgG-bispecific Antibodies J. Biol. Chem. 2012 287: 43331-43339]. Alternately, other selectively pairing molecular pairs such as the barnase barstar pair [Deyev, S. M., Waibel, R., Lebedenko, E. N., Schubiger, A. P., and PlUckthun, A. (2003). Design of multivalent complexes using the barnase*barstar module. Nat Biotechnol 21, 1486-1492], DNA strand pairs [Zahida N. Chaudri, Michael Bartlet-Jones, George Panayotou, Thomas Klonisch, Ivan M. Roitt, Torben Lund, Peter J. Delves, Dual specificity antibodies using a double-stranded oligonucleotide bridge, FEBS Letters, Volume 450, Issues 1-2, 30 Apr. 1999, Pages 23-26], split fluorescent protein pairs [Ulrich Brinkmann, Alexander Haas. Fluorescent antibody fusion protein, its production and use, WO 2011135040 A1] can also be employed.

### Methods of Preparing the TAA Presentation Inducer Constructs

The TAA presentation inducer constructs described herein may be produced using recombinant methods and compositions, e.g., as described in U.S. Pat. No. 4,816,567.

Certain embodiments thus relate to one or more nucleic acids encoding a TAA presentation inducer construct described herein. Such nucleic acid may encode an amino acid sequence corresponding to the at least one ISR-binding construct and/or the at least one TAA-binding construct, and may further include linkers and scaffolds if present in the TAA presentation inducer construct.

Certain embodiments relate to one or more vectors (e.g., expression vectors) comprising nucleic acid encoding a TAA presentation inducer construct described herein. In some embodiments, the nucleic acid encoding the TAA presentation inducer construct is included in a multicistronic vector. In other embodiments, each polypeptide chain of the TAA presentation inducer construct is encoded by a separate vector. It is further contemplated that combinations of vectors may comprise nucleic acid encoding a single TAA presentation inducer construct.

Certain embodiments relate to host cells comprising such nucleic acid or one or more vectors comprising the nucleic acid. In some embodiments, for example, where the TAA presentation inducer construct is a multispecific or bispecific antibody, a host cell comprises (e.g., has been transformed with): (1) a vector comprising a nucleic acid that encodes an amino acid sequence comprising the VL of the antigen-binding domain and an amino acid sequence comprising the VH of the antigen-binding domain, or (2) a first vector comprising a nucleic acid that encodes an amino acid sequence comprising the VL of the antigen-binding domain and a second vector comprising a nucleic acid that encodes an amino acid sequence comprising the VH of the antigen-binding domain. In some embodiments, the host cell is eukaryotic, e.g. a Chinese Hamster Ovary (CHO) cell, or human embryonic kidney (HEK) cell, or lymphoid cell (e.g., Y0, NS0, Sp20 cell).

Certain embodiments relate to a method of making a TAA presentation inducer construct, wherein the method comprises culturing a host cell comprising nucleic acid encoding the TAA presentation inducer construct, as described above, under conditions suitable for expression of the TAA presentation inducer construct, and optionally recovering the TAA presentation inducer construct from the host cell (or host cell culture medium).

For recombinant production of the TAA presentation inducer construct, nucleic acid encoding a TAA presentation inducer construct, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the TAA presentation inducer construct).

The term “substantially purified” refers to a construct described herein, or variant thereof, that may be substantially or essentially free of components that normally accompany or interact with the protein as found in its naturally occurring environment, i.e. a native cell, or host cell in the case of recombinantly produced construct. In certain embodiments, a construct that is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein. When the construct is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the construct is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells.

In certain embodiments, the term “substantially purified” as applied to a construct comprising a heteromultimer Fc and produced by the methods described herein, has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Suitable host cells for cloning or expression of TAA presentation inducer construct-encoding vectors include prokaryotic or eukaryotic cells described herein.

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, and the like.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, and the like) phylogenetic domain, or the Archaea (including but not limited to, Methanococcus jannaschii, Methanobacterium thermoautotrophicum, Halobacterium such as Haloferax vokanii and Halobacterium species NRC-1, Archaeoglobus fulgidus, Pyrococcus furiosus, Pyrococcus horikoshii, Aeuropyrum pernix, and the like) phylogenetic domain.

For example, a TAA presentation inducer construct may be produced in bacteria, in particular when glycosylation and Fc effector function are not needed. For expression of antigen-binding construct fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antigen-binding construct may be isolated from the bacterial cell paste in a soluble fraction and can be further purified.

In addition to prokaryotes, eukaryotic microbes such as filamentous fungi or yeast are suitable cloning or expression hosts for TAA presentation inducer construct-encoding vectors, including fungi and yeast strains whose glycosylation pathways have been “humanized,” resulting in the production of an antigen-binding construct with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antigen-binding constructs are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antigen-binding constructs in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TM cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antigen-binding construct production, see, e.g., Yazaki and Wu, Methods in Molecular Biology, Vol. 248 (B. K. C. Lo, ed., Humana Press, Totowa, N.J.), pp. 255-268 (2003).

In some embodiments, the TAA presentation inducer constructs described herein are produced in stable mammalian cells, by a method comprising: transfecting at least one stable mammalian cell with: nucleic acid encoding the TAA presentation inducer construct, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the antigen-binding construct in the expressed product.

In some embodiments, in the method of producing a TAA presentation inducer construct in stable mammalian cells, the expression product of the stable mammalian cell comprises a larger percentage of the desired glycosylated antigen-binding construct as compared to the monomeric heavy or light chain polypeptides, or other antibodies.

If required, the TAA presentation inducer constructs can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can used for purification of antigen-binding constructs. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni⁺² affinity chromatography if a His-tag is employed, or immobilized anti-flag antibody if a flag-tag is used. For general guidance in suitable purification techniques, see, e.g. incorporated entirely by reference Protein Purification: Principles and Practice, 3^{rd} Ed., Scopes, Springer-Verlag, NY, 1994, incorporated entirely by reference. The degree of purification necessary will vary depending on the use of the antigen-binding constructs. In some instances no purification is necessary.

In certain embodiments, the TAA presentation inducer constructs may be purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In some embodiments, the TAA presentation inducer constructs are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, the TAA presentation inducer constructs can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4-aminobutyric acid, Abu, 2-amino butyric acid, g-Abu, eAhx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as α-methyl amino acids, C α-methyl amino acids, N α-methyl amino acids, and amino acid analogs in general. Furthermore, the amino acid can be D (dextrorotary) or L (levorotary).

### Post-Translational Modifications

In certain embodiments, the TAA presentation inducer constructs described herein are differentially modified during or after translation.

The term “modified,” as used herein, refers to any changes made to a given polypeptide, such as changes to the length of the polypeptide, the amino acid sequence, chemical structure, co-translational modification, or post-translational modification of a polypeptide.

The term “post-translationally modified” refers to any modification of a natural or non-natural amino acid that occurs to such an amino acid after it has been incorporated into a polypeptide chain. The term encompasses, by way of example only, co-translational in vivo modifications, co-translational in vitro modifications (such as in a cell-free translation system), post-translational in vivo modifications, and post-translational in vitro modifications.

In some embodiments, the TAA presentation inducer constructs may comprise a modification that is: glycosylation, acetylation, phosphorylation, amidation, derivatization by known protecting/blocking groups, proteolytic cleavage or linkage to an antibody molecule or antigen-binding construct or other cellular ligand, or a combination of these modifications. In some embodiments, the TAA presentation inducer construct is chemically modified by known techniques, including but not limited, to specific chemical cleavage by cyanogen bromide, trypsin, chymotrypsin, papain, V8 protease, NaBH₄; acetylation, formylation, oxidation, reduction; and metabolic synthesis in the presence of tunicamycin.

Additional optional post-translational modifications of antigen-binding constructs include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The antigen-binding constructs described herein are modified with a detectable label, such as an enzymatic, fluorescent, isotopic or affinity label to allow for detection and isolation of the protein. In certain embodiments, examples of suitable enzyme labels include horseradish peroxidase, alkaline phosphatase, beta-galactosidase, or acetylcholinesterase; examples of suitable prosthetic group complexes include streptavidin biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include iodine, carbon, sulfur, tritium, indium, technetium, thallium, gallium, palladium, molybdenum, xenon, fluorine.

In some embodiments, antigen-binding constructs described herein may be attached to macrocyclic chelators that associate with radiometal ions.

In some embodiments, the TAA presentation inducer constructs described herein may be modified by either natural processes, such as post-translational processing, or by chemical modification techniques which are well known in the art. In certain embodiments, the same type of modification may be present in the same or varying degrees at several sites in a given polypeptide. In certain embodiments, polypeptides from antigen-binding constructs described herein are branched, for example, as a result of ubiquitination, and in some embodiments are cyclic, with or without branching. Cyclic, branched, and branched cyclic polypeptides are a result from posttranslation natural processes or made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York, pgs. 1-12 (1983); Seifter et al., Meth. Enzymol. 182:626-646 (1990); Rattan et al., Ann. N.Y. Acad. Sci. 663:48-62 (1992)).

In certain embodiments, antigen-binding constructs described herein may be attached to solid supports, which are particularly useful for immunoassays or purification of polypeptides that are bound by, that bind to, or associate with proteins described herein. Such solid supports include, but are not limited to, glass, cellulose, polyacrylamide, nylon, polystyrene, polyvinyl chloride or polypropylene.

In cases where the TAA presentation inducer construct comprises at least one ISR-binding construct or at least one TAA-binding construct that is not a peptide or polypeptide, the ISR-binding construct and/or a TAA-binding construct may be chemically conjugated to each other, or to the linker or scaffold, if present.

### Additional Optional Modifications

In one embodiment, the TAA presentation inducer construct described herein can be further modified (i.e., by the covalent attachment of various types of molecules) such that covalent attachment does not interfere with or affect the ability of the TAA presentation inducer to bind to the ISR or TAA, or negatively affect its stability. Such modifications include, for example, but not by way of limitation, glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formylation, metabolic synthesis of tunicamycin, etc.

In another embodiment, the TAA presentation inducer construct described herein can be conjugated (directly or indirectly) to a therapeutic agent or drug moiety that modifies a given biological response. In certain embodiments the TAA presentation inducer construct is conjugated to a drug, e.g., a toxin, a chemotherapeutic agent, an immune modulator, or a radioisotope. Several methods of conjugating polypeptide to drugs or small molecules are known in the art. For example, methods for the preparation of ADCs (antibody-drug conjugates) are described in U.S. Pat. No. 8,624,003 (pot method), U.S. Pat. No. 8,163,888 (one-step), and U.S. Pat. No. 5,208,020 (two-step method) for example. In some embodiments, the drug is selected from a maytansine, auristatin, calicheamicin, or derivative thereof. In other embodiments, the drug is a maytansine selected from DM1 and DM4. In some embodiments, the drug moiety may be a microtubule polymerization inhibitor or DNA intercalator. In other embodiments, the drug moiety may be an immunostimulatory agent such as a TLR (toll-like receptor) agonist or STING (stimulator of interferon gene) agonist.

In some embodiments, the TAA presentation inducer construct is conjugated to a cytotoxic agent. The term “cytotoxic agent” as used herein refers to a substance that inhibits or prevents the function of cells and/or causes destruction of cells. The term is intended to include radioactive isotopes (e.g. At211, I131, I125, Y90, Re186, Re188, Sm153, Bi212, P32, and Lu177), chemotherapeutic agents, and toxins such as small molecule toxins or enzymatically active toxins of bacterial, fungal, plant or animal origin, including fragments and/or variants thereof.

Therapeutic agents or drug moieties are not to be construed as limited to classical chemical therapeutic agents. For example, the drug moiety can be a protein or polypeptide possessing a desired biological activity. Such proteins can include, for example, a toxin such as abrin, ricin A, Onconase (or another cytotoxic RNase), pseudomonas exotoxin, cholera toxin, or diphtheria toxin; a protein such as tumor necrosis factor, alpha-interferon, beta-interferon, nerve growth factor, platelet derived growth factor, tissue plasminogen activator, an apoptotic agent, e.g., TNF-alpha, TNF-beta, AIM I (see, International Publication No. WO 97/33 899), AIM II (see, International Publication No. WO 97/34911), Fas Ligand (Takahashi et al., 1994, J. Immunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, in an alternate embodiment, the TAA presentation inducer construct can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483; Peterson et al., 1999, Bioconjug. Chem. 10:553; and Zimmerman et al., 1999, Nucl. Med. Biol. 26:943.

In some embodiments, the TAA presentation inducer construct may be expressed as fusion proteins comprising a tag to facilitate purification and/or testing etc. As referred to herein, a “tag” is any added series of amino acids which are provided in a protein at either the C-terminus, the N-terminus, or internally that contributes to the identification or purification of the protein. Suitable tags include but are not limited to tags known to those skilled in the art to be useful in purification and/or testing such as albumin binding domain (ABD), His tag, FLAG tag, glutathione-s-transferase, hemagglutinin (HA) and maltose binding protein. Such tagged proteins can also be engineered to comprise a cleavage site, such as a thrombin, enterokinase or factor×cleavage site, for ease of removal of the tag before, during or after purification.

### Testing the TAA Presentation Inducer Constructs

The ability of the TAA presentation inducer constructs to bind to ISRs and/or TAAs can be tested according to methods known in the art. The ability of a TAA presentation inducer construct to bind to a TAA or ISR can be assessed by antigen-binding assays (where the ISR-binding construct and/or the TAA-binding construct are antibodies or fragments thereof) or cell binding assays. Antigen-binding assays are carried out by incubating the TAA presentation inducer construct with antigen (ISR or TAA), either purified, or in a mixture and assessing the amount of TAA presentation inducer bound to the antigen, compared to controls. The amount of TAA presentation inducer construct bound to the antigen can by assessed by ELISA, or SPR (surface plasmon resonance), for example. Cell binding assays are carried out by incubating the TAA presentation inducer construct with cells that express the ISR or TAA of interest (such cells are commercially available). The amount of TAA presentation inducer construct bound to the cells can be assessed by flow cytometry, for example, and compared to binding observed in the presence of controls. Methods for carrying out these types of assays are well known in the art.

The TAA presentation inducer constructs may be tested to determine if they promote TCDM acquisition by APCs. Suitable assays can involve incubation of labeled tumor cells expressing the TAA of interest with cells expressing the ISR of interest in co-culture. In some cases, the labelled tumor cells are physically separated from the cells expressing the ISR of interest using transwell chambers. At various timepoints after co-culture initiation, the ISR-expressing cells are collected and the label content evaluated by flow cytometry or high-content imaging. Such methods are described in the art, and exemplary methods are described in the Examples.

The TAA presentation inducer constructs may also be tested to determine if they promote TCDM-dependent activation of cells expressing the ISR of interest. In an exemplary assay, MHC presentation of TCDM-derived peptides induced by the TAA presentation inducer construct is evaluated by assessing the ability of ISR-expressing cells to stimulate T cells following co-culture of the ISR-expressing cells with tumor cells expressing the TAA of interest. ISR agonism can be evaluated via supernatant cytokine or cell-surface activation marker quantification at multiple times following initiation of the co-culture. Cytokine production can be quantified via commercially available ELISA or bead-based multiplex systems, while cell-surface activation marker expression can be quantified via flow cytometry or high-content imaging. Methods of assessing TCDM-dependent activation of ISR-expressing cells are well known, and exemplary methods are described in the Examples.

The TAA presentation inducer constructs may also be tested to determine if they induce MHC TAA presentation and polyclonal T cell activation. For example, co-culture of ISR-expressing cells and TAA-expressing tumor cells is carried out as described in the preceding paragraph. Co-culture is carried out as described above, but at various timepoints, antigen presentation is assessed by transferring the ISR-expressing cells to a secondary T cell activation co-culture. After several days, TAA-specific T cell responses are quantified by flow cytometric staining with fluorescent peptide-MHC multimers (ImmuDex). In some cases, T cells can subsequently be transferred to tertiary cultures containing peptide-pulsed allogeneic APCs, and TAA response frequency additionally assessed via cytokine-specific ELISpot.

In vivo effects of the TAA presentation inducer constructs may also be evaluated by standard techniques. For example, the effect of TAA presentation inducer constructs on tumor growth can be examined in various tumor models. Several suitable animal models are known in the art to test the ability of candidate therapies to treat cancers, such as, for example, breast cancers or gastric cancers. Some models are commercially available. In general, these models are mouse xenograft models, where cell line-derived tumors or patient-derived tumors are implanted in mice. The construct to be tested is generally administered after the tumor has been established in the animal, but in some cases, the construct can be administered with the cell line. The volume of the tumor and/or survival of the animal is monitored in order to determine if the construct is able to treat the tumor. The construct may be administered intravenously (i.v.), intraperitoneally (i.p.) or subcutaneously (s.c.). Dosing schedules and amounts vary but can be readily determined by the skilled person. An exemplary dosage would be 10 mg/kg once weekly. Tumor growth can be monitored by standard procedures. For example, when labelled tumor cells have been used, tumor growth may be monitored by appropriate imaging techniques. For solid tumors, tumor size may also be measured by caliper.

### Pharmaceutical Compositions

Certain embodiments relate to pharmaceutical compositions comprising a TAA presentation inducer construct described herein and a pharmaceutically acceptable carrier.

The term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, vehicle, or combination thereof, with which the construct is administered. Such pharmaceutical carriers can be sterile liquids, such as water and oils, including those of petroleum, animal, vegetable or synthetic origin, such as peanut oil, soybean oil, mineral oil, sesame oil and the like. In some aspects, the carrier is a man-made carrier not found in nature. Water can be used as a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Examples of suitable pharmaceutical carriers are described in “Remington's Pharmaceutical Sciences” by E. W. Martin.

The pharmaceutical compositions may be in the form of solutions, suspensions, emulsion, tablets, pills, capsules, powders, sustained-release formulations and the like. The composition may be formulated as a suppository, with traditional binders and carriers such as triglycerides. Oral formulations may include standard carriers such as pharmaceutical grades of mannitol, lactose, starch, magnesium stearate, sodium saccharine, cellulose, magnesium carbonate, and the like.

Pharmaceutical compositions will contain a therapeutically effective amount of the TAA presentation inducer construct, together with a suitable amount of carrier so as to provide the form for proper administration to a patient. The formulation should suit the mode of administration.

In certain embodiments, the composition comprising the TAA presentation inducer construct is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the compositions described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, and the like.

### Methods of Using the TAA Presentation Inducer Constructs

The TAA presentation inducer constructs described herein may be used to induce major histocompatibility complex (MHC) presentation of peptides from one or more tumor-associated antigens (TAAs) by a single ISR-expressing cell simultaneously in a subject. The one or more TAAs may include the TAA that is directly bound by the TAA presentation inducer construct (i.e. the first TAA), as well as additional TAAs that are part of the TCDM that is physically associated with the first TAA (i.e. secondary TAAs). Thus, in one embodiment the TAA presentation inducer constructs can be used in a method of inducing MHC presentation of peptides from one or more secondary TAAs by a single ISR-expressing cell simultaneously in a subject. In an alternative embodiment, the TAA presentation inducer constructs can be used in a method of inducing MHC presentation of peptides from a first TAA and one or more secondary TAAs by a single ISR-expressing cell simultaneously in a subject.

In one embodiment, the TAA presentation inducer constructs may also be used to induce ISR-expressing cell activation in a subject. Upon contact with the TAA presentation inducer, the ISR-expressing cell is activated and subsequently produces cytokines and/or up-regulates co-stimulatory ligands. Thus, in one embodiment, the TAA presentation inducer constructs can be used in a method of inducing ISR-expressing cell activation in a subject.

In one embodiment, the TAA presentation inducer construct may be used to induce a polyclonal T cell response in a subject. In one embodiment, the TAA presentation inducer construct may be used to induce a polyclonal T cell response that is capable of adapting to the heterogeneity and dynamic nature of neoplastic cells. For example, some anti-tumor therapies directed against pre-defined tumor antigens may lose efficacy either because the immune response to the tumor is suppressed, or because changes in the tumor cell result in loss of the pre-defined tumor antigens. Because the TAA presentation inducer construct described herein is capable of directing TCDM to an APC, the TAA presentation inducer may be able to maintain efficacy as an anti-tumor therapy as the TAA composition of the TCDM changes.

In another embodiment, the TAA presentation inducer construct may be used in a method to expand, activate or differentiate T cells specific for two or more TAAs (either two or more secondary TAAs, or the first TAA and one or more secondary TAAs) simultaneously, the method comprising the steps of: obtaining T cells and innate stimulatory receptor (ISR)-expressing cells from a subject; and culturing the T cells and the ISR-expressing cells with the TAA presentation inducer construct in the presence of tumor cell-derived material (TCDM), to produce expanded, activated or differentiated T cells. In further embodiments, the TCDM is from an autologous primary tumor and/or autologous metastatic tissue sample, an allogeneic tumor sample, or from a tumor cell line.

In further embodiments, T cell populations expanded, activated, or differentiated in vitro using a TAA presentation inducer construct may be administered to a subject having cancer, in need of such therapy. Thus, the TAA presentation inducer constructs can be used to prepare T cell populations that have been expanded, activated, or differentiated in vitro by the methods described herein, and such T cell populations administered to a subject having cancer.

In yet another embodiment, the TAA presentation inducer construct may be used in a method of identifying tumor-associated antigens in tumor cell-derived material (TCDM), the method comprising isolating T cells and enriched innate stimulatory receptor (ISR)-expressing cells from a subject; culturing the ISR-expressing cells and the T cells with the TAA presentation inducer construct in the presence of tumor cell-derived material (TCDM), to produce TAA presentation inducer construct-activated ISR-expressing cells, and determining the sequence of TAA peptides eluted from MHC complexes of the TAA presentation inducer construct-activated ISR-expressing cells; and identifying the TAAs corresponding to the TAA peptides.

In another embodiment, the TAA presentation inducer construct may be used in a method of identifying T cell receptor (TCR) target polypeptides, the method comprising isolating T cells and enriched innate stimulatory receptor (ISR)-expressing cells from a subject; culturing the ISR-expressing cells and the T cells with the TAA presentation inducer construct in the presence of tumor cell-derived material (TCDM), to produce TAA presentation inducer construct-activated ISR-expressing cells and activated T cells, and screening the activated T cells against a library of candidate TAAs to identify the TCR target polypeptides.

The methods described above include the performance of steps that are well known in the art. For example, the step of isolating T cells and/or ISR-expressing cells can be performed as described in the Examples, or by other methods known in the art, for example those described in Tomlinson et al. (2012) J. of Tissue Eng. 4 (1):1-14. Sequencing of peptides can be performed by any number of methods known in the art. Screening of activated T cells to identify TCR targets can also be achieved by a number of methods known in the art.

In certain embodiments, provided is a method of treating a cancer comprising administering to a subject in which such treatment, prevention or amelioration is desired, an TAA presentation inducer construct described herein, in an amount effective to treat, prevent or ameliorate the cancer. In other embodiments, there is provided a method of using the TAA presentation inducer construct in the preparation of a medicament for the treatment, prevention, or amelioration of cancer in a subject.

The term “subject” refers to an animal, in some embodiments a mammal, which is the object of treatment, observation or experiment. An animal may be a human, a non-human primate, a companion animal (e.g., dogs, cats, and the like), farm animal (e.g., cows, sheep, pigs, horses, and the like) or a laboratory animal (e.g., rats, mice, guinea pigs, and the like).

The term “mammal” as used herein includes but is not limited to humans, non-human primates, canines, felines, murines, bovines, equines, and porcines.

“Treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishing of any direct or indirect pathological consequences of the disease, preventing metastasis, decreasing the rate of disease progression, amelioration or palliation of the disease state, and remission or improved prognosis. In some embodiments, TAA presentation inducer constructs described herein are used to delay development of a disease or disorder. In one embodiment, TAA presentation inducer constructs and methods described herein effect tumor regression. In one embodiment, TAA presentation inducer constructs and methods described herein effect inhibition of tumor/cancer growth.

Desirable effects of treatment include, but are not limited to, one or more of preventing occurrence or recurrence of disease, alleviation of symptoms, diminishment of any direct or indirect pathological consequences of the disease, preventing metastasis, decreasing the rate of disease progression, amelioration or palliation of the disease state, improved survival, and remission or improved prognosis. In some embodiments, TAA presentation inducer constructs described herein are used to delay development of a disease or to slow the progression of a disease.

The term “effective amount” as used herein refers to that amount of construct being administered, which will accomplish the goal of the recited method, e.g., relieve to some extent one or more of the symptoms of the disease, condition or disorder being treated. The amount of the composition described herein which will be effective in the treatment, inhibition and prevention of a disease or disorder associated with aberrant expression and/or activity of a therapeutic protein can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

The TAA presentation inducer construct is administered to a subject. Various delivery systems are known and can be used to administer an TAA presentation inducer construct formulation described herein, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, in certain embodiments, it is desirable to introduce the TAA presentation inducer construct compositions described herein into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it is desirable to administer the TAA presentation inducer constructs, or compositions described herein locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an TAA presentation inducer construct, described herein, care must be taken to use materials to which the protein does not absorb.

In another embodiment, the TAA presentation inducer constructs or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the TAA presentation inducer constructs or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, e.g., the brain, thus requiring only a fraction of the systemic dose (see, e.g., Goodson, in Medical Applications of Controlled Release, vol. 2, pp. 115-138 (1984)).

In a specific embodiment comprising a nucleic acid encoding TAA presentation inducer constructs described herein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The amount of the TAA presentation inducer construct which will be effective in the treatment, inhibition and prevention of a disease or disorder can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

The TAA presentation inducer constructs described herein may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred.

The TAA presentation inducer constructs described herein may be used in the treatment of cancer. In some embodiments, the TAA presentation inducer construct may be used in the treatment of a patient who has undergone one or more alternate forms of anti-cancer therapy. In some embodiments, the patient has relapsed or failed to respond to one or more alternate forms of anti-cancer therapy. In other embodiments, the TAA presentation inducer construct is administered to a patient in combination with one or more alternate forms of anti-cancer therapy. In other embodiments, the TAA presentation inducer construct is administered to a patient that has become refractory to treatment with one or more alternate forms of anti-cancer therapy.

### Kits and Articles of Manufacture

Also described herein are kits comprising one or more TAA presentation inducer constructs. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the TAA presentation inducer construct.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilized form and the kit can additionally contain a suitable solvent for reconstitution of the lyophilized components. Irrespective of the number or type of containers, the kits described herein also may comprise an instrument for assisting with the administration of the composition to a patient. Such an instrument may be an inhalant, nasal spray device, syringe, pipette, forceps, measured spoon, eye dropper or similar medically approved delivery vehicle.

Certain embodiments relate to an article of manufacture containing materials useful for treatment of a patient as described herein. The article of manufacture comprises a container and a label or package insert on or associated with the container. Suitable containers include, for example, bottles, vials, syringes, intravenous solution bags, etc. The containers may be formed from a variety of materials such as glass or plastic. The container holds a composition comprising the TAA presentation inducer construct which is by itself or combined with another composition effective for treating the patient and may have a sterile access port (for example the container may be an intravenous solution bag or a vial having a stopper pierceable by a hypodermic injection needle). The label or package insert indicates that the composition is used for treating the condition of choice. In some embodiments, the article of manufacture may comprise (a) a first container with a composition contained therein, wherein the composition comprises a TAA presentation inducer construct described herein; and (b) a second container with a composition contained therein, wherein the composition in the second container comprises a further cytotoxic or otherwise therapeutic agent. In such embodiments, the article of manufacture may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. The article of manufacture may optionally further include other materials desirable from a commercial and user standpoint, including other buffers, diluents, filters, needles, and syringes.

### Polypeptides and Polynucleotides

As described herein, the TAA presentation inducer constructs comprise at least one polypeptide. Certain embodiments relate to polynucleotides encoding such polypeptides described herein.

The TAA presentation inducer constructs, polypeptides and polynucleotides described herein are typically isolated. As used herein, “isolated” means an agent (e.g., a polypeptide or polynucleotide) that has been identified and separated and/or recovered from a component of its natural cell culture environment. Contaminant components of its natural environment are materials that would interfere with diagnostic or therapeutic uses for the TAA presentation inducer construct, and may include enzymes, hormones, and other proteinaceous or non-proteinaceous solutes. Isolated also refers to an agent that has been synthetically produced, e.g., via human intervention.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full-length proteins, wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as β-alanine, ornithine, etc.; and chemically synthesized compounds having properties known in the art to be characteristic of amino acids. Examples of non-naturally occurring amino acids include, but are not limited to, α-methyl amino acids (e.g. α-methyl alanine), D-amino acids, histidine-like amino acids (e.g., 2-amino-histidine, β-hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the TAA presentation inducer constructs described herein may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Also included herein are polynucleotides encoding polypeptides of the TAA presentation inducer constructs. The term “polynucleotide” or “nucleotide sequence” is intended to indicate a consecutive stretch of two or more nucleotide molecules. The nucleotide sequence may be of genomic, cDNA, RNA, semisynthetic or synthetic origin, or any combination thereof.

The term “nucleotide sequence” or “nucleic acid sequence” is intended to indicate a consecutive stretch of two or more nucleotide molecules. The nucleotide sequence can be of genomic, cDNA, RNA, semisynthetic or synthetic origin, or any combination thereof.

“Cell”, “host cell”, “cell line” and “cell culture” are used interchangeably herein and all such terms should be understood to include progeny resulting from growth or culturing of a cell. “Transformation” and “transfection” are used interchangeably to refer to the process of introducing a nucleic acid sequence into a cell.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also encompasses every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid.

Conservative substitution tables providing functionally similar amino acids are known to those of ordinary skill in the art. Such conservatively modified variants are in addition to and do not exclude polymorphic variants, interspecies homologs, and alleles described herein. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and [0139] 8) Cysteine (C), Methionine (M) (see, e.g., Creighton, Proteins: Structures and Molecular Properties (W H Freeman & Co.; 2nd edition (December 1993).

The term “identical” in the context of two or more nucleic acids or polypeptide sequences, refers to two or more sequences or subsequences that are the same. Sequences are “substantially identical” if they have a percentage of amino acid residues or nucleotides that are the same (i.e., about 60% identity, about 65%, about 70%, about 75%, about 80%, about 85%, about 90%, or about 95% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide described herein, including homologs from species other than human, may be obtained by a process comprising the steps of screening a library under stringent hybridization conditions with a labeled probe having a polynucleotide sequence described herein or a fragment thereof, and isolating full-length cDNA and genomic clones containing said polynucleotide sequence. Such hybridization techniques are well known to the skilled artisan.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are known to those of ordinary skill in the art. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of an algorithm that is suitable for determining percent sequence identity and sequence similarity are the BLAST and BLAST 2.0 algorithms, which are described in Altschul et al. (1997) Nuc. Acids Res. 25:3389-3402, and Altschul et al. (1990) J. Mol. Biol. 215:403-410, respectively. Software for performing BLAST analyses is publicly available through the National Center for Biotechnology Information available at the World Wide Web at ncbi.nlm.nih.gov. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1992) Proc. Natl. Acad. Sci. USA 89:10915) alignments (B) of 50, expectation (E) of 10, M=5, N=−4, and a comparison of both strands. The BLAST algorithm is typically performed with the “low complexity” filter turned off.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001.

The phrase “selectively (or specifically) hybridizes to” refers to the binding, duplexing, or hybridizing of a molecule only to a particular nucleotide sequence under stringent hybridization conditions when that sequence is present in a complex mixture (including but not limited to, total cellular or library DNA or RNA).

The phrase “stringent hybridization conditions” refers to hybridization of sequences of DNA, RNA, or other nucleic acids, or combinations thereof under conditions of low ionic strength and high temperature as is known in the art. Typically, under stringent conditions a probe will hybridize to its target subsequence in a complex mixture of nucleic acid (including but not limited to, total cellular or library DNA or RNA) but does not hybridize to other sequences in the complex mixture. Stringent conditions are sequence-dependent and will be different in different circumstances. Longer sequences hybridize specifically at higher temperatures. An extensive guide to the hybridization of nucleic acids is found in Tijssen, Laboratory Techniques in Biochemistry and Molecular Biology—Hybridization with Nucleic Probes, “Overview of principles of hybridization and the strategy of nucleic acid assays” (1993).

As used herein, the term “engineer,” and grammatical variations thereof is considered to include any manipulation of a peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. The engineered proteins are expressed and produced by standard molecular biology techniques.

A derivative, or a variant of a polypeptide is said to share “homology” or be “homologous” with the polypeptide if the amino acid sequences of the derivative or variant has at least 50% identity with a 100 amino acid sequence from the original polypeptide. In certain embodiments, the derivative or variant is at least 75% the same as that of either the polypeptide or a fragment of the polypeptide having the same number of amino acid residues as the derivative. In various embodiments, the derivative or variant is at least 85%, 90%, 95% or 99% the same as that of either the polypeptide or a fragment of the polypeptide having the same number of amino acid residues as the derivative.

In some aspects, a TAA presentation inducer construct comprises an amino acid sequence that is at least 80, 85, 90, 91, 92, 93, 94, 95, 96, 97, 98, 99, or 100% identical to a relevant amino acid sequence or fragment thereof set forth in the Tables or accession numbers disclosed herein. In some aspects, an isolated TAA presentation inducer construct comprises an amino acid sequence encoded by a polynucleotide that is at least 80, 85, 90, 91, 92, 93, 94, 95, 96, 97, 98, 99, or 100% identical to a relevant nucleotide sequence or fragment thereof set forth in Tables or accession numbers disclosed herein.

It is to be understood that this disclosure is not limited to the particular protocols; cell lines, constructs, and reagents described herein and as such may vary. It is also to be understood that the terminology used herein is for the purpose of describing particular embodiments only, and is not intended to limit the scope of protection.

All publications and patents mentioned herein are incorporated herein by reference for the purpose of describing and disclosing, for example, the constructs and methodologies that are described in the publications, which might be used in connection with the presently described TAA presentation inducer constructs. The publications discussed herein are provided solely for their disclosure prior to the filing date of the present application. Nothing herein is to be construed as an admission that the inventors are not entitled to antedate such disclosure by virtue of prior invention or for any other reason.

### EXAMPLES

Below are examples of specific embodiments related to the TAA presentation inducer constructs described herein. The examples are offered for illustrative purposes only, and are not intended to limit the scope of the disclosure in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1: Description of TAA Presentation Inducer Constructs

1) TAA presentation inducer constructs that are bispecific antigen-binding constructs are prepared in the following exemplary formats:
a) A hybrid antibody format (hybrid format) in which one antigen-binding domain is an scFv and the other antigen-binding domain is a Fab. These bispecific antigen-binding constructs further comprise a IgG1 heterodimeric Fc having CH3 domain amino acid substitutions that drive heterodimeric association of the two component Fc polypeptides, FcA and FcB. FcA comprises the following amino acid substitutions: T350V_L351Y_F405A_Y407V; and FcB comprises amino acid substitutions: T350V_T366L_K392L_T394W. These constructs may further comprise amino acid modifications that decrease binding of the Fc to FcGR.
   The amino acid residues in the Fc region are identified according to the EU index as in Kabat referring to the numbering of the EU antibody (Edelman et al., 1969, Proc Natl Acad Sci USA 63:78-85). The hybrid antibody format constructs described in this example include 3 polypeptide chains: one Fc polypeptide fused to an scFv that binds one target; a second Fc polypeptide fused to VH-CH1 domains, and a light chain, where the VH-CH1 domains and the light chain form a Fab region that binds to a second target.
b) A full size antibody (FSA) format in which both antigen-binding domains are Fabs. These bispecific antigen-binding constructs also comprise the heterodimeric Fc described above. The FSA format constructs described could include 4 polypeptide chains: an Fc polypeptide fused to VH-CH1 domains, and a light chain, where the VH-CH1 domains and the light chain form a Fab region that binds to one target; and a second Fc polypeptide fused to VH-CH1 domains, and a second light chain, where the VH-CH1 domains and the light chain form a Fab region that binds to a second target. Alternatively, a single, common light chain may be used in each of the target binding paratopes.
c) A dual scFv format in which both antigen-binding domains are scFvs. These bispecific antigen-binding constructs also comprise the heterodimeric Fc described above. Constructs in the dual scFv format include one Fc polypeptide fused to a VL-VH sequence binding to one target, and a second Fc polypeptide fused to a second VL-VH sequence binding a second target.

2) TAA presentation inducer constructs having an ISR-binding construct that is a ligand for the ISR, and a TAA-binding construct that is an antigen-binding domain are also prepared.

A description of exemplary TAA presentation inducer constructs in one or more of the formats described above is provided in Table 1. Her2, ROR1, and PSMA are tumor-associated antigens (TAAs). RSV1 is a DNA-binding protein found in yeast and is included as a negative control for the TAA-binding or ISR-binding portions of the TAA presentation inducer constructs, as indicated in Table 1.

TABLE 1
Exemplary types of TAA presentation inducer constructs

| Construct | | | | |
|---|---|---|---|---|
| Number | TAA | TAA Class | ISR | ISR Family |
| 1 | Her2 | Highly | RSV1 | Neg. control |
| | | expressed | | |
| 2 | ROR1 | Oncofetal | RSV1 | Neg. control |
| 3 | PSMA | Poorly- | RSV1 | Neg. control |
| | | infiltrated | | |
| | | tumor | | |
| 4 | RSV1 | Neg. control | Dectin-1 | C-type lectin |
| 5 | RSV1 | Neg. control | DEC205 | C-type lectin |
| 6 | RSV1 | Neg. control | CD40 | TNFR |
| 7 | RSV1 | Neg. control | LRP-1 | LDLR |
| 8 | Her2 | Highly | Dectin-1 | C-type lectin |
| | | expressed | | |
| 9 | Her2 | Highly | DEC205 | C-type lectin |
| | | expressed | | |
| 10 | Her2 | Highly | CD40 | TNFR |
| | | expressed | | |
| 11 | Her2 | Highly | LRP-1 | LDLR |
| | | expressed | | |
| 12 | ROR1 | Oncofetal | Dectin-1 | C-type lectin |
| 13 | ROR1 | Oncofetal | DEC205 | C-type lectin |
| 14 | ROR1 | Oncofetal | CD40 | TNFR |
| 15 | ROR1 | Oncofetal | LRP-1 | LDLR |
| 16 | PSMA | Poorly- | Dectin-1 | C-type lectin |
| | | infiltrated | | |
| | | tumor | | |
| 17 | PSMA | Poorly- | DEC205 | C-type lectin |
| | | infiltrated | | |
| | | tumor | | |
| 18 | PSMA | Poorly- | CD40 | TNFR |
| | | infiltrated | | |
| | | tumor | | |
| 19 | PSMA | Poorly- | LRP-1 | LDLR |
| | | infiltrated | | |
| | | tumor | | |

### Example 2: Preparation and Purification of TAA Presentation Inducer Constructs

Specific examples of the TAA presentation inducer constructs described in Example 1 were prepared and purified as described below. Description and sequences of the specific TAA presentation inducer constructs prepared is provided in Table 2. Each of the constructs includes 3 polypeptides, A, B, and C. The clone number for each polypeptide is listed in Table 2 and the polypeptide and DNA sequences for each clone are found in Table ZZ. As indicated below, for constructs that do not contain calreticulin (CRT), the ISR-binding construct is a Fab, and the TAA-binding construct is an scFv. For constructs that include CRT, the TAA-binding construct is a Fab. All of the constructs include a heterodimeric Fc including the amino acid modifications in Example 1 that that drive heterodimeric Fc formation, along with the amino acid modifications L234A_L235A_D265S that decrease binding of the Fc to FcγR.

TABLE 2
Description of TAA presentation inducer constructs prepared

| Construct # | Targets | Paratopes | Format | A clone # | B clone # | C clone # |
|---|---|---|---|---|---|---|
| 18508 | Dectin-1 X RSV F | 15E2.5, Palivizumab | Fab x scFv | 12644 | 12645 | 11082 |
| 18509 | Dectin-1 X RSV F | 2D8.2D4, Palivizumab | Fab x scFv | 12646 | 12647 | 11082 |
| 18510 | Dectin-1 X RSV F | 11B6.4, Palivizumab | Fab x scFv | 12648 | 12649 | 11082 |
| 18511 | DEC-205 X RSV F | 3G9, Palivizumab | Fab x scFv | 12650 | 12651 | 11082 |
| 18512 | CD40 X RSV F | 12E12, Palivizumab | Fab x scFv | 12652 | 12653 | 11082 |
| 18513 | HER2 X RSV F | Pertuzumab, Palivizumab | scFv x Fab | 11011 | 11074 | 12654 |
| 18514 | ROR1 X RSV F | R12, Palivizumab | scFv x Fab | 11011 | 11074 | 12655 |
| 18516 | LRP-1RSV F | CRT, Palivizumab | ligand x Fab | 11011 | 11074 | 12667 |
| 18520 | Dectin-1 X HER2 | 15E2.5, Pertuzumab | Fab x scFv | 12644 | 12645 | 12654 |
| 18521 | Dectin-1 X ROR1 | 15E2.5, R12 | Fab x scFv | 12644 | 12645 | 12655 |
| 18523 | Dectin-1 X HER2 | 2D8.2D4, Pertuzumab | Fab x scFv | 12646 | 12647 | 12654 |
| 18524 | Dectin-1 X ROR1 | 2D8.2D4, R12 | Fab x scFv | 12646 | 12647 | 12655 |
| 18526 | Dectin-1 X HER2 | 11B6.4, Pertuzumab | Fab x scFv | 12648 | 12649 | 12654 |
| 18527 | Dectin-1 X ROR1 | 11B6.4, R12 | Fab x scFv | 12648 | 12649 | 12655 |
| 18529 | DEC-205 X HER2 | 3G9, Pertuzumab | Fab x scFv | 12650 | 12651 | 12654 |
| 18530 | DEC-205 X ROR1 | 3G9, R12 | Fab x scFv | 12650 | 12651 | 12655 |
| 18532 | CD40 X HER2 | 12E12, Pertuzumab | Fab x scFv | 12652 | 12653 | 12654 |
| 18533 | CD40 X ROR1 | 12E12, R12 | Fab x scFv | 12652 | 12653 | 12655 |
| 18535 | LRP-1 X HER2 | CRT, Pertuzumab | ligand x Fab | 12657 | 12658 | 12667 |
| 18536 | LRP-1 X ROR1 | CRT, R12 | ligand x Fab | 12659 | 12660 | 12667 |
| 18537 | LRP-1 X PSMA | CRT, MLN2704 | ligand x Fab | 12661 | 12662 | 12667 |

The genes encoding the antibody heavy and light chains were constructed via gene synthesis using codons optimized for human/mammalian expression. The scFv and Fab sequences were generated from the sequences of known antibodies, identified in Table 3.

TABLE 3
References for TAA presentation inducer construct sequences

| Target | Paratope/Antibody clone | Reference |
|---|---|---|
| RSV1 | Palivizumab | US20060115485 |
| Her2 | Pertuzumab | WO2015/077891 |
| ROR1 | R12 | WO2012075158 |
| ROR1 | 2A2 | WO2010124188 |
| PSMA | MLN2704 | U.S. Pat. No. 7,045,605 |
| Dectin-1 | 15E2.5 | WO2008118587 |
| Dectin-1 | 2D8.2D4 | WO2008118587 |
| Dectin-1 | 11B6.4 | WO2008118587 |
| DEC205 | 3G9 | WO2009061996 |
| CD40 | 12E12 | US20100239575A1 |
| LRP-1 | Recombinant human | WO2010030861 |
| | calreticulin | |

CDR sequences, as determined by the IMGT numbering system, for some of the antibody clones listed above are found in Table YY.

The final gene products were sub-cloned into a mammalian expression vector and expressed in CHO (Chinese Hamster Ovary) cells (or a functional equivalent) (Durocher, Y., Perret, S. & Kamen, A. High-level and high-throughput recombinant protein production by transient transfection of suspension-growing CHO cells. Nucleic acids research 30, E9 (2002)).

The CHO cells were transfected in exponential growth phase. In order to determine the optimal concentration range for forming heterodimers, the DNA was transfected in various DNA ratios of the FcA, light chain (LC), and FcB that allow for heterodimer formation. FcA:LC:FcB vector transfection ratios were 1:1:1 for scFv-containing variants. FcA:LC:FcB ratios were 2:1:1 for calreticulin fusion variants. Transfected cells culture medium was collected after several days, centrifuged at 4000 rpm and clarified using a 0.45 micron filter.

TAA presentation inducer constructs were purified from the culture medium via established methods. The clarified culture medium was loaded onto a Mab Select SuRe (GEHealthcare) protein-A column and washed with PBS buffer at pH 7.2, eluted with citrate buffer at pH 3.6, and pooled fractions neutralized with TRIS at pH 11. The protein was desalted using an Econo-Pac 10DG column (Bio-Rad). In some cases, the protein was further purified by protein L chromatography or gel filtration. Purified protein concentrations ranged from 1-4 mg/mL, and total yields ranged between 10-50 mg from 1 L transient transfections.

### Example 3: TAA Presentation Inducer Constructs Promote TCDM Acquisition by Antigen-Presenting Cells (APCs)

The ability of TAA presentation inducer constructs to promote TCDM capture by APCs is assessed in tumor cell APC co-culture systems. The tumor cells used in these co-culture systems are from commercially available tumor cell lines such as SKBr3 (expressing the TAA HER2), SKOV3 (expressing the TAAs HER2 and ROR1), or LNCaP (expressing the TAA PSMA). TCDM is naturally generated in cultures of these cell lines, and in some cases TCDM quantity is further increased by addition of exogenous agents such as docetaxel and/or cyclophosphamide. The APCs are prepared from human blood (for example, PBMCs or purified monocytes), or are derived from blood monocytes by pre-culturing purified monocytes with cytokines or cytokine mixtures (such as GM-CSF, M-CSF, IL-4, TNF, and/or IFN).

In some cases, CFSE (Carboxyfluorescein succinimidyl ester])-labeled tumor cells are physically separated from APCs (such as monocytes, macrophages, or dendritic cells) via transwell chambers (such as Sigma Aldrich Corning HTS Transwell # CLS3385). APCs are cultured with tumor cells in multiplicate at various ratios, such as 1 tumor cell to 0.1, 0.3, 1.0, 3.0, or 10 APCs per well. At various timepoints after co-culture initiation, APCs are collected, and CFSE content evaluated via techniques such as flow cytometry or high-content imaging. In some cases, tumor cell-APC cocultures also contain T cells (for example, tumor cell-PBMC cultures) to allow T cell response assessment as described in Example 5.

TAA presentation inducer constructs such as Constructs 8-11 (Table 1), that bind SKBR3 TCDM (tumor cell-derived material) via Her2 and APCs via diverse ISR classes (see Table 1), can promote APC CFSE positivity (TCDM acquisition). Analogous results are observed for ROR1-binding (Constructs 12-15) and PSMA-binding (Constructs 16-19) constructs in APC-SKOV3 or -LNCaP tumor line co-cultures, respectively. Minimal TCDM acquisition is induced by negative constructs that can bind either a TAA or ISR, but not both (i.e. contain a non-binding, negative control paratope) (Constructs 1-7).

### Example 4: TAA Presentation Inducer Constructs Promote TCDM-Dependent APC Activation

The ability of TAA-mediated accumulation of TAA presentation inducer constructs on TCDM to promote ISR agonism in APC-tumor cell co-cultures can be assessed as follows. The APC-co-cultures are carried out as described in Example 3. ISR agonism can be evaluated via supernatant cytokine or cell-surface activation marker quantification at multiple times following APC-tumor cell co-culture initiation. Cytokine production can be quantified via commercially available ELISA or bead-based multiplex systems, while cell-surface activation marker expression can be quantified via flow cytometry or high-content imaging.

TAA presentation inducer constructs such as Constructs 8-11 (Table 1), that bind SKBR3 TCDM via Her2 and APCs via diverse ISR classes (see Table 1), can promote APC cytokine production and/or co-stimulatory ligand upregulation. Analogous results are observed for ROR1-binding (Constructs 12-15) and PSMA-binding (Constructs 16-19) constructs in APC-SKOV3 or -LNCaP tumor line co-cultures, respectively. Minimal APC activation is induced by negative control constructs that can bind either a TAA or ISR, but not both (i.e. contain a non-binding, negative control paratope) (Constructs 1-7), or by TAA presentation inducer constructs in the absence of TCDM.

### Example 5: TAA Presentation Inducer Constructs Induce MHC TAA Presentation and Polyclonal T Cell Activation

MHC presentation of TCDM-derived peptides induced by TAA presentation inducer constructs is evaluated by assessing APC T cell stimulatory capacity following APC-tumor cell co-culture. APC-tumor cell co-culture is carried out as described in Example 3. At various timepoints following a primary, isolated APC-tumor cell co-culture, antigen presentation is assessed by transferring TCDM+TAA presentation inducer construct-treated APCs to a secondary T cell activation co-culture. After several days, TAA-specific T cell responses are quantified by flow cytometric staining with fluorescent peptide-MHC multimers (ImmuDex). In some cases, T cells are subsequently transferred to tertiary cultures containing peptide-pulsed allogeneic APCs, and TAA response frequency additionally assessed via cytokine-specific ELISpot.

If initial APC-tumor cell co-cultures are performed in transwell plates, tumor cell-containing plate inserts are discarded, and T cells are added to APC-containing wells. In cases of direct APC-tumor cell co-culture (non-transwell), APCs are separated from tumor cells by magnetic bead-based isolation for subsequent secondary T cell co-cultures. T cells may be derived from human blood, disease tissue, or from antigen-specific lines maintained by repeated stimulation of primary cells with defined peptides. As discussed above, in some cases “primary” incubations are tumor cell-PBMC co-cultures (containing tumor cells, APCs, and T cells). In such cases, APC isolation and secondary culture with separately-isolated T cells is not performed, but T cell responses are assessed directly in primary culture systems.

TAA presentation inducer constructs such as Constructs 8-11 (Table 1), that bind SKBR3 TCDM via Her2 and APCs via diverse ISR classes (see Table 1), can promote MHC presentation of peptides derived from multiple TAAs to T cells (e.g. Her2, MUC1, WT1 peptides). Analogous results are observed for ROR1-binding (Constructs 12-15) and PSMA-binding (Constructs 16-19) constructs in APC-SKOV3 or -LNCaP tumor line co-cultures, respectively. Minimal TAA-presentation is induced by control constructs that can bind either a TAA or ISR, but not both (i.e. contain a non-binding, negative control paratope) (Constructs 1-7), or by TAA presentation inducer constructs in the absence of TCDM.

### Example 6: Preparation of Additional TAA Presentation Inducer Constructs

Additional exemplary TAA presentation inducer constructs were designed to examine the effect of multiple valencies for binding the ISR and/or the TAA. The majority of these additional constructs were based on the same targets and paratopes described in Example 2; however, some constructs targeted the TAA mesothelin. These constructs are listed in Table 4, and were designed in a number of general formats as described below and as depicted in FIG. 3:

Format A: A_scFv_B_scFv_Fab, where Heavy Chain A includes an scFv and Heavy Chain B includes an scFv and a Fab. A diagram of this format is depicted in FIG. 3A.
Format B: A_scFv_Fab_B_scFv, where Heavy Chain A includes an scFv and a Fab and Heavy Chain B includes an scFv. A diagram of this format is depicted in FIG. 3B.
Format C: A_Fab_B_scFv_scFv, where Heavy Chain A includes a Fab and Heavy Chain B includes two scFvs. A diagram of this format is depicted in FIG. 3C.
Format D: A_scFv_B_Fab_Fab, where Heavy Chain A includes an scFv and Heavy Chain B includes two Fabs. A diagram of this format is depicted in FIG. 3D.
Format E: Hybrid, where Heavy Chain A includes a Fab and Heavy Chain B includes an scFv. A diagram of this format is depicted in FIG. 3E.
Format F: A_Fab_CRT_B_CRT, where Heavy Chain A includes a Fab and calreticulin and Heavy Chain B includes calreticulin. A diagram of this format is depicted in FIG. 3F.
Format G: A_Fab_CRT_B_CRT_CRT, where Heavy Chain A includes a Fab and calreticulin and Heavy Chain B includes two calreticulin polypeptides. A diagram of this format is depicted in FIG. 3G.

All of the constructs described in this example were prepared with the same symmetric amino acid substitutions in the Fc region described in Example 2 that decrease binding of the Fc to FcgammaR (L234A_L235A_D265S). In all cases, a heterodimeric Fc as described in Example 1 was used in the construct, as noted in Table 4.

Some of the additional constructs described in this example were designed to examine polypeptide variants of calreticulin that could be used in the ISR arm. These constructs are numbered 22252, 22253, and 22254. Construct 22252 includes a full length calreticulin polypeptide (residues 18-413, numbered according to UniProt Sequence ID P27797) with a substitution of the free cysteine at residue 163 with serine. Construct 22253 includes the N-domain of calreticulin (starting at residue 18), in which the P-domain (residues 205-301) is replaced by a GSG linker and the C-terminal amino acid residues from 369 to 417 were deleted (see Chouquet et al., PLoS ONE 6(3): e17886. doi:10.1371/journal.pone.0017886). Construct 22254 contains the N-domain and P-domain, corresponding to residues 18-368.

TABLE 4
Additional constructs, multiple valencies

| TAA Target | ISR Target | Format | Construct # |
|---|---|---|---|
| HER2 | Dectin-1 | A_scFv_B_scFv_Fab_TAA_Trastuzumab_ISR_Dectin-1 | 22211 |
| ROR1 | Dectin-1 | A_scFv_B_scFv_Fab_TAA_ROR1_ISR_Dectin-1 | 22212 |
| Mesothelin | Dectin-1 | A_scFv_B_scFv_Fab_TAA_Mesothelin_ISR_Dectin-1 | 22213 |
| HER2 | DEC-205 | A_scFv_B_scFv_Fab_TAA_Trastuzumab_ISR_DEC-205 | 22214 |
| ROR1 | DEC-205 | A_scFv_B_scFv_Fab_TAA_ROR1_ISR_DEC-205 | 22215 |
| Mesothelin | DEC-205 | A_scFv_B_scFv_Fab_TAA_Mesothelin_ISR_DEC-205 | 22216 |
| HER2 | CD40 | A_scFv_B_scFv_Fab_TAA_Trastuzumab_ISR_CD40 | 22217 |
| ROR1 | CD40 | A_scFv_B_scFv_Fab_TAA_ROR1_ISR_CD40 | 22218 |
| Mesothelin | CD40 | A_scFv_B_scFv_Fab_TAA_Mesothelin_ISR_CD40 | 22219 |
| HER2 | Dectin-1 | A_scFv_Fab_B_scFv_TAA_Trastuzumab_ISR_Dectin-1 | 22220 |
| ROR1 | Dectin-1 | A_scFv_Fab_B_scFv_TAA_ROR1_ISR_Dectin-1 | 22320 |
| Mesothelin | Dectin-1 | A_scFv_Fab_B_scFv_TAA_Mesothelin_ISR_Dectin-1 | 22222 |
| HER2 | DEC-205 | A_scFv_Fab_B_scFv_TAA_HER2_ISR_DEC-205 | 22223 |
| ROR1 | DEC-205 | A_scFv_Fab_B_scFv_TAA_ROR1_ISR_DEC-205 | 22321 |
| Mesothelin | DEC-205 | A_scFv_Fab_B_scFv_TAA_Mesothelin_ISR_DEC-205 | 22225 |
| HER2 | CD40 | A_scFv_Fab_B_scFv_TAA_HER2_ISR_CD40 | 22226 |
| ROR1 | CD40 | A_scFv_Fab_B_scFv_TAA_ROR1_ISR_CD40 | 22322 |
| Mesothelin | CD40 | A_scFv_Fab_B_scFv_TAA_Mesothelin_ISR_CD40 | 22228 |
| HER2 | Dectin-1 | A_Fab_B_scFv_scFv_TAA_HER2_ISR_Dectin-1 | 22151 |
| ROR1 | Dectin-1 | A_Fab_B_scFv_scFv_TAA_ROR1_ISR_Dectin-1 | 22152 |
| Mesothelin | Dectin-1 | A_Fab_B_scFv_scFv_TAA_Mesothelin_ISR_Dectin-1 | 22153 |
| HER2 | DEC-205 | A_Fab_B_scFv_scFv_TAA_HER2_ISR_DEC-205 | 22154 |
| ROR1 | DEC-205 | A_Fab_B_scFv_scFv_TAA_ROR1_ISR_DEC-205 | 22155 |
| Mesothelin | DEC-205 | A_Fab_B_scFv_scFv_TAA_Mesothelin_ISR_DEC-205 | 22156 |
| HER2 | DEC-205 | A_Fab_B_scFv_scFv_TAA_ HER2_ISR_DEC-205 | 22157 |
| ROR1 | DEC-205 | A_Fab_B_scFv_scFv_TAA_ROR1_ISR_DEC-205 | 22158 |
| Mesothelin | DEC-205 | A_Fab_B_scFv_scFv_TAA_Mesothelin_ISR_DEC-205 | 22159 |
| HER2 | Dectin-1 | A_scFv_B_Fab_Fab_TAA_ HER2_ISR_Dectin-1 | 22300 |
| ROR1 | Dectin-1 | A_scFv_B_Fab_Fab_TAA_ROR1_ISR_Dectin-1 | 22301 |
| Mesothelin | Dectin-1 | A_scFv_B_Fab_Fab_TAA_Mesothelin_ISR_Dectin-1 | 22302 |
| HER2 | DEC-205 | A_scFv_B_Fab_Fab_TAA_HER2_ISR_DEC-205 | 22303 |
| ROR1 | DEC-205 | A_scFv_B_Fab_Fab_TAA_ROR1_ISR_DEC-205 | 22304 |
| Mesothelin | DEC-205 | A_scFv_B_Fab_Fab_TAA_Mesothelin_ISR_DEC-205 | 22305 |
| HER2 | CD40 | A_scFv_B_Fab_Fab_TAA_HER2_ISR_CD40 | 22306 |
| ROR1 | CD40 | A_scFv_B_Fab_Fab_TAA_ROR1_ISR_CD40 | 22307 |
| Mesothelin | CD40 | A_scFv_B_Fab_Fab_TAA_Mesothelin_ISR_CD40 | 22308 |
| HER2 | Dectin-1 | hybrid_TAA_HER2_ISR_Dectin-1 | 22262 |
| ROR1 | Dectin-1 | hybrid_TAA_ROR1_ISR_Dectin-1 | 22263 |
| Mesothelin | Dectin-1 | hybrid_TAA_Mesothelin_ISR_Dectin-1 | 22264 |
| HER2 | DEC-205 | hybrid_TAA_HER2_ISR_DEC-205 | 22265 |
| ROR1 | DEC-205 | hybrid_TAA_ROR1_ISR_DEC-205 | 22266 |
| Mesothelin | DEC-205 | hybrid_TAA_Mesothelin_ISR_DEC-205 | 22267 |
| HER2 | CD40 | hybrid_TAA_HER2_ISR_CD40 | 22268 |
| ROR1 | CD40 | hybrid_TAA_ROR1_ISR_CD40 | 22269 |
| Mesothelin | CD40 | hybrid_TAA_Mesothelin_ISR_CD40 | 22270 |
| HER2 | LRP-1 | A_Fab_CRT_B_CRT_TAA_HER2_ISR_CRT | 22247 |
| ROR1 | LRP-1 | A_Fab_CRT_B_CRT_TAA_ROR1_ISR_CRT | 22323 |
| Mesothelin | LRP-1 | A_Fab_CRT_B_CRT_TAA_Mesothelin_ISR_CRT | 22249 |
| HER2 | LRP-1 | A_Fab_CRT_B_CRT_CRT_TAA_HER2_ISR_CRT | 22250 |
| HER2 | LRP-1 | A_Fab_CRT_B_CRT_TAA_HER2_ISR_CRT | 22271 |
| HER2 | LRP-1 | A_Fab_B_CRT-Cys_TAA_HER2_ISR_CRT | 22252 |
| HER2 | LRP-1 | A_Fab_B_CRT_N_TAA_HER2_ISR_CRT | 22253 |
| HER2 | LRP-1 | A_Fab_B_CRT_NP_TAA_HER2_ISR_CRT | 22254 |

The scFv and Fab sequences were generated from the sequences of known antibodies, identified in Table 5. Note that LRP-1 is putatively targeted with calreticulin (CRT) as a ligand, not with an antibody.

TABLE 5
References for TAA presentation inducer construct sequences

| Target | Paratope/Antibody clone | Reference |
|---|---|---|
| ROR1 | R12 | WO2012075158 |
| Mesothelin | RG7787 | U.S. Pat. No. 7,081,518 |
| Dectin-1 | 15E2.5 | WO2008118587 |
| Dectin-1 | 2D8.2D4 | WO2008118587 |
| DEC205 | 3G9 | WO2009061996 |
| CD40 | 12E12 | US20100239575 |
| LRP-1 | Recombinant human | WO2010030861 |
| | calreticulin | |

CDR sequences, as determined by the IMGT numbering system, for the antibody clones listed above are found in Table YY.

The constructs identified in Table 6 were designed as controls.

TABLE 6
Control constructs

| OAA scFv controls | Construct # |
|---|---|
| Trastuzumab | 22255 |
| ROR1 | 22256 |
| Mesothelin | 22257 |
| Dectin-1 | 22272 |
| DEC-205 | 22273 |
| CD40 | 22274 |
| CRT | 22275 |

Table 7 identifies the amino acid and DNA sequences for the constructs described in this example. Each construct is made up of 2 or 3 clones and the amino acid and DNA sequences of the clones are found in Table ZZ.

TABLE 7
Constructs and clone numbers

| Construct # | Chain A | Light chain A | Chain B | Light Chain B |
|---|---|---|---|---|
| 22211 | 16795 | | 16772 | 12645 |
| 22212 | 16711 | | 16772 | 12645 |
| 22213 | 16712 | | 16772 | 12645 |
| 22214 | 16795 | | 16773 | 12651 |
| 22215 | 16711 | | 16773 | 12651 |
| 22216 | 16712 | | 16773 | 12651 |
| 22217 | 16795 | | 16774 | 12653 |
| 22218 | 16711 | | 16774 | 12653 |
| 22219 | 16712 | | 16774 | 12653 |
| 22220 | 16714 | 11150 | 16778 | |
| 22320 | 16811 | 12660 | 16778 | |
| 22222 | 16716 | 10565 | 16778 | |
| 22223 | 16717 | 11150 | 16779 | |
| 22321 | 16812 | 12660 | 16779 | |
| 22225 | 16719 | 10565 | 16779 | |
| 22226 | 16720 | 11150 | 16780 | |
| 22322 | 16813 | 12660 | 16780 | |
| 22228 | 16722 | 10565 | 16780 | |
| 22151 | 16713 | 11150 | 16743 | |
| 22152 | 12659 | 12660 | 16743 | |
| 22153 | 12966 | 10565 | 16743 | |
| 22154 | 16713 | 11150 | 16744 | |
| 22155 | 12659 | 12660 | 16744 | |
| 22156 | 12966 | 10565 | 16744 | |
| 22157 | 16713 | 11150 | 16745 | |
| 22158 | 12659 | 12660 | 16745 | |
| 22159 | 12966 | 10565 | 16745 | |
| 22300 | 16795 | | 16803 | 12645 |
| 22301 | 16711 | | 16803 | 12645 |
| 22302 | 16712 | | 16803 | 12645 |
| 22303 | 16795 | | 16802 | 12651 |
| 22304 | 16711 | | 16802 | 12651 |
| 22305 | 16712 | | 16802 | 12651 |
| 22306 | 16795 | | 16801 | 12653 |
| 22307 | 16711 | | 16801 | 12653 |
| 22308 | 16712 | | 16801 | 12653 |
| 22262 | 16713 | 11150 | 16778 | |
| 22263 | 12659 | 12660 | 16778 | |
| 22264 | 12966 | 10565 | 16778 | |
| 22265 | 16713 | 11150 | 16779 | |
| 22266 | 12659 | 12660 | 16779 | |
| 22267 | 12966 | 10565 | 16779 | |
| 22268 | 16713 | 11150 | 16780 | |
| 22269 | 12659 | 12660 | 16780 | |
| 22270 | 12966 | 10565 | 16780 | |
| 22247 | 16733 | 11150 | 12667 | |
| 22323 | 16814 | 12660 | 12667 | |
| 22249 | 16735 | 10565 | 12667 | |
| 22250 | 16733 | 11150 | 16784 | |
| 22271 | 16713 | 11150 | 12667 | |
| 22252 | 16713 | 11150 | 16781 | |
| 22253 | 16713 | 11150 | 16782 | |
| 22254 | 16713 | 11150 | 16783 | |
| 22255 | 16795 | | 12153 | |
| 22256 | 16711 | | 12153 | |
| 22257 | 16712 | | 12153 | |
| 22272 | 12155 | | 16778 | |
| 22273 | 12155 | | 16779 | |
| 22274 | 12155 | | 16780 | |
| 22275 | 12155 | | 12667 | |

The constructs in Tables 4 and 6 were prepared and expressed as described in Example 2. Constructs 22154-22156 did not express due to cloning errors. For the remainder of the constructs, purified protein concentrations ranged from 0.1-1.2 mg/mL, and total yields ranged between 1-8 mg from 200 mL-500 mL transient transfections.

### Example 7: Preparation of Additional TAA Presentation Inducer Constructs Targeting HER2 and LRP-1

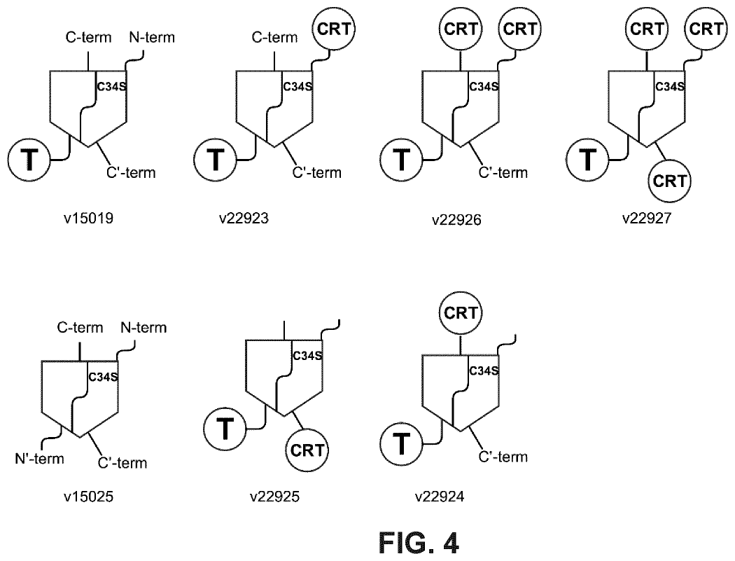

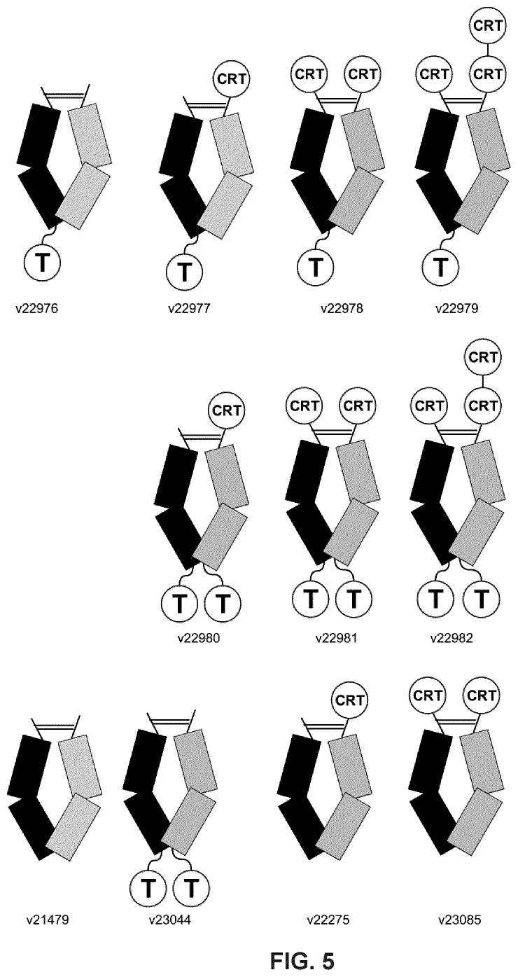

Additional exemplary TAA presentation inducer constructs were designed to examine the effect of multiple valencies for binding the ISR and/or the TAA, and to prepare constructs incorporating a split albumin scaffold instead of an Fc scaffold. These constructs targeted the TAA HER2 and the ISR LRP-1, where the HER2 binding construct was an scFv derived from trastuzumab (TscFv), stabilized with a disulfide at positions vH44-vL100 (using Kabat numbering), and the LRP-1 binding construct was a polypeptide having residues 18-417 of calreticulin (CRT). These constructs were designed in a number of geometries as depicted in FIG. 4 (split albumin scaffold) and FIG. 5 (Fc scaffold).

The split albumin scaffold used in the above molecules was based on the AlbuCORE™ 3 scaffold described in International Publication No. WO 2014/012082, with N-terminal fusions of binding constructs linked to the albumin fragment with a linker (in some cases an AAGG (SEQ ID NO:156) linker), and C-terminal fusions of binding constructs linked to the albumin fragment with a linker (in some cases a GGGS (SEQ ID NO:157) linker). In addition, the N-terminal fragment of albumin included the C34S point mutation.

All of the Fc linkers in this example included the same symmetric amino acid substitutions in the Fc region described in Example 2 that decrease binding of the Fc to FcgammaR (L234A_L235A_D265S). In all cases, a heterodimeric Fc as described in Example 1 was used in the construct, as noted in Table 4. Trastuzumab scFvs were fused to the C-terminus of the Fc polypeptide with a GGGG (SEQ ID NO:158) linker.

Table 8 provides details regarding the components of constructs prepared with the split albumin scaffold, while Table 9 provides details regarding the components prepared with the Fc scaffold. Each construct was made up of two polypeptides, and the clone number of each polypeptide is provided in Table 8 and Table 9. The amino acid and DNA sequences of the clones are found in Table ZZ.

TABLE 8

| | | | N- | N′- | C- | C′- |
|---|---|---|---|---|---|---|
| Construct | Clone A | Clone B | fusion | fusion | fusion | fusion |
| 15019 | 9157 | 9182 | — | TscFv | — | — |
| 22923 | 17858 | 9182 | CRT | TscFv | — | — |
| 22924 | 9157 | 17860 | — | TscFv | CRT | — |
| 22925 | 17862 | 9182 | — | TscFv | — | CRT |
| 22926 | 17858 | 17860 | CRT | TscFv | CRT | — |
| 22927 | 17859 | 17860 | CRT | TscFv | CRT | CRT |
| 15025 | 9157 | 9158 | — | — | — | — |

TABLE 9

| Construct | H1 | H2 | N1 | N2 | C1 | C2 |
|---|---|---|---|---|---|---|
| 22976 | 17901 | 12153 | — | — | TscFv | — |
| 22977 | 17901 | 12667 | — | CRT | TscFv | — |
| 22978 | 17902 | 12667 | CRT | CRT | TscFv | — |
| 22979 | 17902 | 16784 | CRT | CRTCRT | TscFv | — |
| 22980 | 17901 | 17903 | — | CRT | TscFv | TscFv |
| 22981 | 17902 | 17903 | CRT | CRT | TscFv | TscFv |
| 22982 | 17902 | 17904 | CRT | CRTCRT | TscFv | TscFv |
| 23044 | 17901 | 17905 | — | — | TscFv | TscFv |
| 21479 | 12155 | 12153 | — | — | — | — |
| 23085 | 17941 | 12667 | CRT | CRT | — | — |
| 22275 | 12155 | 12667 | — | CRT | — | — |

Fc-based constructs were expressed and purified as described in Example 2.

AlbuCORE™-based constructs were purified as follows. Variants from cell culture medium (200 mL to 2.5 L) were purified batchwise by affinity chromatography using AlbuPure® resin. Endotoxin levels were validated to be below 0.2 EU/ml in all samples. AlbuPure® affinity resin previously kept in storage solution and/or cleaned using a compatible procedure was equilibrated with and then resuspended in a 1:1 ratio of sodium phosphate buffer pH 6.0. The culture supernatant pH is adjusted to 6.0 with 1 M sodium phosphate monobasic buffer. The required volume of resin slurry was added to the culture supernatant feed based on the antibody (or antibody fragment) content and the resin binding capacity (30 mg of human serum albumin/mL of resin). Using an orbital shaker, the resin was maintained in suspension overnight at 2-8° C. The feed was transferred into a chromatography column and flow-through is collected. The resin was then washed with the resin equilibration buffer prior to be washed using sodium phosphate buffer pH 7.8 to remove potential non-specifically bound material. The protein product was eluted, using a sodium octanoate solution and collected in fractions. The protein content of each elution fraction was determined by 280 nm absorbance measurement using a Nanodrop or with a relative colorimetric protein assay. The most concentrated fractions were pooled and then further purified by Size Exclusion Chromatography using a Superdex 200 column, 16 mm in a PBS buffer. The most concentrated fractions were pooled and evaluated by CE-SDS, UPLC-SEC and SDS-PAGE.

Purified protein concentrations ranged from 0.2-6 mg/mL, and total yields ranged between 0.3-120 mg from 200 mL-2500 mL transient transfections.

### Example 8: TAA Presentation Inducer Constructs are Able to Bind Target(s) Transiently Expressed on Cells

To assess the native target binding of selected TAA presentation inducer constructs to their targets of interest, a homogeneous cell binding assay was performed through high content screening using the CellInsight™ platform (Thermo Scientific). The constructs tested are described in Example 6 and include constructs in Formats A to G, as described therein. In summary, constructs contained at least one TAA-binding construct in scFv or Fab form against one of the following tumor-associated antigens: HER2, ROR1 or mesothelin (MSLN), and at least one ISR-binding construct in scFv or Fab form targeting DECTIN-1, DEC205 or CD40. Some of the tested constructs contained an TAA-binding construct in Fab form and one or more recombinant CRT polypeptide as the ISR-binding construct. Binding of constructs to target was assessed in HEK293-6e cells transiently expressing the target of interest.

### Preparation of HEK293-6e Cells Transiently Expressing Targets of Interest

To prepare cells transiently expressing targets of interest, a suspension of HEK293-6e cells (National Research Council) was cultured in 293 Freestyle Media (Gibco, 12338018) with 1% FBS (Corning, 35-015CV). Parental cells were maintained in 250 mL Erlenmeyer flasks (Corning, 431144) at 37° C., 5% CO2 in a rotating humidified incubator at 115 rpm. HEK293-6e cells were re-suspended to 1×10⁶ cells/mL in fresh Freestyle media before transfection. Cells were transfected with 293Fectin™ transfection reagent (Gibco, 12347019) at a ratio of 1 μg/10⁶ cells in Opti-MEM™ Reduced Serum Medium (Gibco, 31985070). The DNA vectors that were used to express targets of interest were pTT5 vectors with full length targets of interest including Human Dectin-1, Human DEC205, Human CD40, Human HER2, Human ROR1 and mock vector containing GFP. The cells were incubated for 24 hours at 37° C. and 5% CO2 in a rotating humidified incubator at 115 rpm.

### Binding Assay

Construct samples were prepared at starting concentrations of 40 nM final in FACS buffer or 1×PBS pH 7.4 (Gibco, 1001023)+2% FBS in Eppendorf tubes. Samples were titrated in duplicate 1:4 down to 0.04 nM directly in the 384-well black optical bottom assay plate (Thermo Fisher, 142761). HEK293-6e cells expressing target of interest were harvested and re-suspended in FACS buffer at 10,000 cells per 30 μl. To visualize cell nuclei as a focusing channel, Vybrant™ DyeCycle™ Violet nuclear stain (Life Tech, V35003) was added to cells at 2 μM final concentration. To detect binding of test construct sample to cells, Goat anti-Human IgG Fc A647 (Jackson ImmunoResearch, 115-605-071) was added to cells at 0.6 μg/mL final. The cells were vortexed briefly to mix and plated at 10,000 cells/well. The plate was incubated at room temperature for 3 hours before scanning. Data analysis was performed on the CellInsight™ with the HCS high content screening platform (Thermo Scientific), using BioApplication “CellViability” with a 10× objective. Samples were scanned on the 385 nm channel to visualize nuclear staining and channel 650 nm to assess cell binding. The mean object average fluorescence intensity of A647 was measured on channel 2 to determine binding intensity on all cell conditions. Fold over mock values were determined by dividing A647 intensity on HEK293-specific cells over A647 intensity from HEK293-mock. All wells were visually inspected to confirm results. All data graphs were prepared using GraphPad Prism 7 software.

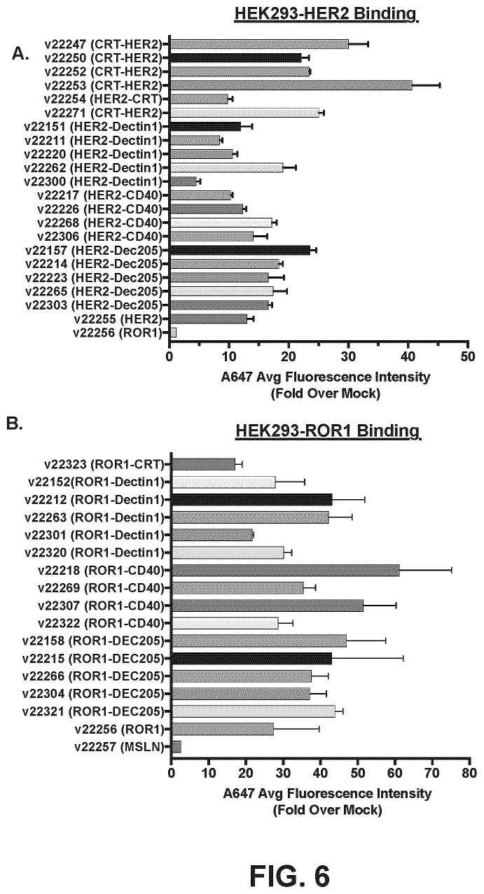

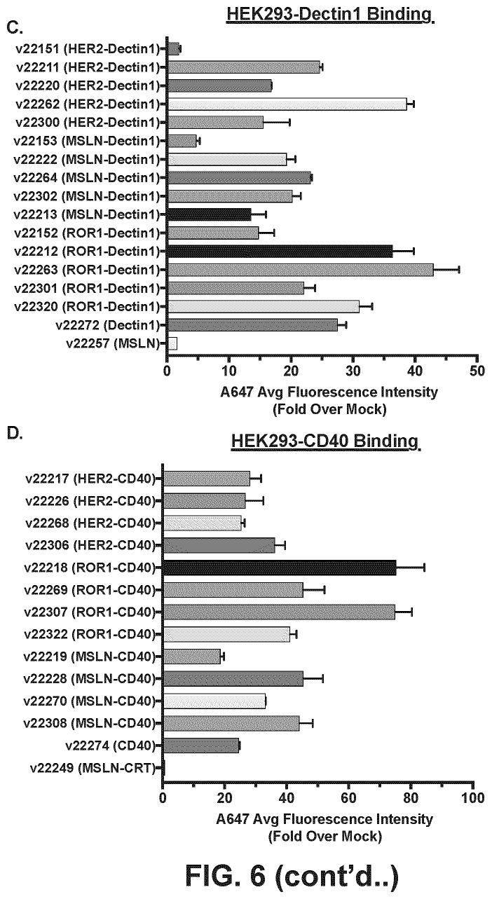

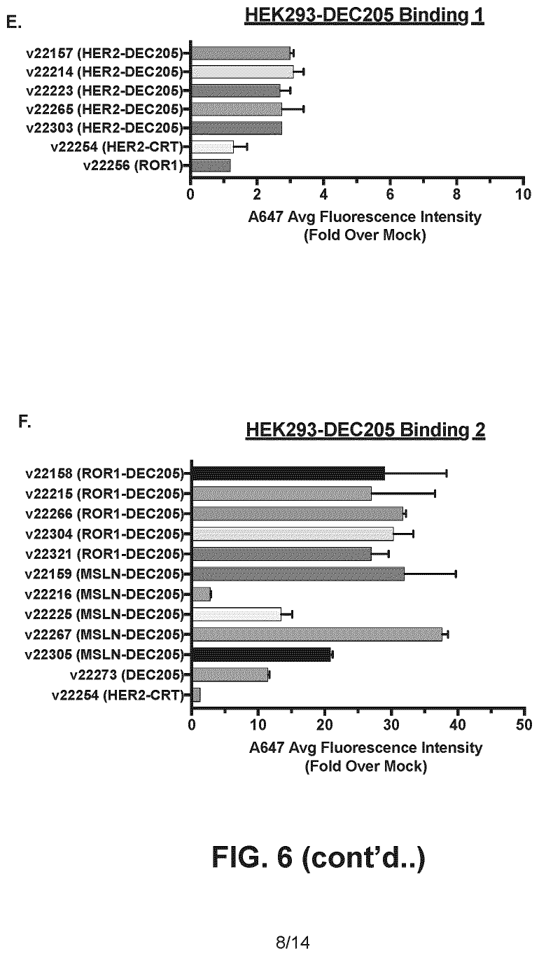

The results of the binding assays are shown in FIG. 6A (HER2 binding), 6B (ROR1 binding), 6C (dectin-1 binding), 6D (CD40 binding), and 6E and 6F (both DEC205 binding). These Figures show the average A647 fluorescence intensity (fold over mock) from constructs tested at 10 nM. As shown in these Figures, all constructs bound to their respective targets transiently expressed in HEK293-6e cells. None of the constructs bound to HEK293-mock cells, as expected.

### Example 9: TAA Presentation Inducer Constructs Targeting Mesothelin are Able to Bind to Mesothelin-Positive NCI-11226 Cells

TAA presentation inducer constructs targeting mesothelin were tested for their ability to bind to cells that naturally express mesothelin. The constructs tested are described in Example 6 and contained at least one TAA-binding construct in scFv or Fab form against MSLN, and at least one ISR-binding construct in scFv or Fab form targeting DECTIN-1, DEC205 or CD40. One of the tested constructs contained an anti-MSLN TAA-binding construct in Fab form and two recombinant CRT polypeptides as the ISR-binding construct. Binding of constructs to MSLN was assessed in mesothelin-positive NCI-H226 cells.

A homogeneous cell binding assay was performed through high content screening using the CellInsight™ platform (Thermo Scientific) to assess native binding of constructs designed to bind mesothelin. Mesothelin-positive NCI-H226 cells (National Research Council, CRL-5826) were cultured in RPMI1640 media (Gibco, A1049101) supplemented with 10% FBS (Corning, 35-015CV) and maintained at 37° C., 5% CO2 in T175 flasks. Construct samples were prepared and incubated with cells, nuclear stain, and secondary reagent as described in Example 8. Irrelevant antibodies with no α-mesothelin binding moiety were included as negative controls. Data analysis was performed on the CellInsight™ with the HCS high content screening platform (Thermo Scientific), using BioApplication “Cell Viability” with a 10× objective. Samples were scanned on the 385 nm channel to visualize nuclear staining and channel 650 nm to assess cell binding. The mean object average fluorescence intensity of A647 was measured on channel 2 to determine binding intensity on NCI-H226 and HEK293-6e control cells. Fold over mock values were determined by dividing A647 intensity on NCI-H226 over A647 intensity from HEK293-mock. All wells were visually inspected to confirm results. All data graphs were prepared using GraphPad Prism 7 software.

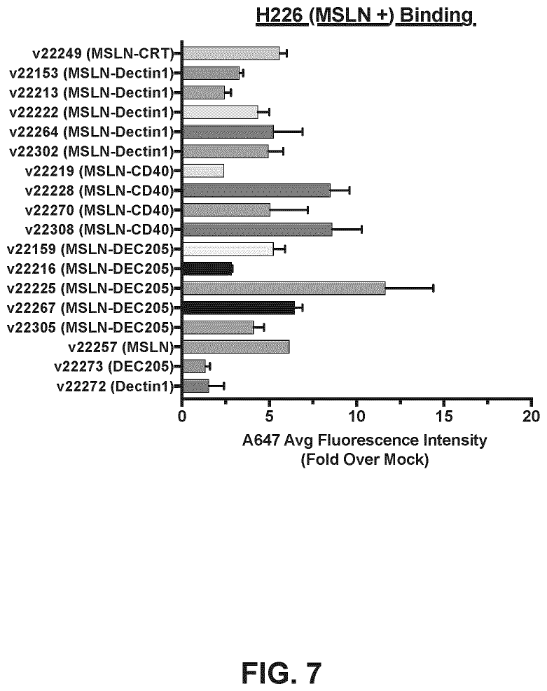

The results are shown in FIG. 7 where the average A647 fluorescence intensity (fold over mock) from constructs tested at 10 nM is provided. All constructs carrying an α-mesothelin-binding construct bound to mesothelin-positive NCI-H226 cells. Irrelevant antibodies without an α-mesothelin-binding construct did not bind to NCI-H226 cells, as expected. None of the samples bound to HEK293-mock negative control cells.

### Example 10: TAA Presentation Inducer Constructs Containing Recombinant Calreticulin Bind to Anti-Calreticulin Antibody as Measured by ELISA

TAA presentation inducer constructs containing a recombinant calreticulin as an LRP-1 targeting moiety underwent quality control by detection of calreticulin with the mouse α-human calreticulin (CRT) antibody MAB3898 (R&D Systems, 326203) by ELISA. Briefly, constructs were coated at 3 μg/mL in 1×PBS at 50 μl/well in 96-well medium binding ELISA plates (Corning 3368). v22152 (ROR1×Dectin1) was included as negative control. Commercial calreticulin was coated as a positive control (Abcam, ab91577). An irrelevant construct without calreticulin served as a negative control. The plates were incubated overnight at 4° C. The following day, the plates were washed 3×200 μl with distilled water using a plate washer (BioTek, 405 LS). The plates were blocked with 200 μl/well of 2% milk in PBS and incubated at room temperature for one hour. The plates were washed as previously described. MAB3898 primary antibody was titrated 1:5 in 2% milk from 10 μg/mL down 4 steps to obtain 2 μg/mL, 0.4 μg/mL, and 0.08 μg/mL with 50 μl/well final. Blank wells containing buffer only were included. After a primary incubation of 1 hr at room temperature, the plates were washed as previously described. Goat anti mouse IgG Fc HRP (Jackson ImmunoResearch, 115-035-071) was used to detect Mouse α-calreticulin binding. Goat anti human IgG Fc HRP (Jackson ImmunoResearch, 109-035-098) was used to confirm coating of constructs to the plate. Both secondary reagents were incubated for 30 minutes at room temperature at 50 μl/well. After incubation, the plates were washed as previously described and 50 μl/well of TMB (Cell Signaling Technology, 7004) was used to visualize binding. After 5 minutes, 1.0 N hydrochloric acid (VWR Analytical, BDH7202-1) was added at 50 μl/well to neutralize the reaction. The plates were scanned on the Synergy H1 plate-reader to measure absorbance at 450 nm.

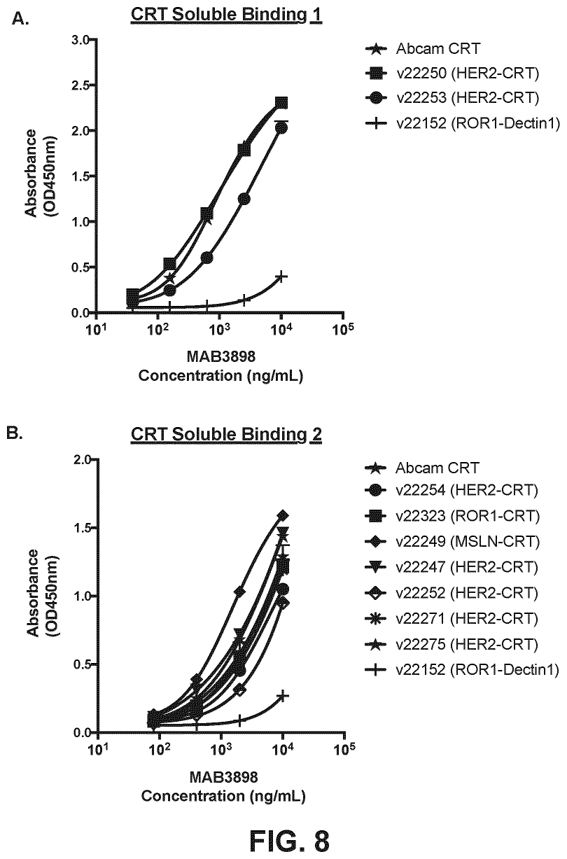

The results are shown in FIGS. 8A and 8B. MAB3898 was successfully able to detect calreticulin in CRT-containing constructs, indicating that recombinant cloning, expression and purification protocols retained normal domain structures. Goat anti Human IgG Fc HRP confirmed an even coating of antibodies to the plate. Positive control Abcam calreticulin was also detected with MAB3898.

### Example 11: TAA Presentation Inducer Constructs are Able to Induce Phagocytosis of Tumor Cell Material

To evaluate the ability of TAA presentation inducer constructs to induce phagocytosis of tumor cell material, a representative number of constructs were assessed in phagocytosis assay. Briefly, the assay measured the ability of THP-1 monocytic cells to phagocytose material from labelled SKBR3 cells. The constructs tested were the HER2×CD40-targeting construct 18532, the HER2×DEC205-targeting construct 18529, and the HER2×LRP-1-targeting construct 18535. Constructs 18532 and 18529 were demonstrated to specifically bind to their appropriate targets according to the method described in Example 8 (data not shown). Recombinant CRT in construct 18535 was quality controlled via demonstrated binding to commercially available anti-calreticulin antibody as described in Example 10 (data not shown).

pHrodo-labeled SKBR3 cells were prepared by adding 1 μl of 1 mg/ml (20 ng/ml for 10⁶ cells) pHrodo dextran to 50 ml of SKBR3 cell suspension and incubating for 30 minutes at room temperature, followed by 3 washes with PBS. 2×10³ pHrodo-labeled SKBR3 cells were added to 2×10⁴ THP-1 cells and cultured for 72h at 37° C. in RPMI1640 medium containing 10% fetal calf serum and the constructs in 384 well microtiter plates. 20 μl detection medium including DyeCycle™ Violet at 2 μM was added to each well, and plates were incubated for 2.5h at 37° C. Plates were imaged and phagocytosis quantified using CellInsight™ Bioapplication (ThermoFisher) instrumentation and software.

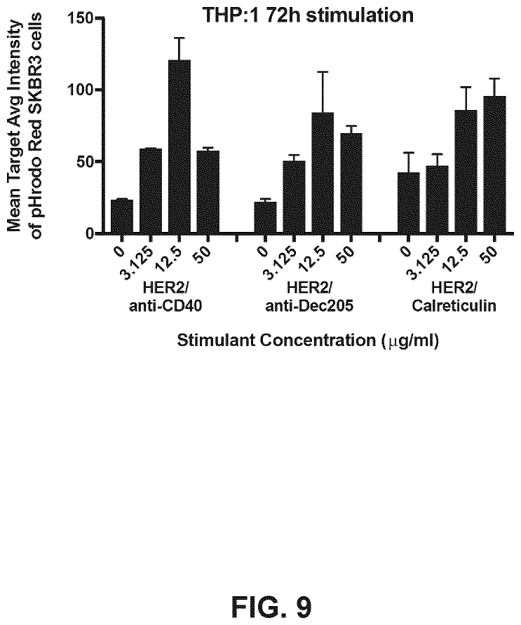

The results are shown in FIG. 9. TAA presentation inducer constructs Her2×CD40 (18532), Her2×Dec205 (18529), and Her2×CRT (18535) potentiated THP-1 cell phagocytosis of SKBR3 tumor material.

### Example 12: TAA Presentation Inducer Constructs are Able to Induce Monocyte Cytokine Production

The ability of TAA presentation inducer constructs to induce monocyte cytokine production (as a measure of APC activation), which is required for optimally productive antigen presentation to cells, was assessed in a system similar to the one described in Example 11.

pHrodo-labeled SKBR3 cells were prepared by adding 1 μl of 1 mg/ml (20 ng/ml for 10⁶ cells) pHrodo dextran to 50 ml of SKBR3 cell suspension and incubating for 30 minutes at room temperature, followed by 3 washes with PBS. 2×10³ pHrodo-labeled SKBR3 cells were added to 2×10⁴ primary human monocytes and cultured for 72h at 37° C. in RPMI1640 medium containing 10% fetal calf serum and the indicated constructs in 384 well microtiter plates. Supernatant cytokines were quantified using Meso Scale Discovery™ immunoassay according to the manufacturer's recommended protocol.

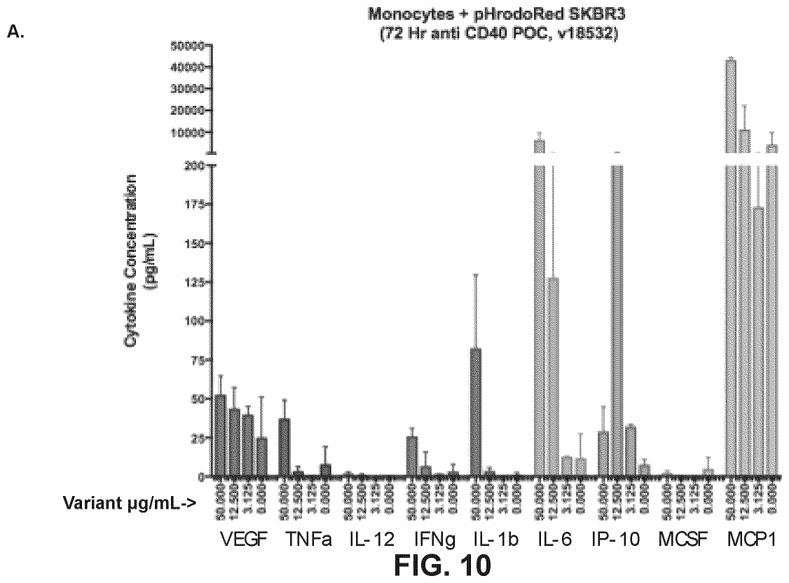

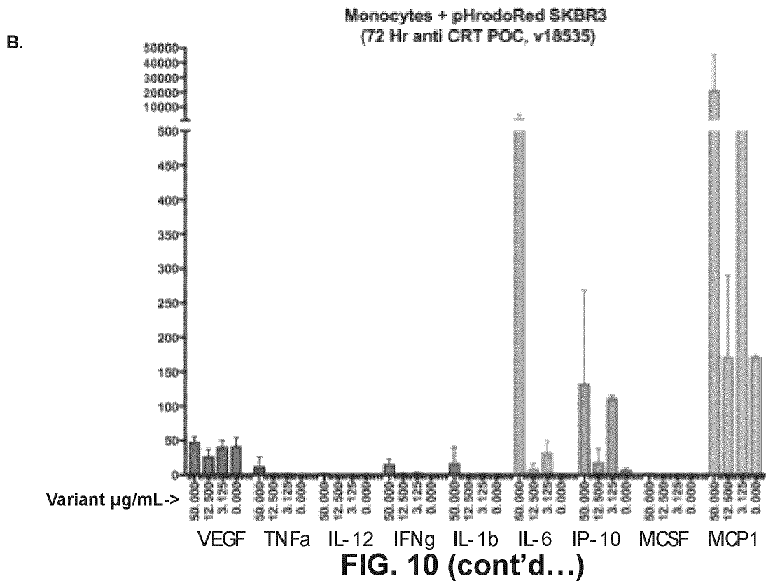

The results are shown in FIG. 10A (Her2×CD40 (v18532)) and FIG. 10B (Her2×CRT (v18535)). Both constructs potentiated primary monocyte cytokine production in the presence of SKBR3 tumor cells.

### Example 13: TAA Presentation Inducer Constructs Promote MHC Presentation of an Intracellular TAA and Trigger Antigen-Specific T Cell Response

MHC presentation of an intracellular TAA induced by TAA presentation inducer constructs was evaluated by assessing the stimulatory effect of APCs on antigen-specific T cells. APCs were first incubated with constructs and tumor cells to allow activation of the APC, uptake of an exogenously-introduced intracellular TAA, MelanA, and cross-presentation of the Melan A peptide on the MHC I complex. T cell populations enriched for Melan A-specific CD8⁺ T cells were subsequently introduced to the culture and T cell responses quantified by measuring the level of secreted IFNγ in the supernatant. TAA presentation inducer constructs tested include those targeting HER2 or Mesothelin (MSLN) as the TAA and targeting Dectin-1 or LRP-1 (via CRT) as the ISR. Two co-culture systems, an APC-tumor cell co-culture followed by an APC-T cell co-culture, were carried out as follows.

### APC-Tumor Cell Co-Culture

APCs (immature DCs) were prepared from human PBMCs (STEMCELL Technologies, cat: 70025.3) using the method described in Wolfl et al., (2014) Nat. Protoc. 9(4):950-966. OVCAR3 cells were used as the tumor cell line. Melan A peptide (ELGIGILTV (SEQ ID NO:159), Genscript) was used as a surrogate intracellular TAA. Since OVCAR3 cells have a low HER2 expression profile, they were transiently transfected with a plasmid encoding human full-length HER2 24 hrs before co-culture. MelanA was introduced into OVCAR3 cells using two methods: one batch of HER2-transfected cells was transiently co-transfected with a plasmid encoding a MelanA-GFP fusion protein 24 hrs before co-culture, while another batch of HER2-transfected cells was electroporated with the MelanA peptide (50 μg/ml) 30 min before co-culture. For non-specific antigen controls, OVCAR3 cells were transfected or electroporated with a GFP plasmid or with the K-ras peptide (KLVVVGAGGV (SEQ ID NO:160), Genscript), respectively. Both plasmid transfections and peptide electroporations were performed using the Neon® Transfection System (ThermoFisher Scientific) with the following parameters: 1050 mV, 30 ms, 2 pulses.

The co-culture was set up in the following order: constructs were diluted in Assay Buffer (AIM-V Serum Free Medium (ThermoFisher, cat: 12055083)+0.5% human AB serum (Zen-Bio, cat: HSER-ABP-100ML)), with 50 ng/ml huIL-7 (peprotech, cat: 200-007) and aliquoted at 30 μl/well into 384-well plates (Thermo Scientific Nunc, cat: 142761). Immature DCs were harvested using a cell scraper and re-suspended in Assay Buffer at 6.67×10⁵ cells/ml. OVCAR3 cells were harvested using Cell Dissociation Buffer (Life Technologies, cat: 13151014) and re-suspended in Assay Buffer at 1.33×10⁵ cells/ml. Immature DCs and OVCAR3 cell suspensions were mixed at a volume ratio of 1:1 and 30 μl of the mixture was added to plates containing the variants. Cells were incubated overnight at 37° C.+5% CO2.

### APC-T Cell Co-Culture

MelanA-enriched CD8⁺ T cells were prepared using a previous protocol with modifications (Pathangey et al., 2016). Briefly, PBMCs were thawed, washed in PBS and re-suspended in Assay Buffer with 40 ng/mL huGM-CSF at 6.0×10⁶ cells/mL and seeded in 48-well plates at 0.5 mL/well. On day 2 of the culture, MelanA peptide was added to wells at 50 μg/mL. After 4 hours, R848 (Invitrogen, tlrl-r-848) was added to the cultures to a final concentration of 3 μg/mL. 30 minutes after the addition of R848, LPS (Sigma, L5293) was added to the cultures to a final concentration of 5 ng/mL. On day 3, cells were washed with PBS, and re-suspended with 12 culture volumes of AIM-V medium with 2% human AB serum and 50 ng/mL huIL-7. Cells were re-seeded in fresh 48-well plates at 1 ml/well to give 1×10⁶ cells/well. Cells were incubated at 37° C.+5% CO2 with further passaging as the medium became yellow. Cells were pooled on Day 14 and the CD8⁺ fraction was isolated using a CD8⁺ T cell isolation Kit (Miltenyi Biotec, cat: 130-096-495). Next, cells were rested overnight at 37° C.+5% CO2 and re-suspended in Assay Buffer at 1.67×10⁶ cells/ml the following day. For the co-culture, 20 μl of the supernatant from the APC-tumor cell co-culture plates were removed and 20 μl of the T cell suspension were added. Cells were incubated at 37° C.+5% CO2 for 48 hrs and culture supernatant was taken to assess IFNγ production using a human IFNγ assay kit (Cisbio, cat: 62HIFNGPEH).

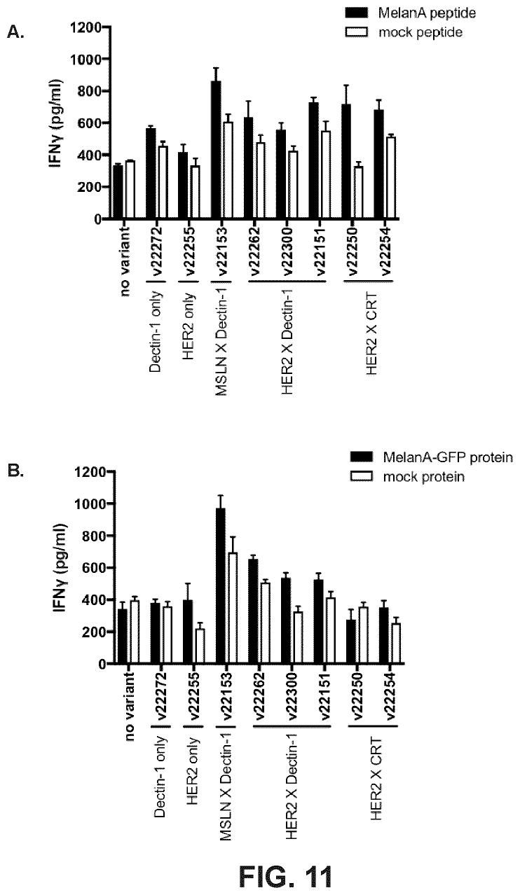

Results are shown in FIG. 11A (OVCAR cells electroporated with MelaA peptide) and FIG. 11B (OVCAR cells transfected with plasmid encoding a MelanA-GFP fusion protein). The constructs were tested at 10 μg/ml. Error bars represent standard errors of the mean of at least two experimental replicates. The MSLN×Dectin-1 construct, v22153, elicited the strongest MelanA-specific T cell response, with ˜1000 pg/ml of secreted IFNγ in the supernatant using both MelanA peptide-containing tumor cells and MelanA-GFP protein-containing tumor cells; responses were more robust in MelanA than control-peptide containing culture systems. Using MelanA peptide-containing cells, one HER2×Dectin-1 variant (v22151) and two HER2×CRT variants (v22250 and v22254) showed antigen-specific T cell activation above background or control peptide conditions. Furthermore, using MelanA-GFP protein-containing cells, three HER2×Dectin-1 variants (v22262, v22300, and v22151) showed such activation. Therefore, TAA presentation inducer multispecific variants specific for Her2 or MSLN promoted APC acquisition of an intracellular tumor cell TAA (MelanA) and promoted presentation to T cells via anti-Dectin-1 or CRT.

For multiple, diverse, target pairs, these results demonstrate that anti-TAA×ISR constructs promote TCDM acquisition by APCs and redirect immune responses toward tumor-derived antigens distinct from those physically bound to the TAA presentation inducer constructs themselves.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

TABLE YY
CDRs

| Paratope/ | | | |
|---|---|---|---|
| Antibody | CDR# | | SEQ ID |
| clone | (IMGT) | Sequence | NO: |
| 12E12 | CDR H1 | GFTFSDYY | 183 |
| | CDR H2 | INSGGGST | 184 |
| | CDR H3 | ARRGLPFHAMDY | 185 |
| | CDR L1 | QGISNY | 186 |
| | CDR L2 | YTS | 187 |
| | CDR L3 | QQFNKLPPT | 188 |
| 3G9 | CDR H1 | GFTFSNYG | 189 |
| | CDR H2 | IWYDGSNK | 190 |
| | CDR H3 | ARDLWGWYFDY | 191 |
| | CDR L1 | QSVSSY | 192 |
| | CDR L2 | DAS | 193 |
| | CDR L3 | QQRRNWPLT | 194 |
| 15E2.5 | CDR H1 | GYTFTTYT | 195 |
| | CDR H2 | INPSSGYT | 196 |
| | CDR H3 | ARERAVLVPYAMDY | 197 |
| | CDR L1 | SSLSY | 198 |
| | CDR L2 | STS | 199 |
| | CDR L3 | QQRSSSPFT | 200 |
| 2D8.2D4 | CDR H1 | GYSFTGYN | 201 |
| | CDR H2 | IDPYYGDT | 202 |
| | CDR H3 | ARPYGSEAYFAY | 203 |
| | CDR L1 | QSISDY | 204 |
| | CDR L2 | YAA | 205 |
| | CDR L3 | QNGHSFPYT | 206 |
| 11B6.4 | CDR H1 | GFSLSNYD | 207 |
| | CDR H2 | MWTGGGA | 208 |
| | CDR H3 | VRDAVRYWNFDV | 209 |
| | CDR L1 | SSVSY | 210 |
| | CDR L2 | ATS | 211 |
| | CDR L3 | QQWSSNPFT | 212 |
| Pertuzu- | CDR H1 | GFTFTDYT | 213 |
| mab | CDR H2 | VNPNSGGS | 214 |
| | CDR H3 | ARNLGPSFYFDY | 215 |
| | CDR L1 | QDVSIG | 216 |
| | CDR L2 | SAS | 217 |
| | CDR L3 | QQYYIYPYT | 218 |
| RG7787 | CDR H1 | GYSFTGYT | 219 |
| | CDR H2 | ITPYNGAS | 220 |
| | CDR H3 | ARGGYDGRGFDY | 221 |
| | CDR L1 | SSVSY | 222 |
| | CDR L2 | DTS | 223 |
| | CDR L3 | QQWSKHPLT | 224 |
| MLN2704 | CDR H1 | GYTFTEYT | 225 |
| | CDR H2 | INPNNGGT | 226 |
| | CDR H3 | AAGWNFDY | 227 |
| | CDR L1 | QDVGTA | 228 |
| | CDR L2 | WAS | 229 |
| | CDR L3 | QQYNSYPLT | 230 |
| R12 | CDR H1 | GFDFSAYY | 231 |
| | CDR H2 | IYPSSGKT | 232 |
| | CDR H3 | ARDSYADDGALFNI | 233 |
| | CDR L1 | SAHKTDT | 234 |
| | CDR L2 | VQSDGSY | 235 |
| | CDR L3 | GADYIGGYV | 236 |

TABLE ZZ
Sequences

| SEQ | | | | |
|---|---|---|---|---|
| ID | Clone | | | |
| NO: | # | Descr. | Sequence | Location |
| 1 | 11074 | Full | DIQMTQSPSTLSASVGDRVTITCKCQLSVGYMHWYQQ | |
| | | | KPGKAPKLLIYDTSKLASGVPSRFSGSGSGTEFTLTISSLQ | |
| | | | PDDFATYYCFQGSGYPFTFGGGTKLEIKRTVAAPSVFIFP | |
| | | | PSDEQLKSGTASVVCLLNNFYPREAKVQWKVDNALQS | |
| | | | GNSQESVTEQDSKDSTYSLSSTLTLSKADYEKHKVYACEV | |
| | | | THQGLSSPVTKSFNRGEC | |
| 2 | 11074 | Full | GATATTCAGATGACCCAGTCTCCCAGCACACTGTCCG | |
| | | | CCTCTGTGGGCGACCGGGTGACCATCACATGCAAGTG | |
| | | | TCAGCTGAGCGTGGGCTACATGCACTGGTATCAGCAG | |
| | | | AAGCCCGGCAAGGCCCCTAAGCTGCTGATCTACGATA | |
| | | | CCAGCAAGCTGGCCTCCGGCGTGCCATCTAGATTCAG | |
| | | | CGGCTCCGGCTCTGGCACCGAGTTTACCCTGACAATC | |
| | | | AGCTCCCTGCAGCCCGACGATTTCGCCACATACTATTG | |
| | | | CTTTCAGGGGAGCGGCTACCCATTCACATTCGGAGGG | |
| | | | GGAACTAAACTGGAAATCAAGAGGACCGTCGCGGCG | |
| | | | CCCAGTGTCTTCATTTTTCCCCCTAGCGACGAACAGCT | |
| | | | GAAGTCTGGGACAGCCAGTGTGGTCTGTCTGCTGAAC | |
| | | | AACTTCTACCCTAGAGAGGCTAAAGTGCAGTGGAAG | |
| | | | GTCGATAACGCACTGCAGTCCGGAAATTCTCAGGAGA | |
| | | | GTGTGACTGAACAGGACTCAAAAGATAGCACCTATTC | |
| | | | CCTGTCAAGCACACTGACTCTGAGCAAGGCCGACTAC | |
| | | | GAGAAGCATAAAGTGTATGCTTGTGAAGTCACCCACC | |
| | | | AGGGGCTGAGTTCACCAGTCACAAAATCATTCAACAG | |
| | | | AGGGGAGTGC | |
| 3 | 11074 | VL | DIQMTQSPSTLSASVGDRVTITCKCQLSVGYMHWYQQ | D1-K106 |
| | | | KPGKAPKLLIYDTSKLASGVPSRFSGSGSGTEFTLTISSLQ | |
| | | | PDDFATYYCFQGSGYPFTFGGGTKLEIK | |
| 4 | 11011 | Full | QVTLRESGPALVKPTQTLTLTCTFSGFSLSTSGMSVGWI | |
| | | | RQPPGKALEWLADIWWDDKKDYNPSLKSRLTISKDTSK | |
| | | | NQVVLKVTNMDPADTATYYCARSMITNWYFDVWGAG | |
| | | | TTVTVSSASTKGPSVFPLAPSSKSTSGGTAALGCLVKDYF | |
| | | | PEPVTVSWNSGALTSGVHTFPAVLQSSGLYSLSSVVTVP | |
| | | | SSSLGTQTYICNVNHKPSNTKVDKKVEPKSCDKTHTCPP | |
| | | | CPAPEAAGGPSVFLFPPKPKDTLMISRTPEVTCVVVSVS | |
| | | | HEDPEVKFNWYVDGVEVHNAKTKPREEQYNSTYRVVS | |
| | | | VLTVLHQDWLNGKEYKCKVSNKALPAPIEKTISKAKGQP | |
| | | | REPQVYVYPPSRDELTKNQVSLTCLVKGFYPSDIAVEWE | |
| | | | SNGQPENNYKTTPPVLDSDGSFALVSKLTVDKSRWQQ | |
| | | | GNVFSCSVMHEALHNHYTQKSLSLSPG | |
| 5 | 11011 | Full | CAGGTGACACTGAGGGAGAGCGGACCAGCCCTGGTG | |
| | | | AAGCCAACCCAGACACTGACCCTGACATGCACCTTCT | |
| | | | CCGGCTTTAGCCTGTCCACATCTGGCATGTCTGTGGG | |
| | | | CTGGATCAGACAGCCACCTGGCAAGGCCCTGGAGTG | |
| | | | GCTGGCCGACATCTGGTGGGACGATAAGAAGGATTA | |
| | | | CAACCCTAGCCTGAAGTCCAGACTGACAATCTCTAAG | |
| | | | GACACCAGCAAGAACCAGGTGGTGCTGAAGGTGACC | |
| | | | AATATGGACCCCGCCGATACAGCCACCTACTATTGTG | |
| | | | CCCGGTCCATGATTACTAACTGGTATTTTGATGTCTGG | |
| | | | GGGGCAGGAACAACCGTGACCGTCTCTTCTGCTAGCA | |
| | | | CTAAGGGGCCTTCCGTGTTTCCACTGGCTCCCTCTAGT | |
| | | | AAATCCACCTCTGGAGGCACAGCTGCACTGGGATGTC | |
| | | | TGGTGAAGGATTACTTCCCTGAACCAGTCACAGTGAG | |
| | | | TTGGAACTCAGGGGCTCTGACAAGTGGAGTCCATACT | |
| | | | TTTCCCGCAGTGCTGCAGTCAAGCGGACTGTACTCCC | |
| | | | TGTCCTCTGTGGTCACCGTGCCTAGTTCAAGCCTGGG | |
| | | | CACCCAGACATATATCTGCAACGTGAATCACAAGCCA | |
| | | | TCAAATACAAAAGTCGACAAGAAAGTGGAGCCCAAG | |
| | | | AGCTGTGATAAAACTCATACCTGCCCACCTTGTCCGG | |
| | | | CGCCAGAGGCTGCAGGAGGACCAAGCGTGTTCCTGT | |
| | | | TTCCACCCAAGCCTAAAGACACACTGATGATTTCCCG | |
| | | | AACCCCCGAAGTCACATGCGTGGTCGTGTCTGTGAGT | |
| | | | CACGAGGACCCTGAAGTCAAGTTCAACTGGTACGTG | |
| | | | GATGGCGTCGAGGTGCATAATGCCAAGACTAAACCT | |
| | | | AGGGAGGAACAGTACAACTCAACCTATCGCGTCGTG | |
| | | | AGCGTCCTGACAGTGCTGCACCAGGATTGGCTGAAC | |
| | | | GGCAAAGAATATAAGTGCAAAGTGAGCAATAAGGCC | |
| | | | CTGCCCGCTCCTATCGAGAAAACCATTTCCAAGGCTA | |
| | | | AAGGGCAGCCTCGCGAACCACAGGTCTACGTGTATCC | |
| | | | TCCAAGCCGGGACGAGCTGACAAAGAACCAGGTCTC | |
| | | | CCTGACTTGTCTGGTGAAAGGGTTTTACCCTAGTGAT | |
| | | | ATCGCTGTGGAGTGGGAATCAAATGGACAGCCAGAG | |
| | | | AACAATTATAAGACTACCCCCCCTGTGCTGGACAGTG | |
| | | | ATGGGTCATTCGCACTGGTCTCCAAGCTGACAGTGGA | |
| | | | CAAATCTCGGTGGCAGCAGGGAAATGTCTTTTCATGT | |
| | | | AGCGTGATGCATGAAGCACTGCACAACCATTACACCC | |
| | | | AGAAGTCACTGTCACTGTCACCAGGA | |
| 6 | 11011 | VH | QVTLRESGPALVKPTQTLTLTCTFSGFSLSTSGMSVGWI | Q1-S120 |
| | | | RQPPGKALEWLADIWWDDKKDYNPSLKSRLTISKDTSK | |
| | | | NQVVLKVTNMDPADTATYYCARSMITNWYFDVWGAG | |
| | | | TTVTVSS | |
| 7 | 12644 | Full | QVQLQQSGAELARPGASVKMSCKASGYTFTTYTMHW | |
| | | | VKQRPGQGLEWIGYINPSSGYTNYNQKFKDKATLTADK | |
| | | | SSSTASMQLSSLTSEDSAVYYCARERAVLVPYAMDYWG | |
| | | | QGTSVTVSSASTKGPSVFPLAPSSKSTSGGTAALGCLVK | |
| | | | DYFPEPVTVSWNSGALTSGVHTFPAVLQSSGLYSLSSVV | |
| | | | TVPSSSLGTQTYICNVNHKPSNTKVDKKVEPKSCDKTHT | |
| | | | CPPCPAPEAAGGPSVFLFPPKPKDTLMISRTPEVTCVVV | |
| | | | SVSHEDPEVKFNWYVDGVEVHNAKTKPREEQYNSTYR | |
| | | | VVSVLTVLHQDWLNGKEYKCKVSNKALPAPIEKTISKAK | |
| | | | GQPREPQVYVYPPSRDELTKNQVSLTCLVKGFYPSDIAV | |
| | | | EWESNGQPENNYKTTPPVLDSDGSFALVSKLTVDKSRW | |
| | | | QQGNVFSCSVMHEALHNHYTQKSLSLSPG | |
| 8 | 12644 | Full | CAGGTGCAGCTGCAGCAGAGCGGAGCCGAGCTGGCC | |
| | | | AGGCCAGGGGCCAGCGTGAAGATGAGCTGCAAGGC | |
| | | | CTCCGGCTACACCTTCACCACATATACAATGCACTGG | |
| | | | GTGAAGCAGCGGCCCGGACAGGGCCTGGAGTGGATC | |
| | | | GGCTACATCAACCCTAGCTCCGGCTACACCAACTATA | |
| | | | ATCAGAAGTTTAAGGACAAGGCCACCCTGACAGCCG | |
| | | | ATAAGTCTAGCTCCACCGCCTCTATGCAGCTGTCTAGC | |
| | | | CTGACAAGCGAGGACTCCGCCGTGTACTATTGTGCCC | |
| | | | GGGAGAGAGCCGTGCTGGTGCCATACGCCATGGATT | |
| | | | ATTGGGGCCAGGGCACCTCCGTGACAGTGTCCTCTGC | |
| | | | TAGCACTAAGGGGCCTTCCGTGTTTCCACTGGCTCCCT | |
| | | | CTAGTAAATCCACCTCTGGAGGCACAGCTGCACTGGG | |
| | | | ATGTCTGGTGAAGGATTACTTCCCTGAACCAGTCACA | |
| | | | GTGAGTTGGAACTCAGGGGCTCTGACAAGTGGAGTC | |
| | | | CATACTTTTCCCGCAGTGCTGCAGTCAAGCGGACTGT | |
| | | | ACTCCCTGTCCTCTGTGGTCACCGTGCCTAGTTCAAGC | |
| | | | CTGGGCACCCAGACATATATCTGCAACGTGAATCACA | |
| | | | AGCCATCAAATACAAAAGTCGACAAGAAAGTGGAGC | |
| | | | CCAAGAGCTGTGATAAAACTCATACCTGCCCACCTTG | |
| | | | TCCGGCGCCAGAGGCTGCAGGAGGACCAAGCGTGTT | |
| | | | CCTGTTTCCACCCAAGCCTAAAGACACACTGATGATTT | |
| | | | CCCGAACCCCCGAAGTCACATGCGTGGTCGTGTCTGT | |
| | | | GAGTCACGAGGACCCTGAAGTCAAGTTCAACTGGTAC | |
| | | | GTGGATGGCGTCGAGGTGCATAATGCCAAGACTAAA | |
| | | | CCTAGGGAGGAACAGTACAACTCAACCTATCGCGTCG | |
| | | | TGAGCGTCCTGACAGTGCTGCACCAGGATTGGCTGAA | |
| | | | CGGCAAAGAATATAAGTGCAAAGTGAGCAATAAGGC | |
| | | | CCTGCCCGCTCCTATCGAGAAAACCATTTCCAAGGCT | |
| | | | AAAGGGCAGCCTCGCGAACCACAGGTCTACGTGTATC | |
| | | | CTCCAAGCCGGGACGAGCTGACAAAGAACCAGGTCT | |
| | | | CCCTGACTTGTCTGGTGAAAGGGTTTTACCCTAGTGA | |
| | | | TATCGCTGTGGAGTGGGAATCAAATGGACAGCCAGA | |
| | | | GAACAATTATAAGACTACCCCCCCTGTGCTGGACAGT | |
| | | | GATGGGTCATTCGCACTGGTCTCCAAGCTGACAGTGG | |
| | | | ACAAATCTCGGTGGCAGCAGGGAAATGTCTTTTCATG | |
| | | | TAGCGTGATGCATGAAGCACTGCACAACCATTACACC | |
| | | | CAGAAGTCACTGTCACTGTCACCAGGA | |
| 9 | 12644 | VH | QVQLQQSGAELARPGASVKMSCKASGYTFTTYTMHW | Q1-S121 |
| | | | VKQRPGQGLEWIGYINPSSGYTNYNQKFKDKATLTADK | |
| | | | SSSTASMQLSSLTSEDSAVYYCARERAVLVPYAMDYWG | |
| | | | QGTSVTVSS | |
| 10 | 12645 | Full | QIVLTQSPAVMSASPGEKVTITCTASSSLSYMHWFQQK | |
| | | | PGTSPKLWLYSTSILASGVPTRFSGSGSGTSYSLTISRME | |
| | | | AEDAATYYCQQRSSSPFTFGSGTKLEIKRTVAAPSVFIFP | |
| | | | PSDEQLKSGTASVVCLLNNFYPREAKVQWKVDNALQS | |
| | | | GNSQESVTEQDSKDSTYSLSSTLTLSKADYEKHKVYACEV | |
| | | | THQGLSSPVTKSFNRGEC | |
| 11 | 12645 | Full | CAGATCGTGCTGACCCAGTCCCCAGCCGTGATGAGCG | |
| | | | CCTCCCCAGGAGAGAAGGTGACCATCACATGCACCGC | |
| | | | CAGCTCCTCTCTGAGCTACATGCACTGGTTCCAGCAG | |
| | | | AAGCCCGGCACATCCCCTAAGCTGTGGCTGTATTCTA | |
| | | | CCAGCATCCTGGCCTCTGGCGTGCCTACAAGGTTTTCC | |
| | | | GGCTCTGGCAGCGGCACATCCTACTCTCTGACCATCA | |
| | | | GCCGGATGGAGGCAGAGGACGCAGCAACCTACTATT | |
| | | | GTCAGCAGAGAAGCTCCTCTCCCTTCACATTTGGCAG | |
| | | | CGGCACCAAGCTGGAGATCAAGCGGACAGTGGCGGC | |
| | | | GCCCAGTGTCTTCATTTTTCCCCCTAGCGACGAACAGC | |
| | | | TGAAGTCTGGGACAGCCAGTGTGGTCTGTCTGCTGAA | |
| | | | CAACTTCTACCCTAGAGAGGCTAAAGTGCAGTGGAA | |
| | | | GGTCGATAACGCACTGCAGTCCGGAAATTCTCAGGA | |
| | | | GAGTGTGACTGAACAGGACTCAAAAGATAGCACCTA | |
| | | | TTCCCTGTCAAGCACACTGACTCTGAGCAAGGCCGAC | |
| | | | TACGAGAAGCATAAAGTGTATGCTTGTGAAGTCACCC | |
| | | | ACCAGGGGCTGAGTTCACCAGTCACAAAATCATTCAA | |
| | | | CAGAGGGGAGTGC | |
| 12 | 12645 | VL | QIVLTQSPAVMSASPGEKVTITCTASSSLSYMHWFQQK | Q1-K106 |
| | | | PGTSPKLWLYSTSILASGVPTRFSGSGSGTSYSLTISRME | |
| | | | AEDAATYYCQQRSSSPFTFGSGTKLEIK | |
| 13 | 12646 | Full | EVQLQQSGPELEKPGASVKISCKASGYSFTGYNMNWVK | |
| | | | QSNGKSLEWIGNIDPYYGDTNYNQKFKGKATLTVDKSS | |
| | | | STAYMHLKSLTSEDSAVYYCARPYGSEAYFAYWGQGTL | |
| | | | VTVSAASTKGPSVFPLAPSSKSTSGGTAALGCLVKDYFPE | |
| | | | PVTVSWNSGALTSGVHTFPAVLQSSGLYSLSSVVTVPSS | |
| | | | SLGTQTYICNVNHKPSNTKVDKKVEPKSCDKTHTCPPCP | |
| | | | APEAAGGPSVFLFPPKPKDTLMISRTPEVTCVVVSVSHE | |
| | | | DPEVKFNWYVDGVEVHNAKTKPREEQYNSTYRVVSVLT | |
| | | | VLHQDWLNGKEYKCKVSNKALPAPIEKTISKAKGQPREP | |
| | | | QVYVYPPSRDELTKNQVSLTCLVKGFYPSDIAVEWESNG | |
| | | | QPENNYKTTPPVLDSDGSFALVSKLTVDKSRWQQGNVF | |
| | | | SCSVMHEALHNHYTQKSLSLSPG | |
| 14 | 12646 | Full | GAGGTGCAGCTGCAGCAGTCTGGACCAGAGCTGGAG | |
| | | | AAGCCTGGGGCCAGCGTGAAGATCAGCTGCAAGGCC | |
| | | | AGCGGCTACTCCTTCACCGGCTATAACATGAATTGGG | |
| | | | TGAAGCAGTCCAACGGCAAGTCTCTGGAGTGGATCG | |
| | | | GCAATATCGACCCATACTATGGCGATACAAACTACAA | |
| | | | TCAGAAGTTTAAGGGCAAGGCCACCCTGACAGTGGA | |
| | | | CAAGAGCTCCTCTACCGCCTATATGCACCTGAAGTCTC | |
| | | | TGACAAGCGAGGATTCCGCCGTGTACTATTGTGCCAG | |
| | | | ACCCTACGGCAGCGAGGCCTACTTCGCCTATTGGGGC | |
| | | | CAGGGCACCCTGGTGACAGTGTCCGCCGCTAGCACTA | |
| | | | AGGGGCCTTCCGTGTTTCCACTGGCTCCCTCTAGTAAA | |
| | | | TCCACCTCTGGAGGCACAGCTGCACTGGGATGTCTGG | |
| | | | TGAAGGATTACTTCCCTGAACCAGTCACAGTGAGTTG | |
| | | | GAACTCAGGGGCTCTGACAAGTGGAGTCCATACTTTT | |
| | | | CCCGCAGTGCTGCAGTCAAGCGGACTGTACTCCCTGT | |
| | | | CCTCTGTGGTCACCGTGCCTAGTTCAAGCCTGGGCAC | |
| | | | CCAGACATATATCTGCAACGTGAATCACAAGCCATCA | |
| | | | AATACAAAAGTCGACAAGAAAGTGGAGCCCAAGAGC | |
| | | | TGTGATAAAACTCATACCTGCCCACCTTGTCCGGCGCC | |
| | | | AGAGGCTGCAGGAGGACCAAGCGTGTTCCTGTTTCCA | |
| | | | CCCAAGCCTAAAGACACACTGATGATTTCCCGAACCC | |
| | | | CCGAAGTCACATGCGTGGTCGTGTCTGTGAGTCACGA | |
| | | | GGACCCTGAAGTCAAGTTCAACTGGTACGTGGATGG | |
| | | | CGTCGAGGTGCATAATGCCAAGACTAAACCTAGGGA | |
| | | | GGAACAGTACAACTCAACCTATCGCGTCGTGAGCGTC | |
| | | | CTGACAGTGCTGCACCAGGATTGGCTGAACGGCAAA | |
| | | | GAATATAAGTGCAAAGTGAGCAATAAGGCCCTGCCC | |
| | | | GCTCCTATCGAGAAAACCATTTCCAAGGCTAAAGGGC | |
| | | | AGCCTCGCGAACCACAGGTCTACGTGTATCCTCCAAG | |
| | | | CCGGGACGAGCTGACAAAGAACCAGGTCTCCCTGAC | |
| | | | TTGTCTGGTGAAAGGGTTTTACCCTAGTGATATCGCT | |
| | | | GTGGAGTGGGAATCAAATGGACAGCCAGAGAACAAT | |
| | | | TATAAGACTACCCCCCCTGTGCTGGACAGTGATGGGT | |
| | | | CATTCGCACTGGTCTCCAAGCTGACAGTGGACAAATC | |
| | | | TCGGTGGCAGCAGGGAAATGTCTTTTCATGTAGCGTG | |
| | | | ATGCATGAAGCACTGCACAACCATTACACCCAGAAGT | |
| | | | CACTGTCACTGTCACCAGGA | |
| 15 | 12646 | VH | EVQLQQSGPELEKPGASVKISCKASGYSFTGYNMNWVK | E1-A119 |
| | | | QSNGKSLEWIGNIDPYYGDTNYNQKFKGKATLTVDKSS | |
| | | | STAYMHLKSLTSEDSAVYYCARPYGSEAYFAYWGQGTL | |
| | | | VTVSA | |
| 16 | 12647 | Full | DIVMTQSPATLSVTPGDRVSLSCRASQSISDYLHWYQQ | |
| | | | KSHESPRLLIKYAAQSISGIPSRFSGSGSGSDFTLSINGVEP | |
| | | | EDVGVYYCQNGHSFPYTFGGGTKLEIKRTVAAPSVFIFPP | |
| | | | SDEQLKSGTASVVCLLNNFYPREAKVQWKVDNALQSG | |
| | | | NSQESVTEQDSKDSTYSLSSTLTLSKADYEKHKVYACEVT | |
| | | | HQGLSSPVTKSFNRGEC | |
| 17 | 12647 | Full | GACATCGTGATGACCCAGTCCCCCGCCACCCTGTCTG | |
| | | | TGACACCTGGCGACCGGGTGAGCCTGTCCTGCAGAG | |
| | | | CCTCTCAGAGCATCTCCGATTACCTGCACTGGTATCAG | |
| | | | CAGAAGTCTCACGAGAGCCCAAGGCTGCTGATCAAG | |
| | | | TACGCCGCCCAGTCTATCAGCGGCATCCCCAGCCGCT | |
| | | | TCTCCGGCTCTGGCAGCGGCTCCGACTTTACCCTGTCC | |
| | | | ATCAACGGCGTGGAGCCTGAGGATGTGGGCGTGTAC | |
| | | | TATTGTCAGAATGGCCACTCTTTCCCCTATACCTTTGG | |
| | | | CGGCGGCACAAAGCTGGAGATCAAGCGGACAGTGGC | |
| | | | GGCGCCCAGTGTCTTCATTTTTCCCCCTAGCGACGAAC | |
| | | | AGCTGAAGTCTGGGACAGCCAGTGTGGTCTGTCTGCT | |
| | | | GAACAACTTCTACCCTAGAGAGGCTAAAGTGCAGTG | |
| | | | GAAGGTCGATAACGCACTGCAGTCCGGAAATTCTCAG | |
| | | | GAGAGTGTGACTGAACAGGACTCAAAAGATAGCACC | |
| | | | TATTCCCTGTCAAGCACACTGACTCTGAGCAAGGCCG | |
| | | | ACTACGAGAAGCATAAAGTGTATGCTTGTGAAGTCAC | |
| | | | CCACCAGGGGCTGAGTTCACCAGTCACAAAATCATTC | |
| | | | AACAGAGGGGAGTGC | |
| 18 | 12647 | VL | DIVMTQSPATLSVTPGDRVSLSCRASQSISDYLHWYQQ | D1-K107 |
| | | | KSHESPRLLIKYAAQSISGIPSRFSGSGSGSDFTLSINGVEP | |
| | | | EDVGVYYCQNGHSFPYTFGGGTKLEIK | |
| 19 | 12648 | Full | QVQLKESGPGLVAPSQSLSITCSVSGFSLSNYDISWIRQP | |
| | | | PGKGLEWLGVMWTGGGANYNSAFMSRLSINKDNSKS | |
| | | | QVFLKMNNLQTDDTAIYYCVRDAVRYWNFDVWGAGT | |
| | | | TVTVSSASTKGPSVFPLAPSSKSTSGGTAALGCLVKDYFP | |
| | | | EPVTVSWNSGALTSGVHTFPAVLQSSGLYSLSSVVTVPS | |
| | | | SSLGTQTYICNVNHKPSNTKVDKKVEPKSCDKTHTCPPC | |
| | | | PAPEAAGGPSVFLFPPKPKDTLMISRTPEVTCVVVSVSH | |
| | | | EDPEVKFNWYVDGVEVHNAKTKPREEQYNSTYRVVSVL | |
| | | | TVLHQDWLNGKEYKCKVSNKALPAPIEKTISKAKGQPRE | |
| | | | PQVYVYPPSRDELTKNQVSLTCLVKGFYPSDIAVEWESN | |
| | | | GQPENNYKTTPPVLDSDGSFALVSKLTVDKSRWQQGN | |
| | | | VFSCSVMHEALHNHYTQKSLSLSPG | |
| 20 | 12648 | Full | CAGGTGCAGCTGAAGGAGTCCGGACCAGGCCTGGTG | |
| | | | GCCCCCTCTCAGAGCCTGTCCATCACCTGCTCTGTGAG | |
| | | | CGGCTTCTCCCTGTCTAACTACGACATCTCCTGGATCA | |
| | | | GGCAGCCACCTGGCAAGGGCCTGGAGTGGCTGGGCG | |
| | | | TGATGTGGACAGGAGGAGGAGCCAACTATAATTCTG | |
| | | | CCTTCATGTCTCGGCTGAGCATCAACAAGGATAATAG | |
| | | | CAAGTCCCAGGTGTTTCTGAAGATGAACAATCTGCAG | |
| | | | ACCGACGATACAGCCATCTACTATTGCGTGCGGGACG | |
| | | | CCGTGAGATACTGGAATTTTGACGTGTGGGGGGCAG | |
| | | | GGACCACAGTGACCGTGAGCTCCGCTAGCACTAAGG | |
| | | | GGCCTTCCGTGTTTCCACTGGCTCCCTCTAGTAAATCC | |
| | | | ACCTCTGGAGGCACAGCTGCACTGGGATGTCTGGTG | |
| | | | AAGGATTACTTCCCTGAACCAGTCACAGTGAGTTGGA | |
| | | | ACTCAGGGGCTCTGACAAGTGGAGTCCATACTTTTCC | |
| | | | CGCAGTGCTGCAGTCAAGCGGACTGTACTCCCTGTCC | |
| | | | TCTGTGGTCACCGTGCCTAGTTCAAGCCTGGGCACCC | |
| | | | AGACATATATCTGCAACGTGAATCACAAGCCATCAAA | |
| | | | TACAAAAGTCGACAAGAAAGTGGAGCCCAAGAGCTG | |
| | | | TGATAAAACTCATACCTGCCCACCTTGTCCGGCGCCA | |
| | | | GAGGCTGCAGGAGGACCAAGCGTGTTCCTGTTTCCAC | |
| | | | CCAAGCCTAAAGACACACTGATGATTTCCCGAACCCC | |
| | | | CGAAGTCACATGCGTGGTCGTGTCTGTGAGTCACGAG | |
| | | | GACCCTGAAGTCAAGTTCAACTGGTACGTGGATGGC | |
| | | | GTCGAGGTGCATAATGCCAAGACTAAACCTAGGGAG | |
| | | | GAACAGTACAACTCAACCTATCGCGTCGTGAGCGTCC | |
| | | | TGACAGTGCTGCACCAGGATTGGCTGAACGGCAAAG | |
| | | | AATATAAGTGCAAAGTGAGCAATAAGGCCCTGCCCG | |
| | | | CTCCTATCGAGAAAACCATTTCCAAGGCTAAAGGGCA | |
| | | | GCCTCGCGAACCACAGGTCTACGTGTATCCTCCAAGC | |
| | | | CGGGACGAGCTGACAAAGAACCAGGTCTCCCTGACTT | |
| | | | GTCTGGTGAAAGGGTTTTACCCTAGTGATATCGCTGT | |
| | | | GGAGTGGGAATCAAATGGACAGCCAGAGAACAATTA | |
| | | | TAAGACTACCCCCCCTGTGCTGGACAGTGATGGGTCA | |
| | | | TTCGCACTGGTCTCCAAGCTGACAGTGGACAAATCTC | |
| | | | GGTGGCAGCAGGGAAATGTCTTTTCATGTAGCGTGAT | |
| | | | GCATGAAGCACTGCACAACCATTACACCCAGAAGTCA | |
| | | | CTGTCACTGTCACCAGGA | |
| 21 | 12648 | VH | QVQLKESGPGLVAPSQSLSITCSVSGFSLSNYDISWIRQP | Q1-S118 |
| | | | PGKGLEWLGVMWTGGGANYNSAFMSRLSINKDNSKS | |
| | | | QVFLKMNNLQTDDTAIYYCVRDAVRYWNFDVWGAGT | |
| | | | TVTVSS | |
| 22 | 12649 | Full | QIVLSQSPAILSASPGEKVTMTCRASSSVSYIHWYQQKP | |
| | | | GSSPKPWIYATSHLASGVPARFSGSGSGTSYSLTISRVEA | |
| | | | EDTATYYCQQWSSNPFTFGSGTKLEIKRTVAAPSVFIFPP | |
| | | | SDEQLKSGTASVVCLLNNFYPREAKVQWKVDNALQSG | |
| | | | NSQESVTEQDSKDSTYSLSSTLTLSKADYEKHKVYACEVT | |
| | | | HQGLSSPVTKSFNRGEC | |
| 23 | 12649 | Full | CAGATCGTGCTGTCCCAGTCTCCAGCCATCCTGAGCG | |
| | | | CCTCCCCAGGAGAGAAGGTGACCATGACATGCAGGG | |
| | | | CCAGCTCCTCTGTGAGCTACATCCACTGGTATCAGCA | |
| | | | GAAGCCTGGCAGCTCCCCCAAGCCTTGGATCTACGCC | |
| | | | ACCTCCCACCTGGCCTCTGGAGTGCCAGCCCGGTTCT | |
| | | | CTGGCAGCGGCTCCGGCACCTCTTATAGCCTGACAAT | |
| | | | CAGCAGAGTGGAGGCCGAGGACACCGCCACATACTA | |
| | | | TTGTCAGCAGTGGTCTAGCAACCCCTTCACCTTTGGCT | |
| | | | CCGGCACAAAGCTGGAGATCAAGCGGACAGTGGCGG | |
| | | | CGCCCAGTGTCTTCATTTTTCCCCCTAGCGACGAACAG | |
| | | | CTGAAGTCTGGGACAGCCAGTGTGGTCTGTCTGCTGA | |
| | | | ACAACTTCTACCCTAGAGAGGCTAAAGTGCAGTGGAA | |
| | | | GGTCGATAACGCACTGCAGTCCGGAAATTCTCAGGA | |
| | | | GAGTGTGACTGAACAGGACTCAAAAGATAGCACCTA | |
| | | | TTCCCTGTCAAGCACACTGACTCTGAGCAAGGCCGAC | |
| | | | TACGAGAAGCATAAAGTGTATGCTTGTGAAGTCACCC | |
| | | | ACCAGGGGCTGAGTTCACCAGTCACAAAATCATTCAA | |
| | | | CAGAGGGGAGTGC | |
| 24 | 12649 | VL | QIVLSQSPAILSASPGEKVTMTCRASSSVSYIHWYQQKP | Q1-K106 |
| | | | GSSPKPWIYATSHLASGVPARFSGSGSGTSYSLTISRVEA | |
| | | | EDTATYYCQQWSSNPFTFGSGTKLE1K | |
| 25 | 11082 | Full | QVTLRESGPALVKPTQTLTLTCTFSGFSLSTSGMSVGWI | |
| | | | RQPPGKALEWLADIWWDDKKDYNPSLKSRLTISKDTSK | |
| | | | NQVVLKVTNMDPADTATYYCARSMITNWYFDVWGAG | |
| | | | TTVTVSSVEGGSGGSGGSGGSGGVDDIQMTQSPSTLSA | |
| | | | SVGDRVTITCKCQLSVGYMHWYQQKPGKAPKLLIYDTS | |
| | | | KLASGVPSRFSGSGSGTEFTLTISSLQPDDFATYYCFQGS | |
| | | | GYPFTFGGGTKLEIKAAEPKSSDKTHTCPPCPAPEAAGG | |
| | | | PSVFLFPPKPKDTLMISRTPEVTCVVVSVSHEDPEVKFN | |
| | | | WYVDGVEVHNAKTKPREEQYNSTYRVVSVLTVLHQDW | |
| | | | LNGKEYKCKVSNKALPAPIEKTISKAKGQPREPQVYVLPP | |
| | | | SRDELTKNQVSLLCLVKGFYPSDIAVEWESNGQPENNYL | |
| | | | TWPPVLDSDGSFFLYSKLTVDKSRWQQGNVFSCSVMH | |
| | | | EALHNHYTQKSLSLSPG | |
| 26 | 11082 | Full | CAGGTGACCCTGAGAGAGAGCGGACCCGCCCTGGTG | |
| | | | AAGCCTACCCAGACACTGACCCTGACATGCACCTTCA | |
| | | | GCGGCTTTAGCCTGTCCACCTCTGGCATGTCCGTGGG | |
| | | | ATGGATCAGGCAGCCACCTGGCAAGGCCCTGGAGTG | |
| | | | GCTGGCCGACATCTGGTGGGACGATAAGAAGGATTA | |
| | | | CAACCCTTCCCTGAAGTCTCGCCTGACAATCTCCAAGG | |
| | | | ACACCTCTAAGAACCAGGTGGTGCTGAAGGTGACCA | |
| | | | ATATGGACCCAGCCGATACAGCCACCTACTATTGTGC | |
| | | | CCGGTCCATGATCACAAATTGGTATTTCGACGTGTGG | |
| | | | GGAGCCGGAACCACAGTGACCGTGAGCTCCGTGGAG | |
| | | | GGAGGCAGCGGAGGCTCCGGAGGCTCTGGAGGCAG | |
| | | | CGGAGGAGTGGACGATATCCAGATGACACAGAGCCC | |
| | | | CTCCACCCTGTCTGCCAGCGTGGGCGACCGGGTGACA | |
| | | | ATCACCTGCAAGTGTCAGCTGTCCGTGGGCTACATGC | |
| | | | ACTGGTATCAGCAGAAGCCTGGCAAGGCCCCAAAGC | |
| | | | TGCTGATCTACGATACCAGCAAGCTGGCCTCCGGCGT | |
| | | | GCCTTCTAGGTTCTCCGGCTCTGGCAGCGGCACAGAG | |
| | | | TTTACACTGACCATCTCTAGCCTGCAGCCAGACGATTT | |
| | | | CGCCACCTACTATTGCTTTCAGGGCAGCGGCTATCCCT | |
| | | | TCACATTTGGCGGCGGCACCAAGCTGGAGATCAAGG | |
| | | | CCGCCGAGCCTAAGTCCTCTGACAAGACACACACCTG | |
| | | | CCCACCCTGTCCGGCGCCAGAGGCAGCAGGAGGACC | |
| | | | AAGCGTGTTCCTGTTTCCACCCAAGCCCAAAGACACC | |
| | | | CTGATGATTAGCCGAACCCCTGAAGTCACATGCGTGG | |
| | | | TCGTGTCCGTGTCTCACGAGGACCCAGAAGTCAAGTT | |
| | | | CAACTGGTACGTGGATGGCGTCGAGGTGCATAATGC | |
| | | | CAAGACAAAACCCCGGGAGGAACAGTACAACAGCAC | |
| | | | CTATAGAGTCGTGTCCGTCCTGACAGTGCTGCACCAG | |
| | | | GATTGGCTGAACGGCAAGGAATATAAGTGCAAAGTG | |
| | | | TCCAATAAGGCCCTGCCCGCTCCTATCGAGAAAACCA | |
| | | | TTTCTAAGGCAAAAGGCCAGCCTCGCGAACCACAGGT | |
| | | | CTACGTGCTGCCTCCATCCCGGGACGAGCTGACAAAG | |
| | | | AACCAGGTCTCTCTGCTGTGCCTGGTGAAAGGCTTCT | |
| | | | ATCCATCAGATATTGCTGTGGAGTGGGAAAGCAATG | |
| | | | GGCAGCCCGAGAACAATTACCTGACTTGGCCCCCTGT | |
| | | | GCTGGACTCTGATGGGAGTTTCTTTCTGTATTCTAAGC | |
| | | | TGACCGTGGATAAAAGTAGGTGGCAGCAGGGAAATG | |
| | | | TCTTTAGTTGTTCAGTGATGCATGAAGCCCTGCATAAC | |
| | | | CACTACACCCAGAAAAGCCTGTCCCTGTCCCCCGGA | |
| 27 | 11082 | VH | QVTLRESGPALVKPTQTLTLTCTFSGFSLSTSGMSVGWI | Q1-S120 |
| | | | RQPPGKALEWLADIWWDDKKDYNPSLKSRLTISKDTSK | |
| | | | NQVVLKVTNMDPADTATYYCARSMITNWYFDVWGAG | |
| | | | TTVTVSS | |
| 28 | 12651 | Full | EIVLTQSPATLSLSPGERATLSCRASQSVSSYLAWYQQKP | |
| | | | GQAPRLLIYDASNRATGIPARFSGSGSGTDFTLTISSLEPE | |
| | | | DFAVYYCQQRRNWPLTFGGGTKVEIKRTVAAPSVFIFPP | |
| | | | SDEQLKSGTASVVCLLNNFYPREAKVQWKVDNALQSG | |
| | | | NSQESVTEQDSKDSTYSLSSTLTLSKADYEKHKVYACEVT | |
| | | | HQGLSSPVTKSFNRGEC | |
| 29 | 12651 | Full | GAGATCGTGCTGACCCAGTCTCCAGCCACACTGTCCC | |
| | | | TGTCTCCAGGAGAGAGGGCCACCCTGAGCTGCAGGG | |
| | | | CCAGCCAGTCCGTGAGCTCCTACCTGGCCTGGTATCA | |
| | | | GCAGAAGCCAGGACAGGCCCCCCGGCTGCTGATCTA | |
| | | | CGACGCCTCCAACAGGGCAACCGGCATCCCCGCAAG | |
| | | | ATTCTCTGGCAGCGGCTCCGGCACAGACTTTACCCTG | |
| | | | ACAATCTCTAGCCTGGAGCCTGAGGATTTCGCCGTGT | |
| | | | ACTATTGTCAGCAGCGGAGAAATTGGCCACTGACCTT | |
| | | | TGGCGGCGGCACAAAGGTGGAGATCAAGAGAACAG | |
| | | | TGGCGGCGCCCAGTGTCTTCATTTTTCCCCCTAGCGAC | |
| | | | GAACAGCTGAAGTCTGGGACAGCCAGTGTGGTCTGT | |
| | | | CTGCTGAACAACTTCTACCCTAGAGAGGCTAAAGTGC | |
| | | | AGTGGAAGGTCGATAACGCACTGCAGTCCGGAAATT | |
| | | | CTCAGGAGAGTGTGACTGAACAGGACTCAAAAGATA | |
| | | | GCACCTATTCCCTGTCAAGCACACTGACTCTGAGCAA | |
| | | | GGCCGACTACGAGAAGCATAAAGTGTATGCTTGTGA | |
| | | | AGTCACCCACCAGGGGCTGAGTTCACCAGTCACAAAA | |
| | | | TCATTCAACAGAGGGGAGTGC | |
| 30 | 12651 | VL | EIVLTQSPATLSLSPGERATLSCRASQSVSSYLAWYQQKP | E1-K107 |
| | | | GQAPRLLIYDASNRATGIPARFSGSGSGTDFTLTISSLEPE | |
| | | | DFAVYYCQQRRNWPLTFGGGTKVEIK | |
| 31 | 12652 | Full | EVKLVESGGGLVQPGGSLKLSCATSGFTFSDYYMYWVR | |
| | | | QTPEKRLEWVAYINSGGGSTYYPDTVKGRFTISRDNAK | |
| | | | NTLYLQMSRLKSEDTAMYYCARRGLPFHAMDYWGQG | |
| | | | TSVTVSSASTKGPSVFPLAPSSKSTSGGTAALGCLVKDYF | |
| | | | PEPVTVSWNSGALTSGVHTFPAVLQSSGLYSLSSVVTVP | |
| | | | SSSLGTQTYICNVNHKPSNTKVDKKVEPKSCDKTHTCPP | |
| | | | CPAPEAAGGPSVFLFPPKPKDTLMISRTPEVTCVVVSVS | |
| | | | HEDPEVKFNWYVDGVEVHNAKTKPREEQYNSTYRVVS | |
| | | | VLTVLHQDWLNGKEYKCKVSNKALPAPIEKTISKAKGQP | |
| | | | REPQVYVYPPSRDELTKNQVSLTCLVKGFYPSDIAVEWE | |
| | | | SNGQPENNYKTTPPVLDSDGSFALVSKLTVDKSRWQQ | |
| | | | GNVFSCSVMHEALHNHYTQKSLSLSPG | |
| 32 | 12652 | Full | GAGGTGAAGCTGGTGGAGAGCGGAGGAGGCCTGGT | |
| | | | GCAGCCAGGAGGCTCTCTGAAGCTGAGCTGCGCCAC | |
| | | | CTCCGGCTTCACATTTTCCGACTACTATATGTACTGGG | |
| | | | TGCGGCAGACCCCAGAGAAGAGGCTGGAGTGGGTG | |
| | | | GCCTATATCAACTCTGGCGGCGGCAGCACCTACTATC | |
| | | | CTGACACAGTGAAGGGCAGGTTCACCATCAGCCGGG | |
| | | | ACAACGCCAAGAATACACTGTACCTGCAGATGTCCCG | |
| | | | GCTGAAGTCTGAGGACACAGCCATGTACTATTGTGCC | |
| | | | CGGAGAGGCCTGCCCTTTCACGCCATGGATTATTGGG | |
| | | | GCCAGGGCACCAGCGTGACAGTGAGCTCCGCTAGCA | |
| | | | CTAAGGGGCCTTCCGTGTTTCCACTGGCTCCCTCTAGT | |
| | | | AAATCCACCTCTGGAGGCACAGCTGCACTGGGATGTC | |
| | | | TGGTGAAGGATTACTTCCCTGAACCAGTCACAGTGAG | |
| | | | TTGGAACTCAGGGGCTCTGACAAGTGGAGTCCATACT | |
| | | | TTTCCCGCAGTGCTGCAGTCAAGCGGACTGTACTCCC | |
| | | | TGTCCTCTGTGGTCACCGTGCCTAGTTCAAGCCTGGG | |
| | | | CACCCAGACATATATCTGCAACGTGAATCACAAGCCA | |
| | | | TCAAATACAAAAGTCGACAAGAAAGTGGAGCCCAAG | |
| | | | AGCTGTGATAAAACTCATACCTGCCCACCTTGTCCGG | |
| | | | CGCCAGAGGCTGCAGGAGGACCAAGCGTGTTCCTGT | |
| | | | TTCCACCCAAGCCTAAAGACACACTGATGATTTCCCG | |
| | | | AACCCCCGAAGTCACATGCGTGGTCGTGTCTGTGAGT | |
| | | | CACGAGGACCCTGAAGTCAAGTTCAACTGGTACGTG | |
| | | | GATGGCGTCGAGGTGCATAATGCCAAGACTAAACCT | |
| | | | AGGGAGGAACAGTACAACTCAACCTATCGCGTCGTG | |
| | | | AGCGTCCTGACAGTGCTGCACCAGGATTGGCTGAAC | |
| | | | GGCAAAGAATATAAGTGCAAAGTGAGCAATAAGGCC | |
| | | | CTGCCCGCTCCTATCGAGAAAACCATTTCCAAGGCTA | |
| | | | AAGGGCAGCCTCGCGAACCACAGGTCTACGTGTATCC | |
| | | | TCCAAGCCGGGACGAGCTGACAAAGAACCAGGTCTC | |
| | | | CCTGACTTGTCTGGTGAAAGGGTTTTACCCTAGTGAT | |
| | | | ATCGCTGTGGAGTGGGAATCAAATGGACAGCCAGAG | |
| | | | AACAATTATAAGACTACCCCCCCTGTGCTGGACAGTG | |
| | | | ATGGGTCATTCGCACTGGTCTCCAAGCTGACAGTGGA | |
| | | | CAAATCTCGGTGGCAGCAGGGAAATGTCTTTTCATGT | |
| | | | AGCGTGATGCATGAAGCACTGCACAACCATTACACCC | |
| | | | AGAAGTCACTGTCACTGTCACCAGGA | |
| 33 | 12652 | VH | EVKLVESGGGLVQPGGSLKLSCATSGFTFSDYYMYWVR | E1-S119 |
| | | | QTPEKRLEWVAYINSGGGSTYYPDTVKGRFTISRDNAK | |
| | | | NTLYLQMSRLKSEDTAMYYCARRGLPFHAMDYWGQG | |
| | | | TSVTVSS | |
| 34 | 12653 | Full | DIQMTQTTSSLSASLGDRVTISCSASQGISNYLNWYQQK | |
| | | | PDGTVKLLIYYTSILHSGVPSRFSGSGSGTDYSLTIGNLEP | |
| | | | EDIATYYCQQFNKLPPTFGGGTKLEIKRTVAAPSVFIFPPS | |
| | | | DEQLKSGTASVVCLLNNFYPREAKVQWKVDNALQSGN | |
| | | | SQESVTEQDSKDSTYSLSSTLTLSKADYEKHKVYACEVTH | |
| | | | QGLSSPVTKSFNRGEC | |
| 35 | 12653 | Full | GACATCCAGATGACCCAGACCACAAGCTCCCTGTCTG | |
| | | | CCAGCCTGGGCGATCGGGTGACAATCTCCTGCTCTGC | |
| | | | CAGCCAGGGCATCTCCAACTACCTGAATTGGTATCAG | |
| | | | CAGAAGCCAGACGGCACCGTGAAGCTGCTGATCTACT | |
| | | | ATACATCCATCCTGCACTCTGGCGTGCCCAGCAGATTC | |
| | | | TCCGGCTCTGGCAGCGGCACCGACTACTCTCTGACAA | |
| | | | TCGGCAACCTGGAGCCCGAGGATATCGCCACCTACTA | |
| | | | TTGTCAGCAGTTCAATAAGCTGCCCCCTACCTTTGGCG | |
| | | | GCGGCACAAAGCTGGAGATCAAGCGGACAGTGGCG | |
| | | | GCGCCCAGTGTCTTCATTTTTCCCCCTAGCGACGAACA | |
| | | | GCTGAAGTCTGGGACAGCCAGTGTGGTCTGTCTGCTG | |
| | | | AACAACTTCTACCCTAGAGAGGCTAAAGTGCAGTGGA | |
| | | | AGGTCGATAACGCACTGCAGTCCGGAAATTCTCAGGA | |
| | | | GAGTGTGACTGAACAGGACTCAAAAGATAGCACCTA | |
| | | | TTCCCTGTCAAGCACACTGACTCTGAGCAAGGCCGAC | |
| | | | TACGAGAAGCATAAAGTGTATGCTTGTGAAGTCACCC | |
| | | | ACCAGGGGCTGAGTTCACCAGTCACAAAATCATTCAA | |
| | | | CAGAGGGGAGTGC | |
| 36 | 12653 | VL | DIQMTQTTSSLSASLGDRVTISCSASQGISNYLNWYQQK | D1-K107 |
| | | | PDGTVKLLIYYTSILHSGVPSRFSGSGSGTDYSLTIGNLEP | |
| | | | EDIATYYCQQFNKLPPTFGGGTKLEIK | |
| 37 | 12654 | Full | DIQMTQSPSSLSASVGDRVTITCKASQDVSIGVAWYQQ | |
| | | | KPGKAPKLLIYSASYRYTGVPSRFSGSGSGTDFTLTISSLQ | |
| | | | PEDFATYYCQQYYIYPATFGQGTKVEIKVEGGSGGSGGS | |
| | | | GGSGGVDEVQLVESGGGLVQPGGSLRLSCAASGFTFAD | |
| | | | YTMDWVRQAPGKGLEWVGDVNPNSGGSIYNQRFKG | |
| | | | RFTFSVDRSKNTLYLQMNSLRAEDTAVYYCARNLGPSFY | |
| | | | FDYWGQGTLVTVSSAAEPKSSDKTHTCPPCPAPEAAGG | |
| | | | PSVFLFPPKPKDTLMISRTPEVTCVVVSVSHEDPEVKFN | |
| | | | WYVDGVEVHNAKTKPREEQYNSTYRVVSVLTVLHQDW | |
| | | | LNGKEYKCKVSNKALPAPIEKTISKAKGQPREPQVYVLPP | |
| | | | SRDELTKNQVSLLCLVKGFYPSDIAVEWESNGQPENNYL | |
| | | | TWPPVLDSDGSFFLYSKLTVDKSRWQQGNVFSCSVMH | |
| | | | EALHNHYTQKSLSLSPG | |
| 38 | 12654 | Full | GATATCCAGATGACACAGAGCCCAAGCTCCCTGTCTG | |
| | | | CCAGCGTGGGCGACAGAGTGACCATCACATGCAAGG | |
| | | | CCAGCCAGGACGTGAGCATCGGAGTGGCCTGGTACC | |
| | | | AGCAGAAGCCAGGCAAGGCCCCCAAGCTGCTGATCT | |
| | | | ATTCCGCCTCTTACAGGTATACCGGAGTGCCATCCCG | |
| | | | CTTCAGCGGCTCCGGCTCTGGAACAGACTTTACCCTG | |
| | | | ACAATCTCTAGCCTGCAGCCCGAGGATTTCGCCACCT | |
| | | | ACTATTGCCAGCAGTACTATATCTACCCTGCCACCTTT | |
| | | | GGCCAGGGCACAAAGGTGGAGATCAAGGTGGAGGG | |
| | | | AGGCTCCGGAGGCTCTGGAGGCAGCGGCGGCTCCGG | |
| | | | AGGAGTGGATGAGGTGCAGCTGGTGGAGAGCGGAG | |
| | | | GAGGCCTGGTGCAGCCTGGAGGCTCTCTGAGGCTGA | |
| | | | GCTGTGCAGCCTCCGGCTTCACCTTTGCCGACTACACA | |
| | | | ATGGATTGGGTGCGCCAGGCACCAGGCAAGGGCCTG | |
| | | | GAGTGGGTGGGCGACGTGAACCCTAATTCTGGCGGC | |
| | | | AGCATCTACAACCAGCGGTTCAAGGGCAGATTCACCT | |
| | | | TTTCTGTGGACAGGAGCAAGAACACACTGTATCTGCA | |
| | | | GATGAACAGCCTGAGGGCCGAGGATACCGCCGTGTA | |
| | | | CTATTGCGCCCGCAATCTGGGCCCAAGCTTCTACTTTG | |
| | | | ACTATTGGGGCCAGGGCACCCTGGTGACAGTGTCCTC | |
| | | | TGCCGCCGAGCCCAAGAGCTCCGATAAGACCCACACA | |
| | | | TGCCCACCTTGTCCGGCGCCAGAGGCCGCCGGAGGA | |
| | | | CCTAGCGTGTTCCTGTTTCCACCCAAGCCAAAGGACA | |
| | | | CCCTGATGATCAGCCGCACCCCTGAGGTGACATGCGT | |
| | | | GGTGGTGAGCGTGTCCCACGAGGACCCAGAGGTGAA | |
| | | | GTTTAACTGGTACGTGGATGGCGTGGAGGTGCACAA | |
| | | | TGCCAAGACAAAGCCCAGAGAGGAGCAGTACAACTC | |
| | | | CACCTATAGAGTGGTGTCTGTGCTGACAGTGCTGCAC | |
| | | | CAGGATTGGCTGAACGGCAAGGAGTATAAGTGCAAG | |
| | | | GTGAGCAATAAGGCCCTGCCTGCCCCAATCGAGAAG | |
| | | | ACCATCTCCAAGGCCAAGGGCCAGCCTCGCGAACCTC | |
| | | | AGGTGTACGTGCTGCCTCCATCCAGAGATGAGCTGAC | |
| | | | AAAGAACCAGGTGTCTCTGCTGTGCCTGGTGAAGGG | |
| | | | CTTCTATCCATCTGACATCGCCGTGGAGTGGGAGAGC | |
| | | | AATGGCCAGCCCGAGAACAATTACCTGACCTGGCCCC | |
| | | | CTGTGCTGGACTCCGATGGCTCTTTCTTTCTGTATAGC | |
| | | | AAGCTGACAGTGGACAAGTCCCGGTGGCAGCAGGGC | |
| | | | AACGTGTTTTCTTGTAGCGTGATGCACGAGGCCCTGC | |
| | | | ACAATCACTACACCCAGAAGTCCCTGAGCTTAAGCCC | |
| | | | CGGC | |
| 39 | 12654 | VL | DIQMTQSPSSLSASVGDRVTITCKASQDVSIGVAWYQQ | D1-K107 |
| | | | KPGKAPKLLIYSASYRYTGVPSRFSGSGSGTDFTLTISSLQ | |
| | | | PEDFATYYCQQYYIYPATFGQGTKVEIK | |
| 40 | 12655 | Full | ELVLTQSPSVSAALGSPAKITCTLSSAHKTDTIDWYQQLQ | |
| | | | GEAPRYLMQVQSDGSYTKRPGVPDRFSGSSSGADRYLII | |
| | | | PSVQADDEADYYCGADYIGGYVFGGGTQLTVTVEGGS | |
| | | | GGSGGSGGSGGVDQEQLVESGGRLVTPGGSLTLSCKAS | |
| | | | GFDFSAYYMSWVRQAPGKGLEWIATIYPSSGKTYYATW | |
| | | | VNGRFTISSDNAQNTVDLQMNSLTAADRATYFCARDSY | |
| | | | ADDGALFNIWGPGTLVTISSAAEPKSSDKTHTCPPCPAP | |
| | | | EAAGGPSVFLFPPKPKDTLMISRTPEVTCVVVSVSHEDP | |
| | | | EVKFNWYVDGVEVHNAKTKPREEQYNSTYRVVSVLTVL | |
| | | | HQDWLNGKEYKCKVSNKALPAPIEKTISKAKGQPREPQ | |
| | | | VYVLPPSRDELTKNQVSLLCLVKGFYPSDIAVEWESNGQ | |
| | | | PENNYLTWPPVLDSDGSFFLYSKLTVDKSRWQQGNVFS | |
| | | | CSVMHEALHNHYTQKSLSLSPG | |
| 41 | 12655 | Full | GAGCTGGTGCTGACACAGTCCCCTTCTGTGAGCGCCG | |
| | | | CCCTGGGCTCCCCAGCCAAGATCACCTGCACACTGAG | |
| | | | CTCCGCCCACAAGACCGACACAATCGATTGGTACCAG | |
| | | | CAGCTGCAGGGAGAGGCACCCAGATATCTGATGCAG | |
| | | | GTGCAGTCTGACGGCAGCTACACCAAGCGGCCCGGA | |
| | | | GTGCCTGACAGATTCTCCGGCTCTAGCTCCGGAGCCG | |
| | | | ATCGCTATCTGATCATCCCATCTGTGCAGGCCGACGA | |
| | | | TGAGGCCGACTACTATTGCGGAGCCGATTACATCGGA | |
| | | | GGATACGTGTTCGGAGGAGGAACCCAGCTGACCGTG | |
| | | | ACAGTGGAGGGAGGCTCCGGAGGCTCTGGAGGCAG | |
| | | | CGGCGGCTCCGGCGGCGTGGACCAGGAGCAGCTGGT | |
| | | | GGAGAGCGGCGGCAGACTGGTGACCCCAGGAGGCT | |
| | | | CCCTGACACTGTCTTGTAAGGCCAGCGGCTTCGATTTT | |
| | | | TCCGCCTACTATATGTCTTGGGTGAGACAGGCACCAG | |
| | | | GCAAGGGCCTGGAGTGGATCGCCACCATCTACCCCTC | |
| | | | TAGCGGCAAGACCTACTATGCCACATGGGTGAACGG | |
| | | | CAGATTCACCATCTCCTCTGACAACGCCCAGAATACA | |
| | | | GTGGATCTGCAGATGAATAGCCTGACCGCCGCCGAC | |
| | | | AGGGCCACATACTTCTGCGCCCGCGATTCCTATGCCG | |
| | | | ACGATGGGGCCCTGTTCAACATCTGGGGCCCTGGCAC | |
| | | | CCTGGTGACAATCAGCTCCGCCGCCGAGCCAAAGTCT | |
| | | | AGCGACAAGACCCACACATGCCCACCTTGTCCGGCGC | |
| | | | CAGAGGCCGCCGGAGGACCAAGCGTGTTCCTGTTTCC | |
| | | | ACCCAAGCCTAAGGATACCCTGATGATCTCCAGAACC | |
| | | | CCAGAGGTGACATGCGTGGTGGTGTCCGTGTCTCACG | |
| | | | AGGACCCCGAGGTGAAGTTTAACTGGTATGTGGATG | |
| | | | GCGTGGAGGTGCACAATGCCAAGACAAAGCCCAGAG | |
| | | | AGGAGCAGTACAATAGCACCTATAGAGTGGTGTCCG | |
| | | | TGCTGACAGTGCTGCACCAGGACTGGCTGAACGGCA | |
| | | | AGGAGTACAAGTGCAAGGTGTCTAATAAGGCCCTGC | |
| | | | CTGCCCCAATCGAGAAGACCATCAGCAAGGCAAAGG | |
| | | | GACAGCCTCGCGAACCACAGGTGTATGTGCTGCCTCC | |
| | | | AAGCCGCGACGAGCTGACAAAGAACCAGGTGTCCCT | |
| | | | GCTGTGCCTGGTGAAGGGCTTCTACCCCTCCGATATC | |
| | | | GCCGTGGAGTGGGAGTCTAATGGCCAGCCTGAGAAC | |
| | | | AATTATCTGACCTGGCCCCCTGTGCTGGACTCTGATG | |
| | | | GCAGCTTCTTTCTGTACTCTAAGCTGACAGTGGATAA | |
| | | | GAGCCGGTGGCAGCAGGGCAACGTGTTTAGCTGTTC | |
| | | | CGTGATGCACGAGGCCCTGCACAATCACTACACCCAG | |
| | | | AAGTCTCTGAGCTTAAGCCCTGGC | |
| 42 | 12655 | VL | ELVLTQSPSVSAALGSPAKITCTLSSAHKTDTIDWYQQLQ | E1-T111 |
| | | | GEAPRYLMQVQSDGSYTKRPGVPDRFSGSSSGADRYLII | |
| | | | PSVQADDEADYYCGADYIGGYVFGGGTQLTVT | |
| 43 | 12655 | VH | QEQLVESGGRLVTPGGSLTLSCKASGFDFSAYYMSWVR | Q130- |
| | | | QAPGKGLEWIATIYPSSGKTYYATWVNGRFTISSDNAQ | S250 |
| | | | NTVDLQMNSLTAADRATYFCARDSYADDGALFNIWGP | |
| | | | GTLVTISS | |
| 44 | 12657 | Full | EVQLVESGGGLVQPGGSLRLSCAASGFTFADYTMDWV | |
| | | | RQAPGKGLEWVGDVNPNSGGSIYNQRFKGRFTFSVDR | |
| | | | SKNTLYLQMNSLRAEDTAVYYCARNLGPSFYFDYWGQ | |
| | | | GTLVTVSSASTKGPSVFPLAPSSKSTSGGTAALGCLVKDY | |
| | | | FPEPVTVSWNSGALTSGVHTFPAVLQSSGLYSLSSVVTV | |
| | | | PSSSLGTQTYICNVNHKPSNTKVDKKVEPKSCDKTHTCP | |
| | | | PCPAPEAAGGPSVFLFPPKPKDTLMISRTPEVTCVVVSV | |
| | | | SHEDPEVKFNWYVDGVEVHNAKTKPREEQYNSTYRVV | |
| | | | SVLTVLHQDWLNGKEYKCKVSNKALPAPIEKTISKAKGQ | |
| | | | PREPQVYVYPPSRDELTKNQVSLTCLVKGFYPSDIAVEW | |
| | | | ESNGQPENNYKTTPPVLDSDGSFALVSKLTVDKSRWQQ | |
| | | | GNVFSCSVMHEALHNHYTQKSLSLSPG | |
| 45 | 12657 | Full | GAGGTGCAGCTGGTGGAATCAGGAGGGGGCCTGGT | |
| | | | GCAGCCCGGAGGGTCTCTGCGACTGTCATGTGCCGCT | |
| | | | TCTGGGTTCACTTTCGCAGACTACACAATGGATTGGG | |
| | | | TGCGACAGGCCCCCGGAAAGGGACTGGAGTGGGTG | |
| | | | GGCGATGTCAACCCTAATTCTGGCGGGAGTATCTACA | |
| | | | ACCAGCGGTTCAAGGGGAGATTCACTTTTTCAGTGGA | |
| | | | CAGAAGCAAAAACACCCTGTATCTGCAGATGAACAGC | |
| | | | CTGAGGGCCGAAGATACCGCTGTCTACTATTGCGCTC | |
| | | | GCAATCTGGGCCCCAGTTTCTACTTTGACTATTGGGG | |
| | | | GCAGGGAACCCTGGTGACAGTCAGCTCCGCTAGCACT | |
| | | | AAGGGGCCTTCCGTGTTTCCACTGGCTCCCTCTAGTAA | |
| | | | ATCCACCTCTGGAGGCACAGCTGCACTGGGATGTCTG | |
| | | | GTGAAGGATTACTTCCCTGAACCAGTCACAGTGAGTT | |
| | | | GGAACTCAGGGGCTCTGACAAGTGGAGTCCATACTTT | |
| | | | TCCCGCAGTGCTGCAGTCAAGCGGACTGTACTCCCTG | |
| | | | TCCTCTGTGGTCACCGTGCCTAGTTCAAGCCTGGGCA | |
| | | | CCCAGACATATATCTGCAACGTGAATCACAAGCCATC | |
| | | | AAATACAAAAGTCGACAAGAAAGTGGAGCCCAAGAG | |
| | | | CTGTGATAAAACTCATACCTGCCCACCTTGTCCGGCGC | |
| | | | CAGAGGCAGCAGGAGGACCAAGCGTGTTCCTGTTTC | |
| | | | CACCCAAGCCCAAAGACACCCTGATGATTAGCCGAAC | |
| | | | CCCTGAAGTCACATGCGTGGTCGTGTCCGTGTCTCAC | |
| | | | GAGGACCCAGAAGTCAAGTTCAACTGGTACGTGGAT | |
| | | | GGCGTCGAGGTGCATAATGCCAAGACAAAACCCCGG | |
| | | | GAGGAACAGTACAACAGCACCTATAGAGTCGTGTCC | |
| | | | GTCCTGACAGTGCTGCACCAGGATTGGCTGAACGGC | |
| | | | AAGGAATATAAGTGCAAAGTGTCCAATAAGGCCCTG | |
| | | | CCCGCTCCTATCGAGAAAACCATTTCTAAGGCAAAAG | |
| | | | GCCAGCCTCGCGAACCACAGGTCTACGTCTACCCCCC | |
| | | | ATCAAGAGATGAACTGACAAAAAATCAGGTCTCTCTG | |
| | | | ACATGCCTGGTCAAAGGATTCTACCCTTCCGACATCG | |
| | | | CCGTGGAGTGGGAAAGTAACGGCCAGCCCGAGAACA | |
| | | | ATTACAAGACCACACCCCCTGTCCTGGACTCTGATGG | |
| | | | GAGTTTCGCTCTGGTGTCAAAGCTGACCGTCGATAAA | |
| | | | AGCCGGTGGCAGCAGGGCAATGTGTTTAGCTGCTCC | |
| | | | GTCATGCACGAAGCCCTGCACAATCACTACACACAGA | |
| | | | AGTCCCTGAGCCTGAGCCCTGGC | |
| 46 | 12657 | VH | EVQLVESGGGLVQPGGSLRLSCAASGFTFADYTMDWV | E1-S119 |
| | | | RQAPGKGLEWVGDVNPNSGGSIYNQRFKGRFTFSVDR | |
| | | | SKNTLYLQMNSLRAEDTAVYYCARNLGPSFYFDYWGQ | |
| | | | GTLVTVSS | |
| 47 | 12658 | Full | DIQMTQSPSSLSASVGDRVTITCKASQDVSIGVAWYQQ | |
| | | | KPGKAPKLLIYSASYRYTGVPSRFSGSGSGTDFTLTISSLQ | |
| | | | PEDFATYYCQQYYIYPATFGQGTKVEIKRTVAAPSVFIFP | |
| | | | PSDEQLKSGTASVVCLLNNFYPREAKVQWKVDNALQS | |
| | | | GNSQESVTEQDSKDSTYSLSSTLTLSKADYEKHKVYACEV | |
| | | | THQGLSSPVTKSFNRGEC | |
| 48 | 12658 | Full | GACATCCAGATGACCCAGTCCCCTAGCTCCCTGTCCG | |
| | | | CCTCTGTGGGCGACAGGGTGACCATCACATGCAAGG | |
| | | | CCTCTCAGGATGTGAGCATCGGAGTGGCATGGTACCA | |
| | | | GCAGAAGCCAGGCAAGGCCCCTAAGCTGCTGATCTAT | |
| | | | AGCGCCTCCTACCGGTATACCGGCGTGCCCTCTAGAT | |
| | | | TCTCTGGCAGCGGCTCCGGCACAGACTTTACCCTGAC | |
| | | | AATCTCTAGCCTGCAGCCAGAGGATTTCGCCACCTAC | |
| | | | TATTGTCAGCAGTACTATATCTACCCCGCCACCTTTGG | |
| | | | CCAGGGCACAAAGGTGGAGATCAAGCGGACAGTGG | |
| | | | CGGCGCCCAGTGTCTTCATTTTTCCCCCTAGCGACGAA | |
| | | | CAGCTGAAGTCTGGGACAGCCAGTGTGGTCTGTCTGC | |
| | | | TGAACAACTTCTACCCTAGAGAGGCTAAAGTGCAGTG | |
| | | | GAAGGTCGATAACGCACTGCAGTCCGGAAATTCTCAG | |
| | | | GAGAGTGTGACTGAACAGGACTCAAAAGATAGCACC | |
| | | | TATTCCCTGTCAAGCACACTGACTCTGAGCAAGGCCG | |
| | | | ACTACGAGAAGCATAAAGTGTATGCTTGTGAAGTCAC | |
| | | | CCACCAGGGGCTGAGTTCACCAGTCACAAAATCATTC | |
| | | | AACAGAGGGGAGTGC | |
| 49 | 12658 | VL | DIQMTQSPSSLSASVGDRVTITCKASQDVSIGVAWYQQ | D1-K107 |
| | | | KPGKAPKLLIYSASYRYTGVPSRFSGSGSGTDFTLTISSLQ | |
| | | | PEDFATYYCQQYYIYPATFGQGTKVEIK | |
| 50 | 12659 | Full | QEQLVESGGRLVTPGGSLTLSCKASGFDFSAYYMSWVR | |
| | | | QAPGKGLEWIATIYPSSGKTYYATWVNGRFTISSDNAQ | |
| | | | NTVDLQMNSLTAADRATYFCARDSYADDGALFNIWGP | |
| | | | GTLVTISSASTKGPSVFPLAPSSKSTSGGTAALGCLVKDYF | |
| | | | PEPVTVSWNSGALTSGVHTFPAVLQSSGLYSLSSVVTVP | |
| | | | SSSLGTQTYICNVNHKPSNTKVDKKVEPKSCDKTHTCPP | |
| | | | CPAPEAAGGPSVFLFPPKPKDTLMISRTPEVTCVVVSVS | |
| | | | HEDPEVKFNWYVDGVEVHNAKTKPREEQYNSTYRVVS | |
| | | | VLTVLHQDWLNGKEYKCKVSNKALPAPIEKTISKAKGQP | |
| | | | REPQVYVYPPSRDELTKNQVSLTCLVKGFYPSDIAVEWE | |
| | | | SNGQPENNYKTTPPVLDSDGSFALVSKLTVDKSRWQQ | |
| | | | GNVFSCSVMHEALHNHYTQKSLSLSPG | |
| 51 | 12659 | Full | CAGGAGCAGCTGGTGGAGTCCGGCGGCAGGCTGGT | |
| | | | GACCCCAGGAGGCAGCCTGACACTGTCCTGCAAGGC | |
| | | | CTCTGGCTTCGACTTTAGCGCCTACTATATGTCCTGGG | |
| | | | TGCGCCAGGCCCCCGGCAAGGGCCTGGAGTGGATCG | |
| | | | CCACCATCTACCCTAGCTCCGGCAAGACCTACTATGCC | |
| | | | ACATGGGTGAACGGCAGATTCACCATCTCTAGCGACA | |
| | | | ACGCCCAGAATACAGTGGATCTGCAGATGAACAGCCT | |
| | | | GACCGCCGCCGACAGGGCAACATACTTCTGTGCCAGA | |
| | | | GATAGCTATGCCGACGATGGGGCCCTGTTCAACATCT | |
| | | | GGGGACCAGGCACCCTGGTGACAATCTCCTCTGCTAG | |
| | | | CACTAAGGGGCCTTCCGTGTTTCCACTGGCTCCCTCTA | |
| | | | GTAAATCCACCTCTGGAGGCACAGCTGCACTGGGATG | |
| | | | TCTGGTGAAGGATTACTTCCCTGAACCAGTCACAGTG | |
| | | | AGTTGGAACTCAGGGGCTCTGACAAGTGGAGTCCAT | |
| | | | ACTTTTCCCGCAGTGCTGCAGTCAAGCGGACTGTACT | |
| | | | CCCTGTCCTCTGTGGTCACCGTGCCTAGTTCAAGCCTG | |
| | | | GGCACCCAGACATATATCTGCAACGTGAATCACAAGC | |
| | | | CATCAAATACAAAAGTCGACAAGAAAGTGGAGCCCA | |
| | | | AGAGCTGTGATAAAACTCATACCTGCCCACCTTGTCC | |
| | | | GGCGCCAGAGGCTGCAGGAGGACCAAGCGTGTTCCT | |
| | | | GTTTCCACCCAAGCCTAAAGACACACTGATGATTTCCC | |
| | | | GAACCCCCGAAGTCACATGCGTGGTCGTGTCTGTGAG | |
| | | | TCACGAGGACCCTGAAGTCAAGTTCAACTGGTACGTG | |
| | | | GATGGCGTCGAGGTGCATAATGCCAAGACTAAACCT | |
| | | | AGGGAGGAACAGTACAACTCAACCTATCGCGTCGTG | |
| | | | AGCGTCCTGACAGTGCTGCACCAGGATTGGCTGAAC | |
| | | | GGCAAAGAATATAAGTGCAAAGTGAGCAATAAGGCC | |
| | | | CTGCCCGCTCCTATCGAGAAAACCATTTCCAAGGCTA | |
| | | | AAGGGCAGCCTCGCGAACCACAGGTCTACGTGTATCC | |
| | | | TCCAAGCCGGGACGAGCTGACAAAGAACCAGGTCTC | |
| | | | CCTGACTTGTCTGGTGAAAGGGTTTTACCCTAGTGAT | |
| | | | ATCGCTGTGGAGTGGGAATCAAATGGACAGCCAGAG | |
| | | | AACAATTATAAGACTACCCCCCCTGTGCTGGACAGTG | |
| | | | ATGGGTCATTCGCACTGGTCTCCAAGCTGACAGTGGA | |
| | | | CAAATCTCGGTGGCAGCAGGGAAATGTCTTTTCATGT | |
| | | | AGCGTGATGCATGAAGCACTGCACAACCATTACACCC | |
| | | | AGAAGTCACTGTCACTGTCACCAGGA | |
| 52 | 12659 | VH | QEQLVESGGRLVTPGGSLTLSCKASGFDFSAYYMSWVR | Q1-S121 |
| | | | QAPGKGLEWIATIYPSSGKTYYATWVNGRFTISSDNAQ | |
| | | | NTVDLQMNSLTAADRATYFCARDSYADDGALFNIWGP | |
| | | | GTLVTISS | |
| 53 | 12660 | Full | ELVLTQSPSVSAALGSPAKITCTLSSAHKTDTIDWYQQLQ | |
| | | | GEAPRYLMQVQSDGSYTKRPGVPDRFSGSSSGADRYLII | |
| | | | PSVQADDEADYYCGADYIGGYVFGGGTQLTVTRTVAAP | |
| | | | SVFIFPPSDEQLKSGTASVVCLLNNFYPREAKVQWKVDN | |
| | | | ALQSGNSQESVTEQDSKDSTYSLSSTLTLSKADYEKHKVY | |
| | | | ACEVTHQGLSSPVTKSFNRGEC | |
| 54 | 12660 | Full | GAGCTGGTGCTGACACAGTCTCCAAGCGTGTCCGCCG | |
| | | | CCCTGGGCAGCCCCGCCAAGATCACCTGCACACTGAG | |
| | | | CTCCGCCCACAAGACCGACACAATCGATTGGTACCAG | |
| | | | CAGCTGCAGGGAGAGGCCCCCCGGTATCTGATGCAG | |
| | | | GTGCAGTCTGACGGCAGCTACACAAAGCGGCCCGGA | |
| | | | GTGCCTGACAGATTCTCCGGCTCTAGCTCCGGAGCCG | |
| | | | ATCGCTATCTGATCATCCCCTCTGTGCAGGCCGACGAT | |
| | | | GAGGCCGACTACTATTGTGGAGCCGATTACATCGGA | |
| | | | GGATACGTGTTCGGAGGAGGAACCCAGCTGACCGTG | |
| | | | ACACGGACCGTGGCGGCGCCCAGTGTCTTCATTTTTC | |
| | | | CCCCTAGCGACGAACAGCTGAAGTCTGGGACAGCCA | |
| | | | GTGTGGTCTGTCTGCTGAACAACTTCTACCCTAGAGA | |
| | | | GGCTAAAGTGCAGTGGAAGGTCGATAACGCACTGCA | |
| | | | GTCCGGAAATTCTCAGGAGAGTGTGACTGAACAGGA | |
| | | | CTCAAAAGATAGCACCTATTCCCTGTCAAGCACACTG | |
| | | | ACTCTGAGCAAGGCCGACTACGAGAAGCATAAAGTG | |
| | | | TATGCTTGTGAAGTCACCCACCAGGGGCTGAGTTCAC | |
| | | | CAGTCACAAAATCATTCAACAGAGGGGAGTGC | |
| 55 | 12660 | VL | ELVLTQSPSVSAALGSPAKITCTLSSAHKTDTIDWYQQLQ | E1-T111 |
| | | | GEAPRYLMQVQSDGSYTKRPGVPDRFSGSSSGADRYLII | |
| | | | PSVQADDEADYYCGADYIGGYVFGGGTQLTVT | |
| 56 | 12667 | Full | EPAVYFKEQFLDGDGWTSRWIESKHKSDFGKFVLSSGK | |
| | | | FYGDEEKDKGLQTSQDARFYALSASFEPFSNKGQTLVV | |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAAAEPKSSDKTHTCPPCPAPEA | |
| | | | AGGPSVFLFPPKPKDTLMISRTPEVTCVVVSVSHEDPEV | |
| | | | KFNWYVDGVEVHNAKTKPREEQYNSTYRVVSVLTVLH | |
| | | | QDWLNGKEYKCKVSNKALPAPIEKTISKAKGQPREPQV | |
| | | | YVLPPSRDELTKNQVSLLCLVKGFYPSDIAVEWESNGQP | |
| | | | ENNYLTWPPVLDSDGSFFLYSKLTVDKSRWQQGNVFSC | |
| | | | SVMHEALHNHYTQKSLSLSPG | |
| 57 | 12667 | Full | GAGCCTGCCGTGTATTTCAAGGAGCAGTTTCTGGACG | |
| | | | GCGATGGCTGGACAAGCAGATGGATCGAGTCTAAGC | |
| | | | ACAAGAGCGACTTCGGCAAGTTTGTGCTGAGCTCCGG | |
| | | | CAAGTTCTATGGCGATGAGGAGAAGGACAAGGGCCT | |
| | | | GCAGACCTCTCAGGATGCCAGGTTTTACGCCCTGTCC | |
| | | | GCCTCTTTCGAGCCCTTCAGCAACAAGGGCCAGACCC | |
| | | | TGGTGGTGCAGTTCACAGTGAAGCACGAGCAGAACA | |
| | | | TCGACTGCGGCGGCGGCTATGTGAAGCTGTTTCCCAA | |
| | | | TAGCCTGGATCAGACCGACATGCACGGCGACTCCGA | |
| | | | GTACAACATCATGTTCGGCCCTGATATCTGCGGCCCA | |
| | | | GGCACAAAGAAGGTGCACGTGATCTTTAATTACAAG | |
| | | | GGCAAGAACGTGCTGATCAATAAGGACATCAGGTGT | |
| | | | AAGGACGATGAGTTCACCCACCTGTACACACTGATCG | |
| | | | TGCGCCCTGACAACACATATGAGGTGAAGATCGATAA | |
| | | | TTCCCAGGTGGAGAGCGGCTCCCTGGAGGACGATTG | |
| | | | GGATTTTCTGCCCCCTAAGAAGATCAAGGACCCCGAT | |
| | | | GCCTCCAAGCCTGAGGACTGGGATGAGCGCGCCAAG | |
| | | | ATCGACGATCCAACCGACTCTAAGCCCGAGGACTGG | |
| | | | GATAAGCCCGAGCACATCCCCGACCCTGATGCCAAGA | |
| | | | AGCCAGAAGACTGGGATGAGGAGATGGATGGCGAG | |
| | | | TGGGAGCCACCCGTGATCCAGAACCCAGAGTACAAG | |
| | | | GGCGAGTGGAAGCCCAGACAGATCGATAATCCTGAC | |
| | | | TATAAGGGCACCTGGATTCACCCTGAGATCGATAACC | |
| | | | CAGAGTACTCCCCAGACCCCTCTATCTACGCCTATGAT | |
| | | | AATTTCGGCGTGCTGGGCCTGGACCTGTGGCAGGTG | |
| | | | AAGAGCGGCACCATCTTCGACAACTTTCTGATCACAA | |
| | | | ATGATGAGGCCTACGCCGAGGAGTTTGGCAACGAGA | |
| | | | CATGGGGCGTGACAAAGGCCGCCGAGAAGCAGATG | |
| | | | AAGGATAAGCAGGACGAGGAGCAGAGGCTGAAGGA | |
| | | | AGAGGAGGAGGACAAGAAGCGCAAGGAGGAGGAG | |
| | | | GAGGCCGAGGATAAGGAGGACGATGAGGACAAGGA | |
| | | | TGAGGACGAGGAGGATGAGGAGGACAAGGAGGAG | |
| | | | GATGAGGAGGAGGACGTGCCAGGACAGGCCGCCGC | |
| | | | CGAGCCCAAGTCTAGCGACAAGACCCACACATGCCCT | |
| | | | CCATGTCCGGCGCCGGAGGCCGCCGGAGGACCTAGC | |
| | | | GTGTTCCTGTTTCCCCCTAAGCCAAAGGATACACTGAT | |
| | | | GATCTCCAGAACCCCTGAGGTGACATGCGTGGTGGT | |
| | | | GTCTGTGAGCCACGAGGACCCAGAGGTGAAGTTCAA | |
| | | | CTGGTATGTGGATGGCGTGGAGGTGCACAATGCCAA | |
| | | | GACCAAGCCCCGGGAGGAGCAGTACAATAGCACCTA | |
| | | | TAGAGTGGTGTCCGTGCTGACAGTGCTGCACCAGGA | |
| | | | CTGGCTGAACGGCAAGGAGTACAAGTGCAAGGTGTC | |
| | | | CAATAAGGCCCTGCCGGCACCTATCGAGAAGACCATC | |
| | | | TCTAAGGCAAAGGGACAGCCACGGGAGCCACAGGTG | |
| | | | TATGTGCTGCCACCCTCTAGAGACGAGCTGACAAAGA | |
| | | | ACCAGGTGAGCCTGCTGTGCCTGGTGAAGGGCTTCTA | |
| | | | CCCATCCGATATCGCCGTGGAGTGGGAGTCTAATGGC | |
| | | | CAGCCCGAGAACAATTATCTGACCTGGCCTCCAGTGC | |
| | | | TGGATAGCGACGGCTCCTTCTTTCTGTACTCTAAGCTG | |
| | | | ACAGTGGACAAGAGCCGGTGGCAGCAGGGCAACGT | |
| | | | GTTTTCCTGTTCTGTGATGCACGAGGCCCTGCACAATC | |
| | | | ACTACACCCAGAAGAGCCTGTCCCTGTCTCCTGGC | |
| 58 | 12667 | Calreticulin | EPAVYFKEQFLDGDGWTSRWIESKHKSDFGKFVLSSGK | E1-A396 |
| | | | FYGDEEKDKGLQTSQDARFYALSASFEPFSNKGQTLVV | |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQA | |
| 59 | 12667 | Calreticulin | GGCGAGCCTGCCGTGTATTTCAAGGAGCAGTTTCTGG | |
| | | | ACGGCGATGGCTGGACAAGCAGATGGATCGAGTCTA | |
| | | | AGCACAAGAGCGACTTCGGCAAGTTTGTGCTGAGCTC | |
| | | | CGGCAAGTTCTATGGCGATGAGGAGAAGGACAAGG | |
| | | | GCCTGCAGACCTCTCAGGATGCCAGGTTTTACGCCCT | |
| | | | GTCCGCCTCTTTCGAGCCCTTCAGCAACAAGGGCCAG | |
| | | | ACCCTGGTGGTGCAGTTCACAGTGAAGCACGAGCAG | |
| | | | AACATCGACTGCGGCGGCGGCTATGTGAAGCTGTTTC | |
| | | | CCAATAGCCTGGATCAGACCGACATGCACGGCGACTC | |
| | | | CGAGTACAACATCATGTTCGGCCCTGATATCTGCGGC | |
| | | | CCAGGCACAAAGAAGGTGCACGTGATCTTTAATTACA | |
| | | | AGGGCAAGAACGTGCTGATCAATAAGGACATCAGGT | |
| | | | GTAAGGACGATGAGTTCACCCACCTGTACACACTGAT | |
| | | | CGTGCGCCCTGACAACACATATGAGGTGAAGATCGAT | |
| | | | AATTCCCAGGTGGAGAGCGGCTCCCTGGAGGACGAT | |
| | | | TGGGATTTTCTGCCCCCTAAGAAGATCAAGGACCCCG | |
| | | | ATGCCTCCAAGCCTGAGGACTGGGATGAGCGCGCCA | |
| | | | AGATCGACGATCCAACCGACTCTAAGCCCGAGGACTG | |
| | | | GGATAAGCCCGAGCACATCCCCGACCCTGATGCCAAG | |
| | | | AAGCCAGAAGACTGGGATGAGGAGATGGATGGCGA | |
| | | | GTGGGAGCCACCCGTGATCCAGAACCCAGAGTACAA | |
| | | | GGGCGAGTGGAAGCCCAGACAGATCGATAATCCTGA | |
| | | | CTATAAGGGCACCTGGATTCACCCTGAGATCGATAAC | |
| | | | CCAGAGTACTCCCCAGACCCCTCTATCTACGCCTATGA | |
| | | | TAATTTCGGCGTGCTGGGCCTGGACCTGTGGCAGGT | |
| | | | GAAGAGCGGCACCATCTTCGACAACTTTCTGATCACA | |
| | | | AATGATGAGGCCTACGCCGAGGAGTTTGGCAACGAG | |
| | | | ACATGGGGCGTGACAAAGGCCGCCGAGAAGCAGAT | |
| | | | GAAGGATAAGCAGGACGAGGAGCAGAGGCTGAAGG | |
| | | | AAGAGGAGGAGGACAAGAAGCGCAAGGAGGAGGA | |
| | | | GGAGGCCGAGGATAAGGAGGACGATGAGGACAAGG | |
| | | | ATGAGGACGAGGAGGATGAGGAGGACAAGGAGGA | |
| | | | GGATGAGGAGGAGGACGTGCCAGGACAGGCC | |
| 60 | 12650 | Full | QVQLVESGGGVVQPGRSLRLSCAASGFTFSNYGMYWV | |
| | | | RQAPGKGLEWVAVIWYDGSNKYYADSVKGRFTISRDN | |
| | | | SKNTLYLQMNSLRAEDTAVYYCARDLWGWYFDYWGQ | |
| | | | GTLVTVSSASTKGPSVFPLAPSSKSTSGGTAALGCLVKDY | |
| | | | FPEPVTVSWNSGALTSGVHTFPAVLQSSGLYSLSSVVTV | |
| | | | PSSSLGTQTYICNVNHKPSNTKVDKKVEPKSCDKTHTCP | |
| | | | PCPAPEAAGGPSVFLFPPKPKDTLMISRTPEVTCVVVSV | |
| | | | SHEDPEVKFNWYVDGVEVHNAKTKPREEQYNSTYRVV | |
| | | | SVLTVLHQDWLNGKEYKCKVSNKALPAPIEKTISKAKGQ | |
| | | | PREPQVYVYPPSRDELTKNQVSLTCLVKGFYPSDIAVEW | |
| | | | ESNGQPENNYKTTPPVLDSDGSFALVSKLTVDKSRWQQ | |
| | | | GNVFSCSVMHEALHNHYTQKSLSLSPG | |
| 61 | 12650 | Full | CAGGTGCAGCTGGTGGAGAGCGGAGGAGGAGTGGT | |
| | | | GCAGCCCGGCAGAAGCCTGCGGCTGAGCTGCGCAGC | |
| | | | CTCCGGCTTCACCTTTTCCAACTACGGCATGTATTGGG | |
| | | | TGCGGCAGGCCCCTGGCAAGGGCCTGGAGTGGGTGG | |
| | | | CCGTGATCTGGTACGACGGCTCCAATAAGTACTATGC | |
| | | | CGATTCTGTGAAGGGCAGGTTCACCATCAGCCGGGA | |
| | | | CAACAGCAAGAATACACTGTATCTGCAGATGAACTCT | |
| | | | CTGCGGGCCGAGGATACAGCCGTGTACTATTGTGCCA | |
| | | | GGGACCTGTGGGGCTGGTACTTTGATTATTGGGGCC | |
| | | | AGGGCACCCTGGTGACAGTGAGCTCCGCTAGCACTA | |
| | | | AGGGGCCTTCCGTGTTTCCACTGGCTCCCTCTAGTAAA | |
| | | | TCCACCTCTGGAGGCACAGCTGCACTGGGATGTCTGG | |
| | | | TGAAGGATTACTTCCCTGAACCAGTCACAGTGAGTTG | |
| | | | GAACTCAGGGGCTCTGACAAGTGGAGTCCATACTTTT | |
| | | | CCCGCAGTGCTGCAGTCAAGCGGACTGTACTCCCTGT | |
| | | | CCTCTGTGGTCACCGTGCCTAGTTCAAGCCTGGGCAC | |
| | | | CCAGACATATATCTGCAACGTGAATCACAAGCCATCA | |
| | | | AATACAAAAGTCGACAAGAAAGTGGAGCCCAAGAGC | |
| | | | TGTGATAAAACTCATACCTGCCCACCTTGTCCGGCGCC | |
| | | | AGAGGCTGCAGGAGGACCAAGCGTGTTCCTGTTTCCA | |
| | | | CCCAAGCCTAAAGACACACTGATGATTTCCCGAACCC | |
| | | | CCGAAGTCACATGCGTGGTCGTGTCTGTGAGTCACGA | |
| | | | GGACCCTGAAGTCAAGTTCAACTGGTACGTGGATGG | |
| | | | CGTCGAGGTGCATAATGCCAAGACTAAACCTAGGGA | |
| | | | GGAACAGTACAACTCAACCTATCGCGTCGTGAGCGTC | |
| | | | CTGACAGTGCTGCACCAGGATTGGCTGAACGGCAAA | |
| | | | GAATATAAGTGCAAAGTGAGCAATAAGGCCCTGCCC | |
| | | | GCTCCTATCGAGAAAACCATTTCCAAGGCTAAAGGGC | |
| | | | AGCCTCGCGAACCACAGGTCTACGTGTATCCTCCAAG | |
| | | | CCGGGACGAGCTGACAAAGAACCAGGTCTCCCTGAC | |
| | | | TTGTCTGGTGAAAGGGTTTTACCCTAGTGATATCGCT | |
| | | | GTGGAGTGGGAATCAAATGGACAGCCAGAGAACAAT | |
| | | | TATAAGACTACCCCCCCTGTGCTGGACAGTGATGGGT | |
| | | | CATTCGCACTGGTCTCCAAGCTGACAGTGGACAAATC | |
| | | | TCGGTGGCAGCAGGGAAATGTCTTTTCATGTAGCGTG | |
| | | | ATGCATGAAGCACTGCACAACCATTACACCCAGAAGT | |
| | | | CACTGTCACTGTCACCAGGA | |
| 62 | 12650 | VH | QVQLVESGGGVVQPGRSLRLSCAASGFTFSNYGMYWV | Q1-S118 |
| | | | RQAPGKGLEWVAVIWYDGSNKYYADSVKGRFTISRDN | |
| | | | SKNTLYLQMNSLRAEDTAVYYCARDLWGWYFDYWGQ | |
| | | | GTLVTVSS | |
| 63 | 12661 | Full | EVQLVQSGPEVKKPGATVKISCKTSGYTFTEYTIHWVKQ | |
| | | | APGKGLEWIGNINPNNGGTTYNQKFEDKATLTVDKSTD | |
| | | | TAYMELSSLRSEDTAVYYCAAGWNFDYWGQGTLLTVS | |
| | | | SASTKGPSVFPLAPSSKSTSGGTAALGCLVKDYFPEPVTV | |
| | | | SWNSGALTSGVHTFPAVLQSSGLYSLSSVVTVPSSSLGT | |
| | | | QTYICNVNHKPSNTKVDKKVEPKSCDKTHTCPPCPAPEA | |
| | | | AGGPSVFLFPPKPKDTLMISRTPEVTCVVVSVSHEDPEV | |
| | | | KFNWYVDGVEVHNAKTKPREEQYNSTYRVVSVLTVLH | |
| | | | QDWLNGKEYKCKVSNKALPAPIEKTISKAKGQPREPQV | |
| | | | YVYPPSRDELTKNQVSLTCLVKGFYPSDIAVEWESNGQP | |
| | | | ENNYKTTPPVLDSDGSFALVSKLTVDKSRWQQGNVFSC | |
| | | | SVMHEALHNHYTQKSLSLSPG | |
| 64 | 12661 | Full | GAGGTCCAGCTGGTCCAGAGCGGCCCCGAGGTGAAG | |
| | | | AAGCCTGGCGCTACTGTGAAGATCTCATGCAAAACAT | |
| | | | CCGGCTACACTTTCACCGAGTACACAATCCACTGGGT | |
| | | | GAAGCAGGCACCCGGAAAAGGCCTGGAATGGATCG | |
| | | | GGAACATTAATCCTAACAATGGCGGGACCACATACAA | |
| | | | CCAGAAGTTCGAGGACAAAGCCACTCTGACCGTGGA | |
| | | | CAAGTCTACAGATACTGCTTATATGGAGCTGAGCTCC | |
| | | | CTGCGGAGCGAAGATACCGCCGTCTACTATTGCGCCG | |
| | | | CTGGATGGAATTTCGATTATTGGGGACAGGGCACCCT | |
| | | | GCTGACAGTCTCAAGCGCTAGCACTAAGGGGCCTTCC | |
| | | | GTGTTTCCACTGGCTCCCTCTAGTAAATCCACCTCTGG | |
| | | | AGGCACAGCTGCACTGGGATGTCTGGTGAAGGATTA | |
| | | | CTTCCCTGAACCAGTCACAGTGAGTTGGAACTCAGGG | |
| | | | GCTCTGACAAGTGGAGTCCATACTTTTCCCGCAGTGC | |
| | | | TGCAGTCAAGCGGACTGTACTCCCTGTCCTCTGTGGT | |
| | | | CACCGTGCCTAGTTCAAGCCTGGGCACCCAGACATAT | |
| | | | ATCTGCAACGTGAATCACAAGCCATCAAATACAAAAG | |
| | | | TCGACAAGAAAGTGGAGCCCAAGAGCTGTGATAAAA | |
| | | | CTCATACCTGCCCACCTTGTCCGGCGCCAGAGGCAGC | |
| | | | AGGAGGACCAAGCGTGTTCCTGTTTCCACCCAAGCCC | |
| | | | AAAGACACCCTGATGATTAGCCGAACCCCTGAAGTCA | |
| | | | CATGCGTGGTCGTGTCCGTGTCTCACGAGGACCCAGA | |
| | | | AGTCAAGTTCAACTGGTACGTGGATGGCGTCGAGGT | |
| | | | GCATAATGCCAAGACAAAACCCCGGGAGGAACAGTA | |
| | | | CAACAGCACCTATAGAGTCGTGTCCGTCCTGACAGTG | |
| | | | CTGCACCAGGATTGGCTGAACGGCAAGGAATATAAG | |
| | | | TGCAAAGTGTCCAATAAGGCCCTGCCCGCTCCTATCG | |
| | | | AGAAAACCATTTCTAAGGCAAAAGGCCAGCCTCGCG | |
| | | | AACCACAGGTCTACGTCTACCCCCCATCAAGAGATGA | |
| | | | ACTGACAAAAAATCAGGTCTCTCTGACATGCCTGGTC | |
| | | | AAAGGATTCTACCCTTCCGACATCGCCGTGGAGTGGG | |
| | | | AAAGTAACGGCCAGCCCGAGAACAATTACAAGACCA | |
| | | | CACCCCCTGTCCTGGACTCTGATGGGAGTTTCGCTCT | |
| | | | GGTGTCAAAGCTGACCGTCGATAAAAGCCGGTGGCA | |
| | | | GCAGGGCAATGTGTTTAGCTGCTCCGTCATGCACGAA | |
| | | | GCCCTGCACAATCACTACACACAGAAGTCCCTGAGCC | |
| | | | TGAGCCCTGGC | |
| 65 | 12661 | VH | EVQLVQSGPEVKKPGATVKISCKTSGYTFTEYTIHWVKQ | E1-S115 |
| | | | APGKGLEWIGNINPNNGGTTYNQKFEDKATLTVDKSTD | |
| | | | TAYMELSSLRSEDTAVYYCAAGWNFDYWGQGTLLTVS | |
| | | | S | |
| 66 | 12662 | Full | DIQMTQSPSSLSTSVGDRVTLTCKASQDVGTAVDWYQ | |
| | | | QKPGPSPKLLIYWASTRHTGIPSRFSGSGSGTDFTLTISSL | |
| | | | QPEDFADYYCQQYNSYPLTFGPGTKVDIKRTVAAPSVFI | |
| | | | FPPSDEQLKSGTASVVCLLNNFYPREAKVQWKVDNALQ | |
| | | | SGNSQESVTEQDSKDSTYSLSSTLTLSKADYEKHKVYACE | |
| | | | VTHQGLSSPVTKSFNRGEC | |
| 67 | 12662 | Full | ATGGCCGTGATGGCACCCCGGACCCTGGTGCTGCTGC | |
| | | | TGAGCGGGGCCCTGGCCCTGACCCAGACATGGGCCG | |
| | | | GCGACATCCAGATGACCCAGTCCCCTAGCTCCCTGTCT | |
| | | | ACAAGCGTGGGCGATAGGGTGACCCTGACATGCAAG | |
| | | | GCCTCCCAGGACGTGGGAACCGCCGTGGATTGGTAC | |
| | | | CAGCAGAAGCCAGGCCCCTCTCCTAAGCTGCTGATCT | |
| | | | ATTGGGCCTCTACCCGGCACACAGGCATCCCTAGCAG | |
| | | | ATTCTCCGGCTCTGGCAGCGGCACAGACTTTACCCTG | |
| | | | ACAATCTCTAGCCTGCAGCCAGAGGACTTCGCCGATT | |
| | | | ACTATTGCCAGCAGTACAACTCCTATCCACTGACCTTT | |
| | | | GGCCCCGGCACAAAGGTGGACATCAAGAGGACCGTG | |
| | | | GCGGCGCCCAGCGTGTTCATCTTTCCCCCTTCCGATGA | |
| | | | GCAGCTGAAGTCCGGCACAGCCTCTGTGGTGTGCCTG | |
| | | | CTGAACAATTTCTACCCCCGCGAGGCCAAGGTGCAGT | |
| | | | GGAAGGTGGACAACGCCCTGCAGTCCGGCAATTCTC | |
| | | | AGGAGAGCGTGACCGAGCAGGACTCCAAGGATTCTA | |
| | | | CATATAGCCTGTCCTCTACCCTGACACTGTCTAAGGCC | |
| | | | GATTACGAGAAGCACAAGGTGTATGCATGCGAGGTG | |
| | | | ACCCACCAGGGCCTGAGCTCCCCTGTGACAAAGAGCT | |
| | | | TTAATCGGGGCGAGTGT | |
| 68 | 12662 | VL | DIQMTQSPSSLSTSVGDRVTLTCKASQDVGTAVDWYQ | D1-K107 |
| | | | QKPGPSPKLLIYWASTRHTGIPSRFSGSGSGTDFTLTISSL | |
| | | | QPEDFADYYCQQYNSYPLTFGPGTKVDIK | |
| 69 | | Human | APELLGGPSVFLFPPKPKDTLMISRTPEVTCVVVDVSHE | |
| | | IgG1 Fc | DPEVKFNWYVDGVEVHNAKTKPREEQYNSTYRVVSVLT | |
| | | sequence | VLHQDWLNGKEYKCKVSNKALPAPIEKTISKAKGQPREP | |
| | | 231- | QVYTLPPSRDELTKNQVSLTCLVKGFYPSDIAVEWESNG | |
| | | 447 (EU | QPENNYKTTPPVLDSDGSFFLYSKLTVDKSRWQQGNVF | |
| | | numbering) | SCSVMHEALHNHYTQKSLSLSPGK | |
| 70 | 10565 | Full | DIQMTQSPSSLSASVGDRVTITCSASSSVSYMHWYQQK | CL = R107- |
| | | | SGKAPKLLIYDTSKLASGVPSRFSGSGSGTDFTLTISSLQP | C213; |
| | | | EDFATYYCQQWSKHPLTFGQGTKLEIKRTVAAPSVFIFP | VL = D1- |
| | | | PSDEQLKSGTASVVCLLNNFYPREAKVQWKVDNALQS | K106 |
| | | | GNSQESVTEQDSKDSTYSLSSTLTLSKADYEKHKVYACEV | |
| | | | THQGLSSPVTKSFNRGEC | |
| 71 | 10565 | Full | GACATCCAGATGACACAGAGCCCAAGCTCCCTGTCCG | |
| | | | CCTCTGTGGGCGATAGAGTGACCATCACATGCAGCGC | |
| | | | CTCTAGCTCCGTGTCCTACATGCACTGGTATCAGCAG | |
| | | | AAGTCCGGCAAGGCCCCCAAGCTGCTGATCTACGACA | |
| | | | CCAGCAAGCTGGCCTCCGGAGTGCCTTCTAGGTTCAG | |
| | | | CGGCTCCGGCTCTGGCACCGACTTTACCCTGACAATCT | |
| | | | CTAGCCTGCAGCCAGAGGATTTCGCCACATACTATTG | |
| | | | TCAGCAGTGGAGCAAGCACCCCCTGACCTTTGGCCAG | |
| | | | GGCACAAAGCTGGAGATCAAGCGGACAGTGGCGGC | |
| | | | GCCCAGTGTCTTCATTTTTCCCCCTAGCGACGAACAGC | |
| | | | TGAAGTCTGGGACAGCCAGTGTGGTCTGTCTGCTGAA | |
| | | | CAACTTCTACCCTAGAGAGGCTAAAGTGCAGTGGAA | |
| | | | GGTCGATAACGCACTGCAGTCCGGAAATTCTCAGGA | |
| | | | GAGTGTGACTGAACAGGACTCAAAAGATAGCACCTA | |
| | | | TTCCCTGTCAAGCACACTGACTCTGAGCAAGGCCGAC | |
| | | | TACGAGAAGCATAAAGTGTATGCTTGTGAAGTCACCC | |
| | | | ACCAGGGGCTGAGTTCACCAGTCACAAAATCATTCAA | |
| | | | CAGAGGGGAGTGC | |
| 72 | 11150 | Full | DIQMTQSPSSLSASVGDRVTITCRASQDVNTAVAWYQ | VL = D1- |
| | | | QKPGKAPKWYSASFLYSGVPSRFSGSRSGTDFTLTISSL | K107; |
| | | | QPEDFATYYCQQHYTTPPTFGQGTKVEIKRTVAAPSVFI | CL = R108- |
| | | | FPPSDEQLKSGTASVVCLLNNFYPREAKVQWKVDNALQ | C214 |
| | | | SGNSQESVTEQDSKDSTYSLSSTLTLSKADYEKHKVYACE | |
| | | | VTHQGLSSPVTKSFNRGEC | |
| 73 | 11150 | Full | GACATCCAGATGACACAGTCCCCAAGCTCCCTGTCCG | |
| | | | CCTCTGTGGGCGACAGGGTGACCATCACATGCCGCGC | |
| | | | CTCTCAGGATGTGAACACCGCCGTGGCCTGGTACCAG | |
| | | | CAGAAGCCAGGCAAGGCCCCCAAGCTGCTGATCTAC | |
| | | | AGCGCCTCCTTCCTGTATTCTGGCGTGCCCAGCCGGTT | |
| | | | TTCTGGCAGCAGATCCGGCACCGACTTCACCCTGACA | |
| | | | ATCTCTAGCCTGCAGCCTGAGGATTTTGCCACATACTA | |
| | | | TTGTCAGCAGCACTATACCACACCCCCTACCTTCGGCC | |
| | | | AGGGCACAAAGGTGGAGATCAAGCGGACAGTGGCG | |
| | | | GCGCCCAGTGTCTTCATTTTTCCCCCTAGCGACGAACA | |
| | | | GCTGAAGTCTGGGACAGCCAGTGTGGTCTGTCTGCTG | |
| | | | AACAACTTCTACCCTAGAGAGGCTAAAGTGCAGTGGA | |
| | | | AGGTCGATAACGCACTGCAGTCCGGAAATTCTCAGGA | |
| | | | GAGTGTGACTGAACAGGACTCAAAAGATAGCACCTA | |
| | | | TTCCCTGTCAAGCACACTGACTCTGAGCAAGGCCGAC | |
| | | | TACGAGAAGCATAAAGTGTATGCTTGTGAAGTCACCC | |
| | | | ACCAGGGGCTGAGTTCACCAGTCACAAAATCATTCAA | |
| | | | CAGAGGGGAGTGC | |
| 74 | 12153 | Full | EPKSSDKTHTCPPCPAPEAAGGPSVFLFPPKPKDTLMISR | |
| | | | TPEVTCVVVSVSHEDPEVKFNWYVDGVEVHNAKTKPR | |
| | | | EEQYNSTYRVVSVLTVLHQDWLNGKEYKCKVSNKALPA | |
| | | | PIEKTISKAKGQPREPQVYVLPPSRDELTKNQVSLLCLVK | |
| | | | GFYPSDIAVEWESNGQPENNYLTWPPVLDSDGSFFLYS | |
| | | | KLTVDKSRWQQGNVFSCSVMHEALHNHYTQKSLSLSP | |
| | | | G | |
| 75 | 12153 | Full | GAGCCAAAGAGCTCCGACAAGACCCACACATGCCCCC | |
| | | | CTTGTCCGGCGCCAGAGGCAGCAGGAGGACCAAGCG | |
| | | | TGTTCCTGTTTCCACCCAAGCCCAAAGACACCCTGATG | |
| | | | ATTAGCCGAACCCCTGAAGTCACATGCGTGGTCGTGT | |
| | | | CCGTGTCTCACGAGGACCCAGAAGTCAAGTTCAACTG | |
| | | | GTACGTGGATGGCGTCGAGGTGCATAATGCCAAGAC | |
| | | | AAAACCCCGGGAGGAACAGTACAACAGCACCTATAG | |
| | | | AGTCGTGTCCGTCCTGACAGTGCTGCACCAGGATTGG | |
| | | | CTGAACGGCAAGGAATATAAGTGCAAAGTGTCCAAT | |
| | | | AAGGCCCTGCCCGCTCCTATCGAGAAAACCATTTCTA | |
| | | | AGGCAAAAGGCCAGCCTCGCGAACCACAGGTCTACG | |
| | | | TGCTGCCTCCATCCCGGGACGAGCTGACAAAGAACCA | |
| | | | GGTCTCTCTGCTGTGCCTGGTGAAAGGCTTCTATCCAT | |
| | | | CAGATATTGCTGTGGAGTGGGAAAGCAATGGGCAGC | |
| | | | CCGAGAACAATTACCTGACTTGGCCCCCTGTGCTGGA | |
| | | | CTCTGATGGGAGTTTCTTTCTGTATTCTAAGCTGACCG | |
| | | | TGGATAAAAGTAGGTGGCAGCAGGGAAATGTCTTTA | |
| | | | GTTGTTCAGTGATGCATGAAGCCCTGCATAACCACTA | |
| | | | CACCCAGAAAAGCCTGTCCCTGTCCCCCGGA | |
| 76 | 12155 | Full | EPKSSDKTHTCPPCPAPEAAGGPSVFLFPPKPKDTLMISR | |
| | | | TPEVTCVVVSVSHEDPEVKFNWYVDGVEVHNAKTKPR | |
| | | | EEQYNSTYRVVSVLTVLHQDWLNGKEYKCKVSNKALPA | |
| | | | PIEKTISKAKGQPREPQVYVYPPSRDELTKNQVSLTCLVK | |
| | | | GFYPSDIAVEWESNGQPENNYKTTPPVLDSDGSFALVS | |
| | | | KLTVDKSRWQQGNVFSCSVMHEALHNHYTQKSLSLSP | |
| | | | G | |
| 77 | 12155 | Full | GAGCCAAAGAGCTCCGACAAGACCCACACATGCCCCC | |
| | | | CTTGTCCGGCGCCAGAGGCTGCAGGAGGACCAAGCG | |
| | | | TGTTCCTGTTTCCACCCAAGCCTAAAGACACACTGATG | |
| | | | ATTTCCCGAACCCCCGAAGTCACATGCGTGGTCGTGT | |
| | | | CTGTGAGTCACGAGGACCCTGAAGTCAAGTTCAACTG | |
| | | | GTACGTGGATGGCGTCGAGGTGCATAATGCCAAGAC | |
| | | | TAAACCTAGGGAGGAACAGTACAACTCAACCTATCGC | |
| | | | GTCGTGAGCGTCCTGACAGTGCTGCACCAGGATTGGC | |
| | | | TGAACGGCAAAGAATATAAGTGCAAAGTGAGCAATA | |
| | | | AGGCCCTGCCCGCTCCTATCGAGAAAACCATTTCCAA | |
| | | | GGCTAAAGGGCAGCCTCGCGAACCACAGGTCTACGT | |
| | | | GTATCCTCCAAGCCGGGACGAGCTGACAAAGAACCA | |
| | | | GGTCTCCCTGACTTGTCTGGTGAAAGGGTTTTACCCT | |
| | | | AGTGATATCGCTGTGGAGTGGGAATCAAATGGACAG | |
| | | | CCAGAGAACAATTATAAGACTACCCCCCCTGTGCTGG | |
| | | | ACAGTGATGGGTCATTCGCACTGGTCTCCAAGCTGAC | |
| | | | AGTGGACAAATCTCGGTGGCAGCAGGGAAATGTCTT | |
| | | | TTCATGTAGCGTGATGCATGAAGCACTGCACAACCAT | |
| | | | TACACCCAGAAGTCACTGTCACTGTCACCAGGA | |
| 78 | 12645 | Full | QIVLTQSPAVMSASPGEKVTITCTASSSLSYMHWFQQK | VL = Q1- |
| | | | PGTSPKLWLYSTSILASGVPTRFSGSGSGTSYSLTISRME | K106; |
| | | | AEDAATYYCQQRSSSPFTFGSGTKLEIKRTVAAPSVFIFP | CL = R107- |
| | | | PSDEQLKSGTASVVCLLNNFYPREAKVQWKVDNALQS | C213 |
| | | | GNSQESVTEQDSKDSTYSLSSTLTLSKADYEKHKVYACEV | |
| | | | THQGLSSPVTKSFNRGEC | |
| 79 | 12645 | Full | CAGATCGTGCTGACCCAGTCCCCAGCCGTGATGAGCG | |
| | | | CCTCCCCAGGAGAGAAGGTGACCATCACATGCACCGC | |
| | | | CAGCTCCTCTCTGAGCTACATGCACTGGTTCCAGCAG | |
| | | | AAGCCCGGCACATCCCCTAAGCTGTGGCTGTATTCTA | |
| | | | CCAGCATCCTGGCCTCTGGCGTGCCTACAAGGTTTTCC | |
| | | | GGCTCTGGCAGCGGCACATCCTACTCTCTGACCATCA | |
| | | | GCCGGATGGAGGCAGAGGACGCAGCAACCTACTATT | |
| | | | GTCAGCAGAGAAGCTCCTCTCCCTTCACATTTGGCAG | |
| | | | CGGCACCAAGCTGGAGATCAAGCGGACAGTGGCGGC | |
| | | | GCCCAGTGTCTTCATTTTTCCCCCTAGCGACGAACAGC | |
| | | | TGAAGTCTGGGACAGCCAGTGTGGTCTGTCTGCTGAA | |
| | | | CAACTTCTACCCTAGAGAGGCTAAAGTGCAGTGGAA | |
| | | | GGTCGATAACGCACTGCAGTCCGGAAATTCTCAGGA | |
| | | | GAGTGTGACTGAACAGGACTCAAAAGATAGCACCTA | |
| | | | TTCCCTGTCAAGCACACTGACTCTGAGCAAGGCCGAC | |
| | | | TACGAGAAGCATAAAGTGTATGCTTGTGAAGTCACCC | |
| | | | ACCAGGGGCTGAGTTCACCAGTCACAAAATCATTCAA | |
| | | | CAGAGGGGAGTGC | |
| 80 | 12651 | Full | EIVLTQSPATLSLSPGERATLSCRASQSVSSYLAWYQQKP | VL = E1- |
| | | | GQAPRLLIYDASNRATGIPARFSGSGSGTDFTLTISSLEPE | K107; |
| | | | DFAVYYCQQRRNWPLTFGGGTKVEIKRTVAAPSVFIFPP | CL = R108- |
| | | | SDEQLKSGTASVVCLLNNFYPREAKVQWKVDNALQSG | C214 |
| | | | NSQESVTEQDSKDSTYSLSSTLTLSKADYEKHKVYACEVT | |
| | | | HQGLSSPVTKSFNRGEC | |
| 81 | 12651 | Full | GAGATCGTGCTGACCCAGTCTCCAGCCACACTGTCCC | |
| | | | TGTCTCCAGGAGAGAGGGCCACCCTGAGCTGCAGGG | |
| | | | CCAGCCAGTCCGTGAGCTCCTACCTGGCCTGGTATCA | |
| | | | GCAGAAGCCAGGACAGGCCCCCCGGCTGCTGATCTA | |
| | | | CGACGCCTCCAACAGGGCAACCGGCATCCCCGCAAG | |
| | | | ATTCTCTGGCAGCGGCTCCGGCACAGACTTTACCCTG | |
| | | | ACAATCTCTAGCCTGGAGCCTGAGGATTTCGCCGTGT | |
| | | | ACTATTGTCAGCAGCGGAGAAATTGGCCACTGACCTT | |
| | | | TGGCGGCGGCACAAAGGTGGAGATCAAGAGAACAG | |
| | | | TGGCGGCGCCCAGTGTCTTCATTTTTCCCCCTAGCGAC | |
| | | | GAACAGCTGAAGTCTGGGACAGCCAGTGTGGTCTGT | |
| | | | CTGCTGAACAACTTCTACCCTAGAGAGGCTAAAGTGC | |
| | | | AGTGGAAGGTCGATAACGCACTGCAGTCCGGAAATT | |
| | | | CTCAGGAGAGTGTGACTGAACAGGACTCAAAAGATA | |
| | | | GCACCTATTCCCTGTCAAGCACACTGACTCTGAGCAA | |
| | | | GGCCGACTACGAGAAGCATAAAGTGTATGCTTGTGA | |
| | | | AGTCACCCACCAGGGGCTGAGTTCACCAGTCACAAAA | |
| | | | TCATTCAACAGAGGGGAGTGC | |
| 82 | 12653 | Full | DIQMTQTTSSLSASLGDRVTISCSASQGISNYLNWYQQK | VL = D1- |
| | | | PDGTVKLLIYYTSILHSGVPSRFSGSGSGTDYSLTIGNLEP | K107; |
| | | | EDIATYYCQQFNKLPPTFGGGTKLEIKRTVAAPSVFIFPPS | CL = R108- |
| | | | DEQLKSGTASVVCLLNNFYPREAKVQWKVDNALQSGN | C214 |
| | | | SQESVTEQDSKDSTYSLSSTLTLSKADYEKHKVYACEVTH | |
| | | | QGLSSPVTKSFNRGEC | |
| 83 | 12653 | Full | GACATCCAGATGACCCAGACCACAAGCTCCCTGTCTG | |
| | | | CCAGCCTGGGCGATCGGGTGACAATCTCCTGCTCTGC | |
| | | | CAGCCAGGGCATCTCCAACTACCTGAATTGGTATCAG | |
| | | | CAGAAGCCAGACGGCACCGTGAAGCTGCTGATCTACT | |
| | | | ATACATCCATCCTGCACTCTGGCGTGCCCAGCAGATTC | |
| | | | TCCGGCTCTGGCAGCGGCACCGACTACTCTCTGACAA | |
| | | | TCGGCAACCTGGAGCCCGAGGATATCGCCACCTACTA | |
| | | | TTGTCAGCAGTTCAATAAGCTGCCCCCTACCTTTGGCG | |
| | | | GCGGCACAAAGCTGGAGATCAAGCGGACAGTGGCG | |
| | | | GCGCCCAGTGTCTTCATTTTTCCCCCTAGCGACGAACA | |
| | | | GCTGAAGTCTGGGACAGCCAGTGTGGTCTGTCTGCTG | |
| | | | AACAACTTCTACCCTAGAGAGGCTAAAGTGCAGTGGA | |
| | | | AGGTCGATAACGCACTGCAGTCCGGAAATTCTCAGGA | |
| | | | GAGTGTGACTGAACAGGACTCAAAAGATAGCACCTA | |
| | | | TTCCCTGTCAAGCACACTGACTCTGAGCAAGGCCGAC | |
| | | | TACGAGAAGCATAAAGTGTATGCTTGTGAAGTCACCC | |
| | | | ACCAGGGGCTGAGTTCACCAGTCACAAAATCATTCAA | |
| | | | CAGAGGGGAGTGC | |
| 84 | 12659 | Full | QEQLVESGGRLVTPGGSLTLSCKASGFDFSAYYMSWVR | VH = Q1- |
| | | | QAPGKGLEWIATIYPSSGKTYYATWVNGRFTISSDNAQ | S121; |
| | | | NTVDLQMNSLTAADRATYFCARDSYADDGALFNIWGP | CH1 = |
| | | | GTLVTISSASTKGPSVFPLAPSSKSTSGGTAALGCLVKDYF | A122-V219 |
| | | | PEPVTVSWNSGALTSGVHTFPAVLQSSGLYSLSSVVTVP | |
| | | | SSSLGTQTYICNVNHKPSNTKVDKKVEPKSCDKTHTCPP | |
| | | | CPAPEAAGGPSVFLFPPKPKDTLMISRTPEVTCVVVSVS | |
| | | | HEDPEVKFNWYVDGVEVHNAKTKPREEQYNSTYRVVS | |
| | | | VLTVLHQDWLNGKEYKCKVSNKALPAPIEKTISKAKGQP | |
| | | | REPQVYVYPPSRDELTKNQVSLTCLVKGFYPSDIAVEWE | |
| | | | SNGQPENNYKTTPPVLDSDGSFALVSKLTVDKSRWQQ | |
| | | | GNVFSCSVMHEALHNHYTQKSLSLSPG | |
| 85 | 12659 | Full | CAGGAGCAGCTGGTGGAGTCCGGCGGCAGGCTGGT | |
| | | | GACCCCAGGAGGCAGCCTGACACTGTCCTGCAAGGC | |
| | | | CTCTGGCTTCGACTTTAGCGCCTACTATATGTCCTGGG | |
| | | | TGCGCCAGGCCCCCGGCAAGGGCCTGGAGTGGATCG | |
| | | | CCACCATCTACCCTAGCTCCGGCAAGACCTACTATGCC | |
| | | | ACATGGGTGAACGGCAGATTCACCATCTCTAGCGACA | |
| | | | ACGCCCAGAATACAGTGGATCTGCAGATGAACAGCCT | |
| | | | GACCGCCGCCGACAGGGCAACATACTTCTGTGCCAGA | |
| | | | GATAGCTATGCCGACGATGGGGCCCTGTTCAACATCT | |
| | | | GGGGACCAGGCACCCTGGTGACAATCTCCTCTGCTAG | |
| | | | CACTAAGGGGCCTTCCGTGTTTCCACTGGCTCCCTCTA | |
| | | | GTAAATCCACCTCTGGAGGCACAGCTGCACTGGGATG | |
| | | | TCTGGTGAAGGATTACTTCCCTGAACCAGTCACAGTG | |
| | | | AGTTGGAACTCAGGGGCTCTGACAAGTGGAGTCCAT | |
| | | | ACTTTTCCCGCAGTGCTGCAGTCAAGCGGACTGTACT | |
| | | | CCCTGTCCTCTGTGGTCACCGTGCCTAGTTCAAGCCTG | |
| | | | GGCACCCAGACATATATCTGCAACGTGAATCACAAGC | |
| | | | CATCAAATACAAAAGTCGACAAGAAAGTGGAGCCCA | |
| | | | AGAGCTGTGATAAAACTCATACCTGCCCACCTTGTCC | |
| | | | GGCGCCAGAGGCTGCAGGAGGACCAAGCGTGTTCCT | |
| | | | GTTTCCACCCAAGCCTAAAGACACACTGATGATTTCCC | |
| | | | GAACCCCCGAAGTCACATGCGTGGTCGTGTCTGTGAG | |
| | | | TCACGAGGACCCTGAAGTCAAGTTCAACTGGTACGTG | |
| | | | GATGGCGTCGAGGTGCATAATGCCAAGACTAAACCT | |
| | | | AGGGAGGAACAGTACAACTCAACCTATCGCGTCGTG | |
| | | | AGCGTCCTGACAGTGCTGCACCAGGATTGGCTGAAC | |
| | | | GGCAAAGAATATAAGTGCAAAGTGAGCAATAAGGCC | |
| | | | CTGCCCGCTCCTATCGAGAAAACCATTTCCAAGGCTA | |
| | | | AAGGGCAGCCTCGCGAACCACAGGTCTACGTGTATCC | |
| | | | TCCAAGCCGGGACGAGCTGACAAAGAACCAGGTCTC | |
| | | | CCTGACTTGTCTGGTGAAAGGGTTTTACCCTAGTGAT | |
| | | | ATCGCTGTGGAGTGGGAATCAAATGGACAGCCAGAG | |
| | | | AACAATTATAAGACTACCCCCCCTGTGCTGGACAGTG | |
| | | | ATGGGTCATTCGCACTGGTCTCCAAGCTGACAGTGGA | |
| | | | CAAATCTCGGTGGCAGCAGGGAAATGTCTTTTCATGT | |
| | | | AGCGTGATGCATGAAGCACTGCACAACCATTACACCC | |
| | | | AGAAGTCACTGTCACTGTCACCAGGA | |
| 86 | 12660 | Full | ELVLTQSPSVSAALGSPAKITCTLSSAHKTDTIDWYQQLQ | VL = E1- |
| | | | GEAPRYLMQVQSDGSYTKRPGVPDRFSGSSSGADRYLII | T111; |
| | | | PSVQADDEADYYCGADYIGGYVFGGGTQLTVTRTVAAP | CL = R112- |
| | | | SVFIFPPSDEQLKSGTASVVCLLNNFYPREAKVQWKVDN | C218 |
| | | | ALQSGNSQESVTEQDSKDSTYSLSSTLTLSKADYEKHKVY | |
| | | | ACEVTHQGLSSPVTKSFNRGEC | |
| 87 | 12660 | Full | GAGCTGGTGCTGACACAGTCTCCAAGCGTGTCCGCCG | |
| | | | CCCTGGGCAGCCCCGCCAAGATCACCTGCACACTGAG | |
| | | | CTCCGCCCACAAGACCGACACAATCGATTGGTACCAG | |
| | | | CAGCTGCAGGGAGAGGCCCCCCGGTATCTGATGCAG | |
| | | | GTGCAGTCTGACGGCAGCTACACAAAGCGGCCCGGA | |
| | | | GTGCCTGACAGATTCTCCGGCTCTAGCTCCGGAGCCG | |
| | | | ATCGCTATCTGATCATCCCCTCTGTGCAGGCCGACGAT | |
| | | | GAGGCCGACTACTATTGTGGAGCCGATTACATCGGA | |
| | | | GGATACGTGTTCGGAGGAGGAACCCAGCTGACCGTG | |
| | | | ACACGGACCGTGGCGGCGCCCAGTGTCTTCATTTTTC | |
| | | | CCCCTAGCGACGAACAGCTGAAGTCTGGGACAGCCA | |
| | | | GTGTGGTCTGTCTGCTGAACAACTTCTACCCTAGAGA | |
| | | | GGCTAAAGTGCAGTGGAAGGTCGATAACGCACTGCA | |
| | | | GTCCGGAAATTCTCAGGAGAGTGTGACTGAACAGGA | |
| | | | CTCAAAAGATAGCACCTATTCCCTGTCAAGCACACTG | |
| | | | ACTCTGAGCAAGGCCGACTACGAGAAGCATAAAGTG | |
| | | | TATGCTTGTGAAGTCACCCACCAGGGGCTGAGTTCAC | |
| | | | CAGTCACAAAATCATTCAACAGAGGGGAGTGC | |
| 88 | 12667 | Full | EPAVYFKEQFLDGDGWTSRWIESKHKSDFGKFVLSSGK | |
| | | | FYGDEEKDKGLQTSQDARFYALSASFEPFSNKGQTLVV | |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAAAEPKSSDKTHTCPPCPAPEA | |
| | | | AGGPSVFLFPPKPKDTLMISRTPEVTCVVVSVSHEDPEV | |
| | | | KFNWYVDGVEVHNAKTKPREEQYNSTYRVVSVLTVLH | |
| | | | QDWLNGKEYKCKVSNKALPAPIEKTISKAKGQPREPQV | |
| | | | YVLPPSRDELTKNQVSLLCLVKGFYPSDIAVEWESNGQP | |
| | | | ENNYLTWPPVLDSDGSFFLYSKLTVDKSRWQQGNVFSC | |
| | | | SVMHEALHNHYTQKSLSLSPG | |
| 89 | 12667 | Full | GAGCCTGCCGTGTATTTCAAGGAGCAGTTTCTGGACG | |
| | | | GCGATGGCTGGACAAGCAGATGGATCGAGTCTAAGC | |
| | | | ACAAGAGCGACTTCGGCAAGTTTGTGCTGAGCTCCGG | |
| | | | CAAGTTCTATGGCGATGAGGAGAAGGACAAGGGCCT | |
| | | | GCAGACCTCTCAGGATGCCAGGTTTTACGCCCTGTCC | |
| | | | GCCTCTTTCGAGCCCTTCAGCAACAAGGGCCAGACCC | |
| | | | TGGTGGTGCAGTTCACAGTGAAGCACGAGCAGAACA | |
| | | | TCGACTGCGGCGGCGGCTATGTGAAGCTGTTTCCCAA | |
| | | | TAGCCTGGATCAGACCGACATGCACGGCGACTCCGA | |
| | | | GTACAACATCATGTTCGGCCCTGATATCTGCGGCCCA | |
| | | | GGCACAAAGAAGGTGCACGTGATCTTTAATTACAAG | |
| | | | GGCAAGAACGTGCTGATCAATAAGGACATCAGGTGT | |
| | | | AAGGACGATGAGTTCACCCACCTGTACACACTGATCG | |
| | | | TGCGCCCTGACAACACATATGAGGTGAAGATCGATAA | |
| | | | TTCCCAGGTGGAGAGCGGCTCCCTGGAGGACGATTG | |
| | | | GGATTTTCTGCCCCCTAAGAAGATCAAGGACCCCGAT | |
| | | | GCCTCCAAGCCTGAGGACTGGGATGAGCGCGCCAAG | |
| | | | ATCGACGATCCAACCGACTCTAAGCCCGAGGACTGG | |
| | | | GATAAGCCCGAGCACATCCCCGACCCTGATGCCAAGA | |
| | | | AGCCAGAAGACTGGGATGAGGAGATGGATGGCGAG | |
| | | | TGGGAGCCACCCGTGATCCAGAACCCAGAGTACAAG | |
| | | | GGCGAGTGGAAGCCCAGACAGATCGATAATCCTGAC | |
| | | | TATAAGGGCACCTGGATTCACCCTGAGATCGATAACC | |
| | | | CAGAGTACTCCCCAGACCCCTCTATCTACGCCTATGAT | |
| | | | AATTTCGGCGTGCTGGGCCTGGACCTGTGGCAGGTG | |
| | | | AAGAGCGGCACCATCTTCGACAACTTTCTGATCACAA | |
| | | | ATGATGAGGCCTACGCCGAGGAGTTTGGCAACGAGA | |
| | | | CATGGGGCGTGACAAAGGCCGCCGAGAAGCAGATG | |
| | | | AAGGATAAGCAGGACGAGGAGCAGAGGCTGAAGGA | |
| | | | AGAGGAGGAGGACAAGAAGCGCAAGGAGGAGGAG | |
| | | | GAGGCCGAGGATAAGGAGGACGATGAGGACAAGGA | |
| | | | TGAGGACGAGGAGGATGAGGAGGACAAGGAGGAG | |
| | | | GATGAGGAGGAGGACGTGCCAGGACAGGCCGCCGC | |
| | | | CGAGCCCAAGTCTAGCGACAAGACCCACACATGCCCT | |
| | | | CCATGTCCGGCGCCGGAGGCCGCCGGAGGACCTAGC | |
| | | | GTGTTCCTGTTTCCCCCTAAGCCAAAGGATACACTGAT | |
| | | | GATCTCCAGAACCCCTGAGGTGACATGCGTGGTGGT | |
| | | | GTCTGTGAGCCACGAGGACCCAGAGGTGAAGTTCAA | |
| | | | CTGGTATGTGGATGGCGTGGAGGTGCACAATGCCAA | |
| | | | GACCAAGCCCCGGGAGGAGCAGTACAATAGCACCTA | |
| | | | TAGAGTGGTGTCCGTGCTGACAGTGCTGCACCAGGA | |
| | | | CTGGCTGAACGGCAAGGAGTACAAGTGCAAGGTGTC | |
| | | | CAATAAGGCCCTGCCGGCACCTATCGAGAAGACCATC | |
| | | | TCTAAGGCAAAGGGACAGCCACGGGAGCCACAGGTG | |
| | | | TATGTGCTGCCACCCTCTAGAGACGAGCTGACAAAGA | |
| | | | ACCAGGTGAGCCTGCTGTGCCTGGTGAAGGGCTTCTA | |
| | | | CCCATCCGATATCGCCGTGGAGTGGGAGTCTAATGGC | |
| | | | CAGCCCGAGAACAATTATCTGACCTGGCCTCCAGTGC | |
| | | | TGGATAGCGACGGCTCCTTCTTTCTGTACTCTAAGCTG | |
| | | | ACAGTGGACAAGAGCCGGTGGCAGCAGGGCAACGT | |
| | | | GTTTTCCTGTTCTGTGATGCACGAGGCCCTGCACAATC | |
| | | | ACTACACCCAGAAGAGCCTGTCCCTGTCTCCTGGC | |
| 90 | 12966 | Full | QVQLVQSGAEVKKPGASVKVSCKASGYSFTGYTMNWV | VH = Q1- |
| | | | RQAPGQGLEWMGLITPYNGASSYNQKFRGKATMTVD | S119; |
| | | | TSTSTVYMELSSLRSEDTAVYYCARGGYDGRGFDYWGQ | CH1 = |
| | | | GTLVTVSSASTKGPSVFPLAPSSKSTSGGTAALGCLVKDY | A120-V217 |
| | | | FPEPVTVSWNSGALTSGVHTFPAVLQSSGLYSLSSVVTV | |
| | | | PSSSLGTQTYICNVNHKPSNTKVDKKVEPKSCDKTHTCP | |
| | | | PCPAPEAAGGPSVFLFPPKPKDTLMISRTPEVTCVVVSV | |
| | | | SHEDPEVKFNWYVDGVEVHNAKTKPREEQYNSTYRVV | |
| | | | SVLTVLHQDWLNGKEYKCKVSNKALPAPIEKTISKAKGQ | |
| | | | PREPQVYVYPPSRDELTKNQVSLTCLVKGFYPSDIAVEW | |
| | | | ESNGQPENNYKTTPPVLDSDGSFALVSKLTVDKSRWQQ | |
| | | | GNVFSCSVMHEALHNHYTQKSLSLSPG | |
| 91 | 12966 | Full | CAGGTGCAGCTGGTGCAGAGCGGAGCCGAGGTGAA | |
| | | | GAAGCCAGGGGCCAGCGTGAAGGTGTCTTGCAAGGC | |
| | | | CTCTGGCTACAGCTTCACAGGCTATACCATGAACTGG | |
| | | | GTGCGGCAGGCCCCCGGACAGGGCCTGGAGTGGATG | |
| | | | GGCCTGATCACACCTTACAACGGGGCCAGCTCCTATA | |
| | | | ATCAGAAGTTTCGGGGCAAGGCCACCATGACAGTGG | |
| | | | ACACCAGCACATCCACCGTGTACATGGAGCTGTCTAG | |
| | | | CCTGAGGTCCGAGGATACCGCCGTGTACTATTGTGCC | |
| | | | AGAGGCGGCTACGACGGCAGAGGCTTTGATTATTGG | |
| | | | GGCCAGGGCACACTGGTGACCGTGTCCTCTGCTAGCA | |
| | | | CTAAGGGGCCTTCCGTGTTTCCACTGGCTCCCTCTAGT | |
| | | | AAATCCACCTCTGGAGGCACAGCTGCACTGGGATGTC | |
| | | | TGGTGAAGGATTACTTCCCTGAACCAGTCACAGTGAG | |
| | | | TTGGAACTCAGGGGCTCTGACAAGTGGAGTCCATACT | |
| | | | TTTCCCGCAGTGCTGCAGTCAAGCGGACTGTACTCCC | |
| | | | TGTCCTCTGTGGTCACCGTGCCTAGTTCAAGCCTGGG | |
| | | | CACCCAGACATATATCTGCAACGTGAATCACAAGCCA | |
| | | | TCAAATACAAAAGTCGACAAGAAAGTGGAGCCCAAG | |
| | | | AGCTGTGATAAAACTCATACCTGCCCACCTTGTCCGG | |
| | | | CGCCAGAGGCTGCAGGAGGACCAAGCGTGTTCCTGT | |
| | | | TTCCACCCAAGCCTAAAGACACACTGATGATTTCCCG | |
| | | | AACCCCCGAAGTCACATGCGTGGTCGTGTCTGTGAGT | |
| | | | CACGAGGACCCTGAAGTCAAGTTCAACTGGTACGTG | |
| | | | GATGGCGTCGAGGTGCATAATGCCAAGACTAAACCT | |
| | | | AGGGAGGAACAGTACAACTCAACCTATCGCGTCGTG | |
| | | | AGCGTCCTGACAGTGCTGCACCAGGATTGGCTGAAC | |
| | | | GGCAAAGAATATAAGTGCAAAGTGAGCAATAAGGCC | |
| | | | CTGCCCGCTCCTATCGAGAAAACCATTTCCAAGGCTA | |
| | | | AAGGGCAGCCTCGCGAACCACAGGTCTACGTGTATCC | |
| | | | TCCAAGCCGGGACGAGCTGACAAAGAACCAGGTCTC | |
| | | | CCTGACTTGTCTGGTGAAAGGGTTTTACCCTAGTGAT | |
| | | | ATCGCTGTGGAGTGGGAATCAAATGGACAGCCAGAG | |
| | | | AACAATTATAAGACTACCCCCCCTGTGCTGGACAGTG | |
| | | | ATGGGTCATTCGCACTGGTCTCCAAGCTGACAGTGGA | |
| | | | CAAATCTCGGTGGCAGCAGGGAAATGTCTTTTCATGT | |
| | | | AGCGTGATGCATGAAGCACTGCACAACCATTACACCC | |
| | | | AGAAGTCACTGTCACTGTCACCAGGA | |
| 92 | 16711 | Full | ELVLTQSPSVSAALGSPAKITCTLSSAHKTDTIDWYQQLQ | VL = E1- |
| | | | GEAPRYLMQVQSDGSYTKRPGVPDRFSGSSSGADRYLII | T111; |
| | | | PSVQADDEADYYCGADYIGGYVFGGGTQLTVTVEGGS | VH = Q130- |
| | | | GGSGGSGGSGGVDQEQLVESGGRLVTPGGSLTLSCKAS | S250 |
| | | | GFDFSAYYMSWVRQAPGKGLEWIATIYPSSGKTYYATW | |
| | | | VNGRFTISSDNAQNTVDLQMNSLTAADRATYFCARDSY | |
| | | | ADDGALFNIWGPGTLVTISSAAEPKSSDKTHTCPPCPAP | |
| | | | EAAGGPSVFLFPPKPKDTLMISRTPEVTCVVVSVSHEDP | |
| | | | EVKFNWYVDGVEVHNAKTKPREEQYNSTYRVVSVLTVL | |
| | | | HQDWLNGKEYKCKVSNKALPAPIEKTISKAKGQPREPQ | |
| | | | VYVYPPSRDELTKNQVSLTCLVKGFYPSDIAVEWESNGQ | |
| | | | PENNYKTTPPVLDSDGSFALVSKLTVDKSRWQQGNVFS | |
| | | | CSVMHEALHNHYTQKSLSLSPG | |
| 93 | 16711 | Full | GAGCTGGTGCTGACACAGTCCCCTTCTGTGAGCGCCG | |
| | | | CCCTGGGCTCCCCAGCCAAGATCACCTGCACACTGAG | |
| | | | CTCCGCCCACAAGACCGACACAATCGATTGGTACCAG | |
| | | | CAGCTGCAGGGAGAGGCACCCAGATATCTGATGCAG | |
| | | | GTGCAGTCTGACGGCAGCTACACCAAGCGGCCCGGA | |
| | | | GTGCCTGACAGATTCTCCGGCTCTAGCTCCGGAGCCG | |
| | | | ATCGCTATCTGATCATCCCATCTGTGCAGGCCGACGA | |
| | | | TGAGGCCGACTACTATTGCGGAGCCGATTACATCGGA | |
| | | | GGATACGTGTTCGGAGGAGGAACCCAGCTGACCGTG | |
| | | | ACAGTGGAGGGAGGCTCCGGAGGCTCTGGAGGCAG | |
| | | | CGGCGGCTCCGGCGGCGTGGACCAGGAGCAGCTGGT | |
| | | | GGAGAGCGGCGGCAGACTGGTGACCCCAGGAGGCT | |
| | | | CCCTGACACTGTCTTGTAAGGCCAGCGGCTTCGATTTT | |
| | | | TCCGCCTACTATATGTCTTGGGTGAGACAGGCACCAG | |
| | | | GCAAGGGCCTGGAGTGGATCGCCACCATCTACCCCTC | |
| | | | TAGCGGCAAGACCTACTATGCCACATGGGTGAACGG | |
| | | | CAGATTCACCATCTCCTCTGACAACGCCCAGAATACA | |
| | | | GTGGATCTGCAGATGAATAGCCTGACCGCCGCCGAC | |
| | | | AGGGCCACATACTTCTGCGCCCGCGATTCCTATGCCG | |
| | | | ACGATGGGGCCCTGTTCAACATCTGGGGCCCTGGCAC | |
| | | | CCTGGTGACAATCAGCTCCGCCGCCGAGCCAAAGTCT | |
| | | | AGCGACAAGACCCACACATGCCCACCTTGTCCGGCGC | |
| | | | CAGAGGCTGCAGGAGGACCAAGCGTGTTCCTGTTTCC | |
| | | | ACCCAAGCCTAAAGACACACTGATGATTTCCCGAACC | |
| | | | CCCGAAGTCACATGCGTGGTCGTGTCTGTGAGTCACG | |
| | | | AGGACCCTGAAGTCAAGTTCAACTGGTACGTGGATG | |
| | | | GCGTCGAGGTGCATAATGCCAAGACTAAACCTAGGG | |
| | | | AGGAACAGTACAACTCAACCTATCGCGTCGTGAGCGT | |
| | | | CCTGACAGTGCTGCACCAGGATTGGCTGAACGGCAA | |
| | | | AGAATATAAGTGCAAAGTGAGCAATAAGGCCCTGCC | |
| | | | CGCTCCTATCGAGAAAACCATTTCCAAGGCTAAAGGG | |
| | | | CAGCCTCGCGAACCACAGGTCTACGTGTATCCTCCAA | |
| | | | GCCGGGACGAGCTGACAAAGAACCAGGTCTCCCTGA | |
| | | | CTTGTCTGGTGAAAGGGTTTTACCCTAGTGATATCGC | |
| | | | TGTGGAGTGGGAATCAAATGGACAGCCAGAGAACAA | |
| | | | TTATAAGACTACCCCCCCTGTGCTGGACAGTGATGGG | |
| | | | TCATTCGCACTGGTCTCCAAGCTGACAGTGGACAAAT | |
| | | | CTCGGTGGCAGCAGGGAAATGTCTTTTCATGTAGCGT | |
| | | | GATGCATGAAGCACTGCACAACCATTACACCCAGAAG | |
| | | | TCACTGTCACTGTCACCAGGA | |
| 94 | 16712 | Full | QVQLVQSGAEVKKPGASVKVSCKASGYSFTGYTMNWV | VH = Q1- |
| | | | RQAPGQGLEWMGLITPYNGASSYNQKFRGKATMTVD | S119; |
| | | | TSTSTVYMELSSLRSEDTAVYYCARGGYDGRGFDYWGQ | VL = D135- |
| | | | GTLVTVSSGGGGSGGGGSGGGGSDIQMTQSPSSLSAS | K240 |
| | | | VGDRVTITCSASSSVSYMHWYQQKSGKAPKLLIYDTSKL | |
| | | | ASGVPSRFSGSGSGTDFTLTISSLQPEDFATYYCQQWSK | |
| | | | HPLTFGQGTKLEIKAAEPKSSDKTHTCPPCPAPEAAGGP | |
| | | | SVFLFPPKPKDTLMISRTPEVTCVVVSVSHEDPEVKFNW | |
| | | | YVDGVEVHNAKTKPREEQYNSTYRVVSVLTVLHQDWL | |
| | | | NGKEYKCKVSNKALPAPIEKTISKAKGQPREPQVYVYPPS | |
| | | | RDELTKNQVSLTCLVKGFYPSDIAVEWESNGQPENNYK | |
| | | | TTPPVLDSDGSFALVSKLTVDKSRWQQGNVFSCSVMH | |
| | | | EALHNHYTQKSLSLSPG | |
| 95 | 16712 | Full | CAGGTGCAGCTGGTGCAGAGCGGAGCCGAGGTGAA | |
| | | | GAAGCCTGGGGCCAGCGTGAAGGTGTCCTGCAAGGC | |
| | | | CTCCGGCTACTCTTTCACAGGCTATACCATGAACTGG | |
| | | | GTGCGGCAGGCCCCAGGACAGGGCCTGGAGTGGAT | |
| | | | GGGCCTGATCACACCCTACAACGGGGCCAGCTCCTAT | |
| | | | AATCAGAAGTTTCGGGGCAAGGCCACCATGACAGTG | |
| | | | GACACCAGCACATCCACCGTGTACATGGAGCTGTCTA | |
| | | | GCCTGAGATCCGAGGATACCGCCGTGTACTATTGCGC | |
| | | | CAGAGGCGGATACGACGGCAGAGGCTTTGATTATTG | |
| | | | GGGCCAGGGCACACTGGTGACCGTGTCCTCTGGCGG | |
| | | | CGGCGGCTCTGGAGGAGGAGGCAGCGGCGGAGGAG | |
| | | | GCTCCGACATCCAGATGACACAGTCCCCAAGCTCCCT | |
| | | | GTCTGCCAGCGTGGGCGATAGGGTGACAATCACCTG | |
| | | | TTCTGCCTCTAGCTCCGTGAGCTACATGCACTGGTATC | |
| | | | AGCAGAAGTCTGGCAAGGCCCCTAAGCTGCTGATCTA | |
| | | | TGACACCTCTAAGCTGGCCAGCGGAGTGCCATCCCGC | |
| | | | TTCTCCGGCTCTGGCAGCGGAACAGACTTTACACTGA | |
| | | | CCATCTCTAGCCTGCAGCCCGAGGATTTCGCCACCTAC | |
| | | | TATTGTCAGCAGTGGAGCAAGCACCCTCTGACATTTG | |
| | | | GCCAGGGCACCAAGCTGGAGATCAAGGCCGCCGAGC | |
| | | | CCAAGTCCTCTGATAAGACACACACCTGCCCCCCTTGT | |
| | | | CCGGCGCCAGAGGCTGCAGGAGGACCAAGCGTGTTC | |
| | | | CTGTTTCCACCCAAGCCTAAAGACACACTGATGATTTC | |
| | | | CCGAACCCCCGAAGTCACATGCGTGGTCGTGTCTGTG | |
| | | | AGTCACGAGGACCCTGAAGTCAAGTTCAACTGGTACG | |
| | | | TGGATGGCGTCGAGGTGCATAATGCCAAGACTAAAC | |
| | | | CTAGGGAGGAACAGTACAACTCAACCTATCGCGTCGT | |
| | | | GAGCGTCCTGACAGTGCTGCACCAGGATTGGCTGAA | |
| | | | CGGCAAAGAATATAAGTGCAAAGTGAGCAATAAGGC | |
| | | | CCTGCCCGCTCCTATCGAGAAAACCATTTCCAAGGCT | |
| | | | AAAGGGCAGCCTCGCGAACCACAGGTCTACGTGTATC | |
| | | | CTCCAAGCCGGGACGAGCTGACAAAGAACCAGGTCT | |
| | | | CCCTGACTTGTCTGGTGAAAGGGTTTTACCCTAGTGA | |
| | | | TATCGCTGTGGAGTGGGAATCAAATGGACAGCCAGA | |
| | | | GAACAATTATAAGACTACCCCCCCTGTGCTGGACAGT | |
| | | | GATGGGTCATTCGCACTGGTCTCCAAGCTGACAGTGG | |
| | | | ACAAATCTCGGTGGCAGCAGGGAAATGTCTTTTCATG | |
| | | | TAGCGTGATGCATGAAGCACTGCACAACCATTACACC | |
| | | | CAGAAGTCACTGTCACTGTCACCAGGA | |
| 96 | 16713 | Full | EVQLVESGGGLVQPGGSLRLSCAASGFNIKDTYIHWVR | VH = E1- |
| | | | QAPGKGLEWVARIYPTNGYTRYADSVKGRFTISADTSK | S120; |
| | | | NTAYLQMNSLRAEDTAVYYCSRWGGDGFYAMDYWG | CH1 = |
| | | | QGTLVTVSSASTKGPSVFPLAPSSKSTSGGTAALGCLVK | A121-V218 |
| | | | DYFPEPVTVSWNSGALTSGVHTFPAVLQSSGLYSLSSVV | |
| | | | TVPSSSLGTQTYICNVNHKPSNTKVDKKVEPKSCDKTHT | |
| | | | CPPCPAPEAAGGPSVFLFPPKPKDTLMISRTPEVTCVVV | |
| | | | SVSHEDPEVKFNWYVDGVEVHNAKTKPREEQYNSTYR | |
| | | | VVSVLTVLHQDWLNGKEYKCKVSNKALPAPIEKTISKAK | |
| | | | GQPREPQVYVYPPSRDELTKNQVSLTCLVKGFYPSDIAV | |
| | | | EWESNGQPENNYKTTPPVLDSDGSFALVSKLTVDKSRW | |
| | | | QQGNVFSCSVMHEALHNHYTQKSLSLSPG | |
| 97 | 16713 | Full | GAGGTGCAGCTGGTGGAGAGCGGCGGCGGCCTGGT | |
| | | | GCAGCCCGGCGGCTCTCTGCGGCTGAGCTGCGCCGC | |
| | | | CTCCGGCTTTAACATCAAGGACACATACATCCACTGG | |
| | | | GTGCGGCAGGCCCCCGGCAAGGGCCTGGAGTGGGT | |
| | | | GGCCAGAATCTATCCTACCAATGGCTACACACGGTAT | |
| | | | GCCGACTCCGTGAAGGGCAGATTCACCATCTCTGCCG | |
| | | | ATACCAGCAAGAACACAGCCTACCTGCAGATGAACAG | |
| | | | CCTGCGGGCCGAGGATACAGCCGTGTACTATTGTTCT | |
| | | | CGCTGGGGCGGCGACGGCTTTTACGCCATGGATTATT | |
| | | | GGGGCCAGGGCACCCTGGTGACAGTGAGCTCCGCTA | |
| | | | GCACTAAGGGGCCTTCCGTGTTTCCACTGGCTCCCTCT | |
| | | | AGTAAATCCACCTCTGGAGGCACAGCTGCACTGGGAT | |
| | | | GTCTGGTGAAGGATTACTTCCCTGAACCAGTCACAGT | |
| | | | GAGTTGGAACTCAGGGGCTCTGACAAGTGGAGTCCA | |
| | | | TACTTTTCCCGCAGTGCTGCAGTCAAGCGGACTGTAC | |
| | | | TCCCTGTCCTCTGTGGTCACCGTGCCTAGTTCAAGCCT | |
| | | | GGGCACCCAGACATATATCTGCAACGTGAATCACAAG | |
| | | | CCATCAAATACAAAAGTCGACAAGAAAGTGGAGCCC | |
| | | | AAGAGCTGTGATAAAACTCATACCTGCCCACCTTGTC | |
| | | | CGGCGCCAGAGGCTGCAGGAGGACCAAGCGTGTTCC | |
| | | | TGTTTCCACCCAAGCCTAAAGACACACTGATGATTTCC | |
| | | | CGAACCCCCGAAGTCACATGCGTGGTCGTGTCTGTGA | |
| | | | GTCACGAGGACCCTGAAGTCAAGTTCAACTGGTACGT | |
| | | | GGATGGCGTCGAGGTGCATAATGCCAAGACTAAACC | |
| | | | TAGGGAGGAACAGTACAACTCAACCTATCGCGTCGTG | |
| | | | AGCGTCCTGACAGTGCTGCACCAGGATTGGCTGAAC | |
| | | | GGCAAAGAATATAAGTGCAAAGTGAGCAATAAGGCC | |
| | | | CTGCCCGCTCCTATCGAGAAAACCATTTCCAAGGCTA | |
| | | | AAGGGCAGCCTCGCGAACCACAGGTCTACGTCTACCC | |
| | | | CCCATCAAGAGATGAACTGACAAAAAATCAGGTCTCT | |
| | | | CTGACATGCCTGGTCAAAGGATTCTACCCTTCCGACAT | |
| | | | CGCCGTGGAGTGGGAAAGTAACGGCCAGCCCGAGAA | |
| | | | CAATTACAAGACCACACCCCCTGTCCTGGACTCTGAT | |
| | | | GGGAGTTTCGCTCTGGTGTCAAAGCTGACCGTCGATA | |
| | | | AAAGCCGGTGGCAGCAGGGCAATGTGTTTAGCTGCT | |
| | | | CCGTCATGCACGAAGCCCTGCACAATCACTACACACA | |
| | | | GAAGTCCCTGAGCCTGAGCCCTGGC | |
| 98 | 16714 | Full | QVQLQQSGAELARPGASVKMSCKASGYTFTTYTMHW | VH = Q1- |
| | | | VKQRPGQGLEWIGYINPSSGYTNYNQKFKDKATLTADK | S121; |
| | | | SSSTASMQLSSLTSEDSAVYYCARERAVLVPYAMDYWG | VL = |
| | | | QGTSVTVSSGGGGSGGGGSGGGGSGGGGSQIVLTQSP | Q142-K247; |
| | | | AVMSASPGEKVTITCTASSSLSYMHWFQQKPGTSPKL | VH = E253- |
| | | | WLYSTSILASGVPTRFSGSGSGTSYSLTISRMEAEDAATY | S372; |
| | | | YCQQRSSSPFTFGSGTKLEIKGGGGSEVQLVESGGGLVQ | CH1 = |
| | | | PGGSLRLSCAASGFNIKDTYIHWVRQAPGKGLEWVARI | A373-V470 |
| | | | YPTNGYTRYADSVKGRFTISADTSKNTAYLQMNSLRAED | |
| | | | TAVYYCSRWGGDGFYAMDYWGQGTLVTVSSASTKGP | |
| | | | SVFPLAPSSKSTSGGTAALGCLVKDYFPEPVTVSWNSGA | |
| | | | LTSGVHTFPAVLQSSGLYSLSSVVTVPSSSLGTQTYICNV | |
| | | | NHKPSNTKVDKKVEPKSCDKTHTCPPCPAPEAAGGPSV | |
| | | | FLFPPKPKDTLMISRTPEVTCVVVSVSHEDPEVKFNWYV | |
| | | | DGVEVHNAKTKPREEQYNSTYRVVSVLTVLHQDWLNG | |
| | | | KEYKCKVSNKALPAPIEKTISKAKGQPREPQVYVYPPSRD | |
| | | | ELTKNQVSLTCLVKGFYPSDIAVEWESNGQPENNYKTTP | |
| | | | PVLDSDGSFALVSKLTVDKSRWQQGNVFSCSVMHEAL | |
| | | | HNHYTQKSLSLSPG | |
| 99 | 16714 | Full | CAGGTGCAGCTGCAGCAGAGCGGAGCCGAGCTGGCC | |
| | | | AGACCTGGGGCCAGCGTGAAGATGTCTTGCAAGGCC | |
| | | | AGCGGCTACACATTCACCACATATACCATGCACTGGG | |
| | | | TGAAGCAGAGACCTGGCCAGGGCCTGGAGTGGATCG | |
| | | | GCTACATCAACCCAAGCTCCGGCTACACCAACTATAA | |
| | | | TCAGAAGTTTAAGGACAAGGCCACCCTGACAGCCGAT | |
| | | | AAGTCTAGCTCCACAGCCTCCATGCAGCTGTCTAGCCT | |
| | | | GACCTCTGAGGACAGCGCCGTGTACTATTGCGCCCGG | |
| | | | GAGAGAGCCGTGCTGGTGCCTTACGCCATGGATTATT | |
| | | | GGGGCCAGGGCACAAGCGTGACCGTGTCCTCTGGAG | |
| | | | GAGGAGGCAGCGGCGGAGGAGGCTCCGGAGGCGGC | |
| | | | GGCTCTGGCGGCGGCGGCAGCCAGATCGTGCTGACC | |
| | | | CAGTCCCCAGCCGTGATGTCTGCCAGCCCAGGAGAG | |
| | | | AAGGTGACCATCACATGTACCGCCAGCTCCTCTCTGA | |
| | | | GCTACATGCACTGGTTCCAGCAGAAGCCCGGCACATC | |
| | | | CCCTAAGCTGTGGCTGTATTCCACCTCTATCCTGGCCT | |
| | | | CCGGCGTGCCCACAAGGTTTAGCGGCTCCGGCTCTGG | |
| | | | CACAAGCTACTCCCTGACCATCTCTAGGATGGAGGCC | |
| | | | GAGGACGCCGCCACCTACTATTGCCAGCAGCGCAGCT | |
| | | | CCTCTCCATTCACATTTGGCAGCGGCACCAAGCTGGA | |
| | | | GATCAAGGGAGGAGGAGGCTCCGAGGTGCAGCTGG | |
| | | | TGGAGTCTGGAGGAGGACTGGTGCAGCCAGGAGGCT | |
| | | | CCCTGCGGCTGTCTTGTGCCGCCAGCGGCTTTAACAT | |
| | | | CAAGGACACATACATCCACTGGGTGAGGCAGGCCCC | |
| | | | CGGCAAGGGACTGGAGTGGGTGGCCCGCATCTATCC | |
| | | | TACAAATGGCTACACCAGATATGCCGACTCCGTGAAG | |
| | | | GGCCGCTTCACCATCTCCGCCGATACATCTAAGAACA | |
| | | | CCGCCTACCTGCAGATGAACAGCCTGCGGGCCGAGG | |
| | | | ATACAGCCGTGTACTATTGTAGCAGATGGGGCGGCG | |
| | | | ACGGCTTTTACGCTATGGACTACTGGGGACAGGGCAC | |
| | | | ACTGGTGACCGTGAGCTCCGCTAGCACTAAGGGGCCT | |
| | | | TCCGTGTTTCCACTGGCTCCCTCTAGTAAATCCACCTC | |
| | | | TGGAGGCACAGCTGCACTGGGATGTCTGGTGAAGGA | |
| | | | TTACTTCCCTGAACCAGTCACAGTGAGTTGGAACTCA | |
| | | | GGGGCTCTGACAAGTGGAGTCCATACTTTTCCCGCAG | |
| | | | TGCTGCAGTCAAGCGGACTGTACTCCCTGTCCTCTGT | |
| | | | GGTCACCGTGCCTAGTTCAAGCCTGGGCACCCAGACA | |
| | | | TATATCTGCAACGTGAATCACAAGCCATCAAATACAA | |
| | | | AAGTCGACAAGAAAGTGGAGCCCAAGAGCTGTGATA | |
| | | | AAACTCATACCTGCCCACCTTGTCCGGCGCCAGAGGC | |
| | | | TGCAGGAGGACCAAGCGTGTTCCTGTTTCCACCCAAG | |
| | | | CCTAAAGACACACTGATGATTTCCCGAACCCCCGAAG | |
| | | | TCACATGCGTGGTCGTGTCTGTGAGTCACGAGGACCC | |
| | | | TGAAGTCAAGTTCAACTGGTACGTGGATGGCGTCGA | |
| | | | GGTGCATAATGCCAAGACTAAACCTAGGGAGGAACA | |
| | | | GTACAACTCAACCTATCGCGTCGTGAGCGTCCTGACA | |
| | | | GTGCTGCACCAGGATTGGCTGAACGGCAAAGAATAT | |
| | | | AAGTGCAAAGTGAGCAATAAGGCCCTGCCCGCTCCTA | |
| | | | TCGAGAAAACCATTTCCAAGGCTAAAGGGCAGCCTCG | |
| | | | CGAACCACAGGTCTACGTCTACCCCCCATCAAGAGAT | |
| | | | GAACTGACAAAAAATCAGGTCTCTCTGACATGCCTGG | |
| | | | TCAAAGGATTCTACCCTTCCGACATCGCCGTGGAGTG | |
| | | | GGAAAGTAACGGCCAGCCCGAGAACAATTACAAGAC | |
| | | | CACACCCCCTGTCCTGGACTCTGATGGGAGTTTCGCTC | |
| | | | TGGTGTCAAAGCTGACCGTCGATAAAAGCCGGTGGC | |
| | | | AGCAGGGCAATGTGTTTAGCTGCTCCGTCATGCACGA | |
| | | | AGCCCTGCACAATCACTACACACAGAAGTCCCTGAGC | |
| | | | CTGAGCCCTGGC | |
| 100 | 16716 | Full | QVQLQQSGAELARPGASVKMSCKASGYTFTTYTMHW | VH = Q1- |
| | | | VKQRPGQGLEWIGYINPSSGYTNYNQKFKDKATLTADK | S121; |
| | | | SSSTASMQLSSLTSEDSAVYYCARERAVLVPYAMDYWG | VL = Q142- |
| | | | QGTSVTVSSGGGGSGGGGSGGGGSGGGGSQIVLTQSP | K247; |
| | | | AVMSASPGEKVTITCTASSSLSYMHWFQQKPGTSPKL | VH = Q253- |
| | | | WLYSTSILASGVPTRFSGSGSGTSYSLTISRMEAEDAATY | S371; |
| | | | YCQQRSSSPFTFGSGTKLEIKGGGGSQVQLVQSGAEVK | CH1 = |
| | | | KPGASVKVSCKASGYSFTGYTMNWVRQAPGQGLEWM | A372-V469 |
| | | | GLITPYNGASSYNQKFRGKATMTVDTSTSTVYMELSSLR | |
| | | | SEDTAVYYCARGGYDGRGFDYWGQGTLVTVSSASTKG | |
| | | | PSVFPLAPSSKSTSGGTAALGCLVKDYFPEPVTVSWNSG | |
| | | | ALTSGVHTFPAVLQSSGLYSLSSVVTVPSSSLGTQTYICN | |
| | | | VNHKPSNTKVDKKVEPKSCDKTHTCPPCPAPEAAGGPS | |
| | | | VFLFPPKPKDTLMISRTPEVTCVVVSVSHEDPEVKFNWY | |
| | | | VDGVEVHNAKTKPREEQYNSTYRVVSVLTVLHQDWLN | |
| | | | GKEYKCKVSNKALPAPIEKTISKAKGQPREPQVYVYPPSR | |
| | | | DELTKNQVSLTCLVKGFYPSDIAVEWESNGQPENNYKT | |
| | | | TPPVLDSDGSFALVSKLTVDKSRWQQGNVFSCSVMHE | |
| | | | ALHNHYTQKSLSLSPG | |
| 101 | 16716 | Full | CAGGTGCAGCTGCAGCAGTCCGGAGCCGAGCTGGCC | |
| | | | AGACCTGGGGCCAGCGTGAAGATGTCCTGCAAGGCC | |
| | | | TCTGGCTACACCTTCACCACATATACAATGCACTGGGT | |
| | | | GAAGCAGCGCCCTGGACAGGGACTGGAGTGGATCG | |
| | | | GCTACATCAACCCAAGCTCCGGCTACACCAACTATAA | |
| | | | TCAGAAGTTTAAGGACAAGGCCACCCTGACAGCCGAT | |
| | | | AAGTCTAGCTCCACCGCCAGCATGCAGCTGTCTAGCC | |
| | | | TGACATCTGAGGACAGCGCCGTGTACTATTGCGCCCG | |
| | | | GGAGAGAGCCGTGCTGGTGCCTTACGCCATGGATTAT | |
| | | | TGGGGCCAGGGCACCTCCGTGACAGTGTCCTCTGGA | |
| | | | GGAGGAGGCTCTGGAGGAGGAGGCAGCGGCGGAG | |
| | | | GAGGCTCCGGCGGCGGCGGCTCTCAGATCGTGCTGA | |
| | | | CCCAGAGCCCAGCCGTGATGAGCGCCTCCCCAGGAG | |
| | | | AGAAGGTGACCATCACATGTACCGCCAGCTCCTCTCT | |
| | | | GTCTTACATGCACTGGTTCCAGCAGAAGCCCGGCACC | |
| | | | AGCCCTAAGCTGTGGCTGTATTCTACAAGCATCCTGG | |
| | | | CCTCCGGAGTGCCAACCCGGTTTTCCGGCTCTGGCAG | |
| | | | CGGCACCTCCTACTCTCTGACAATCTCTAGGATGGAG | |
| | | | GCCGAGGACGCCGCCACCTACTATTGCCAGCAGCGCA | |
| | | | GCTCCTCTCCATTCACCTTTGGCTCCGGCACAAAGCTG | |
| | | | GAGATCAAGGGAGGAGGAGGCAGCCAGGTGCAGCT | |
| | | | GGTGCAGTCCGGAGCCGAGGTGAAGAAGCCAGGGG | |
| | | | CCAGCGTGAAGGTGTCCTGTAAGGCCTCCGGCTACTC | |
| | | | TTTCACCGGCTATACAATGAATTGGGTGAGACAGGCC | |
| | | | CCCGGCCAGGGCCTGGAGTGGATGGGCCTGATCACA | |
| | | | CCTTACAACGGGGCCAGCTCCTATAATCAGAAGTTTC | |
| | | | GGGGCAAGGCCACAATGACCGTGGACACAAGCACCT | |
| | | | CCACAGTGTACATGGAGCTGTCTAGCCTGAGAAGCG | |
| | | | AGGATACCGCCGTGTACTATTGTGCCAGGGGCGGAT | |
| | | | ACGACGGCAGAGGCTTTGACTACTGGGGCCAGGGCA | |
| | | | CCCTGGTGACAGTGTCCTCTGCTAGCACTAAGGGGCC | |
| | | | TTCCGTGTTTCCACTGGCTCCCTCTAGTAAATCCACCT | |
| | | | CTGGAGGCACAGCTGCACTGGGATGTCTGGTGAAGG | |
| | | | ATTACTTCCCTGAACCAGTCACAGTGAGTTGGAACTC | |
| | | | AGGGGCTCTGACAAGTGGAGTCCATACTTTTCCCGCA | |
| | | | GTGCTGCAGTCAAGCGGACTGTACTCCCTGTCCTCTG | |
| | | | TGGTCACCGTGCCTAGTTCAAGCCTGGGCACCCAGAC | |
| | | | ATATATCTGCAACGTGAATCACAAGCCATCAAATACA | |
| | | | AAAGTCGACAAGAAAGTGGAGCCCAAGAGCTGTGAT | |
| | | | AAAACTCATACCTGCCCACCTTGTCCGGCGCCAGAGG | |
| | | | CTGCAGGAGGACCAAGCGTGTTCCTGTTTCCACCCAA | |
| | | | GCCTAAAGACACACTGATGATTTCCCGAACCCCCGAA | |
| | | | GTCACATGCGTGGTCGTGTCTGTGAGTCACGAGGACC | |
| | | | CTGAAGTCAAGTTCAACTGGTACGTGGATGGCGTCGA | |
| | | | GGTGCATAATGCCAAGACTAAACCTAGGGAGGAACA | |
| | | | GTACAACTCAACCTATCGCGTCGTGAGCGTCCTGACA | |
| | | | GTGCTGCACCAGGATTGGCTGAACGGCAAAGAATAT | |
| | | | AAGTGCAAAGTGAGCAATAAGGCCCTGCCCGCTCCTA | |
| | | | TCGAGAAAACCATTTCCAAGGCTAAAGGGCAGCCTCG | |
| | | | CGAACCACAGGTCTACGTCTACCCCCCATCAAGAGAT | |
| | | | GAACTGACAAAAAATCAGGTCTCTCTGACATGCCTGG | |
| | | | TCAAAGGATTCTACCCTTCCGACATCGCCGTGGAGTG | |
| | | | GGAAAGTAACGGCCAGCCCGAGAACAATTACAAGAC | |
| | | | CACACCCCCTGTCCTGGACTCTGATGGGAGTTTCGCTC | |
| | | | TGGTGTCAAAGCTGACCGTCGATAAAAGCCGGTGGC | |
| | | | AGCAGGGCAATGTGTTTAGCTGCTCCGTCATGCACGA | |
| | | | AGCCCTGCACAATCACTACACACAGAAGTCCCTGAGC | |
| | | | CTGAGCCCTGGC | |
| 102 | 16717 | Full | QVQLVESGGGVVQPGRSLRLSCAASGFTFSNYGMYWV | VH = Q1- |
| | | | RQAPGKGLEWVAVIWYDGSNKYYADSVKGRFTISRDN | S118; |
| | | | SKNTLYLQMNSLRAEDTAVYYCARDLWGWYFDYWGQ | VL = E139- |
| | | | GTLVTVSSGGGGSGGGGSGGGGSGGGGSEIVLTQSPA | K245; |
| | | | TLSLSPGERATLSCRASQSVSSYLAWYQQKPGQAPRLLIY | VH = E251- |
| | | | DASNRATGIPARFSGSGSGTDFTLTISSLEPEDFAVYYCQ | S370; |
| | | | QRRNWPLTFGGGTKVEIKGGGGSEVQLVESGGGLVQP | CH1 = |
| | | | GGSLRLSCAASGFNIKDTYIHWVRQAPGKGLEWVARIY | A371-V468 |
| | | | PTNGYTRYADSVKGRFTISADTSKNTAYLQMNSLRAEDT | |
| | | | AVYYCSRWGGDGFYAMDYWGQGTLVTVSSASTKGPS | |
| | | | VFPLAPSSKSTSGGTAALGCLVKDYFPEPVTVSWNSGAL | |
| | | | TSGVHTFPAVLQSSGLYSLSSVVTVPSSSLGTQTYICNVN | |
| | | | HKPSNTKVDKKVEPKSCDKTHTCPPCPAPEAAGGPSVFL | |
| | | | FPPKPKDTLMISRTPEVTCVVVSVSHEDPEVKFNWYVD | |
| | | | GVEVHNAKTKPREEQYNSTYRVVSVLTVLHQDWLNGK | |
| | | | EYKCKVSNKALPAPIEKTISKAKGQPREPQVYVYPPSRDE | |
| | | | LTKNQVSLTCLVKGFYPSDIAVEWESNGQPENNYKTTPP | |
| | | | VLDSDGSFALVSKLTVDKSRWQQGNVFSCSVMHEALH | |
| | | | NHYTQKSLSLSPG | |
| 103 | 16717 | Full | CAGGTGCAGCTGGTGGAGTCCGGCGGCGGCGTGGTG | |
| | | | CAGCCTGGCAGGAGCCTGCGCCTGTCCTGCGCAGCCT | |
| | | | CTGGCTTCACCTTCAGCAACTACGGCATGTATTGGGT | |
| | | | GAGACAGGCCCCTGGCAAGGGACTGGAGTGGGTGG | |
| | | | CCGTGATCTGGTACGACGGCTCTAATAAGTACTATGC | |
| | | | CGATAGCGTGAAGGGCCGGTTCACCATCAGCAGAGA | |
| | | | CAACTCCAAGAATACACTGTATCTGCAGATGAACTCC | |
| | | | CTGCGGGCCGAGGATACCGCCGTGTACTATTGCGCCA | |
| | | | GAGACCTGTGGGGCTGGTACTTTGATTATTGGGGCCA | |
| | | | GGGCACCCTGGTGACAGTGAGCAGCGGAGGAGGAG | |
| | | | GCTCCGGCGGCGGAGGCTCTGGCGGCGGCGGCAGC | |
| | | | GGAGGCGGCGGCTCCGAGATCGTGCTGACCCAGTCT | |
| | | | CCAGCCACACTGTCTCTGAGCCCAGGAGAGAGGGCC | |
| | | | ACCCTGAGCTGTCGCGCCTCCCAGAGCGTGAGCAGCT | |
| | | | ACCTGGCCTGGTATCAGCAGAAGCCAGGACAGGCCC | |
| | | | CTCGGCTGCTGATCTACGACGCCAGCAACAGGGCAAC | |
| | | | CGGCATCCCAGCCAGATTCAGCGGCTCCGGCTCTGGC | |
| | | | ACAGACTTTACCCTGACAATCTCCTCTCTGGAGCCCGA | |
| | | | GGATTTCGCCGTGTACTATTGCCAGCAGCGGAGAAAT | |
| | | | TGGCCTCTGACCTTTGGCGGCGGCACAAAGGTGGAG | |
| | | | ATCAAGGGAGGAGGAGGCTCCGAAGTCCAGCTGGTG | |
| | | | GAGTCTGGAGGAGGACTGGTGCAGCCAGGAGGCTCT | |
| | | | CTGCGGCTGAGCTGTGCCGCCTCCGGCTTTAACATCA | |
| | | | AGGACACCTACATCCACTGGGTGCGGCAGGCCCCTG | |
| | | | GCAAGGGCCTGGAGTGGGTGGCCAGAATCTATCCAA | |
| | | | CCAATGGCTACACAAGATATGCCGACTCCGTGAAGG | |
| | | | GCCGCTTCACCATCTCTGCCGATACCAGCAAGAACAC | |
| | | | AGCCTACCTGCAGATGAATAGCCTGAGGGCCGAGGA | |
| | | | TACAGCCGTGTACTATTGTTCCCGCTGGGGAGGCGAC | |
| | | | GGCTTTTACGCAATGGACTACTGGGGACAGGGCACC | |
| | | | CTGGTCACAGTGAGCTCCGCTAGCACTAAGGGGCCTT | |
| | | | CCGTGTTTCCACTGGCTCCCTCTAGTAAATCCACCTCT | |
| | | | GGAGGCACAGCTGCACTGGGATGTCTGGTGAAGGAT | |
| | | | TACTTCCCTGAACCAGTCACAGTGAGTTGGAACTCAG | |
| | | | GGGCTCTGACAAGTGGAGTCCATACTTTTCCCGCAGT | |
| | | | GCTGCAGTCAAGCGGACTGTACTCCCTGTCCTCTGTG | |
| | | | GTCACCGTGCCTAGTTCAAGCCTGGGCACCCAGACAT | |
| | | | ATATCTGCAACGTGAATCACAAGCCATCAAATACAAA | |
| | | | AGTCGACAAGAAAGTGGAGCCCAAGAGCTGTGATAA | |
| | | | AACTCATACCTGCCCACCTTGTCCGGCGCCAGAGGCT | |
| | | | GCAGGAGGACCAAGCGTGTTCCTGTTTCCACCCAAGC | |
| | | | CTAAAGACACACTGATGATTTCCCGAACCCCCGAAGT | |
| | | | CACATGCGTGGTCGTGTCTGTGAGTCACGAGGACCCT | |
| | | | GAAGTCAAGTTCAACTGGTACGTGGATGGCGTCGAG | |
| | | | GTGCATAATGCCAAGACTAAACCTAGGGAGGAACAG | |
| | | | TACAACTCAACCTATCGCGTCGTGAGCGTCCTGACAG | |
| | | | TGCTGCACCAGGATTGGCTGAACGGCAAAGAATATA | |
| | | | AGTGCAAAGTGAGCAATAAGGCCCTGCCCGCTCCTAT | |
| | | | CGAGAAAACCATTTCCAAGGCTAAAGGGCAGCCTCG | |
| | | | CGAACCACAGGTCTACGTCTACCCCCCATCAAGAGAT | |
| | | | GAACTGACAAAAAATCAGGTCTCTCTGACATGCCTGG | |
| | | | TCAAAGGATTCTACCCTTCCGACATCGCCGTGGAGTG | |
| | | | GGAAAGTAACGGCCAGCCCGAGAACAATTACAAGAC | |
| | | | CACACCCCCTGTCCTGGACTCTGATGGGAGTTTCGCTC | |
| | | | TGGTGTCAAAGCTGACCGTCGATAAAAGCCGGTGGC | |
| | | | AGCAGGGCAATGTGTTTAGCTGCTCCGTCATGCACGA | |
| | | | AGCCCTGCACAATCACTACACACAGAAGTCCCTGAGC | |
| | | | CTGAGCCCTGGC | |
| 104 | 16719 | Full | QVQLVESGGGVVQPGRSLRLSCAASGFTFSNYGMYWV | VH = Q1- |
| | | | RQAPGKGLEWVAVIWYDGSNKYYADSVKGRFTISRDN | S118; |
| | | | SKNTLYLQMNSLRAEDTAVYYCARDLWGWYFDYWGQ | VL = E139- |
| | | | GTLVTVSSGGGGSGGGGSGGGGSGGGGSEIVLTQSPA | K245; |
| | | | TLSLSPGERATLSCRASQSVSSYLAWYQQKPGQAPRLLIY | VH = Q251- |
| | | | DASNRATGIPARFSGSGSGTDFTLTISSLEPEDFAVYYCQ | S369; |
| | | | QRRNWPLTFGGGTKVEIKGGGGSQVQLVQSGAEVKKP | CH1 = |
| | | | GASVKVSCKASGYSFTGYTMNWVRQAPGQGLEWMGL | A370-V467 |
| | | | ITPYNGASSYNQKFRGKATMTVDTSTSTVYMELSSLRSE | |
| | | | DTAVYYCARGGYDGRGFDYWGQGTLVTVSSASTKGPS | |
| | | | VFPLAPSSKSTSGGTAALGCLVKDYFPEPVTVSWNSGAL | |
| | | | TSGVHTFPAVLQSSGLYSLSSVVTVPSSSLGTQTYICNVN | |
| | | | HKPSNTKVDKKVEPKSCDKTHTCPPCPAPEAAGGPSVFL | |
| | | | FPPKPKDTLMISRTPEVTCVVVSVSHEDPEVKFNWYVD | |
| | | | GVEVHNAKTKPREEQYNSTYRVVSVLTVLHQDWLNGK | |
| | | | EYKCKVSNKALPAPIEKTISKAKGQPREPQVYVYPPSRDE | |
| | | | LTKNQVSLTCLVKGFYPSDIAVEWESNGQPENNYKTTPP | |
| | | | VLDSDGSFALVSKLTVDKSRWQQGNVFSCSVMHEALH | |
| | | | NHYTQKSLSLSPG | |
| 105 | 16719 | Full | CAGGTGCAGCTGGTGGAGAGCGGCGGCGGCGTGGT | |
| | | | GCAGCCTGGCAGGTCTCTGCGCCTGAGCTGCGCAGCC | |
| | | | TCCGGCTTCACCTTTTCCAACTACGGCATGTATTGGGT | |
| | | | GCGGCAGGCCCCTGGCAAGGGACTGGAGTGGGTGG | |
| | | | CCGTGATCTGGTACGACGGCTCCAATAAGTACTATGC | |
| | | | CGATTCTGTGAAGGGCCGGTTCACAATCTCTAGAGAC | |
| | | | AACAGCAAGAATACCCTGTATCTGCAGATGAACAGCC | |
| | | | TGCGGGCCGAGGATACCGCCGTGTACTATTGCGCCA | |
| | | | GAGACCTGTGGGGCTGGTACTTTGATTATTGGGGCCA | |
| | | | GGGCACACTGGTGACCGTGAGCAGCGGAGGAGGAG | |
| | | | GCAGCGGAGGAGGAGGCTCCGGAGGCGGCGGCTCT | |
| | | | GGCGGCGGCGGCAGCGAGATCGTGCTGACACAGTCT | |
| | | | CCAGCCACCCTGAGCCTGTCCCCAGGAGAGAGGGCC | |
| | | | ACCCTGTCCTGTCGCGCCTCTCAGAGCGTGTCTAGCTA | |
| | | | CCTGGCCTGGTATCAGCAGAAGCCAGGACAGGCCCC | |
| | | | CCGGCTGCTGATCTACGACGCCTCCAACAGGGCAACA | |
| | | | GGCATCCCAGCACGCTTCTCCGGCTCTGGCAGCGGCA | |
| | | | CCGACTTTACCCTGACAATCTCCTCTCTGGAGCCCGAG | |
| | | | GATTTCGCCGTGTACTATTGCCAGCAGCGGAGAAATT | |
| | | | GGCCTCTGACATTTGGCGGCGGCACCAAGGTGGAGA | |
| | | | TCAAGGGAGGAGGAGGCAGCCAGGTGCAGCTGGTG | |
| | | | CAGTCCGGAGCCGAGGTGAAGAAGCCAGGGGCCAG | |
| | | | CGTGAAGGTGTCTTGTAAGGCCAGCGGCTACTCCTTC | |
| | | | ACAGGCTATACCATGAATTGGGTGCGCCAGGCCCCTG | |
| | | | GACAGGGACTGGAGTGGATGGGCCTGATCACACCAT | |
| | | | ACAACGGGGCCAGCTCCTATAATCAGAAGTTTCGGG | |
| | | | GCAAGGCCACCATGACAGTGGACACCTCCACATCTAC | |
| | | | CGTGTACATGGAGCTGTCTAGCCTGAGAAGCGAAGA | |
| | | | CACCGCCGTGTACTATTGTGCCAGAGGCGGCTACGAC | |
| | | | GGCAGAGGCTTCGACTACTGGGGACAGGGCACACTG | |
| | | | GTCACCGTGTCCTCTGCTAGCACTAAGGGGCCTTCCG | |
| | | | TGTTTCCACTGGCTCCCTCTAGTAAATCCACCTCTGGA | |
| | | | GGCACAGCTGCACTGGGATGTCTGGTGAAGGATTAC | |
| | | | TTCCCTGAACCAGTCACAGTGAGTTGGAACTCAGGGG | |
| | | | CTCTGACAAGTGGAGTCCATACTTTTCCCGCAGTGCT | |
| | | | GCAGTCAAGCGGACTGTACTCCCTGTCCTCTGTGGTC | |
| | | | ACCGTGCCTAGTTCAAGCCTGGGCACCCAGACATATA | |
| | | | TCTGCAACGTGAATCACAAGCCATCAAATACAAAAGT | |
| | | | CGACAAGAAAGTGGAGCCCAAGAGCTGTGATAAAAC | |
| | | | TCATACCTGCCCACCTTGTCCGGCGCCAGAGGCTGCA | |
| | | | GGAGGACCAAGCGTGTTCCTGTTTCCACCCAAGCCTA | |
| | | | AAGACACACTGATGATTTCCCGAACCCCCGAAGTCAC | |
| | | | ATGCGTGGTCGTGTCTGTGAGTCACGAGGACCCTGAA | |
| | | | GTCAAGTTCAACTGGTACGTGGATGGCGTCGAGGTG | |
| | | | CATAATGCCAAGACTAAACCTAGGGAGGAACAGTAC | |
| | | | AACTCAACCTATCGCGTCGTGAGCGTCCTGACAGTGC | |
| | | | TGCACCAGGATTGGCTGAACGGCAAAGAATATAAGT | |
| | | | GCAAAGTGAGCAATAAGGCCCTGCCCGCTCCTATCGA | |
| | | | GAAAACCATTTCCAAGGCTAAAGGGCAGCCTCGCGA | |
| | | | ACCACAGGTCTACGTCTACCCCCCATCAAGAGATGAA | |
| | | | CTGACAAAAAATCAGGTCTCTCTGACATGCCTGGTCA | |
| | | | AAGGATTCTACCCTTCCGACATCGCCGTGGAGTGGGA | |
| | | | AAGTAACGGCCAGCCCGAGAACAATTACAAGACCAC | |
| | | | ACCCCCTGTCCTGGACTCTGATGGGAGTTTCGCTCTG | |
| | | | GTGTCAAAGCTGACCGTCGATAAAAGCCGGTGGCAG | |
| | | | CAGGGCAATGTGTTTAGCTGCTCCGTCATGCACGAAG | |
| | | | CCCTGCACAATCACTACACACAGAAGTCCCTGAGCCT | |
| | | | GAGCCCTGGC | |
| 106 | 16720 | Full | EVKLVESGGGLVQPGGSLKLSCATSGFTFSDYYMYWVR | VH = E1- |
| | | | QTPEKRLEWVAYINSGGGSTYYPDTVKGRFTISRDNAK | S119; |
| | | | NTLYLQMSRLKSEDTAMYYCARRGLPFHAMDYWGQG | VL = D140- |
| | | | TSVTVSSGGGGSGGGGSGGGGSGGGGSDIQMTQTTSS | K246; |
| | | | LSASLGDRVTISCSASQGISNYLNWYQQKPDGTVKLLIYY | VH = E252- |
| | | | TSILHSGVPSRFSGSGSGTDYSLTIGNLEPEDIATYYCQQF | S371; |
| | | | NKLPPTFGGGTKLEIKGGGGSEVQLVESGGGLVQPGGS | CH1 = |
| | | | LRLSCAASGFNIKDTYIHWVRQAPGKGLEWVARIYPTN | A372-V469 |
| | | | GYTRYADSVKGRFTISADTSKNTAYLQMNSLRAEDTAVY | |
| | | | YCSRWGGDGFYAMDYWGQGTLVTVSSASTKGPSVFPL | |
| | | | APSSKSTSGGTAALGCLVKDYFPEPVTVSWNSGALTSGV | |
| | | | HTFPAVLQSSGLYSLSSVVTVPSSSLGTQTYICNVNHKPS | |
| | | | NTKVDKKVEPKSCDKTHTCPPCPAPEAAGGPSVFLFPPK | |
| | | | PKDTLMISRTPEVTCVVVSVSHEDPEVKFNWYVDGVEV | |
| | | | HNAKTKPREEQYNSTYRVVSVLTVLHQDWLNGKEYKCK | |
| | | | VSNKALPAPIEKTISKAKGQPREPQVYVYPPSRDELTKN | |
| | | | QVSLTCLVKGFYPSDIAVEWESNGQPENNYKTTPPVLDS | |
| | | | DGSFALVSKLTVDKSRWQQGNVFSCSVMHEALHNHYT | |
| | | | QKSLSLSPG | |
| 107 | 16720 | Full | GAGGTGAAGCTGGTGGAGTCCGGAGGAGGACTGGT | |
| | | | GCAGCCAGGAGGCTCTCTGAAGCTGAGCTGCGCCAC | |
| | | | CTCCGGCTTCACATTTTCTGACTACTATATGTACTGGG | |
| | | | TGCGGCAGACCCCCGAGAAGAGACTGGAGTGGGTG | |
| | | | GCCTATATCAACTCTGGCGGCGGCAGCACCTACTATC | |
| | | | CTGACACAGTGAAGGGCAGGTTCACCATCTCCCGCGA | |
| | | | TAACGCCAAGAATACACTGTACCTGCAGATGTCCCGG | |
| | | | CTGAAGTCTGAGGACACAGCCATGTACTATTGCGCCC | |
| | | | GGAGAGGCCTGCCTTTTCACGCCATGGATTATTGGGG | |
| | | | CCAGGGCACCAGCGTGACAGTGAGCAGCGGCGGCG | |
| | | | GCGGCTCTGGAGGAGGAGGCAGCGGCGGAGGAGGC | |
| | | | TCCGGAGGAGGCGGCTCTGACATCCAGATGACCCAG | |
| | | | ACCACATCTAGCCTGAGCGCCTCCCTGGGCGATAGGG | |
| | | | TGACAATCTCTTGTAGCGCCTCCCAGGGCATCTCCAAC | |
| | | | TACCTGAATTGGTATCAGCAGAAGCCTGATGGCACCG | |
| | | | TGAAGCTGCTGATCTACTATACAAGCATCCTGCACTCC | |
| | | | GGCGTGCCATCTCGCTTCTCTGGCAGCGGCTCCGGAA | |
| | | | CCGACTACAGCCTGACAATCGGCAACCTGGAGCCAG | |
| | | | AGGATATCGCCACCTACTATTGCCAGCAGTTCAATAA | |
| | | | GCTGCCCCCTACCTTTGGCGGCGGCACAAAGCTGGAG | |
| | | | ATCAAGGGCGGCGGCGGCAGCGAGGTGCAGCTGGT | |
| | | | CGAAAGCGGCGGCGGCCTGGTCCAGCCTGGAGGCAG | |
| | | | CCTGAGGCTGTCCTGTGCCGCCTCTGGCTTTAACATCA | |
| | | | AGGACACCTACATCCACTGGGTGAGGCAGGCCCCAG | |
| | | | GCAAGGGACTGGAGTGGGTGGCCCGCATCTATCCCA | |
| | | | CCAATGGCTACACAAGATATGCCGACAGCGTGAAGG | |
| | | | GCCGCTTCACCATCAGCGCCGATACCTCCAAGAACAC | |
| | | | AGCCTACCTGCAGATGAACAGCCTGCGGGCCGAGGA | |
| | | | TACAGCCGTGTACTATTGTAGCAGATGGGGCGGCGA | |
| | | | CGGCTTTTACGCTATGGACTACTGGGGACAGGGCACC | |
| | | | CTGGTGACAGTGTCCTCTGCTAGCACTAAGGGGCCTT | |
| | | | CCGTGTTTCCACTGGCTCCCTCTAGTAAATCCACCTCT | |
| | | | GGAGGCACAGCTGCACTGGGATGTCTGGTGAAGGAT | |
| | | | TACTTCCCTGAACCAGTCACAGTGAGTTGGAACTCAG | |
| | | | GGGCTCTGACAAGTGGAGTCCATACTTTTCCCGCAGT | |
| | | | GCTGCAGTCAAGCGGACTGTACTCCCTGTCCTCTGTG | |
| | | | GTCACCGTGCCTAGTTCAAGCCTGGGCACCCAGACAT | |
| | | | ATATCTGCAACGTGAATCACAAGCCATCAAATACAAA | |
| | | | AGTCGACAAGAAAGTGGAGCCCAAGAGCTGTGATAA | |
| | | | AACTCATACCTGCCCACCTTGTCCGGCGCCAGAGGCT | |
| | | | GCAGGAGGACCAAGCGTGTTCCTGTTTCCACCCAAGC | |
| | | | CTAAAGACACACTGATGATTTCCCGAACCCCCGAAGT | |
| | | | CACATGCGTGGTCGTGTCTGTGAGTCACGAGGACCCT | |
| | | | GAAGTCAAGTTCAACTGGTACGTGGATGGCGTCGAG | |
| | | | GTGCATAATGCCAAGACTAAACCTAGGGAGGAACAG | |
| | | | TACAACTCAACCTATCGCGTCGTGAGCGTCCTGACAG | |
| | | | TGCTGCACCAGGATTGGCTGAACGGCAAAGAATATA | |
| | | | AGTGCAAAGTGAGCAATAAGGCCCTGCCCGCTCCTAT | |
| | | | CGAGAAAACCATTTCCAAGGCTAAAGGGCAGCCTCG | |
| | | | CGAACCACAGGTCTACGTCTACCCCCCATCAAGAGAT | |
| | | | GAACTGACAAAAAATCAGGTCTCTCTGACATGCCTGG | |
| | | | TCAAAGGATTCTACCCTTCCGACATCGCCGTGGAGTG | |
| | | | GGAAAGTAACGGCCAGCCCGAGAACAATTACAAGAC | |
| | | | CACACCCCCTGTCCTGGACTCTGATGGGAGTTTCGCTC | |
| | | | TGGTGTCAAAGCTGACCGTCGATAAAAGCCGGTGGC | |
| | | | AGCAGGGCAATGTGTTTAGCTGCTCCGTCATGCACGA | |
| | | | AGCCCTGCACAATCACTACACACAGAAGTCCCTGAGC | |
| | | | CTGAGCCCTGGC | |
| 108 | 16722 | Full | EVKLVESGGGLVQPGGSLKLSCATSGFTFSDYYMYWVR | VH = E1- |
| | | | QTPEKRLEWVAYINSGGGSTYYPDTVKGRFTISRDNAK | S119; |
| | | | NTLYLQMSRLKSEDTAMYYCARRGLPFHAMDYWGQG | VL = D140- |
| | | | TSVTVSSGGGGSGGGGSGGGGSGGGGSDIQMTQTTSS | K246; |
| | | | LSASLGDRVTISCSASQGISNYLNWYQQKPDGTVKLLIYY | VH = Q252- |
| | | | TSILHSGVPSRFSGSGSGTDYSLTIGNLEPEDIATYYCQQF | S370; |
| | | | NKLPPTFGGGTKLEIKGGGGSQVQLVQSGAEVKKPGAS | CH1 = |
| | | | VKVSCKASGYSFTGYTMNWVRQAPGQGLEWMGLITP | A371-V468 |
| | | | YNGASSYNQKFRGKATMTVDTSTSTVYMELSSLRSEDT | |
| | | | AVYYCARGGYDGRGFDYWGQGTLVTVSSASTKGPSVF | |
| | | | PLAPSSKSTSGGTAALGCLVKDYFPEPVTVSWNSGALTS | |
| | | | GVHTFPAVLQSSGLYSLSSVVTVPSSSLGTQTYICNVNHK | |
| | | | PSNTKVDKKVEPKSCDKTHTCPPCPAPEAAGGPSVFLFP | |
| | | | PKPKDTLMISRTPEVTCVVVSVSHEDPEVKFNWYVDGV | |
| | | | EVHNAKTKPREEQYNSTYRVVSVLTVLHQDWLNGKEYK | |
| | | | CKVSNKALPAPIEKTISKAKGQPREPQVYVYPPSRDELTK | |
| | | | NQVSLTCLVKGFYPSDIAVEWESNGQPENNYKTTPPVL | |
| | | | DSDGSFALVSKLTVDKSRWQQGNVFSCSVMHEALHNH | |
| | | | YTQKSLSLSPG | |
| 109 | 16722 | Full | GAGGTGAAGCTGGTGGAGTCCGGAGGAGGACTGGT | |
| | | | GCAGCCAGGAGGCTCTCTGAAGCTGAGCTGCGCCAC | |
| | | | CTCCGGCTTCACATTTTCTGACTACTATATGTACTGGG | |
| | | | TGCGGCAGACCCCCGAGAAGAGACTGGAGTGGGTG | |
| | | | GCCTATATCAACTCTGGCGGCGGCAGCACCTACTATC | |
| | | | CTGACACAGTGAAGGGCAGGTTCACCATCTCCCGCGA | |
| | | | TAACGCCAAGAATACACTGTACCTGCAGATGTCCCGG | |
| | | | CTGAAGTCTGAGGACACAGCCATGTACTATTGCGCCC | |
| | | | GGAGAGGCCTGCCTTTTCACGCCATGGATTATTGGGG | |
| | | | CCAGGGCACCAGCGTGACAGTGAGCAGCGGCGGCG | |
| | | | GCGGCTCTGGAGGAGGAGGCAGCGGCGGAGGAGGC | |
| | | | TCCGGAGGAGGCGGCTCTGACATCCAGATGACCCAG | |
| | | | ACCACATCTAGCCTGAGCGCCTCCCTGGGCGATAGGG | |
| | | | TGACAATCTCTTGTAGCGCCTCCCAGGGCATCTCCAAC | |
| | | | TACCTGAATTGGTATCAGCAGAAGCCTGATGGCACCG | |
| | | | TGAAGCTGCTGATCTACTATACAAGCATCCTGCACTCC | |
| | | | GGCGTGCCATCTCGCTTCTCTGGCAGCGGCTCCGGAA | |
| | | | CCGACTACAGCCTGACAATCGGCAACCTGGAGCCAG | |
| | | | AGGATATCGCCACCTACTATTGCCAGCAGTTCAATAA | |
| | | | GCTGCCCCCTACCTTTGGCGGCGGCACAAAGCTGGAG | |
| | | | ATCAAGGGCGGCGGCGGCAGCGAGGTGCAGCTGGT | |
| | | | CGAAAGCGGCGGCGGCCTGGTCCAGCCTGGAGGCAG | |
| | | | CCTGAGGCTGTCCTGTGCCGCCTCTGGCTTTAACATCA | |
| | | | AGGACACCTACATCCACTGGGTGAGGCAGGCCCCAG | |
| | | | GCAAGGGACTGGAGTGGGTGGCCCGCATCTATCCCA | |
| | | | CCAATGGCTACACAAGATATGCCGACAGCGTGAAGG | |
| | | | GCCGCTTCACCATCAGCGCCGATACCTCCAAGAACAC | |
| | | | AGCCTACCTGCAGATGAACAGCCTGCGGGCCGAGGA | |
| | | | TACAGCCGTGTACTATTGTAGCAGATGGGGCGGCGA | |
| | | | CGGCTTTTACGCTATGGACTACTGGGGACAGGGCACC | |
| | | | CTGGTGACAGTGTCCTCTGCTAGCACTAAGGGGCCTT | |
| | | | CCGTGTTTCCACTGGCTCCCTCTAGTAAATCCACCTCT | |
| | | | GGAGGCACAGCTGCACTGGGATGTCTGGTGAAGGAT | |
| | | | TACTTCCCTGAACCAGTCACAGTGAGTTGGAACTCAG | |
| | | | GGGCTCTGACAAGTGGAGTCCATACTTTTCCCGCAGT | |
| | | | GCTGCAGTCAAGCGGACTGTACTCCCTGTCCTCTGTG | |
| | | | GTCACCGTGCCTAGTTCAAGCCTGGGCACCCAGACAT | |
| | | | ATATCTGCAACGTGAATCACAAGCCATCAAATACAAA | |
| | | | AGTCGACAAGAAAGTGGAGCCCAAGAGCTGTGATAA | |
| | | | AACTCATACCTGCCCACCTTGTCCGGCGCCAGAGGCT | |
| | | | GCAGGAGGACCAAGCGTGTTCCTGTTTCCACCCAAGC | |
| | | | CTAAAGACACACTGATGATTTCCCGAACCCCCGAAGT | |
| | | | CACATGCGTGGTCGTGTCTGTGAGTCACGAGGACCCT | |
| | | | GAAGTCAAGTTCAACTGGTACGTGGATGGCGTCGAG | |
| | | | GTGCATAATGCCAAGACTAAACCTAGGGAGGAACAG | |
| | | | TACAACTCAACCTATCGCGTCGTGAGCGTCCTGACAG | |
| | | | TGCTGCACCAGGATTGGCTGAACGGCAAAGAATATA | |
| | | | AGTGCAAAGTGAGCAATAAGGCCCTGCCCGCTCCTAT | |
| | | | CGAGAAAACCATTTCCAAGGCTAAAGGGCAGCCTCG | |
| | | | CGAACCACAGGTCTACGTCTACCCCCCATCAAGAGAT | |
| | | | GAACTGACAAAAAATCAGGTCTCTCTGACATGCCTGG | |
| | | | TCAAAGGATTCTACCCTTCCGACATCGCCGTGGAGTG | |
| | | | GGAAAGTAACGGCCAGCCCGAGAACAATTACAAGAC | |
| | | | CACACCCCCTGTCCTGGACTCTGATGGGAGTTTCGCTC | |
| | | | TGGTGTCAAAGCTGACCGTCGATAAAAGCCGGTGGC | |
| | | | AGCAGGGCAATGTGTTTAGCTGCTCCGTCATGCACGA | |
| | | | AGCCCTGCACAATCACTACACACAGAAGTCCCTGAGC | |
| | | | CTGAGCCCTGGC | |
| 110 | 16733 | Full | EVQLVESGGGLVQPGGSLRLSCAASGFNIKDTYIHWVR | VH = E1- |
| | | | QAPGKGLEWVARIYPTNGYTRYADSVKGRFTISADTSK | S120; |
| | | | NTAYLQMNSLRAEDTAVYYCSRWGGDGFYAMDYWG | CH1 = |
| | | | QGTLVTVSSASTKGPSVFPLAPSSKSTSGGTAALGCLVK | A121-V218 |
| | | | DYFPEPVTVSWNSGALTSGVHTFPAVLQSSGLYSLSSVV | |
| | | | TVPSSSLGTQTYICNVNHKPSNTKVDKKVEPKSCDKTHT | |
| | | | GGGGSEPAVYFKEQFLDGDGWTSRWIESKHKSDFGKF | |
| | | | VLSSGKFYGDEEKDKGLQTSQDARFYALSASFEPFSNKG | |
| | | | QTLVVQFTVKHEQNIDCGGGYVKLFPNSLDQTDMHGD | |
| | | | SEYNIMFGPDICGPGTKKVHVIFNYKGKNVLINKDIRCKD | |
| | | | DEFTHLYTLIVRPDNTYEVKIDNSQVESGSLEDDWDFLP | |
| | | | PKKIKDPDASKPEDWDERAKIDDPTDSKPEDWDKPEHI | |
| | | | PDPDAKKPEDWDEEMDGEWEPPVIQNPEYKGEWKPR | |
| | | | QIDNPDYKGTWIHPEIDNPEYSPDPSIYAYDNFGVLGLD | |
| | | | LWQVKSGTIFDNFLITNDEAYAEEFGNETWGVTKAAEK | |
| | | | QMKDKQDEEQRLKEEEEDKKRKEEEEAEDKEDDEDKDE | |
| | | | DEEDEEDKEEDEEEDVPGQAAAEPKSSDKTHTCPPCPA | |
| | | | PEAAGGPSVFLFPPKPKDTLMISRTPEVTCVVVSVSHED | |
| | | | PEVKFNWYVDGVEVHNAKTKPREEQYNSTYRVVSVLTV | |
| | | | LHQDWLNGKEYKCKVSNKALPAPIEKTISKAKGQPREPQ | |
| | | | VYVYPPSRDELTKNQVSLTCLVKGFYPSDIAVEWESNGQ | |
| | | | PENNYKTTPPVLDSDGSFALVSKLTVDKSRWQQGNVFS | |
| | | | CSVMHEALHNHYTQKSLSLSPG | |
| 111 | 16733 | Full | GAGGTGCAGCTGGTGGAGAGCGGCGGCGGCCTGGT | |
| | | | GCAGCCCGGCGGCTCTCTGCGGCTGAGCTGCGCCGC | |
| | | | CTCCGGCTTTAACATCAAGGACACATACATCCACTGG | |
| | | | GTGCGGCAGGCCCCCGGCAAGGGCCTGGAGTGGGT | |
| | | | GGCCAGAATCTATCCTACCAATGGCTACACACGGTAT | |
| | | | GCCGACTCCGTGAAGGGCAGATTCACCATCTCTGCCG | |
| | | | ATACCAGCAAGAACACAGCCTACCTGCAGATGAACAG | |
| | | | CCTGCGGGCCGAGGATACAGCCGTGTACTATTGTTCT | |
| | | | CGCTGGGGCGGCGACGGCTTTTACGCCATGGATTATT | |
| | | | GGGGCCAGGGCACCCTGGTGACAGTGAGCTCCGCTA | |
| | | | GCACTAAGGGGCCTTCCGTGTTTCCACTGGCTCCCTCT | |
| | | | AGTAAATCCACCTCTGGAGGCACAGCTGCACTGGGAT | |
| | | | GTCTGGTGAAGGATTACTTCCCTGAACCAGTCACAGT | |
| | | | GAGTTGGAACTCAGGGGCTCTGACAAGTGGAGTCCA | |
| | | | TACTTTTCCCGCAGTGCTGCAGTCAAGCGGACTGTAC | |
| | | | TCCCTGTCCTCTGTGGTCACCGTGCCTAGTTCAAGCCT | |
| | | | GGGCACCCAGACATATATCTGCAACGTGAATCACAAG | |
| | | | CCATCAAATACAAAAGTCGACAAGAAGGTGGAGCCT | |
| | | | AAGAGCTGCGACAAGACCCACACCGGAGGAGGAGG | |
| | | | CTCCGAGCCAGCCGTGTATTTCAAGGAGCAGTTTCTG | |
| | | | GACGGCGATGGCTGGACCAGCAGGTGGATCGAGTCC | |
| | | | AAGCACAAGTCTGACTTCGGCAAGTTTGTGCTGAGCT | |
| | | | CCGGCAAGTTCTATGGCGATGAGGAGAAGGACAAGG | |
| | | | GCCTGCAGACAAGCCAGGATGCCCGCTTTTACGCCCT | |
| | | | GTCCGCCTCTTTCGAGCCCTTTTCCAACAAGGGCCAG | |
| | | | ACCCTGGTGGTGCAGTTCACAGTGAAGCACGAGCAG | |
| | | | AACATCGACTGTGGCGGCGGCTATGTGAAGCTGTTTC | |
| | | | CTAATTCCCTGGATCAGACCGACATGCACGGCGACTC | |
| | | | TGAGTACAACATCATGTTCGGCCCTGATATCTGCGGC | |
| | | | CCAGGCACAAAGAAGGTGCACGTGATCTTTAATTACA | |
| | | | AGGGCAAGAACGTGCTGATCAATAAGGACATCCGGT | |
| | | | GTAAGGACGATGAGTTCACCCACCTGTACACACTGAT | |
| | | | CGTGAGACCAGACAACACCTATGAGGTGAAGATCGA | |
| | | | TAATAGCCAGGTGGAGAGCGGCTCCCTGGAGGACGA | |
| | | | TTGGGATTTTCTGCCCCCTAAGAAGATCAAGGACCCC | |
| | | | GATGCCTCTAAGCCTGAGGACTGGGATGAGCGGGCC | |
| | | | AAGATCGACGATCCAACAGACTCCAAGCCCGAGGAC | |
| | | | TGGGATAAGCCCGAGCACATCCCAGACCCCGATGCCA | |
| | | | AGAAGCCAGAAGACTGGGATGAGGAGATGGATGGC | |
| | | | GAGTGGGAGCCACCCGTGATCCAGAACCCTGAGTAC | |
| | | | AAGGGCGAGTGGAAGCCCAGACAGATCGATAATCCT | |
| | | | GACTATAAGGGCACCTGGATTCACCCTGAGATCGATA | |
| | | | ACCCAGAGTACAGCCCTGACCCATCCATCTACGCCTAT | |
| | | | GATAATTTCGGCGTGCTGGGACTGGACCTGTGGCAG | |
| | | | GTGAAGTCCGGCACCATCTTCGACAACTTTCTGATCAC | |
| | | | AAATGATGAGGCCTACGCCGAGGAGTTTGGCAACGA | |
| | | | GACCTGGGGCGTGACAAAGGCCGCCGAGAAGCAGAT | |
| | | | GAAGGATAAGCAGGACGAGGAGCAGAGGCTGAAGG | |
| | | | AAGAAGAGGAGGACAAGAAGCGCAAGGAGGAGGA | |
| | | | GGAGGCCGAGGATAAGGAGGACGATGAGGACAAGG | |
| | | | ATGAGGACGAGGAGGATGAGGAGGACAAGGAGGA | |
| | | | GGATGAGGAGGAGGACGTGCCAGGACAGGCCGCCG | |
| | | | CCGAGCCCAAGTCTAGCGACAAGACCCACACATGCCC | |
| | | | TCCATGTCCGGCGCCAGAGGCCGCCGGAGGACCTTCC | |
| | | | GTGTTCCTGTTTCCCCCTAAGCCAAAGGATACCCTGAT | |
| | | | GATCTCTAGAACCCCAGAGGTGACATGCGTGGTGGT | |
| | | | GTCTGTGAGCCACGAGGACCCCGAGGTGAAGTTCAA | |
| | | | CTGGTATGTGGATGGCGTGGAGGTGCACAATGCCAA | |
| | | | GACAAAGCCTAGGGAGGAGCAGTACAATTCTACCTAT | |
| | | | AGAGTGGTGAGCGTGCTGACAGTGCTGCACCAGGAC | |
| | | | TGGCTGAACGGCAAGGAGTACAAGTGTAAGGTGTCT | |
| | | | AATAAGGCCCTGCCAGCCCCCATCGAGAAGACCATCA | |
| | | | GCAAGGCCAAGGGCCAGCCTCGCGAACCACAGGTCT | |
| | | | ACGTCTACCCCCCATCAAGAGATGAACTGACAAAAAA | |
| | | | TCAGGTCTCTCTGACATGCCTGGTCAAAGGATTCTACC | |
| | | | CTTCCGACATCGCCGTGGAGTGGGAAAGTAACGGCC | |
| | | | AGCCCGAGAACAATTACAAGACCACACCCCCTGTCCT | |
| | | | GGACTCTGATGGGAGTTTCGCTCTGGTGTCAAAGCTG | |
| | | | ACCGTCGATAAAAGCCGGTGGCAGCAGGGCAATGTG | |
| | | | TTTAGCTGCTCCGTCATGCACGAAGCCCTGCACAATC | |
| | | | ACTACACACAGAAGTCCCTGAGCCTGAGCCCTGGC | |
| 112 | 16735 | Full | QVQLVQSGAEVKKPGASVKVSCKASGYSFTGYTMNWV | VH = Q1- |
| | | | RQAPGQGLEWMGLITPYNGASSYNQKFRGKATMTVD | S119; |
| | | | TSTSTVYMELSSLRSEDTAVYYCARGGYDGRGFDYWGQ | CH1 = |
| | | | GTLVTVSSASTKGPSVFPLAPSSKSTSGGTAALGCLVKDY | A120-V217 |
| | | | FPEPVTVSWNSGALTSGVHTFPAVLQSSGLYSLSSVVTV | |
| | | | PSSSLGTQTYICNVNHKPSNTKVDKKVEPKSCDKTHTGG | |
| | | | GGSEPAVYFKEQFLDGDGWTSRWIESKHKSDFGKFVLS | |
| | | | SGKFYGDEEKDKGLQTSQDARFYALSASFEPFSNKGQTL | |
| | | | VVQFTVKHEQNIDCGGGYVKLFPNSLDQTDMHGDSEY | |
| | | | NIMFGPDICGPGTKKVHVIFNYKGKNVLINKDIRCKDDE | |
| | | | FTHLYTLIVRPDNTYEVKIDNSQVESGSLEDDWDFLPPK | |
| | | | KIKDPDASKPEDWDERAKIDDPTDSKPEDWDKPEHIPD | |
| | | | PDAKKPEDWDEEMDGEWEPPVIQNPEYKGEWKPRQI | |
| | | | DNPDYKGTWIHPEIDNPEYSPDPSIYAYDNFGVLGLDL | |
| | | | WQVKSGTIFDNFLITNDEAYAEEFGNETWGVTKAAEKQ | |
| | | | MKDKQDEEQRLKEEEEDKKRKEEEEAEDKEDDEDKDED | |
| | | | EEDEEDKEEDEEEDVPGQAAAEPKSSDKTHTCPPCPAPE | |
| | | | AAGGPSVFLFPPKPKDTLMISRTPEVTCVVVSVSHEDPE | |
| | | | VKFNWYVDGVEVHNAKTKPREEQYNSTYRVVSVLTVLH | |
| | | | QDWLNGKEYKCKVSNKALPAPIEKTISKAKGQPREPQV | |
| | | | YVYPPSRDELTKNQVSLTCLVKGFYPSDIAVEWESNGQP | |
| | | | ENNYKTTPPVLDSDGSFALVSKLTVDKSRWQQGNVFSC | |
| | | | SVMHEALHNHYTQKSLSLSPG | |
| 113 | 16735 | Full | CAGGTGCAGCTGGTGCAGAGCGGAGCCGAGGTGAA | |
| | | | GAAGCCAGGGGCCAGCGTGAAGGTGTCTTGCAAGGC | |
| | | | CTCTGGCTACAGCTTCACAGGCTATACCATGAACTGG | |
| | | | GTGCGGCAGGCCCCCGGACAGGGCCTGGAGTGGATG | |
| | | | GGCCTGATCACACCTTACAACGGGGCCAGCTCCTATA | |
| | | | ATCAGAAGTTTCGGGGCAAGGCCACCATGACAGTGG | |
| | | | ACACCAGCACATCCACCGTGTACATGGAGCTGTCTAG | |
| | | | CCTGAGGTCCGAGGATACCGCCGTGTACTATTGTGCC | |
| | | | AGAGGCGGCTACGACGGCAGAGGCTTTGATTATTGG | |
| | | | GGCCAGGGCACACTGGTGACCGTGTCCTCTGCTAGCA | |
| | | | CTAAGGGGCCTTCCGTGTTTCCACTGGCTCCCTCTAGT | |
| | | | AAATCCACCTCTGGAGGCACAGCTGCACTGGGATGTC | |
| | | | TGGTGAAGGATTACTTCCCTGAACCAGTCACAGTGAG | |
| | | | TTGGAACTCAGGGGCTCTGACAAGTGGAGTCCATACT | |
| | | | TTTCCCGCAGTGCTGCAGTCAAGCGGACTGTACTCCC | |
| | | | TGTCCTCTGTGGTCACCGTGCCTAGTTCAAGCCTGGG | |
| | | | CACCCAGACATATATCTGCAACGTGAATCACAAGCCA | |
| | | | TCAAATACAAAAGTCGACAAGAAGGTGGAGCCCAAG | |
| | | | TCTTGCGACAAGACCCACACCGGAGGAGGAGGCAGC | |
| | | | GAGCCTGCCGTGTATTTCAAGGAGCAGTTTCTGGACG | |
| | | | GCGATGGATGGACCAGCCGGTGGATCGAGTCTAAGC | |
| | | | ACAAGAGCGACTTCGGCAAGTTTGTGCTGAGCTCCGG | |
| | | | CAAGTTCTATGGCGATGAGGAGAAGGACAAGGGCCT | |
| | | | GCAGACATCCCAGGATGCCCGGTTCTACGCCCTGTCC | |
| | | | GCCTCTTTCGAGCCATTTTCTAACAAGGGCCAGACCCT | |
| | | | GGTGGTGCAGTTCACAGTGAAGCACGAGCAGAACAT | |
| | | | CGACTGTGGCGGCGGCTATGTGAAGCTGTTTCCCAAT | |
| | | | AGCCTGGATCAGACCGACATGCACGGCGACTCCGAG | |
| | | | TACAACATCATGTTCGGCCCTGATATCTGCGGCCCAG | |
| | | | GCACAAAGAAGGTGCACGTGATCTTTAATTACAAGG | |
| | | | GCAAGAACGTGCTGATCAATAAGGACATCAGGTGTA | |
| | | | AGGACGATGAGTTCACCCACCTGTACACACTGATCGT | |
| | | | GCGCCCTGACAACACCTATGAGGTGAAGATCGATAAT | |
| | | | TCTCAGGTGGAGAGCGGCTCCCTGGAGGACGATTGG | |
| | | | GATTTTCTGCCCCCTAAGAAGATCAAGGACCCCGATG | |
| | | | CCAGCAAGCCTGAGGACTGGGATGAGAGGGCCAAG | |
| | | | ATCGACGATCCAACAGACTCCAAGCCCGAGGACTGG | |
| | | | GATAAGCCTGAGCACATCCCCGACCCTGATGCCAAGA | |
| | | | AGCCAGAGGACTGGGATGAGGAGATGGATGGCGAG | |
| | | | TGGGAGCCACCCGTGATCCAGAACCCCGAGTACAAG | |
| | | | GGCGAGTGGAAGCCCAGACAGATCGATAATCCTGAC | |
| | | | TATAAGGGCACCTGGATTCACCCTGAGATCGATAACC | |
| | | | CAGAGTACTCCCCAGACCCCTCTATCTACGCCTATGAT | |
| | | | AATTTCGGCGTGCTGGGCCTGGACCTGTGGCAGGTG | |
| | | | AAGTCCGGCACCATCTTCGACAACTTTCTGATCACAAA | |
| | | | TGATGAGGCCTATGCCGAGGAGTTTGGCAATGAGAC | |
| | | | CTGGGGCGTGACAAAGGCCGCCGAGAAGCAGATGA | |
| | | | AGGATAAGCAGGACGAGGAGCAGCGGCTGAAGGAA | |
| | | | GAAGAGGAGGACAAGAAGAGAAAGGAGGAGGAGG | |
| | | | AGGCCGAGGATAAGGAGGACGATGAGGACAAGGAT | |
| | | | GAGGACGAGGAGGATGAGGAGGACAAGGAGGAGG | |
| | | | ATGAGGAGGAGGACGTGCCAGGACAGGCCGCCGCC | |
| | | | GAGCCCAAGTCTAGCGACAAGACCCACACATGCCCTC | |
| | | | CATGTCCGGCGCCAGAGGCTGCAGGAGGACCAAGCG | |
| | | | TGTTCCTGTTTCCACCCAAGCCTAAAGACACACTGATG | |
| | | | ATTTCCCGAACCCCCGAAGTCACATGCGTGGTCGTGT | |
| | | | CTGTGAGTCACGAGGACCCTGAAGTCAAGTTCAACTG | |
| | | | GTACGTGGATGGCGTCGAGGTGCATAATGCCAAGAC | |
| | | | TAAACCTAGGGAGGAACAGTACAACTCAACCTATCGC | |
| | | | GTCGTGAGCGTCCTGACAGTGCTGCACCAGGATTGGC | |
| | | | TGAACGGCAAAGAATATAAGTGCAAAGTGAGCAATA | |
| | | | AGGCCCTGCCCGCTCCTATCGAGAAAACCATTTCCAA | |
| | | | GGCTAAAGGGCAGCCTCGCGAACCACAGGTCTACGT | |
| | | | GTATCCTCCAAGCCGGGACGAGCTGACAAAGAACCA | |
| | | | GGTCTCCCTGACTTGTCTGGTGAAAGGGTTTTACCCT | |
| | | | AGTGATATCGCTGTGGAGTGGGAATCAAATGGACAG | |
| | | | CCAGAGAACAATTATAAGACTACCCCCCCTGTGCTGG | |
| | | | ACAGTGATGGGTCATTCGCACTGGTCTCCAAGCTGAC | |
| | | | AGTGGACAAATCTCGGTGGCAGCAGGGAAATGTCTT | |
| | | | TTCATGTAGCGTGATGCATGAAGCACTGCACAACCAT | |
| | | | TACACCCAGAAGTCACTGTCACTGTCACCAGGA | |
| 114 | 16743 | Full | QVQLQQSGAELARPGASVKMSCKASGYTFTTYTMHW | VH = Q1- |
| | | | VKQRPGQGLEWIGYINPSSGYTNYNQKFKDKATLTADK | S121; |
| | | | SSSTASMQLSSLTSEDSAVYYCARERAVLVPYAMDYWG | VL = Q142- |
| | | | QGTSVTVSSGGGGSGGGGSGGGGSGGGGSQIVLTQSP | K247; |
| | | | AVMSASPGEKVTITCTASSSLSYMHWFQQKPGTSPKL | VH = Q486- |
| | | | WLYSTSILASGVPTRFSGSGSGTSYSLTISRMEAEDAATY | S606; |
| | | | YCQQRSSSPFTFGSGTKLEIKAAEPKSSDKTHTCPPCPAP | VL = Q627- |
| | | | EAAGGPSVFLFPPKPKDTLMISRTPEVTCVVVSVSHEDP | K732 |
| | | | EVKFNWYVDGVEVHNAKTKPREEQYNSTYRVVSVLTVL | |
| | | | HQDWLNGKEYKCKVSNKALPAPIEKTISKAKGQPREPQ | |
| | | | VYVLPPSRDELTKNQVSLLCLVKGFYPSDIAVEWESNGQ | |
| | | | PENNYLTWPPVLDSDGSFFLYSKLTVDKSRWQQGNVFS | |
| | | | CSVMHEALHNHYTQKSLSLSPGGGGGSQVQLQQSGAE | |
| | | | LARPGASVKMSCKASGYTFTTYTMHWVKQRPGQGLE | |
| | | | WIGYINPSSGYTNYNQKFKDKATLTADKSSSTASMQLSS | |
| | | | LTSEDSAVYYCARERAVLVPYAMDYWGQGTSVTVSSG | |
| | | | GGGSGGGGSGGGGSGGGGSQIVLTQSPAVMSASPGE | |
| | | | KVTITCTASSSLSYMHWFQQKPGTSPKLWLYSTSILASG | |
| | | | VPTRFSGSGSGTSYSLTISRMEAEDAATYYCQQRSSSPFT | |
| | | | FGSGTKLEIK | |
| 115 | 16743 | Full | CAGGTGCAGCTGCAGCAGTCCGGAGCCGAGCTGGCC | |
| | | | AGACCCGGAGCCAGCGTGAAGATGTCCTGCAAGGCC | |
| | | | TCTGGCTACACCTTCACCACATATACAATGCACTGGGT | |
| | | | GAAGCAGAGACCCGGACAGGGACTGGAGTGGATCG | |
| | | | GATACATCAACCCTAGCTCCGGCTACACCAACTATAAT | |
| | | | CAGAAGTTTAAGGACAAGGCCACCCTGACAGCCGAT | |
| | | | AAGTCTAGCTCCACCGCCAGCATGCAGCTGTCTAGCC | |
| | | | TGACAAGCGAGGACTCCGCCGTGTACTATTGTGCCCG | |
| | | | GGAGAGAGCCGTGCTGGTGCCATACGCCATGGATTA | |
| | | | TGGGGCCAGGGCACCTCCGTGACAGTGTCCTCTGGA | |
| | | | GGAGGAGGCAGCGGGGGAGGAGGCTCCGGAGGCG | |
| | | | GCGGCTCTGGCGGCGGCGGCAGCCAGATCGTGCTGA | |
| | | | CCCAGAGCCCCGCCGTGATGTCTGCCAGCCCTGGAGA | |
| | | | GAAGGTGACCATCACATGCACCGCCAGCTCCTCTCTG | |
| | | | AGCTACATGCACTGGTTCCAGCAGAAGCCAGGCACCT | |
| | | | CCCCCAAGCTGTGGCTGTATTCCACATCTATCCTGGCC | |
| | | | TCCGGAGTGCCAACCAGGTTTAGCGGCTCCGGCTCTG | |
| | | | GCACCAGCTACTCCCTGACAATCAGCAGGATGGAGG | |
| | | | CAGAGGACGCAGCAACCTACTATTGTCAGCAGCGCA | |
| | | | GCTCCTCTCCATTCACCTTTGGCAGCGGCACAAAGCT | |
| | | | GGAGATCAAGGCCGCCGAGCCCAAGAGCTCCGACAA | |
| | | | GACACACACCTGCCCACCTTGTCCGGCGCCAGAGGCC | |
| | | | GCCGGAGGACCTTCCGTGTTCCTGTTTCCACCCAAGC | |
| | | | CAAAGGATACCCTGATGATCAGCAGGACCCCAGAGG | |
| | | | TGACATGCGTGGTGGTGTCTGTGAGCCACGAGGACC | |
| | | | CTGAGGTGAAGTTTAACTGGTACGTGGATGGCGTGG | |
| | | | AGGTGCACAATGCCAAGACAAAGCCTCGGGAGGAGC | |
| | | | AGTACAACTCTACCTATAGAGTGGTGAGCGTGCTGAC | |
| | | | AGTGCTGCACCAGGACTGGCTGAACGGCAAGGAGTA | |
| | | | TAAGTGCAAGGTGTCCAATAAGGCCCTGCCTGCCCCA | |
| | | | ATCGAGAAGACCATCTCTAAGGCCAAGGGCCAGCCTC | |
| | | | GCGAACCTCAGGTGTACGTGCTGCCTCCATCCCGCGA | |
| | | | CGAGCTGACAAAGAACCAGGTGTCTCTGCTGTGCCTG | |
| | | | GTGAAGGGCTTCTATCCTTCTGATATCGCCGTGGAGT | |
| | | | GGGAGAGCAATGGCCAGCCAGAGAACAATTACCTGA | |
| | | | CCTGGCCCCCTGTGCTGGACTCTGATGGCAGCTTCTTT | |
| | | | CTGTATTCCAAGCTGACAGTGGATAAGTCTCGGTGGC | |
| | | | AGCAGGGCAACGTGTTTTCCTGCTCTGTGATGCACGA | |
| | | | GGCCCTGCACAATCACTACACCCAGAAGAGCCTGAGC | |
| | | | TTAAGCCCTGGAGGAGGAGGAGGCAGCCAGGTCCAG | |
| | | | CTGCAGCAGAGCGGAGCCGAGCTGGCCAGGCCAGG | |
| | | | AGCCAGCGTCAAGATGTCCTGTAAAGCCTCTGGATAT | |
| | | | ACCTTCACCACCTACACCATGCATTGGGTCAAGCAGC | |
| | | | GCCCAGGCCAGGGCCTGGAGTGGATCGGCTATATCA | |
| | | | ATCCCTCTAGCGGCTACACAAATTACAACCAGAAGTT | |
| | | | TAAGGATAAGGCCACACTGACCGCCGATAAGTCCTCT | |
| | | | AGCACAGCCAGCATGCAGCTGTCCTCTCTGACCTCCG | |
| | | | AGGACTCTGCCGTGTACTATTGTGCAAGGGAGAGGG | |
| | | | CCGTGCTGGTCCCTTATGCTATGGACTACTGGGGACA | |
| | | | GGGCACCTCCGTCACAGTGAGCTCTGGCGGAGGAGG | |
| | | | CTCCGGAGGAGGAGGCTCTGGAGGAGGCGGCAGCG | |
| | | | GCGGCGGCGGCTCCCAGATCGTGCTGACTCAGAGCC | |
| | | | CAGCCGTGATGAGCGCCTCCCCAGGAGAGAAGGTGA | |
| | | | CAATCACCTGCACAGCCTCTAGCTCCCTGTCTTATATG | |
| | | | CATTGGTTCCAGCAGAAGCCTGGCACAAGCCCAAAGC | |
| | | | TGTGGCTGTATTCTACCAGCATCCTGGCCTCCGGCGT | |
| | | | CCCAACACGGTTTTCCGGCTCTGGCAGCGGCACCTCC | |
| | | | TACTCTCTGACCATTTCCAGAATGGAGGCAGAGGATG | |
| | | | CCGCCACTTATTATTGTCAGCAGAGATCTAGCTCCCCT | |
| | | | TTCACCTTTGGCAGCGGAACCAAACTGGAGATCAAG | |
| 116 | 16744 | Full | QIVLTQSPAVMSASPGEKVTITCTASSSLSYMHWFQQK | VL = Q1- |
| | | | PGTSPKLWLYSTSILASGVPTRFSGSGSGTSYSLTISRME | K106; |
| | | | AEDAATYYCQQRSSSPFTFGSGTKLEIKGGGGSGGGGS | VH = Q127- |
| | | | GGGGSGGGGSQVQLVESGGGVVQPGRSLRLSCAASGF | S244; |
| | | | TFSNYGMYWVRQAPGKGLEWVAVIWYDGSNKYYADS | VL = Q483- |
| | | | VKGRFTISRDNSKNTLYLQMNSLRAEDTAVYYCARDLW | K588; |
| | | | GWYFDYWGQGTLVTVSSAAEPKSSDKTHTCPPCPAPE | VH = Q609- |
| | | | AAGGPSVFLFPPKPKDTLMISRTPEVTCVVVSVSHEDPE | S726 |
| | | | VKFNWYVDGVEVHNAKTKPREEQYNSTYRVVSVLTVLH | |
| | | | QDWLNGKEYKCKVSNKALPAPIEKTISKAKGQPREPQV | |
| | | | YVLPPSRDELTKNQVSLLCLVKGFYPSDIAVEWESNGQP | |
| | | | ENNYLTWPPVLDSDGSFFLYSKLTVDKSRWQQGNVFSC | |
| | | | SVMHEALHNHYTQKSLSLSPGGGGGSQIVLTQSPAVMS | |
| | | | ASPGEKVTITCTASSSLSYMHWFQQKPGTSPKLWLYSTS | |
| | | | ILASGVPTRFSGSGSGTSYSLTISRMEAEDAATYYCQQRS | |
| | | | SSPFTFGSGTKLEIKGGGGSGGGGSGGGGSGGGGSQV | |
| | | | QLVESGGGVVQPGRSLRLSCAASGFTFSNYGMYWVRQ | |
| | | | APGKGLEWVAVIWYDGSNKYYADSVKGRFTISRDNSKN | |
| | | | TLYLQMNSLRAEDTAVYYCARDLWGWYFDYWGQGTL | |
| | | | VTVSS | |
| 117 | 16744 | Full | CAGATCGTGCTGACACAGTCCCCCGCCGTGATGAGCG | |
| | | | CCTCCCCTGGAGAGAAGGTGACCATCACATGCACCGC | |
| | | | CAGCTCCTCTCTGTCTTACATGCACTGGTTCCAGCAGA | |
| | | | AGCCAGGCACCAGCCCCAAGCTGTGGCTGTATTCTAC | |
| | | | AAGCATCCTGGCCTCCGGAGTGCCTACCCGGTTTTCC | |
| | | | GGCTCTGGCAGCGGCACCTCCTACTCTCTGACAATCA | |
| | | | GCAGGATGGAGGCAGAGGACGCAGCAACCTACTATT | |
| | | | GCCAGCAGAGAAGCTCCTCTCCATTCACCTTTGGCAG | |
| | | | CGGCACAAAGCTGGAGATCAAGGGAGGAGGAGGCT | |
| | | | CCGGGGGAGGAGGCTCTGGCGGCGGCGGCAGCGGA | |
| | | | GGCGGCGGCTCCCAGGTGCAGCTGGTGGAGTCCGGC | |
| | | | GGCGGCGTGGTGCAGCCCGGCAGAAGCCTGAGACTG | |
| | | | TCCTGTGCCGCCTCTGGCTTCACCTTTAGCAACTACGG | |
| | | | CATGTATTGGGTGAGACAGGCACCTGGCAAGGGACT | |
| | | | GGAGTGGGTGGCCGTGATCTGGTACGACGGCTCTAA | |
| | | | TAAGTACTATGCCGATAGCGTGAAGGGCCGGTTCACA | |
| | | | ATCAGCAGAGACAACTCCAAGAATACCCTGTATCTGC | |
| | | | AGATGAACAGCCTGAGGGCCGAGGATACCGCCGTGT | |
| | | | ACTATTGCGCCCGCGACCTGTGGGGCTGGTACTTTGA | |
| | | | TTATTGGGGCCAGGGCACCCTGGTGACAGTGAGCTCC | |
| | | | GCCGCCGAGCCAAAGTCTAGCGACAAGACACACACC | |
| | | | TGCCCACCTTGTCCGGCGCCAGAGGCCGCCGGAGGA | |
| | | | CCTAGCGTGTTCCTGTTTCCACCCAAGCCAAAGGATA | |
| | | | CCCTGATGATCAGCAGGACCCCAGAGGTGACATGCG | |
| | | | TGGTGGTGAGCGTGTCCCACGAGGACCCCGAGGTGA | |
| | | | AGTTCAACTGGTACGTGGATGGCGTGGAGGTGCACA | |
| | | | ATGCCAAGACAAAGCCTCGGGAGGAGCAGTACAATA | |
| | | | GCACCTATAGAGTGGTGTCCGTGCTGACAGTGCTGCA | |
| | | | CCAGGACTGGCTGAACGGCAAGGAGTACAAGTGCAA | |
| | | | GGTGAGCAATAAGGCCCTGCCTGCCCCAATCGAGAA | |
| | | | GACCATCTCCAAGGCCAAGGGCCAGCCTCGCGAACCT | |
| | | | CAGGTGTACGTGCTGCCTCCAAGCAGAGACGAGCTG | |
| | | | ACAAAGAACCAGGTGTCCCTGCTGTGCCTGGTGAAG | |
| | | | GGCTTCTATCCCTCCGATATCGCCGTGGAGTGGGAGT | |
| | | | CTAATGGCCAGCCTGAGAACAATTACCTGACCTGGCC | |
| | | | CCCTGTGCTGGACTCCGATGGCTCTTTCTTTCTGTATT | |
| | | | CCAAGCTGACAGTGGATAAGTCTAGGTGGCAGCAGG | |
| | | | GCAACGTGTTTTCTTGCAGCGTGATGCACGAGGCCCT | |
| | | | GCACAATCACTACACCCAGAAGTCCCTGAGCTTAAGC | |
| | | | CCAGGAGGAGGAGGAGGCAGCCAGATCGTGCTGAC | |
| | | | CCAGTCCCCAGCCGTGATGTCCGCCTCTCCAGGAGAG | |
| | | | AAGGTGACAATCACCTGTACAGCCTCCTCTAGCCTGT | |
| | | | CCTATATGCATTGGTTCCAGCAGAAGCCTGGCACATC | |
| | | | TCCAAAGCTGTGGCTGTATAGCACCTCCATCCTGGCCT | |
| | | | CCGGCGTCCCAACACGCTTTTCTGGCAGCGGCTCCGG | |
| | | | CACCTCTTACAGCCTGACCATTAGCAGGATGGAGGCC | |
| | | | GAGGATGCCGCCACTTATTATTGCCAGCAGCGGAGCT | |
| | | | CTAGCCCTTTCACCTTTGGCTCCGGAACCAAGCTGGA | |
| | | | GATCAAGGGCGGCGGCGGCTCTGGAGGAGGAGGCA | |
| | | | GCGGAGGAGGAGGCTCCGGCGGCGGCGGCTCTCAG | |
| | | | GTCCAGCTGGTCGAGTCCGGAGGAGGAGTGGTGCAG | |
| | | | CCAGGCAGGTCTCTGAGGCTGAGCTGTGCAGCCTCCG | |
| | | | GCTTCACCTTTAGCAATTACGGAATGTATTGGGTGCG | |
| | | | GCAGGCACCAGGCAAGGGCCTGGAATGGGTCGCCGT | |
| | | | GATCTGGTATGATGGCTCTAATAAGTATTACGCTGAC | |
| | | | AGCGTGAAGGGCAGGTTCACCATCTCCCGCGACAAC | |
| | | | AGCAAGAATACATTATATCTGCAAATGAACAGCCTGA | |
| | | | GAGCTGAAGACACCGCCGTGTACTATTGTGCTAGAGA | |
| | | | CCTGTGGGGATGGTATTTCGACTACTGGGGACAGGG | |
| | | | CACCCTGGTCACAGTGTCCTCT | |
| 118 | 16745 | Full | QVQLVESGGGVVQPGRSLRLSCAASGFTFSNYGMYWV | VH = Q1- |
| | | | RQAPGKGLEWVAVIWYDGSNKYYADSVKGRFTISRDN | S118; |
| | | | SKNTLYLQMNSLRAEDTAVYYCARDLWGWYFDYWGQ | VL = E139- |
| | | | GTLVTVSSGGGGSGGGGSGGGGSGGGGSEIVLTQSPA | K245; |
| | | | TLSLSPGERATLSCRASQSVSSYLAWYQQKPGQAPRLLIY | VH = Q484- |
| | | | DASNRATGIPARFSGSGSGTDFTLTISSLEPEDFAVYYCQ | S601; |
| | | | QRRNWPLTFGGGTKVEIKAAEPKSSDKTHTCPPCPAPE | VL = E622- |
| | | | AAGGPSVFLFPPKPKDTLMISRTPEVTCVVVSVSHEDPE | K728 |
| | | | VKFNWYVDGVEVHNAKTKPREEQYNSTYRVVSVLTVLH | |
| | | | QDWLNGKEYKCKVSNKALPAPIEKTISKAKGQPREPQV | |
| | | | YVLPPSRDELTKNQVSLLCLVKGFYPSDIAVEWESNGQP | |
| | | | ENNYLTWPPVLDSDGSFFLYSKLTVDKSRWQQGNVFSC | |
| | | | SVMHEALHNHYTQKSLSLSPGGGGGSQVQLVESGGGV | |
| | | | VQPGRSLRLSCAASGFTFSNYGMYWVRQAPGKGLEWV | |
| | | | AVIWYDGSNKYYADSVKGRFTISRDNSKNTLYLQMNSL | |
| | | | RAEDTAVYYCARDLWGWYFDYWGQGTLVTVSSGGGG | |
| | | | SGGGGSGGGGSGGGGSEIVLTQSPATLSLSPGERATLSC | |
| | | | RASQSVSSYLAWYQQKPGQAPRLLIYDASNRATGIPARF | |
| | | | SGSGSGTDFTLTISSLEPEDFAVYYCQQRRNWPLTFGGG | |
| | | | TKVEIK | |
| 119 | 16745 | Full | CAGGTGCAGCTGGTGGAGTCCGGAGGAGGAGTGGT | |
| | | | GCAGCCTGGCCGGTCCCTGAGACTGTCTTGCGCAGCC | |
| | | | AGCGGCTTCACCTTCAGCAACTACGGCATGTATTGGG | |
| | | | TGAGGCAGGCACCAGGCAAGGGACTGGAGTGGGTG | |
| | | | GCCGTGATCTGGTACGACGGCAGCAATAAGTACTATG | |
| | | | CCGATTCCGTGAAGGGCCGGTTCACCATCTCCAGAGA | |
| | | | CAACTCTAAGAATACACTGTATCTGCAGATGAACTCC | |
| | | | CTGAGGGCCGAGGATACCGCCGTGTACTATTGCGCCC | |
| | | | GCGACCTGTGGGGCTGGTACTTTGATTATTGGGGCCA | |
| | | | GGGCACCCTGGTGACAGTGAGCAGCGGCGGCGGCG | |
| | | | GCTCTGGAGGAGGAGGCAGCGGGGGAGGAGGCTCC | |
| | | | GGAGGAGGCGGCTCTGAGATCGTGCTGACCCAGTCT | |
| | | | CCCGCCACACTGTCTCTGAGCCCTGGAGAGAGGGCCA | |
| | | | CCCTGAGCTGTAGAGCCTCCCAGAGCGTGAGCAGCTA | |
| | | | CCTGGCCTGGTATCAGCAGAAGCCAGGCCAGGCCCC | |
| | | | CAGACTGCTGATCTACGACGCCAGCAACAGGGCAAC | |
| | | | CGGCATCCCTGCCAGATTCAGCGGCTCCGGCTCTGGC | |
| | | | ACAGACTTTACCCTGACAATCTCCTCTCTGGAGCCTGA | |
| | | | GGATTTCGCCGTGTACTATTGCCAGCAGCGGAGAAAT | |
| | | | TGGCCACTGACCTTTGGCGGCGGCACAAAGGTGGAG | |
| | | | ATCAAGGCCGCCGAGCCAAAGAGCTCCGACAAGACC | |
| | | | CACACATGCCCACCTTGTCCGGCGCCAGAGGCCGCCG | |
| | | | GAGGACCTTCCGTGTTCCTGTTTCCACCCAAGCCAAA | |
| | | | GGATACCCTGATGATCAGCAGAACCCCAGAGGTGAC | |
| | | | ATGCGTGGTGGTGAGCGTGTCCCACGAGGACCCCGA | |
| | | | GGTGAAGTTCAACTGGTACGTGGATGGCGTGGAGGT | |
| | | | GCACAATGCCAAGACAAAGCCCAGAGAGGAGCAGTA | |
| | | | CAACTCCACCTATAGAGTGGTGTCTGTGCTGACAGTG | |
| | | | CTGCACCAGGACTGGCTGAACGGCAAGGAGTACAAG | |
| | | | TGCAAGGTGAGCAATAAGGCCCTGCCTGCCCCAATCG | |
| | | | AGAAGACCATCTCCAAGGCCAAGGGCCAGCCTCGCG | |
| | | | AACCTCAGGTGTACGTGCTGCCTCCATCCAGAGACGA | |
| | | | GCTGACAAAGAACCAGGTGTCTCTGCTGTGCCTGGTG | |
| | | | AAGGGCTTCTATCCCTCTGATATCGCCGTGGAGTGGG | |
| | | | AGAGCAATGGCCAGCCTGAGAACAATTACCTGACCTG | |
| | | | GCCCCCTGTGCTGGACTCTGATGGCAGCTTCTTTCTGT | |
| | | | ATTCTAAGCTGACAGTGGATAAGAGCAGGTGGCAGC | |
| | | | AGGGCAACGTGTTTTCTTGCAGCGTGATGCACGAGGC | |
| | | | CCTGCACAATCACTACACCCAGAAGTCCCTGAGCTTA | |
| | | | AGCCCAGGAGGAGGAGGAGGCTCCCAGGTCCAGCTG | |
| | | | GTCGAGTCTGGCGGCGGAGTGGTGCAGCCCGGCAGG | |
| | | | AGCCTGAGGCTGTCCTGTGCAGCCTCTGGCTTCACAT | |
| | | | TTTCCAACTACGGAATGTATTGGGTGCGCCAGGCCCC | |
| | | | TGGCAAGGGCCTGGAATGGGTCGCCGTGATCTGGTA | |
| | | | TGATGGCAGCAATAAGTATTACGCTGACTCCGTGAAG | |
| | | | GGCAGGTTCACCATCAGCCGCGACAACTCCAAAAACA | |
| | | | CCCTGTATCTGCAGATGAATAGCCTGAGAGCTGAAGA | |
| | | | CACCGCCGTGTACTATTGTGCTAGAGACCTGTGGGGA | |
| | | | TGGTATTTCGACTACTGGGGACAGGGCACCCTGGTCA | |
| | | | CAGTGTCTAGCGGCGGCGGCGGCAGCGGCGGCGGA | |
| | | | GGCTCCGGAGGGGGCGGCTCTGGCGGCGGCGGCAG | |
| | | | CGAAATCGTGCTGACTCAGTCCCCAGCCACACTGTCC | |
| | | | CTGTCTCCAGGCGAAAGGGCCACCCTGAGCTGCAGG | |
| | | | GCCAGCCAGTCCGTGTCCTCTTACCTGGCTTGGTACCA | |
| | | | GCAGAAGCCTGGACAGGCACCACGGCTGCTGATCTA | |
| | | | CGATGCCAGCAATAGAGCAACCGGCATCCCTGCACGC | |
| | | | TTCTCTGGCAGCGGCTCCGGAACCGACTTTACCCTGA | |
| | | | CCATTAGCTCCCTGGAGCCCGAAGACTTCGCCGTGTA | |
| | | | CTATTGTCAGCAGAGGCGCAATTGGCCTCTGACCTTT | |
| | | | GGCGGAGGAACCAAAGTGGAGATCAAG | |
| 120 | 16772 | Full | QVQLQQSGAELARPGASVKMSCKASGYTFTTYTMHW | VH = Q1- |
| | | | VKQRPGQGLEWIGYINPSSGYTNYNQKFKDKATLTADK | S121; |
| | | | SSSTASMQLSSLTSEDSAVYYCARERAVLVPYAMDYWG | VL = Q142- |
| | | | QGTSVTVSSGGGGSGGGGSGGGGSGGGGSQIVLTQSP | K247; |
| | | | AVMSASPGEKVTITCTASSSLSYMHWFQQKPGTSPKL | VH = Q253- |
| | | | WLYSTSILASGVPTRFSGSGSGTSYSLTISRMEAEDAATY | S373; |
| | | | YCQQRSSSPFTFGSGTKLEIKGGGGSQVQLQQSGAELA | CH1 = |
| | | | RPGASVKMSCKASGYTFTTYTMHWVKQRPGQGLEWI | A374-V471 |
| | | | GYINPSSGYTNYNQKFKDKATLTADKSSSTASMQLSSLT | |
| | | | SEDSAVYYCARERAVLVPYAMDYWGQGTSVTVSSASTK | |
| | | | GPSVFPLAPSSKSTSGGTAALGCLVKDYFPEPVTVSWNS | |
| | | | GALTSGVHTFPAVLQSSGLYSLSSVVTVPSSSLGTQTYIC | |
| | | | NVNHKPSNTKVDKKVEPKSCDKTHTCPPCPAPEAAGGP | |
| | | | SVFLFPPKPKDTLMISRTPEVTCVVVSVSHEDPEVKFNW | |
| | | | YVDGVEVHNAKTKPREEQYNSTYRVVSVLTVLHQDWL | |
| | | | NGKEYKCKVSNKALPAPIEKTISKAKGQPREPQVYVLPPS | |
| | | | RDELTKNQVSLLCLVKGFYPSDIAVEWESNGQPENNYLT | |
| | | | WPPVLDSDGSFFLYSKLTVDKSRWQQGNVFSCSVMHE | |
| | | | ALHNHYTQKSLSLSPG | |
| 121 | 16772 | Full | CAGGTGCAGCTGCAGCAGTCCGGAGCCGAGCTGGCC | |
| | | | AGACCTGGGGCCAGCGTGAAGATGTCTTGCAAGGCC | |
| | | | AGCGGCTACACATTCACCACATATACCATGCACTGGG | |
| | | | TGAAGCAGCGCCCTGGACAGGGACTGGAGTGGATCG | |
| | | | GCTACATCAACCCAAGCTCCGGCTACACAAACTATAA | |
| | | | TCAGAAGTTTAAGGACAAGGCCACCCTGACAGCCGAT | |
| | | | AAGTCTAGCTCCACAGCCAGCATGCAGCTGTCTAGCC | |
| | | | TGACCAGCGAGGACTCCGCCGTGTACTATTGCGCCCG | |
| | | | GGAGAGAGCCGTGCTGGTGCCTTACGCCATGGATTAT | |
| | | | TGGGGCCAGGGCACATCTGTGACCGTGTCCTCTGGCG | |
| | | | GCGGCGGCTCCGGAGGCGGCGGCTCTGGAGGAGGA | |
| | | | GGCAGCGGCGGAGGAGGCTCCCAGATCGTGCTGACC | |
| | | | CAGAGCCCAGCCGTGATGAGCGCCTCCCCAGGAGAG | |
| | | | AAGGTGACCATCACATGTACCGCCAGCTCCTCTCTGTC | |
| | | | CTACATGCACTGGTTCCAGCAGAAGCCCGGCACATCT | |
| | | | CCTAAGCTGTGGCTGTATTCTACCAGCATCCTGGCCA | |
| | | | GCGGCGTGCCAACACGGTTTTCCGGCTCTGGCAGCG | |
| | | | GCACATCCTACTCTCTGACCATCTCCAGGATGGAGGC | |
| | | | AGAGGACGCAGCAACCTACTATTGCCAGCAGCGCAG | |
| | | | CTCCTCTCCATTCACATTTGGCTCCGGCACCAAGCTGG | |
| | | | AGATCAAGGGAGGAGGAGGCTCTCAGGTCCAGCTGC | |
| | | | AGCAGAGCGGAGCCGAGCTGGCCCGGCCCGGGGCC | |
| | | | AGCGTCAAAATGTCTTGTAAAGCCAGCGGATATACAT | |
| | | | TCACCACCTACACTATGCATTGGGTCAAGCAGAGACC | |
| | | | CGGCCAGGGCCTGGAGTGGATCGGATACATCAATCC | |
| | | | TAGCTCCGGCTACACCAATTACAACCAGAAGTTTAAG | |
| | | | GATAAGGCCACACTGACCGCCGATAAATCCAGCTCCA | |
| | | | CCGCCTCCATGCAGCTGTCCTCCCTGACATCTGAGGA | |
| | | | CAGCGCCGTGTACTATTGTGCCAGGGAGAGGGCCGT | |
| | | | GCTGGTCCCATATGCTATGGACTACTGGGGCCAGGGC | |
| | | | ACAAGCGTGACCGTGTCCTCTGCTAGCACCAAGGGAC | |
| | | | CATCCGTGTTCCCACTGGCACCAAGCTCCAAGTCTACA | |
| | | | AGCGGAGGAACCGCCGCCCTGGGCTGTCTGGTGAAG | |
| | | | GATTACTTCCCAGAGCCCGTGACCGTGTCTTGGAACA | |
| | | | GCGGGGCCCTGACCAGCGGAGTGCACACCTTTCCTGC | |
| | | | CGTGCTGCAGTCTAGCGGCCTGTATAGCCTGTCCTCT | |
| | | | GTGGTCACAGTGCCAAGCTCCTCTCTGGGCACACAGA | |
| | | | CCTACATCTGCAACGTGAATCACAAGCCATCCAATAC | |
| | | | CAAGGTCGACAAGAAGGTGGAGCCCAAGTCTTGTGA | |
| | | | TAAGACACACACCTGCCCACCTTGTCCGGCGCCAGAG | |
| | | | GCCGCCGGAGGACCAAGCGTGTTCCTGTTTCCACCCA | |
| | | | AGCCTAAGGACACACTGATGATCAGCAGGACACCAG | |
| | | | AGGTGACCTGCGTGGTGGTGTCCGTGTCTCACGAGG | |
| | | | ACCCCGAGGTGAAGTTTAACTGGTACGTGGATGGCG | |
| | | | TGGAGGTGCACAATGCCAAGACCAAGCCAAGGGAGG | |
| | | | AGCAGTATAACTCTACATACCGCGTGGTGAGCGTGCT | |
| | | | GACCGTGCTGCACCAGGATTGGCTGAACGGCAAGGA | |
| | | | GTACAAGTGCAAGGTGAGCAATAAGGCCCTGCCCGC | |
| | | | CCCTATCGAGAAGACAATCTCCAAGGCCAAGGGCCA | |
| | | | GCCTCGCGAACCACAGGTGTATGTGCTGCCTCCATCT | |
| | | | AGAGACGAGCTGACCAAGAACCAGGTGAGCCTGCTG | |
| | | | TGCCTGGTGAAGGGCTTCTACCCCAGCGATATCGCCG | |
| | | | TGGAGTGGGAGTCCAATGGCCAGCCTGAGAACAATT | |
| | | | ATCTGACATGGCCCCCTGTGCTGGACTCCGATGGCTC | |
| | | | TTTCTTTCTGTACTCCAAGCTGACCGTGGACAAGTCTC | |
| | | | GCTGGCAGCAGGGCAACGTGTTTAGCTGTTCCGTGAT | |
| | | | GCACGAGGCCCTGCACAATCACTACACCCAGAAGTCT | |
| | | | CTGAGCTTAAGCCCTGGC | |
| 122 | 16773 | Full | QVQLVESGGGVVQPGRSLRLSCAASGFTFSNYGMYWV | VH = Q1- |
| | | | RQAPGKGLEWVAVIWYDGSNKYYADSVKGRFTISRDN | S118; |
| | | | SKNTLYLQMNSLRAEDTAVYYCARDLWGWYFDYWGQ | VL = E139- |
| | | | GTLVTVSSGGGGSGGGGSGGGGSGGGGSEIVLTQSPA | K245; |
| | | | TLSLSPGERATLSCRASQSVSSYLAWYQQKPGQAPRLLIY | VH = Q251- |
| | | | DASNRATGIPARFSGSGSGTDFTLTISSLEPEDFAVYYCQ | S368; |
| | | | QRRNWPLTFGGGTKVEIKGGGGSQVQLVESGGGVVQ | CH1 = |
| | | | PGRSLRLSCAASGFTFSNYGMYWVRQAPGKGLEWVAV | A369-V466 |
| | | | IWYDGSNKYYADSVKGRFTISRDNSKNTLYLQMNSLRA | |
| | | | EDTAVYYCARDLWGWYFDYWGQGTLVTVSSASTKGPS | |
| | | | VFPLAPSSKSTSGGTAALGCLVKDYFPEPVTVSWNSGAL | |
| | | | TSGVHTFPAVLQSSGLYSLSSVVTVPSSSLGTQTYICNVN | |
| | | | HKPSNTKVDKKVEPKSCDKTHTCPPCPAPEAAGGPSVFL | |
| | | | FPPKPKDTLMISRTPEVTCVVVSVSHEDPEVKFNWYVD | |
| | | | GVEVHNAKTKPREEQYNSTYRVVSVLTVLHQDWLNGK | |
| | | | EYKCKVSNKALPAPIEKTISKAKGQPREPQVYVLPPSRDE | |
| | | | LTKNQVSLLCLVKGFYPSDIAVEWESNGQPENNYLTWP | |
| | | | PVLDSDGSFFLYSKLTVDKSRWQQGNVFSCSVMHEALH | |
| | | | NHYTQKSLSLSPG | |
| 123 | 16773 | Full | CAGGTGCAGCTGGTGGAGTCCGGCGGCGGCGTGGTG | |
| | | | CAGCCAGGCAGGAGCCTGCGCCTGTCCTGCGCAGCCT | |
| | | | CTGGCTTCACATTTTCTAACTACGGCATGTATTGGGTG | |
| | | | AGACAGGCCCCAGGCAAGGGACTGGAGTGGGTGGC | |
| | | | CGTGATCTGGTACGACGGCTCTAATAAGTACTATGCC | |
| | | | GATAGCGTGAAGGGCAGGTTCACCATCAGCCGCGAC | |
| | | | AACTCCAAGAATACACTGTATCTGCAGATGAACTCCC | |
| | | | TGAGGGCCGAGGATACCGCCGTGTACTATTGCGCCC | |
| | | | GCGACCTGTGGGGCTGGTACTTTGATTATTGGGGCCA | |
| | | | GGGCACCCTGGTGACAGTGAGCAGCGGAGGAGGAG | |
| | | | GCTCCGGCGGCGGAGGCTCTGGCGGCGGCGGCAGC | |
| | | | GGAGGCGGCGGCTCCGAGATCGTGCTGACCCAGTCT | |
| | | | CCAGCCACACTGTCTCTGAGCCCAGGAGAGAGGGCC | |
| | | | ACCCTGAGCTGTCGCGCCTCCCAGAGCGTGAGCAGCT | |
| | | | ACCTGGCCTGGTATCAGCAGAAGCCAGGACAGGCCC | |
| | | | CTCGGCTGCTGATCTACGACGCCAGCAACAGGGCAAC | |
| | | | CGGCATCCCCGCAAGATTCAGCGGCTCCGGCTCTGGC | |
| | | | ACAGACTTTACCCTGACAATCTCCTCTCTGGAGCCTGA | |
| | | | GGATTTCGCCGTGTACTATTGCCAGCAGCGGAGAAAT | |
| | | | TGGCCACTGACCTTTGGCGGCGGCACAAAGGTGGAG | |
| | | | ATCAAGGGAGGAGGAGGCTCCCAGGTCCAGCTGGTC | |
| | | | GAGTCTGGAGGAGGAGTGGTGCAGCCCGGCAGAAG | |
| | | | CCTGCGGCTGAGCTGTGCAGCCTCCGGCTTCACCTTTT | |
| | | | CCAATTATGGCATGTATTGGGTGCGGCAGGCCCCTGG | |
| | | | CAAGGGCCTGGAATGGGTCGCCGTGATCTGGTATGA | |
| | | | TGGCAGCAATAAGTATTACGCCGATTCCGTGAAGGGC | |
| | | | CGGTTCACCATCTCTAGAGACAACAGCAAGAATACAC | |
| | | | TGTACCTGCAGATGAATAGCCTGCGGGCCGAGGATA | |
| | | | CAGCCGTGTACTATTGTGCCAGAGACCTGTGGGGATG | |
| | | | GTATTTCGACTACTGGGGACAGGGCACCCTGGTCACA | |
| | | | GTGAGCTCCGCTAGCACCAAGGGACCATCCGTGTTCC | |
| | | | CACTGGCACCAAGCTCCAAGTCTACAAGCGGAGGAA | |
| | | | CCGCCGCCCTGGGCTGTCTGGTGAAGGATTACTTCCC | |
| | | | AGAGCCCGTGACCGTGTCTTGGAACAGCGGGGCCCT | |
| | | | GACCAGCGGAGTGCACACCTTTCCTGCCGTGCTGCAG | |
| | | | TCTAGCGGCCTGTATAGCCTGTCCTCTGTGGTCACAG | |
| | | | TGCCAAGCTCCTCTCTGGGCACACAGACCTACATCTG | |
| | | | CAACGTGAATCACAAGCCATCCAATACCAAGGTCGAC | |
| | | | AAGAAGGTGGAGCCCAAGTCTTGTGATAAGACACAC | |
| | | | ACCTGCCCACCTTGTCCGGCGCCAGAGGCCGCCGGA | |
| | | | GGACCAAGCGTGTTCCTGTTTCCACCCAAGCCTAAGG | |
| | | | ACACACTGATGATCAGCAGGACACCAGAGGTGACCT | |
| | | | GCGTGGTGGTGTCCGTGTCTCACGAGGACCCCGAGG | |
| | | | TGAAGTTTAACTGGTACGTGGATGGCGTGGAGGTGC | |
| | | | ACAATGCCAAGACCAAGCCAAGGGAGGAGCAGTATA | |
| | | | ACTCTACATACCGCGTGGTGAGCGTGCTGACCGTGCT | |
| | | | GCACCAGGATTGGCTGAACGGCAAGGAGTACAAGTG | |
| | | | CAAGGTGAGCAATAAGGCCCTGCCCGCCCCTATCGAG | |
| | | | AAGACAATCTCCAAGGCCAAGGGCCAGCCTCGCGAA | |
| | | | CCACAGGTGTATGTGCTGCCTCCATCTAGAGACGAGC | |
| | | | TGACCAAGAACCAGGTGAGCCTGCTGTGCCTGGTGA | |
| | | | AGGGCTTCTACCCCAGCGATATCGCCGTGGAGTGGG | |
| | | | AGTCCAATGGCCAGCCTGAGAACAATTATCTGACATG | |
| | | | GCCCCCTGTGCTGGACTCCGATGGCTCTTTCTTTCTGT | |
| | | | ACTCCAAGCTGACCGTGGACAAGTCTCGCTGGCAGCA | |
| | | | GGGCAACGTGTTTAGCTGTTCCGTGATGCACGAGGCC | |
| | | | CTGCACAATCACTACACCCAGAAGTCTCTGAGCTTAA | |
| | | | GCCCTGGC | |
| 124 | 16774 | Full | EVKLVESGGGLVQPGGSLKLSCATSGFTFSDYYMYWVR | VH = E1- |
| | | | QTPEKRLEWVAYINSGGGSTYYPDTVKGRFTISRDNAK | S119; |
| | | | NTLYLQMSRLKSEDTAMYYCARRGLPFHAMDYWGQG | VL = D140- |
| | | | TSVTVSSGGGGSGGGGSGGGGSGGGGSDIQMTQTTSS | K246; |
| | | | LSASLGDRVTISCSASQGISNYLNWYQQKPDGTVKLLIYY | VH = E252- |
| | | | TSILHSGVPSRFSGSGSGTDYSLTIGNLEPEDIATYYCQQF | S370; |
| | | | NKLPPTFGGGTKLEIKGGGGSEVKLVESGGGLVQPGGSL | CH1 = |
| | | | KLSCATSGFTFSDYYMYWVRQTPEKRLEWVAYINSGGG | A371-V468 |
| | | | STYYPDTVKGRFTISRDNAKNTLYLQMSRLKSEDTAMYY | |
| | | | CARRGLPFHAMDYWGQGTSVTVSSASTKGPSVFPLAPS | |
| | | | SKSTSGGTAALGCLVKDYFPEPVTVSWNSGALTSGVHTF | |
| | | | PAVLQSSGLYSLSSVVTVPSSSLGTQTYICNVNHKPSNTK | |
| | | | VDKKVEPKSCDKTHTCPPCPAPEAAGGPSVFLFPPKPKD | |
| | | | TLMISRTPEVTCVVVSVSHEDPEVKFNWYVDGVEVHNA | |
| | | | KTKPREEQYNSTYRVVSVLTVLHQDWLNGKEYKCKVSN | |
| | | | KALPAPIEKTISKAKGQPREPQVYVLPPSRDELTKNQVSL | |
| | | | LCLVKGFYPSDIAVEWESNGQPENNYLTWPPVLDSDGS | |
| | | | FFLYSKLTVDKSRWQQGNVFSCSVMHEALHNHYTQKSL | |
| | | | SLSPG | |
| 125 | 16774 | Full | GAGGTGAAGCTGGTGGAGTCCGGAGGAGGACTGGT | |
| | | | GCAGCCTGGAGGCTCTCTGAAGCTGAGCTGCGCCACC | |
| | | | TCCGGCTTCACATTTTCTGACTACTATATGTACTGGGT | |
| | | | GCGGCAGACCCCTGAGAAGAGACTGGAGTGGGTGG | |
| | | | CCTATATCAACTCTGGCGGCGGCAGCACCTACTATCC | |
| | | | AGACACAGTGAAGGGCCGGTTCACCATCTCCAGAGA | |
| | | | TAACGCCAAGAATACACTGTACCTGCAGATGTCCCGG | |
| | | | CTGAAGTCTGAGGACACAGCCATGTACTATTGCGCCC | |
| | | | GGAGAGGCCTGCCTTTTCACGCCATGGATTATTGGGG | |
| | | | CCAGGGCACCAGCGTGACAGTGAGCAGCGGAGGAG | |
| | | | GAGGCTCCGGCGGCGGAGGCTCTGGCGGCGGCGGC | |
| | | | AGCGGAGGCGGCGGCTCCGACATCCAGATGACCCAG | |
| | | | ACCACATCTAGCCTGAGCGCCTCCCTGGGCGATAGGG | |
| | | | TGACAATCTCTTGTAGCGCCTCCCAGGGCATCTCTAAC | |
| | | | TACCTGAATTGGTATCAGCAGAAGCCAGACGGCACC | |
| | | | GTGAAGCTGCTGATCTACTATACAAGCATCCTGCACT | |
| | | | CCGGCGTGCCCTCTCGCTTTTCTGGCAGCGGCTCCGG | |
| | | | AACCGACTACAGCCTGACAATCGGCAACCTGGAGCCA | |
| | | | GAGGATATCGCCACCTACTATTGCCAGCAGTTCAATA | |
| | | | AGCTGCCCCCTACCTTTGGCGGCGGCACAAAGCTGGA | |
| | | | GATCAAGGGAGGAGGAGGCTCTGAAGTCAAGCTGGT | |
| | | | GGAGAGTGGCGGAGGACTGGTGCAGCCAGGAGGCA | |
| | | | GCCTGAAGCTGTCCTGTGCCACCTCTGGCTTCACCTTC | |
| | | | AGCGATTATTACATGTACTGGGTGAGGCAGACCCCAG | |
| | | | AGAAGCGCCTGGAATGGGTCGCCTATATCAATAGCG | |
| | | | GCGGCGGCTCCACCTACTATCCTGACACAGTGAAGGG | |
| | | | CAGGTTCACCATCTCCCGCGATAATGCTAAAAACACC | |
| | | | CTGTACCTGCAGATGTCTAGGCTGAAGAGCGAGGAC | |
| | | | ACCGCCATGTACTATTGTGCAAGGCGCGGCCTGCCAT | |
| | | | TTCACGCAATGGATTACTGGGGCCAGGGCACCTCCGT | |
| | | | GACAGTGTCCTCTGCTAGCACCAAGGGACCATCCGTG | |
| | | | TTCCCACTGGCACCAAGCTCCAAGTCTACAAGCGGAG | |
| | | | GAACCGCCGCCCTGGGCTGTCTGGTGAAGGATTACTT | |
| | | | CCCAGAGCCCGTGACCGTGTCTTGGAACAGCGGGGC | |
| | | | CCTGACCAGCGGAGTGCACACCTTTCCTGCCGTGCTG | |
| | | | CAGTCTAGCGGCCTGTATAGCCTGTCCTCTGTGGTCA | |
| | | | CAGTGCCAAGCTCCTCTCTGGGCACACAGACCTACAT | |
| | | | CTGCAACGTGAATCACAAGCCATCCAATACCAAGGTC | |
| | | | GACAAGAAGGTGGAGCCCAAGTCTTGTGATAAGACA | |
| | | | CACACCTGCCCACCTTGTCCGGCGCCAGAGGCCGCCG | |
| | | | GAGGACCAAGCGTGTTCCTGTTTCCACCCAAGCCTAA | |
| | | | GGACACACTGATGATCAGCAGGACACCAGAGGTGAC | |
| | | | CTGCGTGGTGGTGTCCGTGTCTCACGAGGACCCCGAG | |
| | | | GTGAAGTTTAACTGGTACGTGGATGGCGTGGAGGTG | |
| | | | CACAATGCCAAGACCAAGCCAAGGGAGGAGCAGTAT | |
| | | | AACTCTACATACCGCGTGGTGAGCGTGCTGACCGTGC | |
| | | | TGCACCAGGATTGGCTGAACGGCAAGGAGTACAAGT | |
| | | | GCAAGGTGAGCAATAAGGCCCTGCCCGCCCCTATCGA | |
| | | | GAAGACAATCTCCAAGGCCAAGGGCCAGCCTCGCGA | |
| | | | ACCACAGGTGTATGTGCTGCCTCCATCTAGAGACGAG | |
| | | | CTGACCAAGAACCAGGTGAGCCTGCTGTGCCTGGTG | |
| | | | AAGGGCTTCTACCCCAGCGATATCGCCGTGGAGTGG | |
| | | | GAGTCCAATGGCCAGCCTGAGAACAATTATCTGACAT | |
| | | | GGCCCCCTGTGCTGGACTCCGATGGCTCTTTCTTTCTG | |
| | | | TACTCCAAGCTGACCGTGGACAAGTCTCGCTGGCAGC | |
| | | | AGGGCAACGTGTTTAGCTGTTCCGTGATGCACGAGGC | |
| | | | CCTGCACAATCACTACACCCAGAAGTCTCTGAGCTTA | |
| | | | AGCCCTGGC | |
| 126 | 16778 | Full | QVQLQQSGAELARPGASVKMSCKASGYTFTTYTMHW | VH = Q1- |
| | | | VKQRPGQGLEWIGYINPSSGYTNYNQKFKDKATLTADK | S121; |
| | | | SSSTASMQLSSLTSEDSAVYYCARERAVLVPYAMDYWG | VL = Q142- |
| | | | QGTSVTVSSGGGGSGGGGSGGGGSGGGGSQIVLTQSP | K247 |
| | | | AVMSASPGEKVTITCTASSSLSYMHWFQQKPGTSPKL | |
| | | | WLYSTSILASGVPTRFSGSGSGTSYSLTISRMEAEDAATY | |
| | | | YCQQRSSSPFTFGSGTKLEIKAAEPKSSDKTHTCPPCPAP | |
| | | | EAAGGPSVFLFPPKPKDTLMISRTPEVTCVVVSVSHEDP | |
| | | | EVKFNWYVDGVEVHNAKTKPREEQYNSTYRVVSVLTVL | |
| | | | HQDWLNGKEYKCKVSNKALPAPIEKTISKAKGQPREPQ | |
| | | | VYVLPPSRDELTKNQVSLLCLVKGFYPSDIAVEWESNGQ | |
| | | | PENNYLTWPPVLDSDGSFFLYSKLTVDKSRWQQGNVFS | |
| | | | CSVMHEALHNHYTQKSLSLSPG | |
| 127 | 16778 | Full | CAGGTGCAGCTGCAGCAGTCCGGAGCCGAGCTGGCC | |
| | | | CGCCCCGGGGCCAGCGTGAAGATGTCTTGCAAGGCC | |
| | | | AGCGGCTACACATTCACCACATATACCATGCACTGGG | |
| | | | TGAAGCAGAGACCCGGACAGGGACTGGAGTGGATC | |
| | | | GGATACATCAACCCTAGCTCCGGCTACACAAACTATA | |
| | | | ATCAGAAGTTTAAGGACAAGGCCACCCTGACAGCCG | |
| | | | ATAAGTCTAGCTCCACAGCCAGCATGCAGCTGTCTAG | |
| | | | CCTGACCTCTGAGGACAGCGCCGTGTACTATTGTGCC | |
| | | | CGGGAGAGAGCCGTGCTGGTGCCTTACGCCATGGAT | |
| | | | TATTGGGGCCAGGGCACATCCGTGACCGTGTCCTCTG | |
| | | | GCGGCGGCGGCTCCGGAGGCGGCGGCTCTGGAGGA | |
| | | | GGAGGCAGCGGCGGAGGAGGCTCCCAGATCGTGCT | |
| | | | GACCCAGAGCCCTGCCGTGATGTCTGCCAGCCCAGGA | |
| | | | GAGAAGGTGACCATCACATGCACCGCCAGCTCCTCTC | |
| | | | TGTCTTACATGCACTGGTTCCAGCAGAAGCCAGGCAC | |
| | | | AAGCCCCAAGCTGTGGCTGTATTCCACCTCTATCCTGG | |
| | | | CCTCCGGAGTGCCAACACGGTTTAGCGGCTCCGGCTC | |
| | | | TGGCACAAGCTATTCCCTGACCATCTCTCGGATGGAG | |
| | | | GCAGAGGACGCAGCAACCTACTATTGTCAGCAGAGA | |
| | | | AGCTCCTCTCCATTCACATTTGGCAGCGGCACCAAGCT | |
| | | | GGAGATCAAGGCCGCCGAGCCCAAGAGCTCCGATAA | |
| | | | GACACACACCTGCCCCCCTTGTCCGGCGCCAGAGGCC | |
| | | | GCCGGAGGACCAAGCGTGTTCCTGTTTCCACCCAAGC | |
| | | | CTAAGGACACACTGATGATCAGCAGGACACCAGAGG | |
| | | | TGACCTGCGTGGTGGTGTCCGTGTCTCACGAGGACCC | |
| | | | CGAGGTGAAGTTTAACTGGTACGTGGATGGCGTGGA | |
| | | | GGTGCACAATGCCAAGACCAAGCCAAGGGAGGAGCA | |
| | | | GTATAACTCTACATACCGCGTGGTGAGCGTGCTGACC | |
| | | | GTGCTGCACCAGGATTGGCTGAACGGCAAGGAGTAC | |
| | | | AAGTGCAAGGTGAGCAATAAGGCCCTGCCCGCCCCT | |
| | | | ATCGAGAAGACAATCTCCAAGGCCAAGGGCCAGCCT | |
| | | | CGCGAACCACAGGTGTATGTGCTGCCTCCATCTAGAG | |
| | | | ACGAGCTGACCAAGAACCAGGTGAGCCTGCTGTGCC | |
| | | | TGGTGAAGGGCTTCTACCCCAGCGATATCGCCGTGGA | |
| | | | GTGGGAGTCCAATGGCCAGCCTGAGAACAATTATCTG | |
| | | | ACATGGCCCCCTGTGCTGGACTCCGATGGCTCTTTCTT | |
| | | | TCTGTACTCCAAGCTGACCGTGGACAAGTCTCGCTGG | |
| | | | CAGCAGGGCAACGTGTTTAGCTGTTCCGTGATGCACG | |
| | | | AGGCCCTGCACAATCACTACACCCAGAAGTCTCTGAG | |
| | | | CTTAAGCCCTGGC | |
| 128 | 16779 | Full | QVQLVESGGGVVQPGRSLRLSCAASGFTFSNYGMYWV | VH = Q1- |
| | | | RQAPGKGLEWVAVIWYDGSNKYYADSVKGRFTISRDN | S118; |
| | | | SKNTLYLQMNSLRAEDTAVYYCARDLWGWYFDYWGQ | VL = E139- |
| | | | GTLVTVSSGGGGSGGGGSGGGGSGGGGSEIVLTQSPA | K245 |
| | | | TLSLSPGERATLSCRASQSVSSYLAWYQQKPGQAPRLLIY | |
| | | | DASNRATGIPARFSGSGSGTDFTLTISSLEPEDFAVYYCQ | |
| | | | QRRNWPLTFGGGTKVEIKAAEPKSSDKTHTCPPCPAPE | |
| | | | AAGGPSVFLFPPKPKDTLMISRTPEVTCVVVSVSHEDPE | |
| | | | VKFNWYVDGVEVHNAKTKPREEQYNSTYRVVSVLTVLH | |
| | | | QDWLNGKEYKCKVSNKALPAPIEKTISKAKGQPREPQV | |
| | | | YVLPPSRDELTKNQVSLLCLVKGFYPSDIAVEWESNGQP | |
| | | | ENNYLTWPPVLDSDGSFFLYSKLTVDKSRWQQGNVFSC | |
| | | | SVMHEALHNHYTQKSLSLSPG | |
| 129 | 16779 | Full | CAGGTGCAGCTGGTGGAGTCCGGAGGAGGAGTGGT | |
| | | | GCAGCCTGGCAGGAGCCTGCGCCTGTCCTGTGCAGCC | |
| | | | TCTGGCTTCACATTTTCTAACTACGGCATGTATTGGGT | |
| | | | GAGGCAGGCCCCTGGCAAGGGACTGGAGTGGGTGG | |
| | | | CCGTGATCTGGTACGACGGCAGCAATAAGTACTATGC | |
| | | | CGATTCCGTGAAGGGCCGGTTCACCATCAGCAGAGA | |
| | | | CAACTCCAAGAATACACTGTATCTGCAGATGAACAGC | |
| | | | CTGAGGGCCGAGGATACCGCCGTGTACTATTGCGCCC | |
| | | | GCGACCTGTGGGGCTGGTACTTTGATTATTGGGGCCA | |
| | | | GGGCACCCTGGTGACAGTGAGCTCCGGCGGCGGCGG | |
| | | | CTCTGGAGGAGGAGGCAGCGGCGGAGGAGGCTCCG | |
| | | | GAGGAGGCGGCTCTGAGATCGTGCTGACCCAGTCTC | |
| | | | CTGCCACACTGTCTCTGAGCCCAGGAGAGAGGGCCA | |
| | | | CCCTGAGCTGTAGGGCCTCCCAGAGCGTGAGCAGCT | |
| | | | ACCTGGCCTGGTATCAGCAGAAGCCAGGACAGGCCC | |
| | | | CCCGGCTGCTGATCTACGACGCCTCCAACAGGGCAAC | |
| | | | CGGCATCCCAGCCAGATTCAGCGGCTCCGGCTCTGGC | |
| | | | ACAGACTTTACCCTGACAATCTCCTCTCTGGAGCCCGA | |
| | | | GGATTTCGCCGTGTACTATTGCCAGCAGCGGAGAAAT | |
| | | | TGGCCTCTGACCTTTGGCGGCGGCACAAAGGTGGAG | |
| | | | ATCAAGGCCGCCGAGCCCAAGAGCTCCGATAAGACC | |
| | | | CACACATGCCCCCCTTGTCCGGCGCCAGAGGCCGCCG | |
| | | | GAGGACCAAGCGTGTTCCTGTTTCCACCCAAGCCTAA | |
| | | | GGACACACTGATGATCAGCAGGACACCAGAGGTGAC | |
| | | | CTGCGTGGTGGTGTCCGTGTCTCACGAGGACCCCGAG | |
| | | | GTGAAGTTTAACTGGTACGTGGATGGCGTGGAGGTG | |
| | | | CACAATGCCAAGACCAAGCCAAGGGAGGAGCAGTAT | |
| | | | AACTCTACATACCGCGTGGTGAGCGTGCTGACCGTGC | |
| | | | TGCACCAGGATTGGCTGAACGGCAAGGAGTACAAGT | |
| | | | GCAAGGTGAGCAATAAGGCCCTGCCCGCCCCTATCGA | |
| | | | GAAGACAATCTCCAAGGCCAAGGGCCAGCCTCGCGA | |
| | | | ACCACAGGTGTATGTGCTGCCTCCATCTAGAGACGAG | |
| | | | CTGACCAAGAACCAGGTGAGCCTGCTGTGCCTGGTG | |
| | | | AAGGGCTTCTACCCCAGCGATATCGCCGTGGAGTGG | |
| | | | GAGTCCAATGGCCAGCCTGAGAACAATTATCTGACAT | |
| | | | GGCCCCCTGTGCTGGACTCCGATGGCTCTTTCTTTCTG | |
| | | | TACTCCAAGCTGACCGTGGACAAGTCTCGCTGGCAGC | |
| | | | AGGGCAACGTGTTTAGCTGTTCCGTGATGCACGAGGC | |
| | | | CCTGCACAATCACTACACCCAGAAGTCTCTGAGCTTA | |
| | | | AGCCCTGGC | |
| 130 | 16780 | Full | EVKLVESGGGLVQPGGSLKLSCATSGFTFSDYYMYWVR | VH = E1- |
| | | | QTPEKRLEWVAYINSGGGSTYYPDTVKGRFTISRDNAK | S119; |
| | | | NTLYLQMSRLKSEDTAMYYCARRGLPFHAMDYWGQG | VL = D140- |
| | | | TSVTVSSGGGGSGGGGSGGGGSGGGGSDIQMTQTTSS | K246 |
| | | | LSASLGDRVTISCSASQGISNYLNWYQQKPDGTVKLLIYY | |
| | | | TSILHSGVPSRFSGSGSGTDYSLTIGNLEPEDIATYYCQQF | |
| | | | NKLPPTFGGGTKLEIKAAEPKSSDKTHTCPPCPAPEAAG | |
| | | | GPSVFLFPPKPKDTLMISRTPEVTCVVVSVSHEDPEVKF | |
| | | | NWYVDGVEVHNAKTKPREEQYNSTYRVVSVLTVLHQD | |
| | | | WLNGKEYKCKVSNKALPAPIEKTISKAKGQPREPQVYVL | |
| | | | PPSRDELTKNQVSLLCLVKGFYPSDIAVEWESNGQPEN | |
| | | | NYLTWPPVLDSDGSFFLYSKLTVDKSRWQQGNVFSCSV | |
| | | | MHEALHNHYTQKSLSLSPG | |
| 131 | 16780 | Full | GAGGTGAAGCTGGTGGAGAGCGGCGGCGGCCTGGT | |
| | | | GCAGCCAGGAGGCTCTCTGAAGCTGAGCTGCGCCAC | |
| | | | CTCCGGCTTCACATTTTCTGACTACTATATGTACTGGG | |
| | | | TGCGGCAGACCCCCGAGAAGAGACTGGAGTGGGTG | |
| | | | GCCTATATCAACTCTGGCGGCGGCAGCACCTACTATC | |
| | | | CTGACACAGTGAAGGGCAGGTTCACCATCAGCCGCG | |
| | | | ATAACGCCAAGAATACACTGTACCTGCAGATGTCCAG | |
| | | | ACTGAAGTCTGAGGACACAGCCATGTACTATTGTGCC | |
| | | | CGGAGAGGCCTGCCTTTTCACGCCATGGATTATTGGG | |
| | | | GCCAGGGCACCTCCGTGACAGTGAGCAGCGGAGGAG | |
| | | | GAGGCAGCGGAGGAGGAGGCTCCGGCGGCGGCGGC | |
| | | | TCTGGAGGAGGAGGCAGCGACATCCAGATGACCCAG | |
| | | | ACCACATCTAGCCTGAGCGCCTCCCTGGGCGATAGGG | |
| | | | TGACAATCTCTTGCAGCGCCTCCCAGGGCATCAGCAA | |
| | | | CTACCTGAATTGGTATCAGCAGAAGCCTGACGGCACC | |
| | | | GTGAAGCTGCTGATCTACTATACAAGCATCCTGCACT | |
| | | | CCGGCGTGCCATCTCGGTTTTCTGGCAGCGGCTCCGG | |
| | | | AACCGACTACTCCCTGACAATCGGCAACCTGGAGCCA | |
| | | | GAGGATATCGCCACCTACTATTGTCAGCAGTTCAATA | |
| | | | AGCTGCCCCCTACCTTTGGCGGCGGCACAAAGCTGGA | |
| | | | GATCAAGGCCGCCGAGCCCAAGTCCTCTGATAAGACC | |
| | | | CACACATGCCCACCCTGTCCGGCGCCAGAGGCCGCCG | |
| | | | GAGGACCAAGCGTGTTCCTGTTTCCACCCAAGCCTAA | |
| | | | GGACACACTGATGATCAGCAGGACACCAGAGGTGAC | |
| | | | CTGCGTGGTGGTGTCCGTGTCTCACGAGGACCCCGAG | |
| | | | GTGAAGTTTAACTGGTACGTGGATGGCGTGGAGGTG | |
| | | | CACAATGCCAAGACCAAGCCAAGGGAGGAGCAGTAT | |
| | | | AACTCTACATACCGCGTGGTGAGCGTGCTGACCGTGC | |
| | | | TGCACCAGGATTGGCTGAACGGCAAGGAGTACAAGT | |
| | | | GCAAGGTGAGCAATAAGGCCCTGCCCGCCCCTATCGA | |
| | | | GAAGACAATCTCCAAGGCCAAGGGCCAGCCTCGCGA | |
| | | | ACCACAGGTGTATGTGCTGCCTCCATCTAGAGACGAG | |
| | | | CTGACCAAGAACCAGGTGAGCCTGCTGTGCCTGGTG | |
| | | | AAGGGCTTCTACCCCAGCGATATCGCCGTGGAGTGG | |
| | | | GAGTCCAATGGCCAGCCTGAGAACAATTATCTGACAT | |
| | | | GGCCCCCTGTGCTGGACTCCGATGGCTCTTTCTTTCTG | |
| | | | TACTCCAAGCTGACCGTGGACAAGTCTCGCTGGCAGC | |
| | | | AGGGCAACGTGTTTAGCTGTTCCGTGATGCACGAGGC | |
| | | | CCTGCACAATCACTACACCCAGAAGTCTCTGAGCTTA | |
| | | | AGCCCTGGC | |
| 132 | 16781 | Full | EPAVYFKEQFLDGDGWTSRWIESKHKSDFGKFVLSSGK | Calreticulin = |
| | | | FYGDEEKDKGLQTSQDARFYALSASFEPFSNKGQTLVV | E1-A397 |
| | | | QFTVKHEQNIDCGGGYVKLFPNSLDQTDMHGDSEYNI | |
| | | | MFGPDICGPGTKKVHVIFNYKGKNVLINKDIRSKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAAAEPKSSDKTHTCPPCPAPEA | |
| | | | AGGPSVFLFPPKPKDTLMISRTPEVTCVVVSVSHEDPEV | |
| | | | KFNWYVDGVEVHNAKTKPREEQYNSTYRVVSVLTVLH | |
| | | | QDWLNGKEYKCKVSNKALPAPIEKTISKAKGQPREPQV | |
| | | | YVLPPSRDELTKNQVSLLCLVKGFYPSDIAVEWESNGQP | |
| | | | ENNYLTWPPVLDSDGSFFLYSKLTVDKSRWQQGNVFSC | |
| | | | SVMHEALHNHYTQKSLSLSPG | |
| 133 | 16781 | Full | GAGCCAGCCGTGTATTTCAAGGAGCAGTTTCTGGACG | |
| | | | GCGATGGCTGGACCTCTAGGTGGATCGAGTCTAAGC | |
| | | | ACAAGAGCGACTTCGGCAAGTTTGTGCTGAGCTCCGG | |
| | | | CAAGTTCTATGGCGATGAGGAGAAGGACAAGGGCCT | |
| | | | GCAGACATCTCAGGATGCCCGGTTTTACGCCCTGTCC | |
| | | | GCCTCTTTCGAGCCCTTCAGCAACAAGGGCCAGACCC | |
| | | | TGGTGGTGCAGTTCACAGTGAAGCACGAGCAGAACA | |
| | | | TCGACTGCGGCGGCGGCTATGTGAAGCTGTTTCCCAA | |
| | | | TAGCCTGGATCAGACCGACATGCACGGCGACTCCGA | |
| | | | GTACAACATCATGTTCGGCCCCGATATCTGTGGCCCT | |
| | | | GGCACAAAGAAGGTGCACGTGATCTTTAATTACAAG | |
| | | | GGCAAGAACGTGCTGATCAATAAGGACATCAGGAGC | |
| | | | AAGGACGATGAGTTCACCCACCTGTACACACTGATCG | |
| | | | TGCGCCCTGACAACACCTATGAGGTGAAGATCGATAA | |
| | | | TTCCCAGGTGGAGAGCGGCTCCCTGGAGGACGATTG | |
| | | | GGATTTTCTGCCCCCTAAGAAGATCAAGGACCCAGAT | |
| | | | GCCTCCAAGCCCGAGGACTGGGATGAGCGCGCCAAG | |
| | | | ATCGACGATCCTACAGACTCTAAGCCAGAGGACTGG | |
| | | | GATAAGCCCGAGCACATCCCCGACCCTGATGCCAAGA | |
| | | | AGCCTGAGGACTGGGATGAGGAGATGGATGGCGAG | |
| | | | TGGGAGCCACCCGTGATCCAGAACCCCGAGTACAAG | |
| | | | GGCGAGTGGAAGCCACGGCAGATCGATAATCCCGAC | |
| | | | TATAAGGGCACCTGGATTCACCCCGAGATCGATAACC | |
| | | | CTGAGTACTCCCCAGACCCCTCTATCTACGCCTATGAT | |
| | | | AATTTCGGCGTGCTGGGCCTGGACCTGTGGCAGGTG | |
| | | | AAGTCCGGCACCATCTTCGACAACTTTCTGATCACAAA | |
| | | | TGATGAGGCCTATGCCGAGGAGTTTGGCAATGAGAC | |
| | | | CTGGGGCGTGACAAAGGCCGCCGAGAAGCAGATGA | |
| | | | AGGATAAGCAGGACGAGGAGCAGCGGCTGAAGGAA | |
| | | | GAGGAGGAGGACAAGAAGAGAAAGGAGGAGGAGG | |
| | | | AGGCCGAGGATAAGGAGGACGATGAGGACAAGGAT | |
| | | | GAGGACGAGGAGGATGAGGAGGACAAGGAGGAGG | |
| | | | ATGAGGAGGAGGACGTGCCTGGACAGGCCGCCGCC | |
| | | | GAGCCAAAGTCTAGCGACAAGACCCACACATGCCCTC | |
| | | | CATGTCCGGCGCCAGAGGCCGCCGGAGGACCAAGCG | |
| | | | TGTTCCTGTTTCCACCCAAGCCTAAGGACACACTGATG | |
| | | | ATCAGCAGGACACCAGAGGTGACCTGCGTGGTGGTG | |
| | | | TCCGTGTCTCACGAGGACCCCGAGGTGAAGTTTAACT | |
| | | | GGTACGTGGATGGCGTGGAGGTGCACAATGCCAAGA | |
| | | | CCAAGCCAAGGGAGGAGCAGTATAACTCTACATACC | |
| | | | GCGTGGTGAGCGTGCTGACCGTGCTGCACCAGGATT | |
| | | | GGCTGAACGGCAAGGAGTACAAGTGCAAGGTGAGC | |
| | | | AATAAGGCCCTGCCCGCCCCTATCGAGAAGACAATCT | |
| | | | CCAAGGCCAAGGGCCAGCCTCGCGAACCACAGGTGT | |
| | | | ATGTGCTGCCTCCATCTAGAGACGAGCTGACCAAGAA | |
| | | | CCAGGTGAGCCTGCTGTGCCTGGTGAAGGGCTTCTAC | |
| | | | CCCAGCGATATCGCCGTGGAGTGGGAGTCCAATGGC | |
| | | | CAGCCTGAGAACAATTATCTGACATGGCCCCCTGTGC | |
| | | | TGGACTCCGATGGCTCTTTCTTTCTGTACTCCAAGCTG | |
| | | | ACCGTGGACAAGTCTCGCTGGCAGCAGGGCAACGTG | |
| | | | TTTAGCTGTTCCGTGATGCACGAGGCCCTGCACAATC | |
| | | | ACTACACCCAGAAGTCTCTGAGCTTAAGCCCTGGC | |
| 134 | 16782 | Full | EPAVYFKEQFLDGDGWTSRWIESKHKSDFGKFVLSSGK | Calreticulin = |
| | | | FYGDEEKDKGLQTSQDARFYALSASFEPFSNKGQTLVV | E1-K258 |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPGSGD | |
| | | | PSIYAYDNFGVLGLDLWQVKSGTIFDNFLITNDEAYAEEF | |
| | | | GNETWGVTKAAEKQMKDKQDEEQRLKGGGGSEPKSS | |
| | | | DKTHTCPPCPAPEAAGGPSVFLFPPKPKDTLMISRTPEV | |
| | | | TCVVVSVSHEDPEVKFNWYVDGVEVHNAKTKPREEQY | |
| | | | NSTYRVVSVLTVLHQDWLNGKEYKCKVSNKALPAPIEKT | |
| | | | ISKAKGQPREPQVYVLPPSRDELTKNQVSLLCLVKGFYPS | |
| | | | DIAVEWESNGQPENNYLTWPPVLDSDGSFFLYSKLTVD | |
| | | | KSRWQQGNVFSCSVMHEALHNHYTQKSLSLSPG | |
| 135 | 16782 | Full | GAGCCCGCCGTGTACTTCAAGGAGCAGTTTCTGGACG | |
| | | | GCGATGGATGGACCAGCCGGTGGATCGAGTCTAAGC | |
| | | | ACAAGAGCGATTTCGGCAAGTTTGTGCTGAGCTCCGG | |
| | | | CAAGTTCTACGGCGACGAAGAGAAGGATAAGGGCCT | |
| | | | GCAGACATCTCAGGACGCCAGGTTTTATGCCCTGTCC | |
| | | | GCCTCTTTCGAGCCCTTCAGCAACAAGGGCCAGACCC | |
| | | | TGGTGGTGCAGTTCACAGTGAAGCACGAGCAGAACA | |
| | | | TCGATTGCGGCGGCGGCTACGTGAAGCTGTTTCCCAA | |
| | | | TAGCCTGGACCAGACCGATATGCACGGCGATTCCGA | |
| | | | GTATAACATCATGTTCGGCCCTGACATCTGCGGCCCA | |
| | | | GGCACAAAGAAGGTGCACGTGATCTTTAATTACAAG | |
| | | | GGCAAGAACGTGCTGATCAATAAGGACATCCGGTGT | |
| | | | AAGGACGATGAGTTCACCCACCTGTACACACTGATCG | |
| | | | TGAGACCTGATAACACCTATGAGGTGAAGATCGACA | |
| | | | ATTCCCAGGTGGAGAGCGGCTCCCTGGAGGACGATT | |
| | | | GGGACTTCCTGCCCGGCTCCGGCGATCCTTCTATCTAC | |
| | | | GCCTATGACAACTTTGGCGTGCTGGGCCTGGATCTGT | |
| | | | GGCAGGTGAAGTCTGGCACCATCTTCGATAACTTTCT | |
| | | | GATCACAAATGACGAGGCCTATGCCGAGGAGTTTGG | |
| | | | CAATGAGACCTGGGGCGTGACAAAGGCCGCCGAGAA | |
| | | | GCAGATGAAGGACAAGCAGGATGAGGAGCAGCGGC | |
| | | | TGAAGGGAGGAGGAGGCTCCGAGCCAAAGTCTAGC | |
| | | | GACAAGACCCACACATGCCCCCCTTGTCCGGCGCCAG | |
| | | | AGGCCGCCGGAGGACCAAGCGTGTTCCTGTTTCCACC | |
| | | | CAAGCCTAAGGACACACTGATGATCAGCAGGACACC | |
| | | | AGAGGTGACCTGCGTGGTGGTGTCCGTGTCTCACGA | |
| | | | GGACCCCGAGGTGAAGTTTAACTGGTACGTGGATGG | |
| | | | CGTGGAGGTGCACAATGCCAAGACCAAGCCAAGGGA | |
| | | | GGAGCAGTATAACTCTACATACCGCGTGGTGAGCGT | |
| | | | GCTGACCGTGCTGCACCAGGATTGGCTGAACGGCAA | |
| | | | GGAGTACAAGTGCAAGGTGAGCAATAAGGCCCTGCC | |
| | | | CGCCCCTATCGAGAAGACAATCTCCAAGGCCAAGGG | |
| | | | CCAGCCTCGCGAACCACAGGTGTATGTGCTGCCTCCA | |
| | | | TCTAGAGACGAGCTGACCAAGAACCAGGTGAGCCTG | |
| | | | CTGTGCCTGGTGAAGGGCTTCTACCCCAGCGATATCG | |
| | | | CCGTGGAGTGGGAGTCCAATGGCCAGCCTGAGAACA | |
| | | | ATTATCTGACATGGCCCCCTGTGCTGGACTCCGATGG | |
| | | | CTCTTTCTTTCTGTACTCCAAGCTGACCGTGGACAAGT | |
| | | | CTCGCTGGCAGCAGGGCAACGTGTTTAGCTGTTCCGT | |
| | | | GATGCACGAGGCCCTGCACAATCACTACACCCAGAAG | |
| | | | TCTCTGAGCTTAAGCCCTGGC | |
| 136 | 16783 | Full | EPAVYFKEQFLDGDGWTSRWIESKHKSDFGKFVLSSGK | Calreticulin = |
| | | | FYGDEEKDKGLQTSQDARFYALSASFEPFSNKGQTLVV | E1-K352 |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKGGGGSEPKSSDKTHTCPPCPAPEAAGGP | |
| | | | SVFLFPPKPKDTLMISRTPEVTCVVVSVSHEDPEVKFNW | |
| | | | YVDGVEVHNAKTKPREEQYNSTYRVVSVLTVLHQDWL | |
| | | | NGKEYKCKVSNKALPAPIEKTISKAKGQPREPQVYVLPPS | |
| | | | RDELTKNQVSLLCLVKGFYPSDIAVEWESNGQPENNYLT | |
| | | | WPPVLDSDGSFFLYSKLTVDKSRWQQGNVFSCSVMHE | |
| | | | ALHNHYTQKSLSLSPG | |
| 137 | 16783 | Full | GAGCCAGCCGTGTATTTCAAGGAGCAGTTTCTGGACG | |
| | | | GCGATGGCTGGACCTCTCGGTGGATCGAGTCTAAGC | |
| | | | ACAAGAGCGATTTCGGCAAGTTTGTGCTGAGCTCCGG | |
| | | | CAAGTTCTATGGCGACGAGGAGAAGGATAAGGGCCT | |
| | | | GCAGACATCTCAGGACGCCCGCTTTTACGCCCTGTCC | |
| | | | GCCTCTTTCGAGCCCTTTAGCAACAAGGGCCAGACCC | |
| | | | TGGTGGTGCAGTTCACAGTGAAGCACGAGCAGAACA | |
| | | | TCGACTGCGGCGGCGGCTATGTGAAGCTGTTTCCTAA | |
| | | | TAGCCTGGACCAGACCGATATGCACGGCGATTCCGA | |
| | | | GTACAACATCATGTTCGGACCAGACATCTGCGGACCT | |
| | | | GGAACAAAGAAGGTGCACGTGATCTTTAATTACAAG | |
| | | | GGCAAGAACGTGCTGATCAATAAGGATATCCGGTGT | |
| | | | AAGGACGATGAGTTCACCCACCTGTACACACTGATCG | |
| | | | TGAGACCAGATAACACCTATGAGGTGAAGATCGACA | |
| | | | ATTCCCAGGTGGAGAGCGGCTCCCTGGAGGACGATT | |
| | | | GGGACTTTCTGCCCCCTAAGAAGATCAAGGACCCAGA | |
| | | | TGCCTCCAAGCCCGAGGACTGGGATGAGAGAGCCAA | |
| | | | GATCGACGATCCTACAGATTCTAAGCCAGAGGACTGG | |
| | | | GATAAGCCTGAGCACATCCCCGACCCTGATGCCAAGA | |
| | | | AGCCTGAAGACTGGGATGAGGAGATGGACGGCGAG | |
| | | | TGGGAGCCACCCGTGATCCAGAACCCCGAGTACAAG | |
| | | | GGCGAGTGGAAGCCAAGGCAGATCGACAATCCCGAT | |
| | | | TATAAGGGCACCTGGATTCACCCCGAGATCGACAACC | |
| | | | CTGAGTACTCCCCAGATCCCTCTATCTACGCCTATGAC | |
| | | | AATTTCGGCGTGCTGGGCCTGGATCTGTGGCAGGTG | |
| | | | AAGAGCGGCACCATCTTCGATAACTTTCTGATCACAA | |
| | | | ATGACGAGGCCTATGCCGAGGAGTTTGGCAATGAGA | |
| | | | CCTGGGGCGTGACAAAGGCCGCCGAGAAGCAGATGA | |
| | | | AGGACAAGCAGGATGAAGAGCAGCGGCTGAAGGGA | |
| | | | GGAGGAGGCTCCGAGCCCAAGTCTAGCGACAAGACC | |
| | | | CACACATGCCCTCCATGTCCGGCGCCAGAGGCCGCCG | |
| | | | GAGGACCAAGCGTGTTCCTGTTTCCACCCAAGCCTAA | |
| | | | GGACACACTGATGATCAGCAGGACACCAGAGGTGAC | |
| | | | CTGCGTGGTGGTGTCCGTGTCTCACGAGGACCCCGAG | |
| | | | GTGAAGTTTAACTGGTACGTGGATGGCGTGGAGGTG | |
| | | | CACAATGCCAAGACCAAGCCAAGGGAGGAGCAGTAT | |
| | | | AACTCTACATACCGCGTGGTGAGCGTGCTGACCGTGC | |
| | | | TGCACCAGGATTGGCTGAACGGCAAGGAGTACAAGT | |
| | | | GCAAGGTGAGCAATAAGGCCCTGCCCGCCCCTATCGA | |
| | | | GAAGACAATCTCCAAGGCCAAGGGCCAGCCTCGCGA | |
| | | | ACCACAGGTGTATGTGCTGCCTCCATCTAGAGACGAG | |
| | | | CTGACCAAGAACCAGGTGAGCCTGCTGTGCCTGGTG | |
| | | | AAGGGCTTCTACCCCAGCGATATCGCCGTGGAGTGG | |
| | | | GAGTCCAATGGCCAGCCTGAGAACAATTATCTGACAT | |
| | | | GGCCCCCTGTGCTGGACTCCGATGGCTCTTTCTTTCTG | |
| | | | TACTCCAAGCTGACCGTGGACAAGTCTCGCTGGCAGC | |
| | | | AGGGCAACGTGTTTAGCTGTTCCGTGATGCACGAGGC | |
| | | | CCTGCACAATCACTACACCCAGAAGTCTCTGAGCTTA | |
| | | | AGCCCTGGC | |
| 138 | 16784 | Full | EPAVYFKEQFLDGDGWTSRWIESKHKSDFGKFVLSSGK | |
| | | | FYGDEEKDKGLQTSQDARFYALSASFEPFSNKGQTLVV | |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAGGGGSEPAVYFKEQFLDGDG | |
| | | | WTSRWIESKHKSDFGKFVLSSGKFYGDEEKDKGLQTSQ | |
| | | | DARFYALSASFEPFSNKGQTLVVQFTVKHEQNIDCGGG | |
| | | | YVKLFPNSLDQTDMHGDSEYNIMFGPDICGPGTKKVHV | |
| | | | IFNYKGKNVLINKDIRCKDDEFTHLYTLIVRPDNTYEVKID | |
| | | | NSQVESGSLEDDWDFLPPKKIKDPDASKPEDWDERAKI | |
| | | | DDPTDSKPEDWDKPEHIPDPDAKKPEDWDEEMDGEW | |
| | | | EPPVIQNPEYKGEWKPRQIDNPDYKGTWIHPEIDNPEY | |
| | | | SPDPSIYAYDNFGVLGLDLWQVKSGTIFDNFLITNDEAY | |
| | | | AEEFGNETWGVTKAAEKQMKDKQDEEQRLKEEEEDKK | |
| | | | RKEEEEAEDKEDDEDKDEDEEDEEDKEEDEEEDVPGQA | |
| | | | AAEPKSSDKTHTCPPCPAPEAAGGPSVFLFPPKPKDTLM | |
| | | | ISRTPEVTCVVVSVSHEDPEVKFNWYVDGVEVHNAKTK | |
| | | | PREEQYNSTYRVVSVLTVLHQDWLNGKEYKCKVSNKAL | |
| | | | PAPIEKTISKAKGQPREPQVYVLPPSRDELTKNQVSLLCL | |
| | | | VKGFYPSDIAVEWESNGQPENNYLTWPPVLDSDGSFFL | |
| | | | YSKLTVDKSRWQQGNVFSCSVMHEALHNHYTQKSLSLS | |
| | | | PG | |
| 139 | 16784 | Full | GAGCCTGCCGTGTACTTCAAGGAGCAGTTTCTGGACG | |
| | | | GCGATGGCTGGACCAGCAGGTGGATCGAGTCTAAGC | |
| | | | ACAAGAGCGACTTCGGCAAGTTTGTGCTGAGCTCCGG | |
| | | | CAAGTTCTACGGCGACGAGGAGAAGGATAAGGGCCT | |
| | | | GCAGACATCTCAGGATGCCAGGTTTTATGCCCTGAGC | |
| | | | GCCTCCTTCGAGCCCTTTAGCAACAAGGGCCAGACCC | |
| | | | TGGTGGTGCAGTTCACAGTGAAGCACGAGCAGAACA | |
| | | | TCGACTGCGGCGGCGGCTACGTGAAGCTGTTTCCTAA | |
| | | | TTCCCTGGACCAGACCGATATGCACGGCGACTCTGAG | |
| | | | TATAACATCATGTTCGGCCCAGATATCTGCGGCCCCG | |
| | | | GCACAAAGAAGGTGCACGTGATCTTTAATTATAAGGG | |
| | | | CAAGAACGTGCTGATCAATAAGGACATCCGGTGTAA | |
| | | | GGACGATGAGTTCACCCACCTGTACACACTGATCGTG | |
| | | | AGACCTGACAACACCTATGAGGTGAAGATCGATAATA | |
| | | | GCCAGGTGGAGTCTGGCAGCCTGGAGGACGATTGGG | |
| | | | ATTTTCTGCCCCCTAAGAAGATCAAGGACCCTGATGC | |
| | | | CAGCAAGCCAGAGGACTGGGATGAGAGAGCCAAGA | |
| | | | TCGACGATCCCACAGACTCCAAGCCTGAGGACTGGG | |
| | | | ATAAGCCAGAGCACATCCCTGACCCAGATGCCAAGAA | |
| | | | GCCCGAGGACTGGGATGAGGAGATGGATGGCGAGT | |
| | | | GGGAGCCACCCGTGATCCAGAACCCAGAGTACAAGG | |
| | | | GCGAGTGGAAGCCCAGGCAGATCGACAATCCTGATT | |
| | | | ATAAGGGCACCTGGATTCACCCAGAGATCGACAACCC | |
| | | | CGAGTACTCCCCCGATCCTTCTATCTACGCCTATGACA | |
| | | | ATTTCGGCGTGCTGGGCCTGGACCTGTGGCAGGTGA | |
| | | | AGTCCGGCACCATCTTCGATAACTTTCTGATCACAAAT | |
| | | | GACGAGGCCTACGCCGAGGAGTTTGGCAACGAGACC | |
| | | | TGGGGCGTGACAAAGGCCGCCGAGAAGCAGATGAA | |
| | | | GGACAAGCAGGATGAAGAGCAGCGGCTGAAGGAAG | |
| | | | AGGAGGAGGACAAGAAGAGAAAGGAGGAGGAGGA | |
| | | | GGCCGAGGATAAGGAGGACGATGAGGACAAGGATG | |
| | | | AGGACGAGGAGGACGAGGAGGATAAGGAGGAGGA | |
| | | | CGAGGAGGAGGATGTGCCAGGACAGGCCGGAGGCG | |
| | | | GAGGCTCCGAGCCTGCCGTGTATTTCAAGGAACAGTT | |
| | | | TCTGGATGGCGACGGCTGGACCTCTCGCTGGATCGA | |
| | | | GAGCAAGCACAAGTCTGATTTTGGCAAGTTTGTGCTG | |
| | | | TCTAGTGGCAAGTTCTACGGCGACGAAGAAAAAGAC | |
| | | | AAAGGCCTGCAGACATCCCAGGATGCCCGGTTTTATG | |
| | | | CCCTGTCCGCCTCTTTCGAGCCATTTTCTAATAAGGGA | |
| | | | CAGACCCTGGTCGTCCAGTTCACAGTCAAACATGAGC | |
| | | | AGAACATCGACTGTGGAGGAGGATATGTGAAGCTGT | |
| | | | TTCCCAATAGCCTGGATCAGACTGATATGCACGGCGA | |
| | | | CTCCGAATACAACATCATGTTCGGCCCTGATATCTGCG | |
| | | | GCCCAGGAACAAAGAAGGTCCACGTGATCTTTAATTA | |
| | | | CAAAGGCAAGAACGTGCTGATCAATAAGGATATCAG | |
| | | | ATGCAAAGATGACGAGTTCACCCACCTGTATACACTG | |
| | | | ATCGTGCGCCCCGATAATACTTACGAAGTCAAAATTG | |
| | | | ACAACAGCCAGGTGGAGAGCGGCTCCCTGGAAGATG | |
| | | | ATTGGGACTTCCTGCCTCCCAAGAAGATCAAGGACCC | |
| | | | CGACGCCTCTAAGCCTGAGGATTGGGACGAGCGCGC | |
| | | | CAAGATCGACGATCCAACAGACAGCAAGCCCGAGGA | |
| | | | TTGGGACAAGCCTGAGCACATCCCAGATCCCGACGCC | |
| | | | AAGAAGCCAGAGGATTGGGACGAAGAAATGGACGG | |
| | | | AGAGTGGGAGCCCCCTGTGATCCAGAACCCTGAGTAT | |
| | | | AAGGGCGAGTGGAAGCCACGGCAGATCGACAATCCC | |
| | | | GATTACAAAGGAACCTGGATTCACCCTGAGATCGATA | |
| | | | ACCCAGAGTATTCTCCTGACCCAAGCATCTACGCCTAT | |
| | | | GATAACTTTGGCGTGCTGGGCTTAGACCTGTGGCAGG | |
| | | | TCAAATCCGGCACCATCTTCGACAACTTTCTGATTACC | |
| | | | AATGATGAAGCTTATGCTGAAGAGTTTGGAAATGAA | |
| | | | ACTTGGGGAGTCACCAAAGCCGCCGAGAAACAGATG | |
| | | | AAAGATAAACAGGACGAGGAGCAGAGGCTGAAGGA | |
| | | | AGAAGAGGAGGACAAGAAGCGCAAAGAAGAAGAAG | |
| | | | AAGCTGAAGACAAGGAGGACGATGAGGATAAGGAC | |
| | | | GAGGATGAAGAAGATGAAGAAGACAAAGAAGAAGA | |
| | | | TGAGGAGGAGGATGTGCCTGGACAGGCCGCCGCCGA | |
| | | | GCCAAAGTCCTCTGACAAGACCCACACATGCCCACCC | |
| | | | TGTCCGGCGCCAGAGGCCGCCGGAGGACCAAGCGTG | |
| | | | TTCCTGTTTCCACCCAAGCCTAAGGACACACTGATGAT | |
| | | | CAGCAGGACACCAGAGGTGACCTGCGTGGTGGTGTC | |
| | | | CGTGTCTCACGAGGACCCCGAGGTGAAGTTTAACTGG | |
| | | | TACGTGGATGGCGTGGAGGTGCACAATGCCAAGACC | |
| | | | AAGCCAAGGGAGGAGCAGTATAACTCTACATACCGC | |
| | | | GTGGTGAGCGTGCTGACCGTGCTGCACCAGGATTGG | |
| | | | CTGAACGGCAAGGAGTACAAGTGCAAGGTGAGCAAT | |
| | | | AAGGCCCTGCCCGCCCCTATCGAGAAGACAATCTCCA | |
| | | | AGGCCAAGGGCCAGCCTCGCGAACCACAGGTGTATG | |
| | | | TGCTGCCTCCATCTAGAGACGAGCTGACCAAGAACCA | |
| | | | GGTGAGCCTGCTGTGCCTGGTGAAGGGCTTCTACCCC | |
| | | | AGCGATATCGCCGTGGAGTGGGAGTCCAATGGCCAG | |
| | | | CCTGAGAACAATTATCTGACATGGCCCCCTGTGCTGG | |
| | | | ACTCCGATGGCTCTTTCTTTCTGTACTCCAAGCTGACC | |
| | | | GTGGACAAGTCTCGCTGGCAGCAGGGCAACGTGTTT | |
| | | | AGCTGTTCCGTGATGCACGAGGCCCTGCACAATCACT | |
| | | | ACACCCAGAAGTCTCTGAGCTTAAGCCCTGGC | |
| 140 | 16795 | Full | DIQMTQSPSSLSASVGDRVTITCRASQDVNTAVAWYQ | VL = D1- |
| | | | QKPGKAPKLLIYSASFLYSGVPSRFSGSRSGTDFTLTISSL | K107; |
| | | | QPEDFATYYCQQHYTTPPTFGQGTKVEIKGGSGGGSGG | VH = E128- |
| | | | GSGGGSGGGSGEVQLVESGGGLVQPGGSLRLSCAASG | S247 |
| | | | FNIKDTYIHWVRQAPGKGLEWVARIYPTNGYTRYADSV | |
| | | | KGRFTISADTSKNTAYLQMNSLRAEDTAVYYCSRWGGD | |
| | | | GFYAMDYWGQGTLVTVSSAAEPKSSDKTHTCPPCPAP | |
| | | | EAAGGPSVFLFPPKPKDTLMISRTPEVTCVVVSVSHEDP | |
| | | | EVKFNWYVDGVEVHNAKTKPREEQYNSTYRVVSVLTVL | |
| | | | HQDWLNGKEYKCKVSNKALPAPIEKTISKAKGQPREPQ | |
| | | | VYVYPPSRDELTKNQVSLTCLVKGFYPSDIAVEWESNGQ | |
| | | | PENNYKTTPPVLDSDGSFALVSKLTVDKSRWQQGNVFS | |
| | | | CSVMHEALHNHYTQKSLSLSPG | |
| 141 | 16795 | Full | GACATCCAGATGACACAGAGCCCAAGCTCCCTGTCTG | |
| | | | CCAGCGTGGGCGACAGGGTGACCATCACATGCAGGG | |
| | | | CCTCCCAGGATGTGAACACCGCCGTGGCCTGGTACCA | |
| | | | GCAGAAGCCTGGCAAGGCCCCAAAGCTGCTGATCTA | |
| | | | CTCCGCCTCTTTCCTGTATTCCGGCGTGCCTTCTCGGT | |
| | | | TTAGCGGCTCCAGATCTGGCACCGACTTCACCCTGAC | |
| | | | AATCTCTAGCCTGCAGCCAGAGGATTTTGCCACATAC | |
| | | | TATTGCCAGCAGCACTATACCACACCCCCTACCTTCGG | |
| | | | CCAGGGCACAAAGGTGGAGATCAAGGGAGGCAGCG | |
| | | | GAGGAGGCTCCGGAGGAGGCTCTGGCGGAGGCAGC | |
| | | | GGCGGCGGCTCCGGCGAGGTGCAGCTGGTGGAGAG | |
| | | | CGGCGGCGGCCTGGTGCAGCCTGGAGGCTCTCTGAG | |
| | | | GCTGAGCTGTGCAGCCTCCGGCTTTAACATCAAGGAC | |
| | | | ACCTACATCCACTGGGTGCGGCAGGCACCTGGCAAG | |
| | | | GGACTGGAGTGGGTGGCCAGAATCTATCCAACCAAT | |
| | | | GGCTACACACGGTATGCCGACTCCGTGAAGGGCCGG | |
| | | | TTCACCATCTCTGCCGATACCAGCAAGAACACAGCCT | |
| | | | ACCTGCAGATGAATAGCCTGCGGGCCGAGGATACAG | |
| | | | CCGTGTACTATTGCTCCAGATGGGGCGGCGACGGCTT | |
| | | | CTACGCCATGGATTATTGGGGCCAGGGCACCCTGGTG | |
| | | | ACAGTGTCCTCTGCCGCCGAGCCCAAGAGCTCCGACA | |
| | | | AGACCCACACATGCCCACCATGTCCGGCGCCAGAGGC | |
| | | | TGCAGGAGGACCAAGCGTGTTCCTGTTTCCACCCAAG | |
| | | | CCTAAAGACACACTGATGATTTCCCGAACCCCCGAAG | |
| | | | TCACATGCGTGGTCGTGTCTGTGAGTCACGAGGACCC | |
| | | | TGAAGTCAAGTTCAACTGGTACGTGGATGGCGTCGA | |
| | | | GGTGCATAATGCCAAGACTAAACCTAGGGAGGAACA | |
| | | | GTACAACTCAACCTATCGCGTCGTGAGCGTCCTGACA | |
| | | | GTGCTGCACCAGGATTGGCTGAACGGCAAAGAATAT | |
| | | | AAGTGCAAAGTGAGCAATAAGGCCCTGCCCGCTCCTA | |
| | | | TCGAGAAAACCATTTCCAAGGCTAAAGGGCAGCCTCG | |
| | | | CGAACCACAGGTCTACGTGTATCCTCCAAGCCGGGAC | |
| | | | GAGCTGACAAAGAACCAGGTCTCCCTGACTTGTCTGG | |
| | | | TGAAAGGGTTTTACCCTAGTGATATCGCTGTGGAGTG | |
| | | | GGAATCAAATGGACAGCCAGAGAACAATTATAAGAC | |
| | | | TACCCCCCCTGTGCTGGACAGTGATGGGTCATTCGCA | |
| | | | CTGGTCTCCAAGCTGACAGTGGACAAATCTCGGTGGC | |
| | | | AGCAGGGAAATGTCTTTTCATGTAGCGTGATGCATGA | |
| | | | AGCACTGCACAACCATTACACCCAGAAGTCACTGTCA | |
| | | | CTGTCACCAGGA | |
| 142 | 16801 | Full | EVKLVESGGGLVQPGGSLKLSCATSGFTFSDYYMYWVR | VH = E1- |
| | | | QTPEKRLEWVAYINSGGGSTYYPDTVKGRFTISRDNAK | S119; |
| | | | NTLYLQMSRLKSEDTAMYYCARRGLPFHAMDYWGQG | CH1 = A120- |
| | | | TSVTVSSASTKGPSVFPLAPSSKSTSGGTAALGCLVKDYF | V217; |
| | | | PEPVTVSWNSGALTSGVHTFPAVLQSSGLYSLSSVVTVP | VH = E233- |
| | | | SSSLGTQTYICNVNHKPSNTKVDKKVEPKSCDKTHTGGG | S351; |
| | | | GSEVKLVESGGGLVQPGGSLKLSCATSGFTFSDYYMYW | CH1 = A352- |
| | | | VRQTPEKRLEWVAYINSGGGSTYYPDTVKGRFTISRDNA | V449 |
| | | | KNTLYLQMSRLKSEDTAMYYCARRGLPFHAMDYWGQ | |
| | | | GTSVTVSSASTKGPSVFPLAPSSKSTSGGTAALGCLVKDY | |
| | | | FPEPVTVSWNSGALTSGVHTFPAVLQSSGLYSLSSVVTV | |
| | | | PSSSLGTQTYICNVNHKPSNTKVDKKVEPKSCDKTHTCP | |
| | | | PCPAPEAAGGPSVFLFPPKPKDTLMISRTPEVTCVVVSV | |
| | | | SHEDPEVKFNWYVDGVEVHNAKTKPREEQYNSTYRVV | |
| | | | SVLTVLHQDWLNGKEYKCKVSNKALPAPIEKTISKAKGQ | |
| | | | PREPQVYVLPPSRDELTKNQVSLLCLVKGFYPSDIAVEW | |
| | | | ESNGQPENNYLTWPPVLDSDGSFFLYSKLTVDKSRWQ | |
| | | | QGNVFSCSVMHEALHNHYTQKSLSLSPG | |
| 143 | 16801 | Full | GAGGTGAAGCTGGTGGAGAGCGGAGGAGGACTGGT | |
| | | | GCAGCCAGGAGGCTCTCTGAAGCTGAGCTGCGCCAC | |
| | | | CTCCGGCTTCACATTTTCCGACTACTATATGTACTGGG | |
| | | | TGCGGCAGACCCCAGAGAAGAGACTGGAGTGGGTG | |
| | | | GCCTATATCAACTCTGGCGGCGGCAGCACCTACTATC | |
| | | | CCGACACAGTGAAGGGCCGGTTTACCATCTCCAGAGA | |
| | | | TAACGCCAAGAATACACTGTACCTGCAGATGTCCAGG | |
| | | | CTGAAGTCTGAGGACACCGCCATGTACTATTGCGCAC | |
| | | | GGAGAGGCCTGCCATTCCACGCAATGGATTATTGGG | |
| | | | GCCAGGGCACCAGCGTGACAGTGAGCTCCGCCTCCA | |
| | | | CAAAGGGACCTAGCGTGTTCCCACTGGCCCCCTCTAG | |
| | | | CAAGTCCACCTCTGGAGGAACAGCCGCCCTGGGCTGT | |
| | | | CTGGTGAAGGACTACTTCCCCGAGCCTGTGACCGTGA | |
| | | | GCTGGAACTCCGGGGCCCTGACCAGCGGAGTGCACA | |
| | | | CATTTCCCGCCGTGCTGCAGTCCTCTGGCCTGTACTCT | |
| | | | CTGAGCTCCGTGGTGACCGTGCCTTCTAGCTCCCTGG | |
| | | | GCACCCAGACATATATCTGCAACGTGAATCACAAGCC | |
| | | | TTCTAATACAAAGGTGGACAAGAAGGTGGAGCCAAA | |
| | | | GAGCTGTGATAAGACCCACACAGGAGGAGGAGGCA | |
| | | | GCGAAGTCAAGCTGGTGGAGTCTGGCGGCGGCCTGG | |
| | | | TCCAGCCTGGAGGCAGCCTGAAGCTGTCCTGCGCCAC | |
| | | | CTCTGGCTTCACATTTTCTGATTATTACATGTACTGGG | |
| | | | TGAGGCAGACCCCTGAGAAGCGCCTGGAATGGGTCG | |
| | | | CCTATATCAATAGCGGCGGCGGCTCCACCTACTATCC | |
| | | | AGACACAGTGAAGGGCAGGTTCACCATCAGCCGCGA | |
| | | | TAATGCTAAAAACACCCTGTACCTGCAGATGTCTCGG | |
| | | | CTGAAGAGCGAGGACACAGCCATGTACTATTGTGCA | |
| | | | AGGCGCGGCCTGCCATTTCACGCAATGGATTACTGGG | |
| | | | GCCAGGGCACCTCCGTGACAGTGTCTAGCGCTAGCAC | |
| | | | CAAGGGACCATCCGTGTTCCCACTGGCACCAAGCTCC | |
| | | | AAGTCTACAAGCGGAGGAACCGCCGCCCTGGGCTGT | |
| | | | CTGGTGAAGGATTACTTCCCAGAGCCCGTGACCGTGT | |
| | | | CTTGGAACAGCGGGGCCCTGACCAGCGGAGTGCACA | |
| | | | CCTTTCCTGCCGTGCTGCAGTCTAGCGGCCTGTATAG | |
| | | | CCTGTCCTCTGTGGTCACAGTGCCAAGCTCCTCTCTGG | |
| | | | GCACACAGACCTACATCTGCAACGTGAATCACAAGCC | |
| | | | ATCCAATACCAAGGTCGACAAGAAGGTGGAGCCCAA | |
| | | | GTCTTGTGATAAGACACACACCTGCCCACCTTGTCCG | |
| | | | GCGCCAGAGGCCGCCGGAGGACCAAGCGTGTTCCTG | |
| | | | TTTCCACCCAAGCCTAAGGACACACTGATGATCAGCA | |
| | | | GGACACCAGAGGTGACCTGCGTGGTGGTGTCCGTGT | |
| | | | CTCACGAGGACCCCGAGGTGAAGTTTAACTGGTACGT | |
| | | | GGATGGCGTGGAGGTGCACAATGCCAAGACCAAGCC | |
| | | | AAGGGAGGAGCAGTATAACTCTACATACCGCGTGGT | |
| | | | GAGCGTGCTGACCGTGCTGCACCAGGATTGGCTGAA | |
| | | | CGGCAAGGAGTACAAGTGCAAGGTGAGCAATAAGGC | |
| | | | CCTGCCCGCCCCTATCGAGAAGACAATCTCCAAGGCC | |
| | | | AAGGGCCAGCCTCGCGAACCACAGGTGTATGTGCTG | |
| | | | CCTCCATCTAGAGACGAGCTGACCAAGAACCAGGTG | |
| | | | AGCCTGCTGTGCCTGGTGAAGGGCTTCTACCCCAGCG | |
| | | | ATATCGCCGTGGAGTGGGAGTCCAATGGCCAGCCTG | |
| | | | AGAACAATTATCTGACATGGCCCCCTGTGCTGGACTC | |
| | | | CGATGGCTCTTTCTTTCTGTACTCCAAGCTGACCGTGG | |
| | | | ACAAGTCTCGCTGGCAGCAGGGCAACGTGTTTAGCTG | |
| | | | TTCCGTGATGCACGAGGCCCTGCACAATCACTACACC | |
| | | | CAGAAGTCTCTGAGCTTAAGCCCTGGC | |
| 144 | 16802 | Full | QVQLVESGGGVVQPGRSLRLSCAASGFTFSNYGMYWV | VH = Q1- |
| | | | RQAPGKGLEWVAVIWYDGSNKYYADSVKGRFTISRDN | S118; |
| | | | SKNTLYLQMNSLRAEDTAVYYCARDLWGWYFDYWGQ | CH1 = A119- |
| | | | GTLVTVSSASTKGPSVFPLAPSSKSTSGGTAALGCLVKDY | V216; |
| | | | FPEPVTVSWNSGALTSGVHTFPAVLQSSGLYSLSSVVTV | VH = Q232- |
| | | | PSSSLGTQTYICNVNHKPSNTKVDKKVEPKSCDKTHTGG | S349; |
| | | | GGSQVQLVESGGGVVQPGRSLRLSCAASGFTFSNYGM | CH1 = A350- |
| | | | YWVRQAPGKGLEWVAVIWYDGSNKYYADSVKGRFTIS | V447 |
| | | | RDNSKNTLYLQMNSLRAEDTAVYYCARDLWGWYFDY | |
| | | | WGQGTLVTVSSASTKGPSVFPLAPSSKSTSGGTAALGCL | |
| | | | VKDYFPEPVTVSWNSGALTSGVHTFPAVLQSSGLYSLSS | |
| | | | VVTVPSSSLGTQTYICNVNHKPSNTKVDKKVEPKSCDKT | |
| | | | HTCPPCPAPEAAGGPSVFLFPPKPKDTLMISRTPEVTCV | |
| | | | VVSVSHEDPEVKFNWYVDGVEVHNAKTKPREEQYNST | |
| | | | YRVVSVLTVLHQDWLNGKEYKCKVSNKALPAPIEKTISK | |
| | | | AKGQPREPQVYVLPPSRDELTKNQVSLLCLVKGFYPSDI | |
| | | | AVEWESNGQPENNYLTWPPVLDSDGSFFLYSKLTVDKS | |
| | | | RWQQGNVFSCSVMHEALHNHYTQKSLSLSPG | |
| 145 | 16802 | Full | CAGGTGCAGCTGGTGGAGTCCGGAGGAGGAGTGGT | |
| | | | GCAGCCAGGCCGGTCTCTGAGACTGAGCTGCGCAGC | |
| | | | CTCCGGCTTCACCTTCAGCAACTACGGCATGTATTGG | |
| | | | GTGAGGCAGGCCCCTGGCAAGGGACTGGAGTGGGT | |
| | | | GGCCGTGATCTGGTACGACGGCTCTAATAAGTACTAT | |
| | | | GCCGATAGCGTGAAGGGCCGGTTTACCATCTCTAGAG | |
| | | | ACAACAGCAAGAATACACTGTATCTGCAGATGAACAG | |
| | | | CCTGCGGGCCGAGGATACCGCCGTGTACTATTGCGCC | |
| | | | AGAGACCTGTGGGGCTGGTACTTCGATTATTGGGGCC | |
| | | | AGGGCACCCTGGTGACAGTGAGCTCCGCCAGCACAA | |
| | | | AGGGACCATCCGTGTTTCCACTGGCCCCCTCTAGCAA | |
| | | | GTCCACCTCTGGAGGAACAGCCGCCCTGGGCTGTCTG | |
| | | | GTGAAGGACTACTTCCCCGAGCCTGTGACCGTGAGCT | |
| | | | GGAACTCCGGGGCCCTGACCAGCGGAGTGCACACAT | |
| | | | TTCCCGCCGTGCTGCAGTCCTCTGGCCTGTACTCTCTG | |
| | | | AGCTCCGTGGTGACCGTGCCTTCTAGCTCCCTGGGCA | |
| | | | CCCAGACATATATCTGCAACGTGAATCACAAGCCTTCT | |
| | | | AATACAAAGGTGGACAAGAAGGTGGAGCCAAAGAG | |
| | | | CTGTGATAAGACCCACACAGGAGGAGGAGGCTCCCA | |
| | | | GGTCCAGCTGGTCGAGTCTGGCGGCGGCGTCGTGCA | |
| | | | GCCAGGCAGGTCCCTGCGCCTGTCTTGCGCAGCCAGC | |
| | | | GGCTTCACCTTTTCCAACTACGGAATGTATTGGGTGC | |
| | | | GGCAGGCCCCCGGCAAGGGCCTGGAATGGGTCGCCG | |
| | | | TGATCTGGTATGATGGCAGCAATAAGTATTACGCCGA | |
| | | | TTCCGTGAAGGGCAGGTTCACCATCTCCCGCGACAAC | |
| | | | TCTAAGAATACACTGTACCTGCAGATGAATAGCCTGA | |
| | | | GGGCTGAAGACACCGCCGTGTACTACTGTGCCCGCG | |
| | | | ACCTGTGGGGATGGTATTTTGACTACTGGGGACAGG | |
| | | | GCACCCTGGTCACAGTGTCTAGCGCTAGCACCAAGGG | |
| | | | ACCATCCGTGTTCCCACTGGCACCAAGCTCCAAGTCTA | |
| | | | CAAGCGGAGGAACCGCCGCCCTGGGCTGTCTGGTGA | |
| | | | AGGATTACTTCCCAGAGCCCGTGACCGTGTCTTGGAA | |
| | | | CAGCGGGGCCCTGACCAGCGGAGTGCACACCTTTCCT | |
| | | | GCCGTGCTGCAGTCTAGCGGCCTGTATAGCCTGTCCT | |
| | | | CTGTGGTCACAGTGCCAAGCTCCTCTCTGGGCACACA | |
| | | | GACCTACATCTGCAACGTGAATCACAAGCCATCCAAT | |
| | | | ACCAAGGTCGACAAGAAGGTGGAGCCCAAGTCTTGT | |
| | | | GATAAGACACACACCTGCCCACCTTGTCCGGCGCCAG | |
| | | | AGGCCGCCGGAGGACCAAGCGTGTTCCTGTTTCCACC | |
| | | | CAAGCCTAAGGACACACTGATGATCAGCAGGACACC | |
| | | | AGAGGTGACCTGCGTGGTGGTGTCCGTGTCTCACGA | |
| | | | GGACCCCGAGGTGAAGTTTAACTGGTACGTGGATGG | |
| | | | CGTGGAGGTGCACAATGCCAAGACCAAGCCAAGGGA | |
| | | | GGAGCAGTATAACTCTACATACCGCGTGGTGAGCGT | |
| | | | GCTGACCGTGCTGCACCAGGATTGGCTGAACGGCAA | |
| | | | GGAGTACAAGTGCAAGGTGAGCAATAAGGCCCTGCC | |
| | | | CGCCCCTATCGAGAAGACAATCTCCAAGGCCAAGGG | |
| | | | CCAGCCTCGCGAACCACAGGTGTATGTGCTGCCTCCA | |
| | | | TCTAGAGACGAGCTGACCAAGAACCAGGTGAGCCTG | |
| | | | CTGTGCCTGGTGAAGGGCTTCTACCCCAGCGATATCG | |
| | | | CCGTGGAGTGGGAGTCCAATGGCCAGCCTGAGAACA | |
| | | | ATTATCTGACATGGCCCCCTGTGCTGGACTCCGATGG | |
| | | | CTCTTTCTTTCTGTACTCCAAGCTGACCGTGGACAAGT | |
| | | | CTCGCTGGCAGCAGGGCAACGTGTTTAGCTGTTCCGT | |
| | | | GATGCACGAGGCCCTGCACAATCACTACACCCAGAAG | |
| | | | TCTCTGAGCTTAAGCCCTGGC | |
| 146 | 16803 | Full | QVQLQQSGAELARPGASVKMSCKASGYTFTTYTMHW | VH = Q1- |
| | | | VKQRPGQGLEWIGYINPSSGYTNYNQKFKDKATLTADK | S121; |
| | | | SSSTASMQLSSLTSEDSAVYYCARERAVLVPYAMDYWG | CH1 = A122- |
| | | | QGTSVTVSSASTKGPSVFPLAPSSKSTSGGTAALGCLVK | V219; |
| | | | DYFPEPVTVSWNSGALTSGVHTFPAVLQSSGLYSLSSVV | VH = Q235- |
| | | | TVPSSSLGTQTYICNVNHKPSNTKVDKKVEPKSCDKTHT | S355; |
| | | | GGGGSQVQLQQSGAELARPGASVKMSCKASGYTFTTY | CH1 = A356- |
| | | | TMHWVKQRPGQGLEWIGYINPSSGYTNYNQKFKDKAT | V453 |
| | | | LTADKSSSTASMQLSSLTSEDSAVYYCARERAVLVPYAM | |
| | | | DYWGQGTSVTVSSASTKGPSVFPLAPSSKSTSGGTAAL | |
| | | | GCLVKDYFPEPVTVSWNSGALTSGVHTFPAVLQSSGLYS | |
| | | | LSSVVTVPSSSLGTQTYICNVNHKPSNTKVDKKVEPKSC | |
| | | | DKTHTCPPCPAPEAAGGPSVFLFPPKPKDTLMISRTPEV | |
| | | | TCVVVSVSHEDPEVKFNWYVDGVEVHNAKTKPREEQY | |
| | | | NSTYRVVSVLTVLHQDWLNGKEYKCKVSNKALPAPIEKT | |
| | | | ISKAKGQPREPQVYVLPPSRDELTKNQVSLLCLVKGFYPS | |
| | | | DIAVEWESNGQPENNYLTWPPVLDSDGSFFLYSKLTVD | |
| | | | KSRWQQGNVFSCSVMHEALHNHYTQKSLSLSPG | |
| 147 | 16803 | Full | CAGGTGCAGCTGCAGCAGTCCGGAGCCGAGCTGGCC | |
| | | | AGACCCGGGGCCAGCGTGAAGATGAGCTGCAAGGCC | |
| | | | TCCGGCTACACCTTCACCACATATACAATGCACTGGGT | |
| | | | GAAGCAGAGACCCGGACAGGGACTGGAGTGGATCG | |
| | | | GATACATCAACCCTAGCTCCGGCTACACCAACTATAAT | |
| | | | CAGAAGTTTAAGGACAAGGCCACCCTGACAGCCGAT | |
| | | | AAGTCTAGCTCCACCGCCTCCATGCAGCTGTCTAGCCT | |
| | | | GACATCTGAGGACAGCGCCGTGTACTATTGCGCCCGG | |
| | | | GAGAGAGCCGTGCTGGTGCCATACGCCATGGATTATT | |
| | | | GGGGCCAGGGCACCAGCGTGACAGTGTCCTCTGCCT | |
| | | | CTACCAAGGGCCCTAGCGTGTTTCCACTGGCCCCCAG | |
| | | | CTCCAAGAGCACCTCCGGAGGAACAGCCGCCCTGGG | |
| | | | CTGTCTGGTGAAGGACTATTTCCCCGAGCCAGTGACA | |
| | | | GTGTCCTGGAACTCTGGGGCCCTGACCAGCGGAGTG | |
| | | | CACACATTTCCTGCCGTGCTGCAGTCTAGCGGCCTGT | |
| | | | ACAGCCTGTCCTCTGTGGTGACCGTGCCAAGCTCCTCT | |
| | | | CTGGGCACCCAGACATATATCTGCAACGTGAATCACA | |
| | | | AGCCTAGCAATACAAAGGTGGACAAGAAGGTGGAGC | |
| | | | CAAAGTCCTGTGATAAGACCCACACAGGAGGAGGAG | |
| | | | GCTCCCAGGTCCAGCTGCAGCAGTCTGGAGCCGAGCT | |
| | | | GGCCAGGCCAGGGGCCAGCGTCAAAATGTCCTGTAA | |
| | | | AGCCTCCGGATATACCTTCACCACCTACACCATGCATT | |
| | | | GGGTCAAGCAGCGCCCAGGCCAGGGCCTGGAGTGG | |
| | | | ATCGGCTACATCAATCCCTCCAGCGGATATACTAATTA | |
| | | | CAACCAGAAGTTTAAGGATAAAGCCACCCTGACAGCC | |
| | | | GATAAATCCAGCTCCACCGCCTCCATGCAACTGTCTA | |
| | | | GCCTGACAAGCGAGGACTCCGCCGTGTACTATTGTGC | |
| | | | CAGGGAGAGGGCCGTGCTGGTCCCTTATGCTATGGA | |
| | | | CTACTGGGGACAGGGCACCAGCGTCACAGTGTCCTCT | |
| | | | GCTAGCACCAAGGGACCATCCGTGTTCCCACTGGCAC | |
| | | | CAAGCTCCAAGTCTACAAGCGGAGGAACCGCCGCCCT | |
| | | | GGGCTGTCTGGTGAAGGATTACTTCCCAGAGCCCGTG | |
| | | | ACCGTGTCTTGGAACAGCGGGGCCCTGACCAGCGGA | |
| | | | GTGCACACCTTTCCTGCCGTGCTGCAGTCTAGCGGCC | |
| | | | TGTATAGCCTGTCCTCTGTGGTCACAGTGCCAAGCTCC | |
| | | | TCTCTGGGCACACAGACCTACATCTGCAACGTGAATC | |
| | | | ACAAGCCATCCAATACCAAGGTCGACAAGAAGGTGG | |
| | | | AGCCCAAGTCTTGTGATAAGACACACACCTGCCCACC | |
| | | | TTGTCCGGCGCCAGAGGCCGCCGGAGGACCAAGCGT | |
| | | | GTTCCTGTTTCCACCCAAGCCTAAGGACACACTGATG | |
| | | | ATCAGCAGGACACCAGAGGTGACCTGCGTGGTGGTG | |
| | | | TCCGTGTCTCACGAGGACCCCGAGGTGAAGTTTAACT | |
| | | | GGTACGTGGATGGCGTGGAGGTGCACAATGCCAAGA | |
| | | | CCAAGCCAAGGGAGGAGCAGTATAACTCTACATACC | |
| | | | GCGTGGTGAGCGTGCTGACCGTGCTGCACCAGGATT | |
| | | | GGCTGAACGGCAAGGAGTACAAGTGCAAGGTGAGC | |
| | | | AATAAGGCCCTGCCCGCCCCTATCGAGAAGACAATCT | |
| | | | CCAAGGCCAAGGGCCAGCCTCGCGAACCACAGGTGT | |
| | | | ATGTGCTGCCTCCATCTAGAGACGAGCTGACCAAGAA | |
| | | | CCAGGTGAGCCTGCTGTGCCTGGTGAAGGGCTTCTAC | |
| | | | CCCAGCGATATCGCCGTGGAGTGGGAGTCCAATGGC | |
| | | | CAGCCTGAGAACAATTATCTGACATGGCCCCCTGTGC | |
| | | | TGGACTCCGATGGCTCTTTCTTTCTGTACTCCAAGCTG | |
| | | | ACCGTGGACAAGTCTCGCTGGCAGCAGGGCAACGTG | |
| | | | TTTAGCTGTTCCGTGATGCACGAGGCCCTGCACAATC | |
| | | | ACTACACCCAGAAGTCTCTGAGCTTAAGCCCTGGC | |
| 148 | 16811 | Full | QVQLQQSGAELARPGASVKMSCKASGYTFTTYTMHW | VH = Q1- |
| | | | VKQRPGQGLEWIGYINPSSGYTNYNQKFKDKATLTADK | S121; |
| | | | SSSTASMQLSSLTSEDSAVYYCARERAVLVPYAMDYWG | VL = Q142- |
| | | | QGTSVTVSSGGGGSGGGGSGGGGSGGGGSQIVLTQSP | K247; |
| | | | AVMSASPGEKVTITCTASSSLSYMHWFQQKPGTSPKL | VH = Q253- |
| | | | WLYSTSILASGVPTRFSGSGSGTSYSLTISRMEAEDAATY | S373; |
| | | | YCQQRSSSPFTFGSGTKLEIKGGGGSQEQLVESGGRLVT | CH1 = A374- |
| | | | PGGSLTLSCKASGFDFSAYYMSWVRQAPGKGLEWIATI | V471 |
| | | | YPSSGKTYYATWVNGRFTISSDNAQNTVDLQMNSLTA | |
| | | | ADRATYFCARDSYADDGALFNIWGPGTLVTISSASTKGP | |
| | | | SVFPLAPSSKSTSGGTAALGCLVKDYFPEPVTVSWNSGA | |
| | | | LTSGVHTFPAVLQSSGLYSLSSVVTVPSSSLGTQTYICNV | |
| | | | NHKPSNTKVDKKVEPKSCDKTHTCPPCPAPEAAGGPSV | |
| | | | FLFPPKPKDTLMISRTPEVTCVVVSVSHEDPEVKFNWYV | |
| | | | DGVEVHNAKTKPREEQYNSTYRVVSVLTVLHQDWLNG | |
| | | | KEYKCKVSNKALPAPIEKTISKAKGQPREPQVYVYPPSRD | |
| | | | ELTKNQVSLTCLVKGFYPSDIAVEWESNGQPENNYKTTP | |
| | | | PVLDSDGSFALVSKLTVDKSRWQQGNVFSCSVMHEAL | |
| | | | HNHYTQKSLSLSPG | |
| 149 | 16811 | Full | CAGGTGCAGCTGCAGCAGAGCGGAGCCGAGCTGGCC | |
| | | | AGACCTGGGGCCAGCGTGAAGATGAGCTGCAAGGCC | |
| | | | TCCGGCTACACATTCACCACATATACCATGCACTGGGT | |
| | | | GAAGCAGCGCCCTGGACAGGGACTGGAGTGGATCG | |
| | | | GCTACATCAACCCAAGCTCCGGCTACACAAACTATAA | |
| | | | TCAGAAGTTTAAGGACAAGGCCACCCTGACAGCCGAT | |
| | | | AAGTCTAGCTCCACAGCCTCCATGCAGCTGTCTAGCCT | |
| | | | GACCAGCGAGGACTCCGCCGTGTACTATTGCGCCCGG | |
| | | | GAGAGAGCCGTGCTGGTGCCTTACGCCATGGATTATT | |
| | | | GGGGCCAGGGCACAAGCGTGACCGTGTCCTCTGGCG | |
| | | | GCGGCGGCTCTGGAGGAGGAGGCAGCGGCGGAGGA | |
| | | | GGCTCCGGAGGCGGCGGCTCTCAGATCGTGCTGACC | |
| | | | CAGTCCCCAGCCGTGATGAGCGCCTCCCCAGGAGAG | |
| | | | AAGGTGACCATCACATGTACCGCCAGCTCCTCTCTGTC | |
| | | | CTACATGCACTGGTTCCAGCAGAAGCCCGGCACATCT | |
| | | | CCTAAGCTGTGGCTGTATTCTACCAGCATCCTGGCCTC | |
| | | | TGGCGTGCCAACACGGTTTTCCGGCTCTGGCAGCGGC | |
| | | | ACATCCTACTCTCTGACCATCTCCAGGATGGAGGCAG | |
| | | | AGGACGCAGCAACCTACTATTGCCAGCAGCGCAGCTC | |
| | | | CTCTCCATTCACATTTGGCAGCGGCACCAAGCTGGAG | |
| | | | ATCAAGGGAGGAGGAGGCTCTCAGGAGCAGCTGGT | |
| | | | GGAGAGCGGCGGCAGACTGGTGACACCAGGAGGCT | |
| | | | CTCTGACCCTGAGCTGTAAGGCCTCCGGCTTCGACTTC | |
| | | | AGCGCCTACTATATGTCCTGGGTGAGACAGGCCCCCG | |
| | | | GCAAGGGCCTGGAATGGATCGCCACCATCTATCCTAG | |
| | | | CTCCGGCAAGACATACTATGCCACCTGGGTGAACGGC | |
| | | | AGATTCACCATCTCTAGCGACAACGCCCAGAATACAG | |
| | | | TGGATCTGCAGATGAATAGCCTGACAGCCGCCGACA | |
| | | | GGGCCACCTACTTCTGTGCCCGCGATTCCTATGCCGA | |
| | | | CGATGGGGCCCTGTTCAACATCTGGGGCCCTGGCACA | |
| | | | CTGGTGACCATCTCCTCTGCTAGCACTAAGGGGCCTT | |
| | | | CCGTGTTTCCACTGGCTCCCTCTAGTAAATCCACCTCT | |
| | | | GGAGGCACAGCTGCACTGGGATGTCTGGTGAAGGAT | |
| | | | TACTTCCCTGAACCAGTCACAGTGAGTTGGAACTCAG | |
| | | | GGGCTCTGACAAGTGGAGTCCATACTTTTCCCGCAGT | |
| | | | GCTGCAGTCAAGCGGACTGTACTCCCTGTCCTCTGTG | |
| | | | GTCACCGTGCCTAGTTCAAGCCTGGGCACCCAGACAT | |
| | | | ATATCTGCAACGTGAATCACAAGCCATCAAATACAAA | |
| | | | AGTCGACAAGAAAGTGGAGCCCAAGAGCTGTGATAA | |
| | | | AACTCATACCTGCCCACCTTGTCCGGCGCCAGAGGCT | |
| | | | GCAGGAGGACCAAGCGTGTTCCTGTTTCCACCCAAGC | |
| | | | CTAAAGACACACTGATGATTTCCCGAACCCCCGAAGT | |
| | | | CACATGCGTGGTCGTGTCTGTGAGTCACGAGGACCCT | |
| | | | GAAGTCAAGTTCAACTGGTACGTGGATGGCGTCGAG | |
| | | | GTGCATAATGCCAAGACTAAACCTAGGGAGGAACAG | |
| | | | TACAACTCAACCTATCGCGTCGTGAGCGTCCTGACAG | |
| | | | TGCTGCACCAGGATTGGCTGAACGGCAAAGAATATA | |
| | | | AGTGCAAAGTGAGCAATAAGGCCCTGCCCGCTCCTAT | |
| | | | CGAGAAAACCATTTCCAAGGCTAAAGGGCAGCCTCG | |
| | | | CGAACCACAGGTCTACGTCTACCCCCCATCAAGAGAT | |
| | | | GAACTGACAAAAAATCAGGTCTCTCTGACATGCCTGG | |
| | | | TCAAAGGATTCTACCCTTCCGACATCGCCGTGGAGTG | |
| | | | GGAAAGTAACGGCCAGCCCGAGAACAATTACAAGAC | |
| | | | CACACCCCCTGTCCTGGACTCTGATGGGAGTTTCGCTC | |
| | | | TGGTGTCAAAGCTGACCGTCGATAAAAGCCGGTGGC | |
| | | | AGCAGGGCAATGTGTTTAGCTGCTCCGTCATGCACGA | |
| | | | AGCCCTGCACAATCACTACACACAGAAGTCCCTGAGC | |
| | | | CTGAGCCCTGGC | |
| 150 | 16812 | Full | QVQLVESGGGVVQPGRSLRLSCAASGFTFSNYGMYWV | VH = Q1- |
| | | | RQAPGKGLEWVAVIWYDGSNKYYADSVKGRFTISRDN | S118; |
| | | | SKNTLYLQMNSLRAEDTAVYYCARDLWGWYFDYWGQ | VL = E139- |
| | | | GTLVTVSSGGGGSGGGGSGGGGSGGGGSEIVLTQSPA | K245; |
| | | | TLSLSPGERATLSCRASQSVSSYLAWYQQKPGQAPRLLIY | VH = Q251- |
| | | | DASNRATGIPARFSGSGSGTDFTLTISSLEPEDFAVYYCQ | S371; |
| | | | QRRNWPLTFGGGTKVEIKGGGGSQEQLVESGGRLVTP | CH1 = A372- |
| | | | GGSLTLSCKASGFDFSAYYMSWVRQAPGKGLEWIATIY | V469 |
| | | | PSSGKTYYATWVNGRFTISSDNAQNTVDLQMNSLTAA | |
| | | | DRATYFCARDSYADDGALFNIWGPGTLVTISSASTKGPS | |
| | | | VFPLAPSSKSTSGGTAALGCLVKDYFPEPVTVSWNSGAL | |
| | | | TSGVHTFPAVLQSSGLYSLSSVVTVPSSSLGTQTYICNVN | |
| | | | HKPSNTKVDKKVEPKSCDKTHTCPPCPAPEAAGGPSVFL | |
| | | | FPPKPKDTLMISRTPEVTCVVVSVSHEDPEVKFNWYVD | |
| | | | GVEVHNAKTKPREEQYNSTYRVVSVLTVLHQDWLNGK | |
| | | | EYKCKVSNKALPAPIEKTISKAKGQPREPQVYVYPPSRDE | |
| | | | LTKNQVSLTCLVKGFYPSDIAVEWESNGQPENNYKTTPP | |
| | | | VLDSDGSFALVSKLTVDKSRWQQGNVFSCSVMHEALH | |
| | | | NHYTQKSLSLSPG | |
| 151 | 16812 | Full | CAGGTGCAGCTGGTGGAGTCCGGCGGCGGCGTGGTG | |
| | | | CAGCCTGGCAGGTCCCTGCGCCTGTCTTGCGCAGCCA | |
| | | | GCGGCTTCACCTTCAGCAACTACGGCATGTATTGGGT | |
| | | | GCGGCAGGCCCCTGGCAAGGGACTGGAGTGGGTGG | |
| | | | CCGTGATCTGGTACGACGGCAGCAATAAGTACTATGC | |
| | | | CGATTCCGTGAAGGGCCGGTTCACCATCTCCAGAGAC | |
| | | | AACTCTAAGAATACACTGTATCTGCAGATGAACTCCCT | |
| | | | GCGGGCCGAGGATACCGCCGTGTACTATTGCGCCAG | |
| | | | AGACCTGTGGGGCTGGTACTTTGATTATTGGGGCCAG | |
| | | | GGCACCCTGGTGACAGTGAGCAGCGGAGGAGGAGG | |
| | | | CAGCGGAGGAGGAGGCTCCGGAGGCGGCGGCTCTG | |
| | | | GCGGCGGCGGCAGCGAGATCGTGCTGACCCAGTCCC | |
| | | | CAGCCACACTGAGCCTGTCCCCAGGAGAGAGGGCCA | |
| | | | CCCTGTCTTGTCGCGCCTCTCAGAGCGTGTCTAGCTAC | |
| | | | CTGGCCTGGTATCAGCAGAAGCCAGGACAGGCCCCC | |
| | | | CGGCTGCTGATCTACGACGCCAGCAACAGGGCAACC | |
| | | | GGCATCCCAGCCAGATTCTCCGGCTCTGGCAGCGGCA | |
| | | | CAGACTTTACCCTGACAATCTCCTCTCTGGAGCCCGAG | |
| | | | GATTTCGCCGTGTACTATTGCCAGCAGCGGAGAAATT | |
| | | | GGCCTCTGACCTTTGGCGGCGGCACAAAGGTGGAGA | |
| | | | TCAAGGGAGGAGGAGGCTCTCAGGAGCAGCTGGTG | |
| | | | GAGAGCGGCGGCAGACTGGTGACCCCAGGAGGCAG | |
| | | | CCTGACACTGTCCTGTAAGGCCTCTGGCTTCGATTTTT | |
| | | | CCGCCTACTATATGTCTTGGGTGAGACAGGCCCCTGG | |
| | | | CAAGGGCCTGGAGTGGATCGCCACCATCTACCCAAGC | |
| | | | TCCGGCAAGACCTACTATGCCACATGGGTGAACGGCA | |
| | | | GATTCACCATCTCTAGCGACAACGCCCAGAATACAGT | |
| | | | GGATCTGCAGATGAACAGCCTGACCGCCGCCGACAG | |
| | | | GGCAACATACTTCTGTGCCCGCGATAGCTATGCCGAC | |
| | | | GATGGGGCCCTGTTCAACATCTGGGGACCAGGCACC | |
| | | | CTGGTGACAATCTCCTCTGCTAGCACTAAGGGGCCTT | |
| | | | CCGTGTTTCCACTGGCTCCCTCTAGTAAATCCACCTCT | |
| | | | GGAGGCACAGCTGCACTGGGATGTCTGGTGAAGGAT | |
| | | | TACTTCCCTGAACCAGTCACAGTGAGTTGGAACTCAG | |
| | | | GGGCTCTGACAAGTGGAGTCCATACTTTTCCCGCAGT | |
| | | | GCTGCAGTCAAGCGGACTGTACTCCCTGTCCTCTGTG | |
| | | | GTCACCGTGCCTAGTTCAAGCCTGGGCACCCAGACAT | |
| | | | ATATCTGCAACGTGAATCACAAGCCATCAAATACAAA | |
| | | | AGTCGACAAGAAAGTGGAGCCCAAGAGCTGTGATAA | |
| | | | AACTCATACCTGCCCACCTTGTCCGGCGCCAGAGGCT | |
| | | | GCAGGAGGACCAAGCGTGTTCCTGTTTCCACCCAAGC | |
| | | | CTAAAGACACACTGATGATTTCCCGAACCCCCGAAGT | |
| | | | CACATGCGTGGTCGTGTCTGTGAGTCACGAGGACCCT | |
| | | | GAAGTCAAGTTCAACTGGTACGTGGATGGCGTCGAG | |
| | | | GTGCATAATGCCAAGACTAAACCTAGGGAGGAACAG | |
| | | | TACAACTCAACCTATCGCGTCGTGAGCGTCCTGACAG | |
| | | | TGCTGCACCAGGATTGGCTGAACGGCAAAGAATATA | |
| | | | AGTGCAAAGTGAGCAATAAGGCCCTGCCCGCTCCTAT | |
| | | | CGAGAAAACCATTTCCAAGGCTAAAGGGCAGCCTCG | |
| | | | CGAACCACAGGTCTACGTCTACCCCCCATCAAGAGAT | |
| | | | GAACTGACAAAAAATCAGGTCTCTCTGACATGCCTGG | |
| | | | TCAAAGGATTCTACCCTTCCGACATCGCCGTGGAGTG | |
| | | | GGAAAGTAACGGCCAGCCCGAGAACAATTACAAGAC | |
| | | | CACACCCCCTGTCCTGGACTCTGATGGGAGTTTCGCTC | |
| | | | TGGTGTCAAAGCTGACCGTCGATAAAAGCCGGTGGC | |
| | | | AGCAGGGCAATGTGTTTAGCTGCTCCGTCATGCACGA | |
| | | | AGCCCTGCACAATCACTACACACAGAAGTCCCTGAGC | |
| | | | CTGAGCCCTGGC | |
| 152 | 16813 | Full | EVKLVESGGGLVQPGGSLKLSCATSGFTFSDYYMYWVR | VH = E1- |
| | | | QTPEKRLEWVAYINSGGGSTYYPDTVKGRFTISRDNAK | S119; |
| | | | NTLYLQMSRLKSEDTAMYYCARRGLPFHAMDYWGQG | VL = D140- |
| | | | TSVTVSSGGGGSGGGGSGGGGSGGGGSDIQMTQTTSS | K246; |
| | | | LSASLGDRVTISCSASQGISNYLNWYQQKPDGTVKLLIYY | VH = Q252- |
| | | | TSILHSGVPSRFSGSGSGTDYSLTIGNLEPEDIATYYCQQF | S372; |
| | | | NKLPPTFGGGTKLEIKGGGGSQEQLVESGGRLVTPGGSL | CH1 = A373- |
| | | | TLSCKASGFDFSAYYMSWVRQAPGKGLEWIATIYPSSG | V470 |
| | | | KTYYATWVNGRFTISSDNAQNTVDLQMNSLTAADRAT | |
| | | | YFCARDSYADDGALFNIWGPGTLVTISSASTKGPSVFPL | |
| | | | APSSKSTSGGTAALGCLVKDYFPEPVTVSWNSGALTSGV | |
| | | | HTFPAVLQSSGLYSLSSVVTVPSSSLGTQTYICNVNHKPS | |
| | | | NTKVDKKVEPKSCDKTHTCPPCPAPEAAGGPSVFLFPPK | |
| | | | PKDTLMISRTPEVTCVVVSVSHEDPEVKFNWYVDGVEV | |
| | | | HNAKTKPREEQYNSTYRVVSVLTVLHQDWLNGKEYKCK | |
| | | | VSNKALPAPIEKTISKAKGQPREPQVYVYPPSRDELTKN | |
| | | | QVSLTCLVKGFYPSDIAVEWESNGQPENNYKTTPPVLDS | |
| | | | DGSFALVSKLTVDKSRWQQGNVFSCSVMHEALHNHYT | |
| | | | QKSLSLSPG | |
| 153 | 16813 | Full | GAGGTGAAGCTGGTGGAGTCTGGAGGAGGACTGGT | |
| | | | GCAGCCAGGAGGCAGCCTGAAGCTGTCCTGCGCCAC | |
| | | | CTCTGGCTTCACCTTCAGCGACTACTATATGTACTGGG | |
| | | | TGCGGCAGACCCCCGAGAAGAGACTGGAGTGGGTG | |
| | | | GCCTATATCAACAGCGGCGGCGGCTCCACCTACTATC | |
| | | | CTGACACAGTGAAGGGCAGGTTCACCATCTCCCGCGA | |
| | | | TAACGCCAAGAATACACTGTACCTGCAGATGTCTAGG | |
| | | | CTGAAGAGCGAGGACACAGCCATGTACTATTGCGCCC | |
| | | | GGAGAGGCCTGCCTTTTCACGCCATGGATTATTGGGG | |
| | | | CCAGGGCACCAGCGTGACAGTGAGCAGCGGAGGAG | |
| | | | GAGGCTCCGGCGGCGGAGGCTCTGGCGGCGGCGGC | |
| | | | AGCGGAGGCGGCGGCTCCGACATCCAGATGACCCAG | |
| | | | ACCACATCTAGCCTGTCCGCCTCTCTGGGCGATCGGG | |
| | | | TGACAATCAGCTGTTCCGCCTCTCAGGGCATCTCCAAC | |
| | | | TACCTGAATTGGTATCAGCAGAAGCCTGACGGCACCG | |
| | | | TGAAGCTGCTGATCTACTATACATCCATCCTGCACTCT | |
| | | | GGCGTGCCAAGCAGATTCAGCGGCTCCGGCTCTGGA | |
| | | | ACCGACTACAGCCTGACAATCGGCAACCTGGAGCCA | |
| | | | GAGGATATCGCCACCTACTATTGCCAGCAGTTCAATA | |
| | | | AGCTGCCCCCTACCTTTGGCGGCGGCACAAAGCTGGA | |
| | | | GATCAAGGGAGGAGGAGGCTCCCAGGAGCAGCTGG | |
| | | | TGGAGTCTGGCGGCAGGCTGGTGACCCCAGGAGGCT | |
| | | | CCCTGACACTGTCTTGTAAGGCCAGCGGCTTCGATTTT | |
| | | | TCTGCCTACTATATGAGCTGGGTGCGCCAGGCCCCAG | |
| | | | GCAAGGGACTGGAGTGGATCGCCACCATCTACCCCTC | |
| | | | CTCTGGCAAGACCTACTATGCCACATGGGTGAACGGC | |
| | | | AGATTCACCATCAGCTCCGACAACGCCCAGAATACAG | |
| | | | TGGATCTGCAGATGAATAGCCTGACCGCCGCCGACA | |
| | | | GGGCCACATACTTCTGTGCCCGCGATTCCTATGCCGA | |
| | | | CGATGGGGCCCTGTTCAACATCTGGGGACCAGGCAC | |
| | | | CCTGGTGACAATCTCTAGCGCTAGCACTAAGGGGCCT | |
| | | | TCCGTGTTTCCACTGGCTCCCTCTAGTAAATCCACCTC | |
| | | | TGGAGGCACAGCTGCACTGGGATGTCTGGTGAAGGA | |
| | | | TTACTTCCCTGAACCAGTCACAGTGAGTTGGAACTCA | |
| | | | GGGGCTCTGACAAGTGGAGTCCATACTTTTCCCGCAG | |
| | | | TGCTGCAGTCAAGCGGACTGTACTCCCTGTCCTCTGT | |
| | | | GGTCACCGTGCCTAGTTCAAGCCTGGGCACCCAGACA | |
| | | | TATATCTGCAACGTGAATCACAAGCCATCAAATACAA | |
| | | | AAGTCGACAAGAAAGTGGAGCCCAAGAGCTGTGATA | |
| | | | AAACTCATACCTGCCCACCTTGTCCGGCGCCAGAGGC | |
| | | | TGCAGGAGGACCAAGCGTGTTCCTGTTTCCACCCAAG | |
| | | | CCTAAAGACACACTGATGATTTCCCGAACCCCCGAAG | |
| | | | TCACATGCGTGGTCGTGTCTGTGAGTCACGAGGACCC | |
| | | | TGAAGTCAAGTTCAACTGGTACGTGGATGGCGTCGA | |
| | | | GGTGCATAATGCCAAGACTAAACCTAGGGAGGAACA | |
| | | | GTACAACTCAACCTATCGCGTCGTGAGCGTCCTGACA | |
| | | | GTGCTGCACCAGGATTGGCTGAACGGCAAAGAATAT | |
| | | | AAGTGCAAAGTGAGCAATAAGGCCCTGCCCGCTCCTA | |
| | | | TCGAGAAAACCATTTCCAAGGCTAAAGGGCAGCCTCG | |
| | | | CGAACCACAGGTCTACGTCTACCCCCCATCAAGAGAT | |
| | | | GAACTGACAAAAAATCAGGTCTCTCTGACATGCCTGG | |
| | | | TCAAAGGATTCTACCCTTCCGACATCGCCGTGGAGTG | |
| | | | GGAAAGTAACGGCCAGCCCGAGAACAATTACAAGAC | |
| | | | CACACCCCCTGTCCTGGACTCTGATGGGAGTTTCGCTC | |
| | | | TGGTGTCAAAGCTGACCGTCGATAAAAGCCGGTGGC | |
| | | | AGCAGGGCAATGTGTTTAGCTGCTCCGTCATGCACGA | |
| | | | AGCCCTGCACAATCACTACACACAGAAGTCCCTGAGC | |
| | | | CTGAGCCCTGGC | |
| 154 | 16814 | Full | QEQLVESGGRLVTPGGSLTLSCKASGFDFSAYYMSWVR | VH = Q1- |
| | | | QAPGKGLEWIATIYPSSGKTYYATWVNGRFTISSDNAQ | S121; |
| | | | NTVDLQMNSLTAADRATYFCARDSYADDGALFNIWGP | CH1 = A122- |
| | | | GTLVTISSASTKGPSVFPLAPSSKSTSGGTAALGCLVKDYF | V219 |
| | | | PEPVTVSWNSGALTSGVHTFPAVLQSSGLYSLSSVVTVP | |
| | | | SSSLGTQTYICNVNHKPSNTKVDKKVEPKSCDKTHTGGG | |
| | | | GSEPAVYFKEQFLDGDGWTSRWIESKHKSDFGKFVLSS | |
| | | | GKFYGDEEKDKGLQTSQDARFYALSASFEPFSNKGQTLV | |
| | | | VQFTVKHEQNIDCGGGYVKLFPNSLDQTDMHGDSEYN | |
| | | | IMFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAAAEPKSSDKTHTCPPCPAPEA | |
| | | | AGGPSVFLFPPKPKDTLMISRTPEVTCVVVSVSHEDPEV | |
| | | | KFNWYVDGVEVHNAKTKPREEQYNSTYRVVSVLTVLH | |
| | | | QDWLNGKEYKCKVSNKALPAPIEKTISKAKGQPREPQV | |
| | | | YVYPPSRDELTKNQVSLTCLVKGFYPSDIAVEWESNGQP | |
| | | | ENNYKTTPPVLDSDGSFALVSKLTVDKSRWQQGNVFSC | |
| | | | SVMHEALHNHYTQKSLSLSPG | |
| 155 | 16814 | Full | CAGGAGCAGCTGGTGGAGAGCGGCGGCAGACTGGT | |
| | | | GACCCCAGGAGGCAGCCTGACACTGTCCTGCAAGGC | |
| | | | CTCTGGCTTCGACTTTTCCGCCTACTATATGTCTTGGG | |
| | | | TGCGGCAGGCCCCCGGCAAGGGACTGGAGTGGATCG | |
| | | | CCACCATCTACCCTAGCTCCGGCAAGACCTACTATGCC | |
| | | | ACATGGGTGAACGGCAGATTCACCATCTCTAGCGATA | |
| | | | ACGCCCAGAATACAGTGGACCTGCAGATGAATAGCCT | |
| | | | GACCGCCGCCGACAGGGCAACATACTTCTGCGCCAG | |
| | | | AGATTCCTATGCCGACGATGGGGCCCTGTTCAACATC | |
| | | | TGGGGCCCAGGCACCCTGGTGACAATCTCCTCTGCTA | |
| | | | GCACCAAGGGACCATCCGTGTTTCCACTGGCCCCTAG | |
| | | | CTCCAAGTCCACCTCTGGAGGAACAGCCGCCCTGGGC | |
| | | | TGTCTGGTGAAGGACTATTTCCCCGAGCCTGTGACAG | |
| | | | TGTCCTGGAACTCTGGGGCCCTGACCAGCGGAGTGC | |
| | | | ACACATTTCCTGCCGTGCTGCAGTCTAGCGGCCTGTAT | |
| | | | AGCCTGTCCTCTGTGGTGACCGTGCCAAGCTCCTCTCT | |
| | | | GGGCACCCAGACATACATCTGCAACGTGAATCACAAG | |
| | | | CCAAGCAATACAAAGGTCGACAAGAAGGTGGAGCCC | |
| | | | AAGTCCTGTGATAAGACCCACACCGGCGGAGGAGGC | |
| | | | TCTGAGCCTGCCGTGTACTTCAAGGAGCAGTTTCTGG | |
| | | | ACGGCGATGGCTGGACCTCCAGGTGGATCGAGAGCA | |
| | | | AGCACAAGTCCGACTTCGGCAAGTTTGTGCTGAGCTC | |
| | | | CGGCAAGTTCTATGGCGATGAGGAGAAGGACAAGG | |
| | | | GCCTGCAGACATCCCAGGATGCCCGCTTTTACGCCCT | |
| | | | GAGCGCCTCCTTCGAGCCCTTTTCTAATAAGGGCCAG | |
| | | | ACCCTGGTGGTGCAGTTCACAGTGAAGCACGAGCAG | |
| | | | AACATCGACTGTGGCGGCGGCTATGTGAAGCTGTTTC | |
| | | | CTAATTCTCTGGATCAGACCGACATGCACGGCGACAG | |
| | | | CGAGTACAACATCATGTTCGGCCCAGATATCTGCGGC | |
| | | | CCCGGCACAAAGAAGGTGCACGTGATCTTTAATTATA | |
| | | | AGGGCAAGAACGTGCTGATCAATAAGGACATCAGGT | |
| | | | GTAAGGACGATGAGTTCACCCACCTGTACACACTGAT | |
| | | | CGTGCGCCCAGACAACACCTATGAGGTGAAGATCGA | |
| | | | TAATAGCCAGGTGGAGTCTGGCAGCCTGGAGGACGA | |
| | | | TTGGGATTTTCTGCCCCCTAAGAAGATCAAGGACCCT | |
| | | | GATGCCAGCAAGCCAGAGGACTGGGATGAGCGGGC | |
| | | | CAAGATCGACGATCCCACCGACTCCAAGCCTGAGGAC | |
| | | | TGGGATAAGCCTGAGCACATCCCAGACCCCGATGCCA | |
| | | | AGAAGCCCGAAGACTGGGATGAGGAGATGGATGGC | |
| | | | GAGTGGGAGCCACCCGTGATCCAGAACCCCGAGTAC | |
| | | | AAGGGCGAGTGGAAGCCTAGACAGATCGATAATCCA | |
| | | | GACTATAAGGGCACCTGGATTCACCCAGAGATCGATA | |
| | | | ACCCCGAGTACTCTCCTGACCCAAGCATCTACGCCTAT | |
| | | | GATAATTTCGGCGTGCTGGGCCTGGACCTGTGGCAG | |
| | | | GTGAAGTCCGGCACCATCTTCGACAACTTTCTGATCAC | |
| | | | AAATGATGAGGCCTACGCCGAGGAGTTTGGCAACGA | |
| | | | GACCTGGGGCGTGACAAAGGCCGCCGAGAAGCAGAT | |
| | | | GAAGGATAAGCAGGACGAGGAGCAGAGGCTGAAGG | |
| | | | AAGAGGAGGAGGACAAGAAGCGCAAGGAGGAGGA | |
| | | | GGAGGCCGAGGATAAGGAGGACGATGAGGACAAGG | |
| | | | ATGAGGACGAGGAGGATGAGGAGGACAAGGAGGA | |
| | | | GGATGAGGAGGAGGACGTGCCAGGACAGGCCGCCG | |
| | | | CCGAGCCTAAGTCTAGCGATAAGACCCACACATGCCC | |
| | | | TCCATGTCCGGCGCCAGAGGCTGCAGGAGGACCAAG | |
| | | | CGTGTTCCTGTTTCCACCCAAGCCTAAAGACACACTGA | |
| | | | TGATTTCCCGAACCCCCGAAGTCACATGCGTGGTCGT | |
| | | | GTCTGTGAGTCACGAGGACCCTGAAGTCAAGTTCAAC | |
| | | | TGGTACGTGGATGGCGTCGAGGTGCATAATGCCAAG | |
| | | | ACTAAACCTAGGGAGGAACAGTACAACTCAACCTATC | |
| | | | GCGTCGTGAGCGTCCTGACAGTGCTGCACCAGGATTG | |
| | | | GCTGAACGGCAAAGAATATAAGTGCAAAGTGAGCAA | |
| | | | TAAGGCCCTGCCCGCTCCTATCGAGAAAACCATTTCC | |
| | | | AAGGCTAAAGGGCAGCCTCGCGAACCACAGGTCTAC | |
| | | | GTGTATCCTCCAAGCCGGGACGAGCTGACAAAGAAC | |
| | | | CAGGTCTCCCTGACTTGTCTGGTGAAAGGGTTTTACC | |
| | | | CTAGTGATATCGCTGTGGAGTGGGAATCAAATGGAC | |
| | | | AGCCAGAGAACAATTATAAGACTACCCCCCCTGTGCT | |
| | | | GGACAGTGATGGGTCATTCGCACTGGTCTCCAAGCTG | |
| | | | ACAGTGGACAAATCTCGGTGGCAGCAGGGAAATGTC | |
| | | | TTTTCATGTAGCGTGATGCATGAAGCACTGCACAACC | |
| | | | ATTACACCCAGAAGTCACTGTCACTGTCACCAGGA | |
| 156 | | linker | AAGG | |
| 157 | | linker | GGGS | |
| 158 | | linker | GGGG | |
| 159 | | MelanA | ELGIGILTV | |
| | | peptide | | |
| 160 | | K-ras | KLVVVGAGGV | |
| | | peptide | | |
| 161 | 17904 | Full | EPAVYFKEQFLDGDGWTSRWIESKHKSDFGKFVLSSGK | |
| | | | FYGDEEKDKGLQTSQDARFYALSASFEPFSNKGQTLVV | |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAGGGGSEPAVYFKEQFLDGDG | |
| | | | WTSRWIESKHKSDFGKFVLSSGKFYGDEEKDKGLQTSQ | |
| | | | DARFYALSASFEPFSNKGQTLVVQFTVKHEQNIDCGGG | |
| | | | YVKLFPNSLDQTDMHGDSEYNIMFGPDICGPGTKKVHV | |
| | | | IFNYKGKNVLINKDIRCKDDEFTHLYTLIVRPDNTYEVKID | |
| | | | NSQVESGSLEDDWDFLPPKKIKDPDASKPEDWDERAKI | |
| | | | DDPTDSKPEDWDKPEHIPDPDAKKPEDWDEEMDGEW | |
| | | | EPPVIQNPEYKGEWKPRQIDNPDYKGTWIHPEIDNPEY | |
| | | | SPDPSIYAYDNFGVLGLDLWQVKSGTIFDNFLITNDEAY | |
| | | | AEEFGNETWGVTKAAEKQMKDKQDEEQRLKEEEEDKK | |
| | | | RKEEEEAEDKEDDEDKDEDEEDEEDKEEDEEEDVPGQA | |
| | | | AAEPKSSDKTHTCPPCPAPEAAGGPSVFLFPPKPKDTLM | |
| | | | ISRTPEVTCVVVSVSHEDPEVKFNWYVDGVEVHNAKTK | |
| | | | PREEQYNSTYRVVSVLTVLHQDWLNGKEYKCKVSNKAL | |
| | | | PAPIEKTISKAKGQPREPQVYVLPPSRDELTKNQVSLLCL | |
| | | | VKGFYPSDIAVEWESNGQPENNYLTWPPVLDSDGSFFL | |
| | | | YSKLTVDKSRWQQGNVFSCSVMHEALHNHYTQKSLSLS | |
| | | | PGGGGGDIQMTQSPSSLSASVGDRVTITCRASQDVNTA | |
| | | | VAWYQQKPGKAPKLLIYSASFLYSGVPSRFSGSRSGTDF | |
| | | | TLTISSLQPEDFATYYCQQHYTTPPTFGCGTKVEIKGGSG | |
| | | | GGSGGGSGGGSGGGSGEVQLVESGGGLVQPGGSLRLS | |
| | | | CAASGFNIKDTYIHWVRQAPGKCLEWVARIYPTNGYTR | |
| | | | YADSVKGRFTISADTSKNTAYLQMNSLRAEDTAVYYCSR | |
| | | | WGGDGFYAMDYWGQGTLVTVS | |
| 162 | 17858 | Full | EPAVYFKEQFLDGDGWTSRWIESKHKSDFGKFVLSSGK | |
| | | | FYGDEEKDKGLQTSQDARFYALSASFEPFSNKGQTLVV | |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAAAGGDAHKSEVAHRFKDLGE | |
| | | | ENFKALVLIAFAQYLQQSPFEDHVKLVNEVTEFAKTCVA | |
| | | | DESAENCDKSLHTLFGDKLCTVATLRETYGEMADCCAK | |
| | | | QEPERNECFLQHKDDNPNLPRLVRPEVDVMCTAFHDN | |
| | | | EETFLKKYLYEIARRHPYFYAPELLFFAKRYKAAFTECCQA | |
| | | | ADKAACLLPKLDELRDEGKASSAKQRLKCASLQKFGERA | |
| | | | FKAWAVARLSQRFPKAEFAEVSKLVTDLTKVHTECCHG | |
| | | | DLLECADDRADLAKYICENQDSISSKLKECCEKPLLEKSHC | |
| | | | IAEVENDEMPADLPSLAADFVESKDVCKNYAEAKDVFL | |
| | | | GMFLYEYARRHPDYSVVLLLRLAKTYETTLEKCCAAA | |
| 163 | 17859 | Full | EPAVYFKEQFLDGDGWTSRWIESKHKSDFGKFVLSSGK | |
| | | | FYGDEEKDKGLQTSQDARFYALSASFEPFSNKGQTLVV | |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAAAGGDAHKSEVAHRFKDLGE | |
| | | | ENFKALVLIAFAQYLQQSPFEDHVKLVNEVTEFAKTCVA | |
| | | | DESAENCDKSLHTLFGDKLCTVATLRETYGEMADCCAK | |
| | | | QEPERNECFLQHKDDNPNLPRLVRPEVDVMCTAFHDN | |
| | | | EETFLKKYLYEIARRHPYFYAPELLFFAKRYKAAFTECCQA | |
| | | | ADKAACLLPKLDELRDEGKASSAKQRLKCASLQKFGERA | |
| | | | FKAWAVARLSQRFPKAEFAEVSKLVTDLTKVHTECCHG | |
| | | | DLLECADDRADLAKYICENQDSISSKLKECCEKPLLEKSHC | |
| | | | IAEVENDEMPADLPSLAADFVESKDVCKNYAEAKDVFL | |
| | | | GMFLYEYARRHPDYSVVLLLRLAKTYETTLEKCCAAAGG | |
| | | | GGSEPAVYFKEQFLDGDGWTSRWIESKHKSDFGKFVLS | |
| | | | SGKFYGDEEKDKGLQTSQDARFYALSASFEPFSNKGQTL | |
| | | | VVQFTVKHEQNIDCGGGYVKLFPNSLDQTDMHGDSEY | |
| | | | NIMFGPDICGPGTKKVHVIFNYKGKNVLINKDIRCKDDE | |
| | | | FTHLYTLIVRPDNTYEVKIDNSQVESGSLEDDWDFLPPK | |
| | | | KIKDPDASKPEDWDERAKIDDPTDSKPEDWDKPEHIPD | |
| | | | PDAKKPEDWDEEMDGEWEPPVIQNPEYKGEWKPRQI | |
| | | | DNPDYKGTWIHPEIDNPEYSPDPSIYAYDNFGVLGLDL | |
| | | | WQVKSGTIFDNFLITNDEAYAEEFGNETWGVTKAAEKQ | |
| | | | MKDKQDEEQRLKEEEEDKKRKEEEEAEDKEDDEDKDED | |
| | | | EEDEEDKEEDEEEDVPGQA | |
| 164 | 17860 | Full | DIQMTQSPSSLSASVGDRVTITCRASQDVNTAVAWYQ | |
| | | | QKPGKAPKLLIYSASFLYSGVPSRFSGSRSGTDFTLTISSL | |
| | | | QPEDFATYYCQQHYTTPPTFGCGTKVEIKGGSGGGSGG | |
| | | | GSGGGSGGGSGEVQLVESGGGLVQPGGSLRLSCAASG | |
| | | | FNIKDTYIHWVRQAPGKCLEWVARIYPTNGYTRYADSV | |
| | | | KGRFTISADTSKNTAYLQMNSLRAEDTAVYYCSRWGGD | |
| | | | GFYAMDYWGQGTLVTVSSAAADPHECYAKVFDEFKPL | |
| | | | VEEPQNLIKQNCELFEQLGEYKFQNALLVRYTKKVPQVS | |
| | | | TPTLVEVSRNLGKVGSKCCKHPEAKRMPCAEDYLSVVL | |
| | | | NQLCVLHEKTPVSDRVTKCCTESLVNRRPCFSALEVDET | |
| | | | YVPKEFNAETFTFHADICTLSEKERQIKKQTALVELVKHK | |
| | | | PKATKEQLKAVMDDFAAFVEKCCKADDKETCFAEEGKK | |
| | | | LVAASQAALGLEPAVYFKEQFLDGDGWTSRWIESKHKS | |
| | | | DFGKFVLSSGKFYGDEEKDKGLQTSQDARFYALSASFEP | |
| | | | FSNKGQTLVVQFTVKHEQNIDCGGGYVKLFPNSLDQTD | |
| | | | MHGDSEYNIMFGPDICGPGTKKVHVIFNYKGKNVLINK | |
| | | | DIRCKDDEFTHLYTLIVRPDNTYEVKIDNSQVESGSLEDD | |
| | | | WDFLPPKKIKDPDASKPEDWDERAKIDDPTDSKPEDW | |
| | | | DKPEHIPDPDAKKPEDWDEEMDGEWEPPVIQNPEYKG | |
| | | | EWKPRQIDNPDYKGTWIHPEIDNPEYSPDPSIYAYDNFG | |
| | | | VLGLDLWQVKSGTIFDNFLITNDEAYAEEFGNETWGVT | |
| | | | KAAEKQMKDKQDEEQRLKEEEEDKKRKEEEEAEDKEDD | |
| | | | EDKDEDEEDEEDKEEDEEEDVPGQA | |
| 165 | 9157 | Full | DAHKSEVAHRFKDLGEENFKALVLIAFAQYLQQSPFEDH | |
| | | | VKLVNEVTEFAKTCVADESAENCDKSLHTLFGDKLCTVA | |
| | | | TLRETYGEMADCCAKQEPERNECFLQHKDDNPNLPRLV | |
| | | | RPEVDVMCTAFHDNEETFLKKYLYEIARRHPYFYAPELLF | |
| | | | FAKRYKAAFTECCQAADKAACLLPKLDELRDEGKASSAK | |
| | | | QRLKCASLQKFGERAFKAWAVARLSQRFPKAEFAEVSKL | |
| | | | VTDLTKVHTECCHGDLLECADDRADLAKYICENQDSISS | |
| | | | KLKECCEKPLLEKSHCIAEVENDEMPADLPSLAADFVESK | |
| | | | DVCKNYAEAKDVFLGMFLYEYARRHPDYSVVLLLRLAKT | |
| | | | YETTLEKCCAAA | |
| 166 | 17862 | Full | DAHKSEVAHRFKDLGEENFKALVLIAFAQYLQQSPFEDH | |
| | | | VKLVNEVTEFAKTCVADESAENCDKSLHTLFGDKLCTVA | |
| | | | TLRETYGEMADCCAKQEPERNECFLQHKDDNPNLPRLV | |
| | | | RPEVDVMCTAFHDNEETFLKKYLYEIARRHPYFYAPELLF | |
| | | | FAKRYKAAFTECCQAADKAACLLPKLDELRDEGKASSAK | |
| | | | QRLKCASLQKFGERAFKAWAVARLSQRFPKAEFAEVSKL | |
| | | | VTDLTKVHTECCHGDLLECADDRADLAKYICENQDSISS | |
| | | | KLKECCEKPLLEKSHCIAEVENDEMPADLPSLAADFVESK | |
| | | | DVCKNYAEAKDVFLGMFLYEYARRHPDYSVVLLLRLAKT | |
| | | | YETTLEKCCAAAGGGGSEPAVYFKEQFLDGDGWTSRWI | |
| | | | ESKHKSDFGKFVLSSGKFYGDEEKDKGLQTSQDARFYAL | |
| | | | SASFEPFSNKGQTLVVQFTVKHEQNIDCGGGYVKLFPNS | |
| | | | LDQTDMHGDSEYNIMFGPDICGPGTKKVHVIFNYKGK | |
| | | | NVLINKDIRCKDDEFTHLYTLIVRPDNTYEVKIDNSQVES | |
| | | | GSLEDDWDFLPPKKIKDPDASKPEDWDERAKIDDPTDS | |
| | | | KPEDWDKPEHIPDPDAKKPEDWDEEMDGEWEPPVIQ | |
| | | | NPEYKGEWKPRQIDNPDYKGTWIHPEIDNPEYSPDPSIY | |
| | | | AYDNFGVLGLDLWQVKSGTIFDNFLITNDEAYAEEFGNE | |
| | | | TWGVTKAAEKQMKDKQDEEQRLKEEEEDKKRKEEEEA | |
| | | | EDKEDDEDKDEDEEDEEDKEEDEEEDVPGQA | |
| 167 | 12155 | Full | EPKSSDKTHTCPPCPAPEAAGGPSVFLFPPKPKDTLMISR | |
| | | | TPEVTCVVVSVSHEDPEVKFNWYVDGVEVHNAKTKPR | |
| | | | EEQYNSTYRVVSVLTVLHQDWLNGKEYKCKVSNKALPA | |
| | | | PIEKTISKAKGQPREPQVYVYPPSRDELTKNQVSLTCLVK | |
| | | | GFYPSDIAVEWESNGQPENNYKTTPPVLDSDGSFALVS | |
| | | | KLTVDKSRWQQGNVFSCSVMHEALHNHYTQKSLSLSP | |
| | | | G | |
| 168 | 17901 | Full | EPKSSDKTHTCPPCPAPEAAGGPSVFLFPPKPKDTLMISR | |
| | | | TPEVTCVVVSVSHEDPEVKFNWYVDGVEVHNAKTKPR | |
| | | | EEQYNSTYRVVSVLTVLHQDWLNGKEYKCKVSNKALPA | |
| | | | PIEKTISKAKGQPREPQVYVYPPSRDELTKNQVSLTCLVK | |
| | | | GFYPSDIAVEWESNGQPENNYKTTPPVLDSDGSFALVS | |
| | | | KLTVDKSRWQQGNVFSCSVMHEALHNHYTQKSLSLSP | |
| | | | GGGGGDIQMTQSPSSLSASVGDRVTITCRASQDVNTA | |
| | | | VAWYQQKPGKAPKLLIYSASFLYSGVPSRFSGSRSGTDF | |
| | | | TLTISSLQPEDFATYYCQQHYTTPPTFGCGTKVEIKGGSG | |
| | | | GGSGGGSGGGSGGGSGEVQLVESGGGLVQPGGSLRLS | |
| | | | CAASGFNIKDTYIHWVRQAPGKCLEWVARIYPTNGYTR | |
| | | | YADSVKGRFTISADTSKNTAYLQMNSLRAEDTAVYYCSR | |
| | | | WGGDGFYAMDYWGQGTLVTVSS | |
| 169 | 17902 | Full | EPAVYFKEQFLDGDGWTSRWIESKHKSDFGKFVLSSGK | |
| | | | FYGDEEKDKGLQTSQDARFYALSASFEPFSNKGQTLVV | |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAAAEPKSSDKTHTCPPCPAPEA | |
| | | | AGGPSVFLFPPKPKDTLMISRTPEVTCVVVSVSHEDPEV | |
| | | | KFNWYVDGVEVHNAKTKPREEQYNSTYRVVSVLTVLH | |
| | | | QDWLNGKEYKCKVSNKALPAPIEKTISKAKGQPREPQV | |
| | | | YVYPPSRDELTKNQVSLTCLVKGFYPSDIAVEWESNGQP | |
| | | | ENNYKTTPPVLDSDGSFALVSKLTVDKSRWQQGNVFSC | |
| | | | SVMHEALHNHYTQKSLSLSPGGGGGDIQMTQSPSSLSA | |
| | | | SVGDRVTITCRASQDVNTAVAWYQQKPGKAPKLLIYSA | |
| | | | SFLYSGVPSRFSGSRSGTDFTLTISSLQPEDFATYYCQQH | |
| | | | YTTPPTFGCGTKVEIKGGSGGGSGGGSGGGSGGGSGEV | |
| | | | QLVESGGGLVQPGGSLRLSCAASGFNIKDTYIHWVRQA | |
| | | | PGKCLEWVARIYPTNGYTRYADSVKGRFTISADTSKNTA | |
| | | | YLQMNSLRAEDTAVYYCSRWGGDGFYAMDYWGQGT | |
| | | | LVTVSS | |
| 170 | 17903 | Full | EPAVYFKEQFLDGDGWTSRWIESKHKSDFGKFVLSSGK | |
| | | | FYGDEEKDKGLQTSQDARFYALSASFEPFSNKGQTLVV | |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAAAEPKSSDKTHTCPPCPAPEA | |
| | | | AGGPSVFLFPPKPKDTLMISRTPEVTCVVVSVSHEDPEV | |
| | | | KFNWYVDGVEVHNAKTKPREEQYNSTYRVVSVLTVLH | |
| | | | QDWLNGKEYKCKVSNKALPAPIEKTISKAKGQPREPQV | |
| | | | YVLPPSRDELTKNQVSLLCLVKGFYPSDIAVEWESNGQP | |
| | | | ENNYLTWPPVLDSDGSFFLYSKLTVDKSRWQQGNVFSC | |
| | | | SVMHEALHNHYTQKSLSLSPGGGGGDIQMTQSPSSLSA | |
| | | | SVGDRVTITCRASQDVNTAVAWYQQKPGKAPKLLIYSA | |
| | | | SFLYSGVPSRFSGSRSGTDFTLTISSLQPEDFATYYCQQH | |
| | | | YTTPPTFGCGTKVEIKGGSGGGSGGGSGGGSGGGSGEV | |
| | | | QLVESGGGLVQPGGSLRLSCAASGFNIKDTYIHWVRQA | |
| | | | PGKCLEWVARIYPTNGYTRYADSVKGRFTISADTSKNTA | |
| | | | YLQMNSLRAEDTAVYYCSRWGGDGFYAMDYWGQGT | |
| | | | LVTVSS | |
| 171 | 16784 | Full | EPAVYFKEQFLDGDGWTSRWIESKHKSDFGKFVLSSGK | |
| | | | FYGDEEKDKGLQTSQDARFYALSASFEPFSNKGQTLVV | |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAGGGGSEPAVYFKEQFLDGDG | |
| | | | WTSRWIESKHKSDFGKFVLSSGKFYGDEEKDKGLQTSQ | |
| | | | DARFYALSASFEPFSNKGQTLVVQFTVKHEQNIDCGGG | |
| | | | YVKLFPNSLDQTDMHGDSEYNIMFGPDICGPGTKKVHV | |
| | | | IFNYKGKNVLINKDIRCKDDEFTHLYTLIVRPDNTYEVKID | |
| | | | NSQVESGSLEDDWDFLPPKKIKDPDASKPEDWDERAKI | |
| | | | DDPTDSKPEDWDKPEHIPDPDAKKPEDWDEEMDGEW | |
| | | | EPPVIQNPEYKGEWKPRQIDNPDYKGTWIHPEIDNPEY | |
| | | | SPDPSIYAYDNFGVLGLDLWQVKSGTIFDNFLITNDEAY | |
| | | | AEEFGNETWGVTKAAEKQMKDKQDEEQRLKEEEEDKK | |
| | | | RKEEEEAEDKEDDEDKDEDEEDEEDKEEDEEEDVPGQA | |
| | | | AAEPKSSDKTHTCPPCPAPEAAGGPSVFLFPPKPKDTLM | |
| | | | ISRTPEVTCVVVSVSHEDPEVKFNWYVDGVEVHNAKTK | |
| | | | PREEQYNSTYRVVSVLTVLHQDWLNGKEYKCKVSNKAL | |
| | | | PAPIEKTISKAKGQPREPQVYVLPPSRDELTKNQVSLLCL | |
| | | | VKGFYPSDIAVEWESNGQPENNYLTWPPVLDSDGSFFL | |
| | | | YSKLTVDKSRWQQGNVFSCSVMHEALHNHYTQKSLSLS | |
| | | | PG | |
| 172 | 17905 | Full | EPKSSDKTHTCPPCPAPEAAGGPSVFLFPPKPKDTLMISR | |
| | | | TPEVTCVVVSVSHEDPEVKFNWYVDGVEVHNAKTKPR | |
| | | | EEQYNSTYRVVSVLTVLHQDWLNGKEYKCKVSNKALPA | |
| | | | PIEKTISKAKGQPREPQVYVLPPSRDELTKNQVSLLCLVK | |
| | | | GFYPSDIAVEWESNGQPENNYLTWPPVLDSDGSFFLYS | |
| | | | KLTVDKSRWQQGNVFSCSVMHEALHNHYTQKSLSLSP | |
| | | | GGGGGDIQMTQSPSSLSASVGDRVTITCRASQDVNTA | |
| | | | VAWYQQKPGKAPKLLIYSASFLYSGVPSRFSGSRSGTDF | |
| | | | TLTISSLQPEDFATYYCQQHYTTPPTFGCGTKVEIKGGSG | |
| | | | GGSGGGSGGGSGGGSGEVQLVESGGGLVQPGGSLRLS | |
| | | | CAASGFNIKDTYIHWVRQAPGKCLEWVARIYPTNGYTR | |
| | | | YADSVKGRFTISADTSKNTAYLQMNSLRAEDTAVYYCSR | |
| | | | WGGDGFYAMDYWGQGTLVTVSS | |
| 173 | 17941 | Full | EPAVYFKEQFLDGDGWTSRWIESKHKSDFGKFVLSSGK | |
| | | | FYGDEEKDKGLQTSQDARFYALSASFEPFSNKGQTLVV | |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAAAEPKSSDKTHTCPPCPAPEA | |
| | | | AGGPSVFLFPPKPKDTLMISRTPEVTCVVVSVSHEDPEV | |
| | | | KFNWYVDGVEVHNAKTKPREEQYNSTYRVVSVLTVLH | |
| | | | QDWLNGKEYKCKVSNKALPAPIEKTISKAKGQPREPQV | |
| | | | YVYPPSRDELTKNQVSLTCLVKGFYPSDIAVEWESNGQP | |
| | | | ENNYKTTPPVLDSDGSFALVSKLTVDKSRWQQGNVFSC | |
| | | | SVMHEALHNHYTQKSLSLSPG | |
| 174 | 9158 | Full | AAADPHECYAKVFDEFKPLVEEPQNLIKQNCELFEQLGE | |
| | | | YKFQNALLVRYTKKVPQVSTPTLVEVSRNLGKVGSKCCK | |
| | | | HPEAKRMPCAEDYLSVVLNQLCVLHEKTPVSDRVTKCC | |
| | | | TESLVNRRPCFSALEVDETYVPKEFNAETFTFHADICTLSE | |
| | | | KERQIKKQTALVELVKHKPKATKEQLKAVMDDFAAFVE | |
| | | | KCCKADDKETCFAEEGKKLVAASQAALGL | |
| 175 | 12153 | Full | EPKSSDKTHTCPPCPAPEAAGGPSVFLFPPKPKDTLMISR | |
| | | | TPEVTCVVVSVSHEDPEVKFNWYVDGVEVHNAKTKPR | |
| | | | EEQYNSTYRVVSVLTVLHQDWLNGKEYKCKVSNKALPA | |
| | | | PIEKTISKAKGQPREPQVYVLPPSRDELTKNQVSLLCLVK | |
| | | | GFYPSDIAVEWESNGQPENNYLTWPPVLDSDGSFFLYS | |
| | | | KLTVDKSRWQQGNVFSCSVMHEALHNHYTQKSLSLSP | |
| | | | G | |
| 176 | 12667 | Full | EPAVYFKEQFLDGDGWTSRWIESKHKSDFGKFVLSSGK | |
| | | | FYGDEEKDKGLQTSQDARFYALSASFEPFSNKGQTLVV | |
| | | | QFTVKHEQNIDCGGGYVKLFPNSLDQTDMHGDSEYNI | |
| | | | MFGPDICGPGTKKVHVIFNYKGKNVLINKDIRCKDDEFT | |
| | | | HLYTLIVRPDNTYEVKIDNSQVESGSLEDDWDFLPPKKIK | |
| | | | DPDASKPEDWDERAKIDDPTDSKPEDWDKPEHIPDPD | |
| | | | AKKPEDWDEEMDGEWEPPVIQNPEYKGEWKPRQIDN | |
| | | | PDYKGTWIHPEIDNPEYSPDPSIYAYDNFGVLGLDLWQ | |
| | | | VKSGTIFDNFLITNDEAYAEEFGNETWGVTKAAEKQMK | |
| | | | DKQDEEQRLKEEEEDKKRKEEEEAEDKEDDEDKDEDEE | |
| | | | DEEDKEEDEEEDVPGQAAAEPKSSDKTHTCPPCPAPEA | |
| | | | AGGPSVFLFPPKPKDTLMISRTPEVTCVVVSVSHEDPEV | |
| | | | KFNWYVDGVEVHNAKTKPREEQYNSTYRVVSVLTVLH | |
| | | | QDWLNGKEYKCKVSNKALPAPIEKTISKAKGQPREPQV | |
| | | | YVLPPSRDELTKNQVSLLCLVKGFYPSDIAVEWESNGQP | |
| | | | ENNYLTWPPVLDSDGSFFLYSKLTVDKSRWQQGNVFSC | |
| | | | SVMHEALHNHYTQKSLSLSPG | |
| 177 | 9182 | Full | DIQMTQSPSSLSASVGDRVTITCRASQDVNTAVAWYQ | |
| | | | QKPGKAPKLLIYSASFLYSGVPSRFSGSRSGTDFTLTISSL | |
| | | | QPEDFATYYCQQHYTTPPTFGCGTKVEIKGGSGGGSGG | |
| | | | GSGGGSGGGSGEVQLVESGGGLVQPGGSLRLSCAASG | |
| | | | FNIKDTYIHWVRQAPGKCLEWVARIYPTNGYTRYADSV | |
| | | | KGRFTISADTSKNTAYLQMNSLRAEDTAVYYCSRWGGD | |
| | | | GFYAMDYWGQGTLVTVSSAAADPHECYAKVFDEFKPL | |
| | | | VEEPQNLIKQNCELFEQLGEYKFQNALLVRYTKKVPQVS | |
| | | | TPTLVEVSRNLGKVGSKCCKHPEAKRMPCAEDYLSVVL | |
| | | | NQLCVLHEKTPVSDRVTKCCTESLVNRRPCFSALEVDET | |
| | | | YVPKEFNAETFTFHADICTLSEKERQIKKQTALVELVKHK | |
| | | | PKATKEQLKAVMDDFAAFVEKCCKADDKETCFAEEGKK | |
| | | | LVAASQAALGL | |
| 178 | 9157 | Albucore3A | DAHKSEVAHRFKDLGEENFKALVLIAFAQYLQQSPFEDH | |
| | | Protein | VKLVNEVTEFAKTCVADESAENCDKSLHTLFGDKLCTVA | |
| | | | TLRETYGEMADCCAKQEPERNECFLQHKDDNPNLPRLV | |
| | | | RPEVDVMCTAFHDNEETFLKKYLYEIARRHPYFYAPELLF | |
| | | | FAKRYKAAFTECCQAADKAACLLPKLDELRDEGKASSAK | |
| | | | 1RLKCASLQKFGERAFKAWAVARLSQRFPKAEFAEVSKL | |
| | | | VTDLTKVHTECCHGDLLECADDRADLAKYICENQDSISS | |
| | | | KLKECCEKPLLEKSHCIAEVENDEMPADLPSLAADFVESK | |
| | | | DVCKNYAEAKDVFLGMFLYEYARRHPDYSVVLLLRLAKT | |
| | | | YETTLEKCCAAA | |
| 179 | 9157 | Albucore3A | GATGCTCATAAGAGCGAGGTGGCCCACAGGTTCAAG | |
| | | DNA | GACCTAGGCGAGGAGAACTTTAAGGCCCTGGTGCTG | |
| | | | ATCGCCTTCGCCCAGTACCTGCAGCAGTCCCCCTTTGA | |
| | | | GGACCACGTGAAGCTGGTGAACGAGGTGACCGAGTT | |
| | | | CGCCAAGACATGCGTGGCCGACGAGTCCGCCGAGAA | |
| | | | TTGTGATAAGTCTCTGCACACCCTGTTTGGCGATAAG | |
| | | | CTGTGCACCGTGGCCACACTGAGGGAGACATATGGC | |
| | | | GAGATGGCCGACTGCTGTGCCAAGCAGGAGCCCGAG | |
| | | | CGCAACGAGTGCTTCCTGCAGCACAAGGACGATAACC | |
| | | | CCAATCTGCCTCGGCTGGTGAGACCTGAGGTGGACGT | |
| | | | GATGTGCACCGCCTTCCACGATAATGAGGAGACATTT | |
| | | | CTGAAGAAGTACCTGTATGAGATCGCCCGGAGACAC | |
| | | | CCTTACTTTTATGCCCCAGAGCTGCTGTTCTTTGCCAA | |
| | | | GCGGTACAAGGCCGCCTTCACCGAGTGCTGTCAGGC | |
| | | | AGCAGATAAGGCAGCATGCCTGCTGCCAAAGCTGGA | |
| | | | CGAGCTGCGGGATGAGGGCAAGGCCAGCTCCGCCAA | |
| | | | GCAGAGACTGAAGTGTGCCTCTCTGCAGAAGTTCGG | |
| | | | AGAGCGGGCCTTTAAGGCATGGGCAGTGGCCAGGCT | |
| | | | GTCTCAGCGGTTCCCCAAGGCCGAGTTTGCCGAGGTG | |
| | | | AGCAAGCTGGTGACCGACCTGACAAAGGTGCACACA | |
| | | | GAGTGCTGTCACGGCGACCTGCTGGAGTGCGCCGAC | |
| | | | GATAGAGCCGATCTGGCCAAGTATATCTGTGAGAATC | |
| | | | AGGACTCCATCTCTAGCAAGCTGAAGGAGTGCTGTGA | |
| | | | GAAGCCTCTGCTGGAGAAGTCTCACTGCATCGCCGAG | |
| | | | GTGGAGAACGACGAGATGCCAGCCGATCTGCCAAGC | |
| | | | CTGGCCGCAGACTTTGTGGAGTCCAAGGACGTGTGC | |
| | | | AAGAATTACGCCGAGGCCAAGGACGTGTTCCTGGGC | |
| | | | ATGTTTCTGTACGAGTATGCCCGGCGGCACCCAGACT | |
| | | | ATTCCGTGGTGCTGCTGCTGAGACTGGCTAAAACCTA | |
| | | | CGAAACTACTCTGGAAAAATGTTGTGCCGCGGCC | |
| 180 | 9158 | Albucore3B | DPHECYAKVFDEFKPLVEEPQNLIKQNCELFEQLGEYKF | |
| | | Protein | QNALLVRYTKKVPQVSTPTLVEVSRNLGKVGSKCCKHPE | |
| | | | AKRMPCAEDYLSVVLNQLCVLHEKTPVSDRVTKCCTESL | |
| | | | VNRRPCFSALEVDETYVPKEFNAETFTFHADICTLSEKER | |
| | | | QIKKQTALVELVKHKPKATKEQLKAVMDDFAAFVEKCC | |
| | | | KADDKETCFAEEGKKLVAASQAALGL | |
| 181 | 9158 | Albucore3B | GACCCCCACGAATGCTATGCCAAGGTGTTCGATGAGT | |
| | | DNA | TTAAGCCTCTGGTGGAGGAGCCACAGAACCTGATCAA | |
| | | | GCAGAATTGTGAGCTGTTCGAGCAGCTGGGCGAGTA | |
| | | | CAAGTTTCAGAACGCCCTGCTGGTGAGGTATACCAAG | |
| | | | AAGGTGCCCCAGGTGTCCACCCCTACACTGGTGGAG | |
| | | | GTGTCTCGGAATCTGGGCAAGGTCGGCAGCAAGTGC | |
| | | | TGTAAGCACCCAGAGGCCAAGAGGATGCCCTGCGCC | |
| | | | GAGGACTACCTGTCTGTGGTGCTGAATCAGCTGTGCG | |
| | | | TGCTGCACGAGAAGACCCCCGTGAGCGATAGGGTGA | |
| | | | CCAAGTGCTGTACAGAGTCCCTGGTCAACCGGAGACC | |
| | | | CTGCTTTTCTGCCCTGGAGGTGGACGAGACATATGTG | |
| | | | CCTAAGGAGTTCAATGCCGAGACCTTCACATTTCACG | |
| | | | CCGATATCTGTACCCTGAGCGAGAAGGAGCGCCAGA | |
| | | | TCAAGAAGCAGACAGCCCTGGTGGAGCTGGTGAAGC | |
| | | | ACAAGCCTAAGGCCACCAAGGAGCAGCTGAAGGCCG | |
| | | | TGATGGACGATTTCGCCGCCTTTGTGGAGAAGTGCTG | |
| | | | TAAGGCCGACGATAAGGAGACATGCTTCGCAGAGGA | |
| | | | GGGCAAGAAGCTGGTGGCAGCCTCCCAGGCCGCCCT | |
| | | | AGGCCTG | |
| 182 | 17901 | Trast | DIQMTQSPSSLSASVGDRVTITCRASQDVNTAVAWYQ | |
| | | scFv | QKPGKAPKLLIYSASFLYSGVPSRFSGSRSGTDFTLTISSL | |
| | | | QPEDFATYYCQQHYTTPPTFGCGTKVEIKGGSGGGSGG | |
| | | | GSGGGSGGGSGEVQLVESGGGLVQPGGSLRLSCAASG | |
| | | | FNIKDTYIHWVRQAPGKCLEWVARIYPTNGYTRYADSV | |
| | | | KGRFTISADTSKNTAYLQMNSLRAEDTAVYYCSRWGGD | |
| | | | GFYAMDYWGQGTLVTVSS | |

We claim:

1. A tumor-associated antigen (TAA) presentation inducer construct comprising
a) at least one innate stimulatory receptor (ISR)-binding construct that binds to an ISR expressed on an antigen-presenting cell (APC), and
b) at least one TAA-binding construct that binds directly to a first TAA that is physically associated with tumor cell-derived material (TCDM) comprising one or more other TAAs,
wherein said ISR-binding construct and said TAA-binding construct are linked to each other, and
wherein the TAA presentation inducer construct induces a polyclonal T cell response to the one or more other TAAs.

2. The TAA presentation inducer construct according to claim 1, wherein the ISR is a C-type lectin receptor, a member of the tumor necrosis factor receptor family, or a lipoprotein receptor.

3. The TAA presentation inducer construct according claim 2, wherein the innate stimulatory receptor is a C-type lectin receptor.

4. The TAA presentation inducer construct according to claim 3, wherein the C-type lectin receptor is dectin-1, dectin-2, DEC205, Mincle, or DC-SIGN.

5. The TAA presentation inducer construct according to claim 2, wherein the innate stimulatory receptor is CD40 or LRP-1.

6. The TAA presentation inducer construct according to any one of claims 1 to 5, wherein the first TAA is highly expressed in cancer cells, is a low immunoscore TAA, or is an oncofetal antigen.

7. The TAA presentation inducer construct according to any one of claims 1 to 5, wherein the first TAA is HER2, ROR1, or PSMA.

8. The TAA presentation inducer construct according to any one of claims 1 to 7, wherein the at least one ISR-binding construct and/or the at least one TAA-binding construct is a peptide, or a polypeptide.

9. The TAA presentation inducer construct according to claim 8, wherein the at least one ISR-binding construct is an antigen-binding domain and/or the at least one TAA-binding construct is an antigen-binding domain.

10. The TAA presentation inducer according to any one of claims 1 to 9, wherein the TAA presentation inducer comprises two or more ISR-binding constructs.

11. The TAA presentation inducer according to claim 10, wherein the two or more ISR-binding constructs bind to two or more different ISRs.

12. The TAA presentation inducer according to any one of claims 1 to 9, wherein the TAA presentation inducer comprises two or more TAA-binding constructs.

13. The TAA presentation inducer according to claim 12, wherein the two or more TAA-binding constructs bind to different antigens.

14. The TAA presentation inducer according to any one of claims 1 to 13, wherein the at least one ISR-binding construct and the at least one TAA-binding construct are linked directly to each other.

15. The TAA presentation inducer according to any one of claims 1 to 13, wherein the at least one ISR-binding construct and the at least one TAA-binding construct are linked to each other with a linker.

16. The TAA presentation inducer according to claim 15, wherein the linker is an Fc.

17. The TAA presentation inducer according to any one of claims 1 to 16, wherein the TAA presentation inducer is a bispecific antibody that binds to an ISR and to a TAA.

18. The TAA presentation inducer construct according to any one of claims 1 to 17, wherein the TAA presentation inducer construct is conjugated to a drug.

19. A pharmaceutical composition comprising the TAA presentation inducer construct according to any one of claims 1 to 18.

20. One or more nucleic acids encoding the TAA presentation inducer construct according to any one of claims 1 to 18.

21. One or more vectors comprising the one or more nucleic acids according to claim 20.

22. A host cell comprising the one or more nucleic acids according to claim 20, or the one or more vectors according to claim 21.

23. A method of making the tumor-associated antigen (TAA) presentation inducer construct according to any one of claims 1 to 18, comprising:
a) expressing the one or more nucleic acids of claim 20 or the one or more vectors of claim 21 in a cell.

24. A method of treating cancer comprising administering the tumor-associated antigen (TAA) presentation inducer construct according to any one of claims 1 to 18 to a subject in need thereof.

25. A method of inducing major histocompatibility complex (MHC) presentation of peptides from two or more tumor-associated antigens (TAAs) by a single innate stimulatory receptor-expressing cell simultaneously in a subject, comprising administering to the subject the TAA presentation inducer construct according to any one of claims 1 to 18.

26. A method of inducing innate stimulatory receptor-expressing cell activation in a subject, comprising administering to the subject, the tumor-associated antigen (TAA) presentation inducer construct according to any one of claims 1 to 18.

27. A method of inducing a polyclonal T cell response in a subject, comprising administering to the subject the tumor-associated antigen (TAA) presentation inducer construct according to any one of claims 1 to 18.

28. A method of expanding, activating, or differentiating T cells specific for two or more tumor-associated antigens (TAAs) simultaneously, comprising:
a) obtaining T cells and innate stimulatory receptor (ISR)-expressing cells from a subject; and
b) culturing the T cells and the ISR-expressing cells with the TAA presentation inducer construct according to any one of claims 1 to 18 in the presence of tumor cell-derived material (TCDM), to produce expanded, activated or differentiated T cells.

29. The method according to claim 28, wherein the TCDM is from an autologous tissue sample, or from a tumor cell line.

30. A method of treating cancer in a subject, comprising administering to the subject the expanded, activated or differentiated T cells prepared according to the method of claim 28 or 29.

31. A method of identifying tumor-associated antigens in tumor cell-derived material (TCDM) comprising
a) isolating T cells and enriched innate stimulatory receptor (ISR)-expressing cells from a subject;
b) culturing the ISR-expressing cells and the T cells with the TAA presentation inducer construct according to any one of claims 1 to 18 in the presence of tumor cell-derived material (TCDM), to produce TAA presentation inducer construct-activated ISR-expressing cells, and
c) determining the sequence of TAA peptides eluted from MHC complexes of the TAA presentation inducer construct-activated ISR-expressing cells; and
d) identifying the TAAs corresponding to the TAA peptides.

32. A method of identifying T cell receptor (TCR) target polypeptides, comprising
a) isolating T cells and enriched innate stimulatory receptor (ISR)-expressing cells from a subject;
b) culturing the ISR-expressing cells and the T cells with the TAA presentation inducer construct according to any one of claims 1 to 18 in the presence of tumor cell-derived material (TCDM), to produce TAA presentation inducer construct-activated ISR-expressing cells and activated T cells, and
c) screening the activated T cells against a library of candidate TAAs to identify the TCR target polypeptides.

33. Use of a therapeutically effective amount of the tumor-associated antigen (TAA) presentation inducer construct according to any one of claims 1 to 18 in the treatment of a cancer in a subject in need thereof.

34. Use of the tumor-associated antigen (TAA) presentation inducer construct according to any one of claims 1 to 18 in the preparation of a medicament for the treatment of a cancer in a subject in need thereof.

35. Use of a therapeutically effective amount of the TAA presentation inducer construct according to any one of claims 1 to 18 for induction of major histocompatibility complex (MEW) presentation of peptides from two or more tumor-associated antigens (TAAs) by a single innate stimulatory receptor-expressing cell simultaneously, in a subject in need thereof.

36. Use of the TAA presentation inducer construct according to any one of claims 1 to 18 in the preparation of a medicament for induction of major histocompatibility complex (MHC) presentation of peptides from two or more tumor-associated antigens (TAAs) by a single innate stimulatory receptor-expressing cell simultaneously, in a subject in need thereof.

37. Use of a therapeutically effective amount of the tumor-associated antigen (TAA) presentation inducer construct according to any one of claims 1 to 18 for induction of innate stimulatory receptor-expressing cell activation in a subject in need thereof.

38. Use of the tumor-associated antigen (TAA) presentation inducer construct according to any one of claims 1 to 18 in the preparation of a medicament for induction of innate stimulatory receptor-expressing cell activation in a subject in need thereof.

39. Use of a therapeutically effective amount of the tumor-associated antigen (TAA) presentation inducer construct according to any one of claims 1 to 18 for induction of a polyclonal T cell response in a subject in need thereof.

40. Use of the tumor-associated antigen (TAA) presentation inducer construct according to any one of claims 1 to 18 in the preparation of a medicament for induction of a polyclonal T cell response in a subject in need thereof.

41. Use of a therapeutically effective amount of expanded, activated or differentiated T cells prepared according to the method of claim 28 or 29 in the treatment of a cancer in a subject in need thereof.

42. Use of expanded, activated or differentiated T cells prepared according to the method of claim 28 or 29 in the preparation of a medicament for treating cancer in a subject in need thereof.
